SEQUENCE LISTING (12 sequences)

SEQ ID NO: 1 - DNA, 1515 bp, Homo sapiens
  source 1..1515: mol_type = genomic DNA
aggttcagagagattagccctcttggccaagatcgcacagccagtcagtggcagaggtgggacttgaacccgtcccttgctgttccttgaacttctccacacgacagggctgtccctcagcctacaggtcacccctctagccttgcctgttggctcttcctaatcctttcaggcccagcccagatactttttaaacaagaatgatgagagctaactttttttaaaatgagacggtcttgctctttcacccagagaagactgggtgataacagtggctcaatcttggctcactgcagcctctgcctccagggttcaagctattctcatgccttagcctcctcagtagctgggattacaggggtgcatcactacaccacgctaatttttgtatttttagtagagatgcggttttaccatgttggccaggctggtctcgaactcctgacctcaggtgatccgccagccttggcctcccaaagtgctgggattacaggcgtgagtcactacgcatggctgagagctaacgtttattaagcacccgccactttctagatgaggtccttactgacttccttgggttattttaggtaatcatccccatacctcttacgtggcaggtatgactgttacagagggagaaaccgaggctgagaaaggctaagtagcggccacacctcactcagcaggtgacaggctgcggtcgcactcatgacatagtctgactctagagcaggcctttcctgcccgctgttgctgcctccagaagcctctctgaatccaagggtgggaactattcctccttgccctcgagccttcgccaaagccagggcttggagcggccgtgggctcctcctccttcccccaggctgggctcttcaggggcggaaacctgcctggcttgcctgccttgctcgaccctgtagcccctggcacgaagtaggtgcccgtgatgccggattatttcagcaggagggcaggggtgttcccgatgaagccaggagatccagggaggagaagcgactcccggggccacccatgcttagatctcagagccaggcaagagccaggtcccgggggtccaggagcgcgctcggggctcctcctcgcccgcgccccccgcgcccctcgcgccccccgcgccccccgcgccccctgcgctcgcgtctgcggggccgggaggaaggggcggcgcagccgggccggggcgggcaccgaggcctggcgggcgggcctggggcgctttcgtgcctgccagggaaaggctggcgaggaggctgggggcggacgcagggaggaaagaagcaatggggtgctcttgcgttttgacattttaaaaagaagtttagattcgcttcccggagccgggatggcagcctgcgctatggttggggaccaacgtggtgcacgggcaggggccggggagagaggcggccgcagcgggagcccgggagcgcagggcgggcctggaagagctccgccccaaggggcgcggccaccccggaggcgggcgcacgg

SEQ ID NO: 2 - DNA, 1661 bp, Mus musculus
  source 1..1661: mol_type = genomic DNA
aaataaatcctggttctctgcaagaacagtgagcgctcttcaccactgagctgtctctctagcccggcacctagcttatgtgatgctggaggccatgcctttatgcatgctatgaggtgttctgccagctgaactccattctcagccccattactgttatcactttagctggagactctagagtcgctggctacagtcacagagcctcaggcactatccaaccctgtactgagtaactgcagaacacttcagactacaccttacgcaccgcaccgttagctgtgaggtgggcaggacttcttgtttctctcctacagaaactgaggttcagggaggtggggcctcttggccaagatcacacggctaatcagtacaactggagccccttctgttctttcgaactacttctaccttaaagaagagtttcttctgactgggtcttgctatgtagcccaggctggcctcctgcctcagcttcctgactacagatgactgtcagcacacctgtgtccttgagctttgctgacactgcgcctcagccccctacccttctccagagccacttgtggtgtttcctcttcctcccaggatagcccaaagctttggtaaatggcaataatgaaaagaaccaacatcttttgggtttttgaaacagggtctcactatgtagccttggtctggagcttacatgtggaccaggctggccttggtttcactgacccttctgcctctgcctccgtagtaatggatttacaggtgtgggccaccatatctgactgtaagaccccaacactaattaatacctgccactctgtagatgcaaatccagctatccttggagtgaccatctccatacgaaggcaggtaagactgttatagacaggaaactggggctgagattaaattaacctgcccaaggtcactcaaatgagtgacagccaggagtcacaacaatggttccagcaaaggcctttcttcctctgcccttgctaccgtgacaacccactcccctcaagggacaggaatggaccacagcccagctgtgtgtgatatcagacttctgtgtgtactcagtggcttttaaactgtgtcagacggactcccgactccctcttctcatggcccgagagctctctagagggggagcactactcatttgcctccatagccccagcacagaacaggtgctccataatgcccagttattttacttcttccgggcctcaatgccacgaagcagaggacaagggtgtccccgctgaagccctaaatccctagaaaggagaaatgacccccccaaggcctcccagaaatccatttggggtaacgacaggagaggtccctcctgtagcagagaaggctctcaagctggttgaagccgttctaaagctttctccggtgccagggaaaggacctcgaggaggcagcgggcggctgggggagggatgaaggaacgggatatttttgcgttctgacattaaaagaagagttgattcgcgtcagcatctgttcgggggttggagctgacagcatggggagcggtcccagtcttgcagccctggagcgaagggccgggcctccatgagctccgcccaaaggggcgctaccacccggaggaggccggcgcacggccgctgctcattcattgtcttgacagagca

SEQ ID NO: 3 - DNA, 1562 bp, Rattus norvegicus
  source 1..1562: mol_type = genomic DNA
cttcctcaggctgtgcacgaattctgccagctgagctgcattctcagcccagtactgttagcactttagctgcagactgccgagtccctggccacgatcacagagcctcaggcacaatccaaccctgttgagtaactatagaacactgcaggctacagaacagtcaaatgcttcatttgagcccagacagctgtgaggtgggcagggcgccttgttcctctcctacagaaactgaggcttggagaggaggggcctcttggtcaagatcacacagctaatcggtacgattggagtcaggccccttttgttcctttggactacttctacctggaagaacggtttcctctgacagggtcttgctgtgtagcccaggctgtgagggctcctgcctcagtttcctgagcacagatgactatcagcacacctgtgtctttgagctttgctgtcaccgagcctcagctcctgcccttctccaaagccacatctgctatttccctcttcttcccaggatagcccaaagctttggtaaatgacaattatgaaagggaccaacatcttttaggtttttgaaacagggtctcactatgtagccaggcctggaacttttatgtggaccaggctggcctcggcttcactgtgatccttaggcctctgcctccccagtaatggattcataggtgtgggccaccacattggctgtaagatccaacattagttatcgcctgacgctctgtagatgtgcatccagctatttttggagtgaacgtctccacattgaggcaggtaagactgttagagagggggagttagggctgagattaaattaacctgtccaagtcatctgagtgaccaccaggagtcacaaggttccagcaaaggcctttcttgccctgacaacccaccaccccctaagggacaggaatggttaagagcacttctgcgtgtggtatcagacttctgcgtgtactcagtggcctttaaactgtggcagacatactccccactccacttgccacaggcctgagggctctctagtggcgggagcacgactcacttccctcctcagccccagcacaaaacaggtgctccataatgcccagttattctacttcttccgggcctcaaccccgtgaagcagaggtcaacggcatccccgctgaagccctgaacccctagaaaggagaaatgaaccccccctaaggcatcccagaaatccatctgggtcatcgacaagagagatccttcctgtcgcagagaaggctctcaagctggttgtagcccttctggcgctttctcgggtgccagggaaaggacctcgaggaggctgcgggcggctgggggagggatgaaggaacggatattttttgcgttttgacattaaaagaagagttgattcgcgtcagcatctgttcgggggttggagctgacagcatgggagcgggtcccagctttgccgccccagagcgaagggccgggcctccatgagctccgcccaaaggggcgctgccaccccagaggaggccggcgcacggccgctgctcattcattgtcttgacagagca

SEQ ID NO: 4 - DNA, 722 bp, Homo sapiens
  source 1..722: mol_type = genomic DNA
ttcctccttgccctcgagccttcgccaaagccagggcttggagcggccgtgggctcctcctccttcccccaggctgggctcttcaggggcggaaacctgcctggcttgcctgccttgctcgaccctgtagcccctggcacgaagtaggtgcccgtgatgccggattatttcagcaggagggcaggggtgttcccgatgaagccaggagatccagggaggagaagcgactcccggggccacccatgcttagatctcagagccaggcaagagccaggtcccgggggtccaggagcgcgctcggggctcctcctcgcccgcgccccccgcgcccctcgcgccccccgcgccccccgcgccccctgcgctcgcgtctgcggggccgggaggaaggggcggcgcagccgggccggggcgggcaccgaggcctggcgggcgggcctggggcgctttcgtgcctgccagggaaaggctggcgaggaggctgggggcggacgcagggaggaaagaagcaatggggtgctcttgcgttttgacattttaaaaagaagtttagattcgcttcccggagccgggatggcagcctgcgctatggttggggaccaacgtggtgcacgggcaggggccggggagagaggcggccgcagcgggagcccgggagcgcagggcgggcctggaagagctccgccccaaggggcgcggccaccccggaggcgggcgcacgg

SEQ ID NO: 5 - DNA, 737 bp, Mus musculus
  source 1..737: mol_type = genomic DNA
caaatgagtgacagccaggagtcacaacaatggttccagcaaaggcctttcttcctctgcccttgctaccgtgacaacccactcccctcaagggacaggaatggaccacagcccagctgtgtgtgatatcagacttctgtgtgtactcagtggcttttaaactgtgtcagacggactcccgactccctcttctcatggcccgagagctctctagagggggagcactactcatttgcctccatagccccagcacagaacaggtgctccataatgcccagttattttacttcttccgggcctcaatgccacgaagcagaggacaagggtgtccccgctgaagccctaaatccctagaaaggagaaatgacccccccaaggcctcccagaaatccatttggggtaacgacaggagaggtccctcctgtagcagagaaggctctcaagctggttgaagccgttctaaagctttctccggtgccagggaaaggacctcgaggaggcagcgggcggctgggggagggatgaaggaacgggatatttttgcgttctgacattaaaagaagagttgattcgcgtcagcatctgttcgggggttggagctgacagcatggggagcggtcccagtcttgcagccctggagcgaagggccgggcctccatgagctccgcccaaaggggcgctaccacccggaggaggccggcgcacggccgctgctcattcattgtcttgacagagca

SEQ ID NO: 6 - DNA, 722 bp, Rattus norvegicus
  source 1..722: mol_type = genomic DNA
tctgagtgaccaccaggagtcacaaggttccagcaaaggcctttcttgccctgacaacccaccaccccctaagggacaggaatggttaagagcacttctgcgtgtggtatcagacttctgcgtgtactcagtggcctttaaactgtggcagacatactccccactccacttgccacaggcctgagggctctctagtggcgggagcacgactcacttccctcctcagccccagcacaaaacaggtgctccataatgcccagttattctacttcttccgggcctcaaccccgtgaagcagaggtcaacggcatccccgctgaagccctgaacccctagaaaggagaaatgaaccccccctaaggcatcccagaaatccatctgggtcatcgacaagagagatccttcctgtcgcagagaaggctctcaagctggttgtagcccttctggcgctttctcgggtgccagggaaaggacctcgaggaggctgcgggcggctgggggagggatgaaggaacggatattttttgcgttttgacattaaaagaagagttgattcgcgtcagcatctgttcgggggttggagctgacagcatgggagcgggtcccagctttgccgccccagagcgaagggccgggcctccatgagctccgcccaaaggggcgctgccaccccagaggaggccggcgcacggccgctgctcattcattgtcttgacagagca

SEQ ID NO: 7 - DNA, 22 bp, synthetic construct
  source 1..22: mol_type = other DNA
gtgacagctctatccggtattt

SEQ ID NO: 8 - DNA, 20 bp, synthetic construct
  source 1..20: mol_type = other DNA
acgttgagactccttggaac

SEQ ID NO: 9 - DNA, 20 bp, synthetic construct
  source 1..20: mol_type = other DNA
gggaaacccatcaccatctt

SEQ ID NO: 10 - DNA, 22 bp, synthetic construct
  source 1..22: mol_type = other DNA
ccagtagactccacgacatact

SEQ ID NO: 11 - RNA, 20 nt, synthetic construct
  source 1..20: mol_type = other RNA
tgggatgccaagccttcccg

SEQ ID NO: 12 - RNA, 20 nt, synthetic construct
  source 1..20: mol_type = other RNA
agggcttcgctgccgatatc

(19) United States
(12) Patent Application Publication
(10) Pub. No.: US 2026/0102387 A1
(43) Pub. Date: Apr. 16, 2026

(54) IMMUNOSUPPRESSIVE COMPOUNDS

(71) Applicants: UNIVERSITÄT BASEL, Basel (CH); ETH ZURICH, Zurich (CH)

(72) Inventors: Rajesh JAYACHANDRAN, Basel (CH); Jean PIETERS, Basel (CH); Reto ERNI, Zurich (CH); Tony GEORGIEV, Zurich (CH); Erick M. CARREIRA, Zurich (CH); Pascal GEORGE, Longvilliers (FR); Daniel JIMENEZ TEJA, Cugnaux (FR)

(21) Appl. No.: 19/112,804

(22) PCT Filed: Sep. 30, 2023
(86) PCT No.: PCT/EP2023/077188

§ 371 Date: Mar. 18, 2025

(30) Foreign Application Priority Data
Sep. 30, 2022 (EP) 22199306.6
Sep. 30, 2022 (EP) 22199307.4

(51) Int. Cl. A61K 31/4709; C07D 215/54; C07D 405/12; C07D 409/04; C07D 409/12; C07D 493/04; C12N 15/85; G01N 33/50

(52) U.S. Cl. CPC A61K 31/4709; C07D 215/54; C07D 405/12; C07D 409/04; C07D 409/12; C07D 493/04; C12N 15/85; G01N 33/5023; G01N 2333/4703

(57) ABSTRACT

The present invention relates to a compound of formula (I) or a pharmaceutically acceptable salt, stereoisomer, diastereoisomer, enantiomer, polymorph, racemic mixture, solvate or isomers and mixtures thereof. The invention further relates to a process for the stereoselective preparation of such compounds. The compound of formula (I) can be used as a medicament, in particular for inhibiting coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders. The present invention further relates to a vector comprising a coronin 1 promoter element, wherein in a vertebrate genome, said coronin 1 promoter element starts directly upstream from a transcription starting site (TSS) of a coronin 1 gene and spans a sequence stretch of at least about 700 bp in said genome. The present invention further relates to a method using said vector for identifying immunomodulatory compounds that alter the coronin 1 promoter activity. The present invention further relates to BRD3 as an upstream target responsible for driving the coronin-1 expression and activity in immune cells, and relates to compounds, in particular compound of formula (I), that selectively target bromodomains of BRD3 and thereby deplete coronin 1 levels.

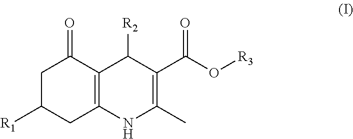

### RELATED APPLICATIONS

This application is a 35 U.S.C. § 371 filing of International Patent Application No. PCT/EP2023/077188, filed Sep. 30, 2023, which claims priority to European Patent Application Nos. 22199307.4 and 22199306.6, filed Sep. 30, 2022, the entire contents of which are incorporated herein by reference.

### SEQUENCE LISTING

The instant application contains a Sequence Listing which has been submitted electronically in XML format and is hereby incorporated by reference in its entirety. Said XML file, created on Mar. 11, 2025, is named 762383_VOS9-028US_ST26.xml and is 18,405 bytes in size.

The present invention relates to immunosuppressive compounds that deplete coronin 1 levels, in particular to coronin 1 promoter inhibitors.

In particular, the present invention relates to a compound of formula (I) and asymmetric (i.e., stereoselective) synthesis methods of the compound of formula (I). The compounds of formula (I) are provided for use as a medicament and are particularly suitable for inhibiting coronin 1 expression through coronin 1 promoter inhibition in the induction of immunosuppression or in the treatment and/or prevention of a disease or disorder selected from the group consisting of transplant rejection, autoimmune diseases, inflammatory diseases, infectious diseases, and lymphoproliferative disorders. The present invention further provides a vector comprising a coronin 1 promoter element, wherein in a vertebrate genome, said coronin 1 promoter element starts directly upstream from a transcription starting site (TSS) of a coronin 1 gene and spans a sequence stretch of 1500 bp or at least about 700 bp in said genome; a method for identifying compounds that modulate coronin 1 promoter activity.

The present invention further relates to the epigenetic reader Bromodomain-containing protein 3 (BRD3) as the upstream target that regulates coronin-1 promoter activity and coronin 1 expression in immune cells, and compounds acting through inhibition of BRD3.

### RELATED ART

T cell homeostasis is central to the ability of vertebrate organisms to mount an effective immune response. Lymphocyte precursors, originating from the bone marrow, home to the thymus, where negative and positive selection results in the production of CD4 or CD8 single positive T lymphocytes. From the thymus, single positive T lymphocytes seed the peripheral organs, where they cycle for prolonged times between the secondary lymphoid organs and the blood in a naive state. Following infection, T cells become activated by dendritic cells within peripheral lymph nodes which induce massive proliferation of so-called effector T cells. After the infection has been cleared, effector cells have to be eliminated in order to maintain peripheral T cell homeostasis.

The signals that are responsible for the selection, proliferation and survival of T cells rely on stimulation of the T cell receptor by major histocompatibility complex (MHC) molecules that are present on antigen presenting molecules. While in the thymus, positive selection selects those thymocytes recognizing self-MHC molecules, negative selection ensures the elimination of those T cells that strongly recognize self-peptides in the context of self-MHC. Together, these selection processes within the thymus ensure the generation of naive, non-autoreactive T cells for population of peripheral organs.

Coronin 1, also known as coronin 1A, (coro1a, CORO1A), IMD8, Coronin-1, Clipin A, P57 or TACO (tryptophan aspartate containing coat protein) is a protein that is transcribed in all cells of the hematopoietic system and neurons (Ferrari, G., et al., Cell, 1999. 97(4): p. 435-47.; Pieters, J., et al., Nat Rev Immunol, 2013. 13(7): p. 510). Coronin 1 is a member of the WD repeat family of coronin proteins that are widely expressed in the eukaryotic kingdom (Gatfield et al., Mol Biol Cell 2005, 16, 2786-2798; Pieters, J., et al., Nat Rev Immunol, 2013. 13(7): p. 510).

A role of coronin 1 was suggested for mycobacterial survival within macrophage phagosomes. Coronin 1 has been shown to inhibit endosomal/lysosomal fusion, and to impart non-fusogenic property to specifically mycobacteria containing phagosomes (Ferrari, G., et al., Cell, 1999. 97(4): p. 435-47; Jayachandran, R., et al., Cell, 2007. 130(1): p. 37-50.).

Studies analyzing complete coronin 1 knock-out mice have shown that this molecule is an important regulator of naive T cell homeostasis and it has been linked to immune deficiencies as well as autoimmune disorders (Mueller, P., et al., Nat Immunol, 2008. 9(4): p. 424-31; Foger, N., et al., Science, 2006. 313(5788): p. 839-42; Shiow, L. R., et al., Nat Immunol, 2008. 9(11): p. 1307-15; Haraldsson, M. K., et al., Immunity, 2008. 28(1): p. 40-51; Siegmund, K., et al., J Immunol, 2011. 186(6): p. 3452-61). T cell-specific coronin 1 knock-out mice were largely resistant to the induction of autoimmunity (Siegmund et al., J. Biol. Chem 2016, 291(42), 22086-22092). Thus, coronin 1 appears to have a predominant T cell-intrinsic role. Furthermore, allografts from MHC mismatched donors were tolerated in coronin 1-deficient mice resulting in long-term survival of transplanted organs along with absence of graft versus host response. Although deletion of coronin 1 results in a state of immunosuppression that results in attenuation of autoimmune responses and allograft rejection, immunity to infectious and foreign antigens are largely maintained in these mice (Pieters et al., Nat Rev Immunol. 2013, 13(7), 510-518; Jayachandran, R., et al., Immunity, 2019. 50(1): p. 152-165; Siegmund, K., et al., J Immunol, 2011. 186(6): p. 3452-61).

The presently used immunosuppressants often target proteins that are widely expressed in our body resulting in numerous side effects and toxicity (Rodriguez-Peralvarez, et al., Curr Opin Organ Transplant, 2014. 19(3): p. 253-60). For example, drugs such as calcineurin inhibitors (cyclosporine/FK506), corticosteroids or sirolimus lead to various side-effects and drug-induced toxicity including cancers, opportunistic infections, hypertension, altered metabolic profile and reduced patient compliance (Dantal, J. and M. Campone, Transplantation, 2016. 100(12): p. 2569-2583; Ross, K., J Natl Cancer Inst, 2007. 99(6): p. 421-2).

The dihydropyridine scaffold is used as heterocyclic structure in medicine for the treatment of several ailments with several functions including, but not limited to, antihypertensive, antitumor and anticonvulsant activities (selected review: V. K. Sharmaa and S. K. Singh, RSC Adv., 2017, 7, 2682-2732). Most commercially used dihydropyridine containing drugs are either achiral or are used as racemates, few are used as single enantiomers. Asymmetric syntheses of dihydropyridines have been developed but routes towards the related diaryl substituted 4,6,7,8-tetrahydroquinolin-5(1H)-one have not been reported.

Bromodomain Extra Terminal (BET) family of proteins are epigenetic readers comprised of four paralog members (BDR2, BRD3, BRD4 and BRDT) that recognize acetylated N-terminal tails of histones and act as readers of lysine acetylation state and interact with components of the transcriptional and chromatin remodeling machinery. These proteins play a critical role in malignant transformations and immune functioning (Gilan, O., et al., Science, 2020. 368(6489): p. 387-394. Faivre, E. J., et al., Nature, 2020. 578 (7794): p. 306-310) and are characterized by the presence two tandem bromodomains, bromodomain 1 (BD1) and bromodomain 2 (BD2), that aid in the docking to acetylated lysine on histones aiding chromatin binding. The BD1 and BD2 domains are highly conserved in evolution and also retain a marked level of homology across the paralogs. Structurally, they are characterized by an evolutionarily conserved sequence of approximately 110 amino acids that folds itself in to four α-helices (αZ, αA, αB, αC) interconnected by two intervening loops (BC loop and ZA loop) that collectively form a hydrophobic binding cavity for interaction with acetylated lysine of histones. Due to the high level of similarity between the paralogs, selectively targeting one particular BET protein has thus far been elusive, with the earlier reported compounds binding either to both the BD1 and BD2 domains of all the four members (pan-BET inhibitors) or binding to one of the BD domains (either BD1 (pan BD1-selective inhibitors) or BD2 (pan BD2-selective inhibitors)) of all the four BET proteins (Wang, N., et al., Signal Transduct Target Ther, 2021. 6(1): p. 23. Qi, J. and Y. Shi, Cancer Cell, 2020. 37(6): p. 764-766). BRD4 is the most well studied member in the family followed by BRD2 and they have been reported to play an important role in cancer development, embryogenesis, sepsis, immune function and fibrosis. BRD4 and BRD2 gene knock out has resulted in embryonic lethality (Houzelstein, D., et al., Mol Cell Biol, 2002. 22(11): p. 3794-802. Shang, E., et al., Dev Dyn, 2009. 238(4): p. 908-17). However, the role for BRD3 is the least characterized and its major function still remains largely unknown, due to functional redundancy with BRD2 resulting in minimal functional changes (Daneshvar, K., et al., Nat Cell Biol, 2020. 22(10): p. 1211-1222. Stonestrom, A. J., et al., Drug Discov Today Technol, 2016. 19: p. 23-28).

WO 2011/127164 discloses certain composition for treating fibrosis.

WO 2006/122156 discloses certain compounds modulating TRPV3 function.

WO 2008/070875 discloses certain polyhydroquinoline compounds and dihydropyridine compounds for inhibiting beta-amyloid production.

WO 2013/009799 discloses certain vitamin D-receptor agonists and their uses.

The catalogue “Aurora Building Blocks 7” dated Apr. 4, 2022, published by Aurora Fine Chemicals Ltd. from Graz, Austria, discloses certain compounds comprising a 2-methyl-5-oxo-1,4,5,6,7,8-hexahydro-3-quinolinecarboxylate substructure.

### SUMMARY OF THE INVENTION

The inventors developed and identified new compounds that deplete coronin 1 in cells, preferably immune cells, by coronin 1 promoter inhibition. Additionally, the absolute configuration of the eutomer was identified thereby separating toxicity of certain compounds from the coronin 1 promotor inhibitory activity. Chiral separation methods and asymmetric synthesis routes for these compounds have been developed. These compounds provide a new approach for inducing immunosuppression, allo-tolerance or prevention and/or treatment of transplant rejection, preferably allograft rejection, autoimmune diseases (selected from the group consisting of (but not limited to) psoriasis, vitiligo, multiple sclerosis, systemic lupus erythematosus, primary sclerosing cholangitis, Hashimoto's thyroiditis, rheumatoid arthritis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis), inflammatory diseases (selected from the group consisting of (but not limited to) inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, cardio vasculopathy, prurigo nodularis, hidradenitis suppurative, fibrotic disorders, allergic disorders, irritant contact dermatitis, eczematous dermatitis, seborrhoeic dermatitis, eosinophilic esophagitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome), infectious diseases (selected from the group consisting of (but not limited to) tuberculosis, preferably caused by mycobacteria, Salmonella sp. infections, Helicobacter sp. infections, retroviral infections, preferably HIV or HTLV, cytomegalo viral infection, Candida infection, Staphylococcus infections, lympho-choriomeningitis viral infections and viral hepatitis) as well as lymphoproliferative disorders (selected from the group consisting of (but not limited to) T cell lymphoma and T cell leukaemia via coronin 1 depletion).

The identified coronin 1 promoter inhibitors were validated for coronin 1 depletion at the mRNA and protein level and showed coronin 1 depletion. For example, compound 11 (methyl-4-(3-hydroxyphenyl)-7-(2-methoxyphenyl)-2-methyl-5-oxo-1,4,5,6,7,8-hexahydro-3-quinolinecarboxylate) elicited about 40% coronin 1 depletion with minimal toxicity both in vitro and in vivo. Hematological parameters and overall health status as assessed, e.g., by body weight changes did not reveal any potential toxicity. Additionally, in vitro studies with human peripheral blood mononuclear cells (PBMCs) revealed that coronin 1 promoter inhibitors deplete coronin 1 levels in human CD4 and CD8 T cells and that these inhibit the production of pro-inflammatory cytokines (Interleukin-2, Tumor necrosis factor and Interferon-gamma) upon T cell receptor stimulation, which reveals immunosuppressive action of the coronin 1 promoter inhibitors. Administration of coronin 1 promoter inhibitors is thus expected to lead to prolonged long-term transplant acceptance and suppression of autoimmune diseases (Jayachandran, R., et al., Cell, 2007. 130(1): p. 37-50; Jayachandran, R., et al., Immunity, 2019. 50: p. 1-15. Siegmund, K., et al., J Immunol, 2011. 186(6): p. 3452-61; Haraldsson, M. K., et al., Immunity, 2008. 28(1): p. 40-51).

Coronin 1, though expressed in diverse immune cell subtypes, is especially required for the survival of peripheral T cells (Pieters, J., et al., Nat Rev Immunol, 2013. 13(7): p. 510). In both coronin 1-deficient mice and humans, T cells are depleted in the peripheral lymphoid organs and blood. Despite this T cell deficiency, coronin 1-deficient mice have normal longevity and show no increase in opportunistic infections or spontaneous cancers. However, the absence of coronin 1 induces a prolonged survival of MHC-mismatched organ transplants and resistance towards development of autoimmune disorders (Jayachandran, R., et al., Immunity, 2019. 50: p. 1-15; Siegmund, K., et al., J Immunol, 2011. 186(6): p. 3452-61). These data further support that depletion of coronin 1 levels in vivo will induce a state of immunosuppression that will result in prolonged survival of organ transplants, suppression of autoimmune diseases and absence of any major complications with infections or malignancies. Side effects and toxicity are expected to be only minimal, since coronin 1 modulators mainly affect the T cell-specific functionality and survival. Moreover, immunity (including T cell-dependent) against microbial pathogens and cancers is being maintained upon coronin 1 ablation (Jayachandran, R., et al., Immunity, 2019. 50: p. 1-15).

The present inventors have further identified bromodomain 3 (BRD3) as an upstream regulator of coronin 1 expression and identified new compounds that selectively target bromodomains of BRD3, to inhibit expression of coronin 1. In other words, the compounds of the present invention deplete coronin 1 in cells, preferably immune cells, by inhibiting BRD3 to suppress coronin 1 promoter activity. Thus, these compounds provide a new approach for inducing immunosuppression, allo-tolerance or prevention and/or treatment of transplant rejection, preferably allograft rejection, autoimmune diseases, inflammatory diseases, infectious diseases as well as lymphoproliferative disorders via targeting of BRD3 which leads to coronin 1 depletion. In addition, as BRD3 has been shown to play a role in certain malignant conditions and their metastasis (BRD3-driven Nuclear protein in Testis (NUT) Midline Carcinoma (NMC), BRD3-driven Ovarian Clear Cell Carcinoma (OCCC)), colorectal carcinoma and rhabdomyosarcoma, these compounds could have potential applications in treating these oncological conditions (Ballenberger, M., et al., Chest, 2022. 161(1): p. e43-e49. French, C. A., et al., Oncogene, 2008. 27(15): p. 2237-42. Roberts, T. C., et al., Sci Rep, 2017. 7(1): p. 6153. Hsu, P L., et al., Sci Adv, 2023. 9, eade3422.)

As coronin 1 is needed also for bacterial survival inside macrophages (e.g. for mycobacteria, helicobacter and salmonella) (Jayachandran, R., et al., Cell, 2007. 130(1): p. 37-50; Jayachandran, R., et al., Immunity, 2019. 50: p. 1-15; Zheng, P. Y. and N. L. Jones, Cell Microbiol, 2003. 5(1): p. 25-40), reduction of coronin 1 is a method for killing bacteria and treating and/or preventing infections and diseases caused by bacteria, such as tuberculosis, gastric ulcer, gastric cancer etc.

Further, the inventors designed a reporter gene-based screening assay to identify compounds that selectively inhibit coronin 1 promoter activity which is measured by reduction of the reporter gene expression (e.g. Green Fluorescence Protein (GFP) signal). Analyzing the inhibition of an unrelated promoter, e.g. early cytomegalovirus promoter that drove a different reporter gene (e.g. Red Fluorescence Protein (RFP)) assessed specificity of the tested compound in selectively inhibiting coronin 1 promoter.

In one aspect, the present invention relates to a vector comprising a coronin 1 (coro1a) promoter element, wherein in a vertebrate genome, said coronin 1 promoter element starts directly upstream from a transcription starting site (TSS) of a coronin 1 gene and spans a sequence stretch of at least about 700 bp in said genome. Preferably, said coronin 1 promoter element spans a sequence stretch of at least 1500 bp in said genome.

In a further aspect, the present invention relates to a method for identification of compounds that modulate coronin 1 promoter activity comprising the steps of:
a. providing a host cell comprising said vector of the present invention, wherein said host cell is capable of expressing said promoter reporter genes of said vector;
b. subjecting said host cells to a compound to be tested; and
c. measuring expression of said coronin 1 promoter reporter gene in said host cell subjected to said compound to be tested.

In a further aspect, the invention relates to a cell comprising the vector of the invention.

In one aspect, the present invention relates to a compound of formula (I)

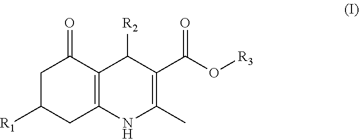

or a pharmaceutically acceptable salt, stereoisomer, diastereoisomer, enantiomer, polymorph, racemic mixture, solvate or isomers and mixtures thereof, wherein
R₁ is selected from phenyl and thienyl wherein said phenyl is optionally substituted with one or more optional substituents independently selected from —OH, —NO₂, halogen and —O—C₁-C₆-alkyl;
R₂ is selected from phenyl and thienyl, wherein said phenyl is optionally substituted with one or more optional substituents independently selected from —OH, —NO₂, -halogen and —O—C₁-C₆-alkyl;
R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Preferably, R₁ is selected from phenyl and thienyl, wherein said phenyl is optionally substituted with one or more —O—C₁-C₆-alkyl. Preferably, R₂ is selected from phenyl and thienyl, wherein said phenyl is optionally substituted with one or more optional substituents independently selected from —OH, —NO₂, and -halogen. Preferably, R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

In a further aspect, the present invention relates to compounds of formula (I)

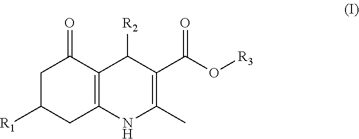

or a pharmaceutically acceptable salt, stereoisomer, diastereoisomer, enantiomer, polymorph, racemic mixture, solvate or isomers and mixtures thereof, wherein
R₁ is selected from phenyl and thienyl, wherein said phenyl is optionally substituted with one or more —O—C₁-C₆-alkyl;
R₂ is 3-hydroxyphenyl;
R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Preferably, R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Preferably the compound of formula (I) is selected from
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl 4-(3-hydroxyphenyl)-2-methyl-5-oxo-7-phenyl-1,4,5,6,7,8-hexahydroquinoline-3-carboxylate (4);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20);
tert-butyl 4-(3-hydroxyphenyl)-7-(2-methoxyphenyl)-2-methyl-5-oxo-1,4,5,6,7,8-hexahydroquinoline-3-carboxylate (21);
methyl 7-(4-chlorophenyl)-4-(3-hydroxyphenyl)-2-methyl-5-oxo-1,4,5,6,7,8-hexahydroquinoline-3-carboxylate (22);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
2,2,6,6-tetramethyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (52);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53);
oxepan-4-yl 4-(3-hydroxyphenyl)-7-(2-methoxyphenyl)-2-methyl-5-oxo-1,4,5,6,7,8-hexahydroquinoline-3-carboxylate (54);
hexahydrofuro[2,3-b]furan-3-yl 4-(3-hydroxyphenyl)-7-(2-methoxyphenyl)-2-methyl-5-oxo-1,4,5,6,7,8-hexahydroquinoline-3-carboxylate (55);
cyclopentyl 4-(3-hydroxyphenyl)-7-(2-methoxyphenyl)-2-methyl-5-oxo-1,4,5,6,7,8-hexahydroquinoline-3-carboxylate (56);
cyclohexyl 4-(3-hydroxyphenyl)-7-(2-methoxyphenyl)-2-methyl-5-oxo-1,4,5,6,7,8-hexahydroquinoline-3-carboxylate (57);
ethyl 4-(3-hydroxyphenyl)-7-(2-methoxyphenyl)-2-methyl-5-oxo-1,4,5,6,7,8-hexahydroquinoline-3-carboxylate (58);
butyl 4-(3-hydroxyphenyl)-7-(2-methoxyphenyl)-2-methyl-5-oxo-1,4,5,6,7,8-hexahydroquinoline-3-carboxylate (59);
neopentyl 4-(3-hydroxyphenyl)-7-(2-methoxyphenyl)-2-methyl-5-oxo-1,4,5,6,7,8-hexahydroquinoline-3-carboxylate (60);
2-ethylbutyl 4-(3-hydroxyphenyl)-7-(2-methoxyphenyl)-2-methyl-5-oxo-1,4,5,6,7,8-hexahydroquinoline-3-carboxylate (61);
2,2-dimethylbutyl 4-(3-hydroxyphenyl)-7-(2-methoxyphenyl)-2-methyl-5-oxo-1,4,5,6,7,8-hexahydroquinoline-3-carboxylate (62);
4,4-dimethylpentyl 4-(3-hydroxyphenyl)-7-(2-methoxyphenyl)-2-methyl-5-oxo-1,4,5,6,7,8-hexahydroquinoline-3-carboxylate (63);
2-(2-ethoxyethoxy)ethyl 4-(3-hydroxyphenyl)-7-(2-methoxyphenyl)-2-methyl-5-oxo-1,4,5,6,7,8-hexahydroquinoline-3-carboxylate (64);
2-(2-(2-(hexyloxy)ethoxy)ethoxy)ethyl 4-(3-hydroxyphenyl)-7-(2-methoxyphenyl)-2-methyl-5-oxo-1,4,5,6,7,8-hexahydroquinoline-3-carboxylate (65);
tetrahydro-2H-pyran-4-yl 4-(4-fluoro-3-hydroxyphenyl)-7-(2-methoxyphenyl)-2-methyl-5-oxo-1,4,5,6,7,8-hexahydroquinoline-3-carboxylate (66), and
tetrahydro-2H-pyran-4-yl 4-(2,4-difluoro-3-hydroxyphenyl)-7-(2-methoxyphenyl)-2-methyl-5-oxo-1,4,5,6,7,8-hexahydroquinoline-3-carboxylate (67).

Particularly preferred compounds of formula (I) are
tetrahydro-2-furanylmethyl (4S, 7R)-4-(3-hydroxyphenyl)-7-(2-methoxyphenyl)-2-methyl-5-oxo-1,4,5,6,7,8-hexahydro-3-quinolinecarboxylate (12e-S, & 12g-R);
tetrahydro-2H-pyran-4-yl (4S, 7R)-4-(3-hydroxyphenyl)-7-(2-methoxyphenyl)-2-methyl-5-oxo-1,4,5,6,7,8-hexahydro-3-quinolinecarboxylate (13a);
tetrahydrofuran-3-yl (4S, 7R)-4-(3-hydroxyphenyl)-7-(2-methoxyphenyl)-2-methyl-5-oxo-1,4,5,6,7,8-hexahydroquinoline-3-carboxylate (50a-S, & 50b-R).

Even more particularly preferred compound of formula (I) is:
tetrahydro-2H-pyran-4-yl (4S,7R)-4-(3-hydroxyphenyl)-7-(2-methoxyphenyl)-2-methyl-5-oxo-1,4,5,6,7,8-hexahydroquinoline-3-carboxylate (13a).

In a further aspect, the present invention relates to a pharmaceutical composition comprising a compound of formula (I) or a pharmaceutically acceptable salt, stereoisomer, diastereoisomer, enantiomer, polymorph, racemic mixture, solvate or isomers and mixtures thereof and a pharmaceutically acceptable carrier.

The present invention further relates to a method of preparing the compound of formula (I), the method comprising the step (b) of asymmetric reduction of the pyridine motive via enantioselective partial transfer hydrogenation.

In a further aspect, the present invention relates to a compound of formula (I) or a pharmaceutically acceptable salt, stereoisomer, diastereoisomer, enantiomer, polymorph, racemic mixture, solvate or isomers and mixtures thereof for use as a medicament.

In a further aspect the present invention relates to a compound of formula (I) or a pharmaceutically acceptable salt, stereoisomer, diastereoisomer, enantiomer, polymorph, racemic mixture, solvate or isomers and mixtures thereof, for inhibiting coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders.

In a further aspect, the present invention relates to a BRD3-selective bromodomain inhibitor for use in the treatment or prevention of a disease that can benefit from BRD3 inhibition, either directly or indirectly via reduced expression of Coronin 1.

In a further aspect, the present invention relates to a compound of formula (I) or a pharmaceutically acceptable salt, stereoisomer, diastereoisomer, enantiomer, polymorph, racemic mixture, solvate or isomers and mixtures thereof for use in the treatment or prevention of a disease that can benefit from BRD3 inhibition, either directly or indirectly via reduced expression of Coronin 1:

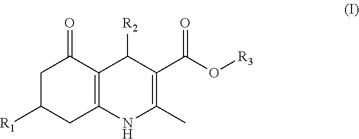

wherein
R₁ is selected from phenyl and thienyl wherein said phenyl is optionally substituted with one or more optional substituents independently selected from —OH, —NO₂, -halogen and —O—C₁-C₆-alkyl;
R₂ is selected from phenyl and thienyl, wherein said phenyl is optionally substituted with one or more optional substituents independently selected from —OH, —NO₂, -halogen and —O—C₁-C₆-alkyl,
preferably, R₂ is selected from phenyl and thienyl, wherein said phenyl is optionally substituted with one or more optional substituents independently selected from —OH, —NO₂, and -halogen;
R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl,

preferably, R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl. Further aspects and embodiments of the present invention will become apparent as this description continues. In the following section, we describe figures that outline the coronin 1 promoter characterization, coronin 1 promoter-based screening assay development, identify the coronin 1 promoter inhibitory compounds and as well their validation, optimization, safety and therapeutic efficacy in an autoimmune-inflammatory model of psoriasis.

### DESCRIPTION OF FIGURES

FIG. 1: Cartoon representation of various coronin 1 promoter constructs generated and characterization of their luciferase activity in J774 macrophages cell line. TSS: Transcription Start Site. J774 macrophages transfected with the indicated plasmid constructs sequence of coronin 1 promoter luciferase were assessed for luciferase activity. Controls: Empty pGL plasmid transfected cells (1^{st} row) and pGL-SV40 Positive control (2^{nd} row).

FIG. 2: Principle of the coronin 1 promoter screening assay of the invention: The promoter of a vertebrate (in the present case murine) coronin 1 gene (coro1a) was cloned upstream of the coding region of destabilized green fluorescence protein (GFP). CMV promoter-driven red fluorescence protein (RFP) served as an internal control for non-specific promoter inhibition. These constructs were stably transfected into rat basophil leukemia (RBL) cells and subjected to treatment with small molecule compounds from chemical libraries.

FIGS. 3A-3C: (FIG. 3A) The selective reduction in coronin 1 promoter driven GFP as imaged by a confocal microscope upon incubation with compound 11 (10 μg/ml) for 48 h. DMSO serves as the negative control. (FIG. 3B) Quantitative PCR analysis of coronin 1 mRNA upon incubation of RBL cells with compound 11 (3.5 μg/ml) for 7 days shows reduction in coronin 1 transcripts. DMSO serves as the vehicle control. (FIG. 3C) Analysis of native coronin 1 promoter inhibition by assessment of coronin 1 protein levels using Western blotting from RBL cell lines treated with compound 11 using a chemiluminescence imager. Compound 11 showed coronin 1 depletion up to a level of about ˜80% and was validated as true modulator of coronin 1 expression.

FIG. 4: Overlay of GFP FACS profiles of GFP-RBL cells treated with calcium channel inhibitors. Flow cytometry-based assessment of GFP fluorescence from RBL-GFP cells that had been incubated with calcium channel blockers Amlodipine (3.125 μM, right panel) and verapamil (8 μM, left panel) for 48 h. Both calcium channel blockers were not inhibiting coronin 1 promoter activity as assessed by GFP reduction.

FIGS. 5A-5B: In vitro and in vivo toxicological assessment of coronin 1 promoter inhibitors reveals them to be safe. In vitro toxicity analysis by Alamar blue assay (FIG. 5A) and MTT assay (FIG. 5B) in RBL cells with coronin 1 expression inhibitor 11 reveals minimal toxicity. Positive control: cycloheximide (1 μg/ml), vehicle control: DMSO.

FIGS. 6A-6E: Assessment of in vivo toxicity by analysis of hematological parameters using ADVIA platform in blood of mice upon on a six-day administration of compound 11 shows good safety and tolerability (150 mg/kg/body weight, BD, IP). The various parameters monitored include the red blood cell (RBC) counts (FIG. 6A), white blood cell (WBC) counts (FIG. 6B), platelet counts (FIG. 6C) and hemoglobin levels (FIG. 6D), relative to vehicle (DMSO) treated group. (FIG. 6E) Compound 11 was administered to mice in vivo every day for 14 days and body weight changes monitored relative to day zero as measure of overall health status and tolerance to compound 11 (150 mg/kg/body weight, BD, IP). DMSO serves as vehicle control.

FIG. 7: In vivo depletion of coronin 1 in mice with compound 11. In vivo administration of 11 depletes coronin 1 levels by 40%. Compound 11 was administered in vivo by sub-cutaneous route in miglyol and kolliphor based vehicle for a duration of 6 days (150 mg/kg/body weight, BD, SC) at the end of which mice were sacrificed and the coronin 1 levels probed in splenic lysate using an infrared dye tagged secondary antibodies and imaged using a Licor system. Left panel: Western blotting using infra-red based Licor imaging system. Actin serves as loading control. Right panel: Quantitation of coronin 1 reduction by ratiometric analysis relative to the actin band intensity.

FIGS. 8A-8C: Depletion of coronin 1 in human PBMCs and suppression of proinflammatory cytokine production by compound 11. (FIG. 8A) Incubation of human peripheral blood mononuclear cells (PBMCs) with coronin 1 expression inhibitor (compound 11, 10 μg/ml) for a duration of 5 days results in coronin 1 depletion as assessed by Western blotting. (FIG. 8B) Flow cytometric analysis of PBMC cell viability upon incubation with coronin 1 expression inhibitor (compound 11, 10-20 μg/ml) for a duration of 5 days. (FIG. 8C) Incubation of PBMCs with coronin 1 expression inhibitor compound 11 (10 μg/ml) for a duration of 4.5 days attenuates immune responses in CD4 and CD8 T cells as assessed by production of interleukin-2 (IL-2) post T cell receptor stimulation with CD3 and CD28 antibodies in human PBMCs as assessed by flow cytometry. DMSO and media serve as internal controls.

FIGS. 9A-9C: Composition of isomers of the individual mixtures and fractions of the parental compounds 11, 12 and 13.

FIG. 10: One of the (4S,7R)-configurated isomers of compound 12 (named Compound 12e) shows inhibition of Mixed Lymphocyte Responses (MLR) with human peripheral blood mononuclear cells as shown by reduction in tritiated-thymidine uptake suggesting its immunosuppressive activity in the context of human alloimmune responses.

FIG. 11: Western blotting of cell lysates and analysis for coronin 1 levels upon incubation with the indicated compounds using Western blotting of RBL cell lines. Compounds 12, 12e and 13a (2 μg/ml) showed coronin 1 depletion by Western blot in RBL cells line.

FIGS. 12A-12B: (FIG. 12A) Compound 13a (50 mg/kg body weight, twice daily, topical route) suppresses autoimmune-inflammatory disease severity as seen from the disease score (assessed from erythema, surface area affected, and psoriatic plaque formation) on day 4 in imiquimod-induced mouse model for psoriasis. DMSO serves as vehicle control. n=7 mice per group. (FIG. 12B) GvHD study with compound 13a (50 mg/kg body weight, twice daily, subcutaneous route). Analysis of suppression of alloantigen-driven expansion of CD4 (left) and CD8 T cells (middle) by cell trace violet dye dilution method and splenomegaly (right panel) on day 7. Every dot represents an individual animal (n=5 mice/group). Representative of 2 independent studies.

FIG. 13: Thermal Proteome Profiling (TPP) identifies BRD3 as the molecular target: The compounds' target of action has been identified using the procedure termed Thermal Proteome Profiling. This procedure identified the compound to interact with BRD3 and stabilizes it significantly with a q-value of 0.0007. The thermal denaturation of the target is increased by over four degrees Celsius in the presence of the compound 12e at 6 μM concentration. There was no significant thermal stabilization of BRD2 (q value NS) and BRD4 (q value NS) noted, despite the detection of higher numbers of unique peptides for BRD2 and BRD4 relative to BRD3.

FIG. 14A-14B: (FIG. 14A) Validation of the TPP identified target using an siRNA-based approach: brd3 siRNAs (target specific siRNA) was transfected in to RBL GFP (Rat Basophil Leukemia cell line expressing green fluorescence protein under the coronin 1 promoter) and 72h later, the GFP fluorescence level was assessed using flow cytometry as a measure of reduction in coronin 1 promoter activity. A significant reduction in coronin 1 promoter driven GFP fluorescence upon transfection with target specific siRNA was seen.

(FIG. 14B) Validation of the TPP identified target using a CRISPR/Cas9-based approach: Analysis of coronin 1 and BRD3 protein expression levels as median fluorescence intensity (MFI) by flow cytometry-based analysis of a series of RBL cell line clones that were gene-edited (using CRISPR/Cas9) or not on the brd3 gene.

FIG. 15: Validation of BRD3 as the target using bromoscan for direct target engagement with the compounds: The direct and competitive binding of the compound 12e and 13a to BRD3 was assessed at Eurofins using the bromoscan platform. This analysis revealed the compounds 12e and 13a to bind to the bromodomains BD1 and BD2 in BRD3 with a higher affinity for the BD2 domain as shown by lower Kd values for the BRD3 BD2 domain.

FIG. 16: Co-crystallization studies of the compound 13a with the bromodomains of BRD3: Crystal structures of the compound 13a bound to BD1 (left, 1.4 Å) and BD2 (right, 2 Å) of human BRD3 reveals the compound to protrude deeply into the histone binding pocket, thus competing sterically with the acetylated lysine of histones that is recognized by this pocket. In BD2, the ligand interacts strongly with a phenol ring with residues His395 and Glu396, both of which are not present in BD1. The protein is in cartoon representation, with the residues involved in ligand binding in stick representation. Double bonds not displayed. Water: ‘w’.

FIG. 17: Psoriasis disease score of wild type K5.Stat3 mice subjected to tape stripping and left either untreated (only tape stripped) or treated with either the vehicle or compound 11 (75 mg/kg/body weight, twice daily). N=6 mice per group.

FIG. 18: DSS-induced colitis model: Wild type mice were either left untreated (UT) or administered 2.5% Dextran Sodium Sulphate (DSS) in drinking water and treated either with the vehicle or compound 12 (100 mg/kg/body weight, twice daily, subcutaneous route) and scored disease activity on day 5 post disease induction. N=4 mice per group.

FIGS. 19A-19C: (FIG. 19A) Methicillin-resistant Staphylococcus aureus (MRSA) infection model study. Wild type mice were infected with MRSA and treated with compound 13a (50 mg/kg/body weight, BD), FK506 (5 mg/kg/body weight, BD) or tofacitinib (50 mg/kg/body weight, BD) subcutaneously and the bacterial burden assessed. (FIG. 19B) Candida albicans infection model study. Wild type mice were infected with C. albicans and treated with compound 13a (50 mg/kg/body weight, BD), FK506 (5 mg/kg/body weight, BD) or dexamethasone (100 mg/kg/body weight, BD) subcutaneously and the fungal burden assessed. (FIG. 19C) Mycobacterial survival in macrophage study. Lysosomal localization of mycobacteria M. bovis-BCG-GFP in compound 11 treated J774 macrophages. Quantification of lysosomal delivery of mycobacteria in macrophages with the indicated concentrations of compound 11. Rapid lysosomal delivery suggests lysosomal degradation and death of the mycobacteria. Three independent experiments, n=50-70 per condition.

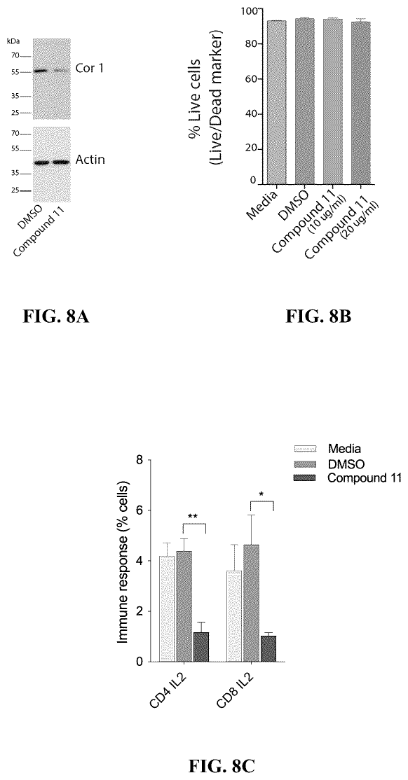

Collectively, the in vitro and in vivo data shown in FIGS. 8 to 19 reveals that the coronin 1 promoter screening assay identified inhibitory compounds that bind to the hydrophobic cavity within the two bromodomains of BRD3, preferably its BD2 bromodomain, regulating the coronin 1 promoter activity and expression of coronin 1 to induce allo-selective and auto-immune selective immunosuppression and anti-inflammatory activities.

### DETAILED DESCRIPTION OF THE INVENTION

Unless defined otherwise, all technical and scientific terms used herein have the same meaning as commonly understood by one of ordinary skill in the art to which this invention belongs.

Throughout this specification and the claims which follow, unless the context requires otherwise, the word “comprise”, and variations such as “comprises” and “comprising”, are to be understood to imply the inclusion of a stated integer or step or group of integers or steps but not the exclusion of any other integer or step or group of integer or step.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 0-10% smaller than the indicated numerical value and having an upper limit that is 0-10% larger than the indicated numerical value.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₈ alkyl” denotes an alkyl group having 1 to 8 carbon atoms. Preferred C₁₋₈ alkyl is a C₁₋₆ alkyl. “C₁-C₆-alkyl”, as used herein, preferably refers to straight chain or branched C₁-C₆-alkyl, which may be, for example, methyl, ethyl, n-propyl, isopropyl, n-butyl, isobutyl, sec-butyl, tert-butyl, straight or branched pentyl, straight or branched hexyl. Preferred C₁-C₆-alkyls are C₁-C₄-alkyls, and further preferably C₁-C₃-alkyls. Unless defined otherwise, the term “alkyl” preferably refers to C₁₅₄ alkyl, more preferably to methyl or ethyl, and even more preferably to methyl.

“O—C₁-C₆-alkyl”, as used herein, preferably refers to a “substituted hydroxyl” of the formula (—OR′), wherein R′ is a C₁-C₆-alkyl, as defined herein, and the oxygen moiety is directly attached to the parent molecule, and thus the term “O—C₁-C₆-alkyl”, as used herein, refers to straight chain or branched C₁-C₆-alkoxy which may be, for example, methoxy, ethoxy, n-propoxy, isopropoxy, n-butoxy, isobutoxy, sec-butoxy, tert-butoxy, straight or branched pentoxy, straight or branched hexyloxy. Preferred O—C₁-C₆-alkyl are O—C₁-C₄-alkyl.

The term “alkylene” preferably refers to an alkanediyl group, i.e. a divalent saturated acyclic hydrocarbon group which may be linear or branched. A “C₁₋₆ alkylene” denotes an alkylene group having 1 to 6 carbon atoms. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₂₋₄ alkylene (including, in particular, linear C₂₋₄ alkylene), more preferably to methylene or ethylene, and even more preferably to methylene.

“Halogen”, as used herein, preferably refers to fluorine (fluoro, —F), chlorine (chloro, —Cl), bromine (bromo, —Br), and iodine (iodo, —I). Preferably it refers to fluorine, chlorine or bromine. This also applies, correspondingly, to halogen in combination with other meanings, such as haloalkyl.

The term “cycloalkyl”, as used herein, preferably refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₆ cycloalkyl. A particularly preferred “cycloalkyl” is a monocyclic saturated hydrocarbon ring having 3 to 6 ring members (e.g., cyclopropyl, cyclobutyl, cyclopentyl or cyclohexyl).

The term “oxygen-containing saturated heterocyclyl”, as used herein, preferably refers fully saturated 5- to 14-membered ring system group comprising 1 to 2 oxygen atoms and not comprising any other atoms beyond C, H and O. Said heterocyclyl may be a single ring or two or more fused rings wherein at least one ring contains an oxygen atom. Preferably, the term “oxygen-containing saturated heterocyclyl”, as used herein, refers to fully saturated 4- to 7-membered single ring system group comprising 1 to 2 oxygen atoms and not comprising any other atoms beyond C, H and O. Examples of monocyclic oxygen-containing saturated heterocyclyl moieties are given as follows: oxetanyl, tetrahydrofuranyl, tetrahydropyranyl, 8-oxabicyclo[3.2.1]octan-3-yl, hexahydrofuro[2,3-b]furan-3-yl and oxepanyl.

Where a group is said to be optionally substituted, it may carry one or more substituents, such as, e.g., one, two, three or four substituents. It will be understood that the maximum number of substituents is limited by the number of attachment sites available on the substituted moiety. Preferably there are optionally 1-4 substituents, more preferably optionally 1-3 substituents, again more preferably optionally 1 or 2 substituents, and most preferably optionally 1 substituent. Where a group is said to be optionally substituted, and where there are more than one substituent for said optional substitution of said group, said more than one substituent can either be the same or different. As a skilled person will understand, the expression “substituted by” such as in “A is substituted by B” does not mean that B replaces A but that at least one hydrogen atom of A is replaced by at least one group B. The expression “substituted by” is thus equivalent to the expression “substituted with”.

Compounds of the invention may have one or more optically active carbon atoms and can thus be present as racemic mixtures, stereoisomers, diastereomers, or enantiomers. All isomeric forms are included in the present invention. The compounds having one or more optically active carbon atoms can be present as an individual stereoisomer, diastereomer, or enantiomer. Alternatively, mixtures thereof can be provided such as racemic mixtures or mixtures containing an excess of one of the stereoisomers, diastereomers, or enantiomers compared to the stereoisomer, diastereomer, or enantiomer having a different orientation at the one or more optically active carbon atoms, preferably the mixtures of the compounds of the invention are characterized by enantiomeric excess of at least 90%, more preferably at least 95%, even more preferably at least 96% of the particular isomer over its enantiomer.

The term “polymorphs” refers to the compounds of the present invention which can exist in two or more crystalline structures. Salts can also be crystalline and may exist as more than one polymorph.

Solvates (including hydrates) as well as anhydrous forms of the salt are also encompassed by the invention. The solvent included in the solvates is not particularly limited and can be any pharmaceutically acceptable solvent. Examples include water as well as C₁₋₄ alcohols (such as methanol or ethanol).

“Pharmaceutically acceptable salts” are defined as derivatives of the compounds of the present invention wherein the parent compound is modified by making acid or base salts thereof. Lists of suitable salts can be found in Remington's Pharmaceutical Sciences, 18^{th} ed., Mack Publishing Company, Easton, PA, 1990, p. 1445, the disclosure of which is hereby incorporated by reference.

The term “treating” and/or “treatment” refers to the management and care of a patient having a pathology such as a viral infection or other condition for which administration of one or more therapeutic compounds is indicated for the purpose of combating or alleviating symptoms and complications of those conditions. Treating includes administering one or more formulations of the present invention to prevent the onset of the symptoms or complications, alleviating the symptoms or complications, or eliminating the disease, condition, or disorder. As used herein, “treatment” or “therapy” refer to both therapeutic treatment and prophylactic or preventative measures. The effect may be therapeutic in terms of partially or completely curing a disease or a condition and/or symptoms attributed to the disease or the condition. The term refers to inhibiting the disease or condition, i.e. arresting its development; or ameliorating the disease or condition, i.e. causing regression or reducing symptoms of the disease or condition.

The term “prevention” as used herein refers to means of preventing or delaying the onset of disease or condition and/or symptoms attributed to the disease or condition.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterized by an increase or decrease of such symptoms or signs which may indicate a “worsening” or “bettering” of the disease. The “worsening” of a disease is characterized by a decreasing ability of a tissue, organ or organism to fulfil its function efficiently, whereas the “bettering” of a disease is typically characterized by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to transplant rejection, autoimmune diseases, inflammatory diseases, infectious diseases, and lymphoproliferative disorders.

As used herein, the terms “subject” or “animal” or “patient” or “mammal,” refers to any subject, particularly a mammalian subject, for whom diagnosis, prognosis, prophylaxis or therapy is desired, for example, a human or a domesticated mammal such as a dog, cat or horse or a food animal such as a cow or sheep or pig, preferably to a human.

As used herein, the term “for use” as used in “composition for use in treatment of a disease” shall disclose also the corresponding method of treatment and the corresponding use of a preparation for the manufacture of a medicament for the treatment of a disease.

A “therapeutically effective amount” or “effective amount” is the amount of a compound or pharmaceutical composition in accordance with the present invention that will elicit the biological or medical response of a subject, preferably a human subject, that is being sought by the researcher, veterinarian, medical doctor or other clinician. The term “therapeutic administration”, as used herein, should refer to the administration of therapeutically effective amount.

The term “promoter” or “promoter sequence” or “promoter element” as used herein refers to a nucleic acid sequence that is capable of directing transcription of a gene. The term coronin 1 (coro1a, e.g., NCBI mouse Gene ID: 12721; human Gene ID: 11151, rat Gene ID: 155151 and other vertebrates) promoter, promoter sequence or promoter element is capable of directing transcription of a coro1a gene. Said coronin 1 promoter is preferably a vertebrate coronin 1 promoter, more preferably a mammalian coronin 1 promoter, again more preferably a human, rat, mouse, bovine, dog, cow, hamster coronin 1 promoter, again more preferably a human, rat, mouse, coronin 1 promoter.

As preferably understood herein, whenever a reference is made to a compound or a pharmaceutically acceptable salt, stereoisomer, diastereoisomer, enantiomer, polymorph, racemic mixture, solvate or isomers and mixtures thereof, preferably a reference is made to a compound or a pharmaceutically acceptable salt, stereoisomer, diastereoisomer, enantiomer, or isomers and mixtures thereof, more preferably a reference is made to a compound or a pharmaceutically acceptable salt, stereoisomer, diastereoisomer, or enantiomer thereof, even more preferably a reference is made to a compound or a pharmaceutically acceptable salt thereof. It is understood that the term “mixtures” covers, but is not limited to, racemic mixtures of the compound or of the pharmaceutically acceptable salt thereof.

As further preferably understood herein, whenever a reference is made to a compound of formula (I), the reference is preferably made to the compound of formula (I) or its pharmaceutically acceptable salt.

In a first aspect, the invention relates to a vector comprising a coronin 1 (coro1a) promoter element, wherein in a vertebrate genome, said coronin 1 promoter element starts directly upstream from a transcription starting site (TSS) of a coronin 1 gene and spans a sequence stretch of at least about 700 bp in said genome.

The term “vector” as used herein refers to at least one nucleic acid molecule. It is used as a vehicle to artificially transfer nucleic acids and genetic material. The vector can be one or more a closed or open nucleic acid molecule being single stranded (ss) or double stranded (ds). In case the vector is ss, length specifications should be understood as nucleotides (nt) or bases; in case the vector is ds, length specifications should be understood as base pairs (bp). The term vector includes an expression cassette, plasmid, viral vector, phagemid, phage, cosmid, and artificial chromosomes (YAC, BAC, and PAC etc.), expression vector and cloning vector. In a preferred embodiment, said vector is a plasmid.

In a preferred embodiment, said vector further comprises a coronin 1 promoter reporter gene, wherein said coronin 1 promoter element is operably linked to said coronin 1 promoter reporter gene.

The expression “operably linked” means that the sequences, such as gene and promoter are a functional combination, i.e. the promoter is suitably positioned in order to regulate and preferably initiate transcription of the encoding gene. A reporter gene is a read-out gene that is operably linked to a promoter to indicate activity of the promoter, preferably in a semi-quantitative or quantitative manner.

Said coronin 1 promoter element coronin 1 promoter element has a length of at least about 700 bp, wherein said coronin 1 promoter element is located directly upstream of a transcription starting site (TSS) of a coronin 1 gene in a vertebrate genome. Said coronin 1 promoter element having a length of at least about 700 bp spans a sequence stretch from a base pair located directly upstream (5′) of the TSS to a base pair located at least about 700 bp upstream of the TSS in the vertebrate genome.

The transcription start site is the sequence where transcription starts and is located at the 5′-end of a gene sequence.

In a more preferred embodiment, said coronin 1 promoter element starts in a vertebrate genome directly upstream from a transcription starting site (TSS) of a coronin 1 gene and spans a sequence stretch of at least about 700 bp to about 3000 bp. In an again more preferred embodiment, said coronin 1 promoter element starts in a vertebrate genome directly upstream from a transcription starting site (TSS) of a coronin 1 gene and spans a sequence stretch of at least about 700 bp to about 1500 bp. In an again more preferred embodiment, said coronin 1 promoter element starts in a vertebrate genome directly upstream from a transcription starting site (TSS) of a coronin 1 gene and spans a sequence stretch of about 700 bp.

In a more preferred embodiment, said coronin 1 promoter element starts in a vertebrate genome directly upstream from a transcription starting site (TSS) of a coronin 1 gene and spans a sequence stretch of at least 700 bp. In a more preferred embodiment, said coronin 1 promoter element starts in a vertebrate genome directly upstream from a transcription starting site (TSS) of a coronin 1 gene and spans a sequence stretch of at least 737 bp. In a more preferred embodiment, said coronin 1 promoter element starts in a vertebrate genome directly upstream from a transcription starting site (TSS) of a coronin 1 gene and spans a sequence stretch of at least 700 bp to 3000 bp. In an again more preferred embodiment, said coronin 1 promoter element starts in a vertebrate genome directly upstream from a transcription starting site (TSS) of a coronin 1 gene and spans a sequence stretch of at least 700 bp to 1530 bp. In an again more preferred embodiment, said coronin 1 promoter element starts in a vertebrate genome directly upstream from a transcription starting site (TSS) of a coronin 1 gene and spans a sequence stretch of at least 737 bp to 1530 bp. In an again more preferred embodiment, said coronin 1 promoter element starts in a vertebrate genome directly upstream from a transcription starting site (TSS) of a coronin 1 gene and spans a sequence stretch of 737 bp.

In a preferred embodiment, said coronin 1 promoter element has a length of about at least about 700 bp to about 3000 bp, wherein said coronin 1 promoter element is located directly upstream of a transcription starting site (TSS) of a coronin 1 gene in a vertebrate genome. In a more preferred embodiment, said coronin 1 promoter element has a length of about at least about 700 bp to about 1500 bp, wherein said coronin 1 promoter element is located directly upstream of a transcription starting site (TSS) of a coronin 1 gene in a vertebrate genome. In an again more preferred embodiment, said coronin 1 promoter element has a length of about 700 bp, wherein said coronin 1 promoter element is located directly upstream of a transcription starting site (TSS) of a coronin 1 gene in a vertebrate genome.

In an again more preferred embodiment, said coronin 1 promoter element has a length of at least 700 bp. In a preferred embodiment, said coronin 1 promoter element has a length of at least 700 bp to 3000 bp. In a more preferred embodiment, said coronin 1 promoter element has a length of at least 700 bp to 1530 bp. In an again more preferred embodiment, said coronin 1 promoter element has a length of 737 bp.

Said coronin 1 promoter is preferably a vertebrate coronin 1 promoter; more preferably a mammalian coronin 1 promoter, again more preferably a human, rat or mouse coronin 1 promoter.

In one embodiment, said vector comprises a coronin 1 promoter which comprises said coronin 1 promoter element, i.e. said coronin 1 promoter element is included in said coronin 1 promoter.

In a preferred embodiment, said coronin 1 promoter element is a sequence having an identity of at least 40%, preferably of at least 50%, more preferably of at least 60%, again more preferably of at least 70%, again more preferably of at least 80%, again more preferably of at least 90%, again more preferably of at least 95%, again more preferably of at least 98%, again more preferably of at least 99%, again more preferably of at least 100% with a sequence selected from the group consisting of SEQ ID NO: 1-6.

In a preferred embodiment, said coronin 1 promoter element is a sequence having an identity of at least 40%, preferably of at least 50%, more preferably of at least 60%, again more preferably of at least 70%, again more preferably of at least 80%, again more preferably of at least 90%, again more preferably of at least 95%, again more preferably of at least 98%, again more preferably of at least 99%, again more preferably of at least 100% with a sequence selected from the group consisting of SEQ ID NO: 4-6.

Preferably, said coronin 1 promoter element having an identity of at least 40%, preferably of at least 50%, more preferably of at least 60%, again more preferably of at least 70%, again more preferably of at least 80%, again more preferably of at least 90%, again more preferably of at least 95%, again more preferably of at least 98%, again more preferably of at least 99%, again more preferably of at least 100% with a sequence of SEQ ID NO: 1-6 or a sequence of SEQ ID NO: 4-6 retains coronin 1 promotor activity i.e., is capable of driving coronin 1 expression within any expression system. Preferably said expression system is a eukaryotic expression system, more preferably a vertebrate expression system, again more preferably a mammalian expression system.

In another preferred embodiment, said coronin 1 promoter element is a sequence selected from the group consisting of SEQ ID NO: 1, 2, 3, 4, 5 and 6. In a further preferred embodiment, said coronin 1 promoter element is a sequence selected from the group consisting of SEQ ID NO: 1, 2 and 3. In an alternative preferred embodiment, said coronin 1 promoter element is a sequence selected from the group consisting of SEQ ID NO: 4, 5 and 6. In again an alternative preferred embodiment, said coronin 1 promoter element is a sequence of SEQ ID NO: 1. In again an alternative preferred embodiment, said coronin 1 promoter element is a sequence of SEQ ID NO: 2. In again an alternative preferred embodiment, said coronin 1 promoter element is a sequence of SEQ ID NO: 3. In again an alternative preferred embodiment, said coronin 1 promoter element is a sequence of SEQ ID NO: 4. In again an alternative preferred embodiment, said coronin 1 promoter element is a sequence of SEQ ID NO: 5. In again an alternative preferred embodiment, said coronin 1 promoter element is a sequence of SEQ ID NO: 6.

In another preferred embodiment, said vector further comprises a second promoter and a second promoter reporter gene. Preferably, said second promoter is different from said coronin 1 promoter.

Said second promoter can be any promoter. Preferably said second promoter is different from said coronin 1 promoter. Preferably said promoter other than the coronin 1 promoter is a constitutively active or ubiquitously active promoter. In a further preferred embodiment, said second promoter is selected from the group consisting of a viral promoter, actin promoter, clathrin promoter and an early Cyto Megalo Virus (CMV) promoter. In a further preferred embodiment, said second promoter is an early Cyto Megalo Virus (CMV) promoter.

In a preferred embodiment, said vector further comprises a second promoter and a second promoter reporter gene, wherein said second promoter is different from said coronin 1 promoter, and said second promoter reporter gene is the same as or different from said coronin 1 promoter reporter gene. More preferably, said second promoter is different from said coronin 1 promoter, and said second promoter reporter gene is different from said coronin 1 promoter reporter gene. Then, an independent read-out of the two reporter genes is possible.

In another embodiment, said second promoter reporter gene is the same as said coronin 1 promoter reporter gene and said vector comprises a first and a second plasmid, wherein said coronin 1 promoter and said coronin promoter reporter gene are on the first plasmid, and said second promoter and said second promoter reporter gene are on a second plasmid.

In a preferred embodiment, said vector comprises a first and a second plasmid, wherein said coronin 1 promoter and said coronin promoter reporter gene are on the first plasmid, and said second promoter and said second promoter reporter gene are on a second plasmid. Preferably, said second promoter is different from said coronin 1 promoter. More preferably, said second promoter is different from said coronin 1 promoter, and said second promoter reporter gene is different from said coronin 1 promoter reporter gene.

In a preferred embodiment, said coronin 1 promoter, said coronin 1 promoter reporter gene, said second promoter and said second promoter reporter gene are on one and the same plasmid. Preferably, said second promoter is different from said coronin 1 promoter. More preferably, said second promoter is different from said coronin 1 promoter, and said second promoter reporter gene is different from said coronin 1 promoter reporter gene.

In a preferred embodiment, said coronin 1 promoter, said coronin 1 promoter reporter gene are included in a first expression cassette, and said second promoter and said second promoter reporter gene are included in a second expression cassette, wherein preferably said second promoter is different from said coronin 1 promoter, more preferably said second promoter is different from said coronin 1 promoter, and said second promoter reporter gene is different from said coronin 1 promoter reporter gene.

In a preferred embodiment, said coronin promoter reporter gene and said second promoter reporter gene are selected from the group consisting of a gene encoding a fluorescent protein, such as a Green Fluorescence Protein (GFP), Red Fluorescence Protein (RFP), destabilized GFP, or destabilized RFP; β-galactosidase; chloramphenicol acetyltransferase; alkaline phosphatase, secreted embryonic alkaline phosphatase (SEAP) and luciferase. In a preferred embodiment, said coronin promoter reporter gene and said second promoter reporter gene are different and selected from the group consisting of a gene encoding a fluorescent protein, such as a Green Fluorescence Protein (GFP), Red Fluorescence Protein (RFP), destabilized GFP, or destabilized RFP; β-galactosidase; chloramphenicol acetyltransferase; alkaline phosphatase, secreted embryonic alkaline phosphatase (SEAP) and luciferase. In a preferred embodiment, said coronin 1 promoter reporter gene and said second promoter reporter gene are based on fluorescence, luminescence or protein expression, more preferably on fluorescence. In a preferred embodiment, said coronin 1 promoter reporter gene and said second promoter reporter gene are different and based on fluorescence, luminescence or protein expression, more preferably on fluorescence.

In a preferred embodiment, one of said coronin promoter reporter gene and said second promoter reporter gene encodes a red fluorescent protein and the other promoter reporter gene encodes a green fluorescent protein. In a particularly preferred embodiment, one of said coronin promoter reporter gene and said second promoter reporter gene encodes GFP, preferably destabilized GFP, and the other encodes RFP, preferably destabilized RFP.

In a preferred embodiment, one of said coronin promoter reporter gene and said second promoter reporter gene encodes a fluorescent or luminescent protein, and said second promoter is an early Cyto Megalo Virus (CMV) promoter. In a preferred embodiment, one of said coronin promoter reporter gene and said second promoter reporter gene encodes a fluorescent protein, and said second promoter is an early Cyto Megalo Virus (CMV) promoter. In a preferred embodiment, one of said coronin promoter reporter gene and said second promoter reporter gene encodes a red fluorescent protein and the other promoter reporter gene encodes a green fluorescent protein, and said second promoter is an early Cyto Megalo Virus (CMV) promoter. In a particularly preferred embodiment, said second promoter is an early Cyto Megalo Virus (CMV) promoter, one of said coronin promoter reporter gene and said second promoter reporter gene encodes GFP, preferably destabilized GFP, and the other encodes RFP, preferably destabilized RFP.

In a preferred embodiment, said coronin 1 promoter element starting in said vertebrate genome directly upstream from a transcription starting site (TSS) of a coronin 1 gene spans a stretch of at least about 700 bp to about 1500 bp in said genome, preferably said coronin 1 promoter element spans a stretch of at least about 700 bp in said genome, and said second promoter is an early Cyto Megalo Virus (CMV) promoter. In a preferred embodiment, said coronin 1 promoter element starting in said vertebrate genome directly upstream from a transcription starting site (TSS) of a coronin 1 gene spans a stretch of at least about 700 bp to about 1500 bp in said genome, preferably said coronin 1 promoter element spans a stretch of at least about 700 bp in said genome, and said second promoter is an early Cyto Megalo Virus (CMV) promoter, and said promoter reporter genes encode a fluorescent or luminescent protein, preferably one of the promoter reporter gene encodes a red fluorescent protein and the other promoter reporter gene encodes a green fluorescent protein.

In a preferred embodiment, said coronin 1 promoter element is a sequence of SEQ ID NO: 4, 5 or 6, and said second promoter is an early Cyto Megalo Virus (CMV) promoter. In a preferred embodiment, said coronin 1 promoter element is a sequence of SEQ ID NO: 4, 5 or 6, and said second promoter is an early Cyto Megalo Virus (CMV) promoter, and said promoter reporter genes encode a fluorescent or luminescent protein, preferably wherein one of the promoter reporter gene encodes a red fluorescent protein and the other promoter reporter gene encodes a green fluorescent protein.

In a particularly preferred embodiment, said vector of the invention comprises a first expression cassette comprising a coronin 1 promoter and a coronin promoter reporter gene, and a second expression cassette comprising a second promoter and a second promoter reporter gene, wherein one of said coronin promoter reporter gene and said second promoter reporter gene encodes GFP, preferably destabilized GFP and the other encodes RFP, preferably destabilized RFP, and said second promoter is an early Cyto Megalo Virus (CMV) promoter, wherein said first and second expression cassettes are either located on the same plasmid or on different plasmids. Further preferably, said first and second expression cassettes are located on different plasmids.

In a preferred embodiment, said vector of the invention is included in a cell, preferably a vertebrate cell, more preferably a mammalian cell, again more preferably a human or mouse cell. In another preferred embodiment, said vector of the invention is included in a mammalian immune cell, more preferably rat basophil leukemia (RBL) cells.

The inventors developed a cell-based screening assay with which compounds can be identified that deplete coronin 1 protein levels by suppressing the promoter activity of the gene encoding coronin 1 (coro1a). In an exemplary embodiment, the assay involves using a genetically engineered immune cell lineage, namely Rat Basophil Leukemia (RBL) cell line in which expression of destabilized Green Fluorescent Protein (GFP) was under the control of coronin 1 gene promoter. The GFP gene was used as promoter reporter gene in order to read-out promoter activity. The coronin 1 promoter elements spanned from about 700 bp to about 3000 bp (737, 1530 bp, 3000 bp) upstream of the transcription start site of coronin 1 gene. As an internal control for non-specific promoter inhibition and to assess cytotoxicity, RBL cells were transfected with a plasmid that drives destabilized Red Fluorescence Protein (RFP) expression through the early Cyto Megalo Virus (CMV) promoter. Any compound that selectively reduces the GFP fluorescence without altering RFP is considered as a coronin 1 expression inhibitor.

In a preferred embodiment, said coronin 1 promoter sequence of the invention is an isolated nucleic acid.

In a further aspect, the invention relates to a cell, preferably a eukaryotic cell, more preferably a mammalian cell comprising the vector of the invention. In a preferred embodiment, the coronin 1 promoter of the vector of the invention could be transcribed and translated. Preferably said mammalian cells of the invention are mammalian immune cells. More preferably said mammalian cells of the invention are rat basophil leukemia (RBL) cells or any cell expressing coronin 1.

In a further aspect, the present invention relates to a method for identification of compounds that modulates coronin 1 promoter activity comprising the steps of:
(i) providing a host cell comprising said vector of the invention, wherein said host cell is capable of expressing said promoter reporter genes;
(ii) subjecting said host cells to a compound to be tested; and
(ii) measuring expression of said coronin 1 promoter reporter gene in said host cell subjected to said compound to be tested.

In a preferred embodiment, the step of providing a host cell comprising the vector of the invention comprises the steps of providing said vector of the invention and transfecting said vector into said host cells capable of expressing said coronin promoter reporter gene and said optional second promoter reporter gene.

Said host cell is subjected to said compound to be tested under conditions suitable for binding of said chemical compound to the coronin 1 promoter.

In a preferred embodiment, said vector used for the method of the invention comprises a second promoter and a second promoter reporter gene. Said host cell is preferably capable of expressing said second promoter reporter gene; and expression of said further coronin 1 promoter reporter gene is preferably measured in said host cell subjected to said compound to be tested and compared with the expression of coronin 1.

In a preferred embodiment, the method of the invention for identification of compounds that inhibit coronin 1 promoter activity further comprises the steps of comparing the expression of said coronin promoter reporter gene with a control value. Said control value can be produced by using a control compound instead of the compound to be tested in the method of the invention, i.e. subjecting said provided host cells to a control compound to be tested; and measuring expression of said coronin promoter reporter gene. Optionally expression of said second promoter reporter gene in said host cell subjected to said control compound to be tested is measured. Thus, specificity of the compound to be tested in inhibiting coronin 1 promoter can be assessed.

Modulation of expression of said optional second promoter reporter gene in said host cell subjected to said compound to be tested indicates unspecific modulation, since said optional second promoter reporter gene is operably linked to a promoter which is not related to the coronin 1 promoter.

Compared to a control value, a decrease of expression of said coronin 1 promoter reporter gene indicates that the tested chemical compound inhibits coronin 1 promoter, whereas no decrease of expression of said coronin 1 promoter reporter gene indicates that the tested chemical compound does not modulate coronin 1 promoter, and an increase of expression of said coronin 1 promoter reporter gene indicates that the tested chemical compound activates the coronin 1 promoter. Any compound that reduces expression of the coronin 1 promoter reporter gene without altering the expression of the second promoter reporter gene is considered as a coronin 1 expression inhibitor.

In a preferred embodiment, said host cell is a eukaryotic cell, preferably a mammalian cell, more preferably a mammalian immune cell. More preferably said mammalian immune cell is human, mouse or rat, preferably rat basophil leukemia (RBL) cell expressing coronin 1.

In a preferred embodiment, said vector is a plasmid wherein said coronin 1 promoter, said coronin promoter reporter gene, said second promoter and said second promoter reporter gene are on one and the same plasmid. In a preferred embodiment, said vector comprises a first and a second plasmid, wherein said coronin 1 promoter and said coronin promoter reporter gene are on a first plasmid and said optional second promoter and said optional second promoter reporter gene are on a second plasmid.

In a further aspect, the present invention relates to a compound of formula (I)

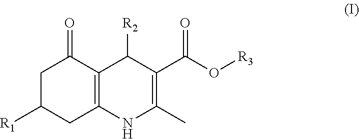

or a pharmaceutically acceptable salt, stereoisomer, diastereoisomer, enantiomer, polymorph, racemic mixture, solvate or isomers and mixtures thereof.

In formula (I), R₁ is selected from phenyl and thienyl, wherein said phenyl is optionally substituted with one or more optional substituents independently selected from —OH, —NO₂, halogen (preferably chloro) and —O—C₁-C₆-alkyl (preferably methoxy), preferably —O—C₁-C₆-alkyl (preferably methoxy). In one embodiment, R₁ is thienyl. In another embodiment, R₁ is phenyl, wherein said phenyl is optionally substituted with one or more optional substituents independently selected from halogen (preferably chloro) and —O—C₁-C₆-alkyl (preferably methoxy), preferably —O—C₁-C₆-alkyl (preferably methoxy). Even more preferably, R₁ is phenyl wherein said phenyl is optionally substituted with one or more —O—C₁-C₆-alkyl (preferably methoxy). Even more preferably, R₁ is selected from 2-methoxyphenyl, and phenyl.

R₂ is selected from phenyl and thienyl, wherein said phenyl is optionally substituted with one or more optional substituents independently selected from —OH, —NO₂, -halogen (preferably fluoro) and —O—C₁-C₆-alkyl (such as —O—CH₃). In one embodiment, R₂ is thienyl. In another embodiment, R₂ is phenyl, wherein said phenyl is optionally substituted with one or more optional substituents independently selected from —OH, —NO₂, -halogen and —O—C₁-C₆-alkyl (such as —O—CH₃). Preferably, R₂ is phenyl, wherein said phenyl is optionally substituted with one or more optional substituents independently selected from —OH, —NO₂, and —O—C₁-C₆-alkyl (such as —O—CH₃), even more preferably R₂ is phenyl, wherein said phenyl is optionally substituted with one or more optional substituents independently selected from —OH, and —O—C₁-C₆-alkyl (such as —O—CH₃). In a further preferred embodiment, R₂ is phenyl, wherein said phenyl is optionally substituted with one or more optional substituents independently selected from —OH. In one embodiment, R₂ is 3-hydroxyphenyl optionally substituted with one or more optional substituents independently selected from —OH, —NO₂, -halogen and —O—C₁-C₆-alkyl (such as —O—CH₃). In a further embodiment, R₂ is 3-hydroxyphenyl optionally substituted with one or more optional substituents independently selected from —OH, —NO₂, and -halogen. Even more preferably R₂ is 3-hydroxyphenyl.

In one embodiment, R₂ is selected from phenyl and thienyl, wherein said phenyl is optionally substituted with one or more optional substituents independently selected from —OH, —NO₂, and -halogen. Preferably, R₂ is phenyl, wherein said phenyl is optionally substituted with one or more optional substituents independently selected from —OH, —NO₂, and -halogen. More preferably R₂ is 3-hydroxyphenyl optionally substituted with one or more optional substituents independently selected from —OH, —NO₂, and -halogen. Even more preferably R₂ is 3-hydroxyphenyl.

R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl. It is to be understood that cycloalkyl is preferably a C₃-C₁₁-cycloalkyl, more preferably a C₃-C₆ cycloalkyl.

Preferably, R₃ is selected from —C₁-C₆-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

More preferably, R₃ is selected from —C₁-C₆-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Even more preferably, R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Even more preferably, R₃ is selected from —C₁-C₆-alkyl, —(C₁-C₆-alkylene)-O—C₁-C₆-alkyl, —C₁-C₆-alkylene-(oxygen-containing saturated heterocyclyl) and oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

In one embodiment, R₃ is selected from —C₁-C₈-alkyl, —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). Preferably, R₃ is selected from —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). More preferably, R₃ is selected from —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). Even more preferably, R₃ is selected from —(C₂-C₄-alkylene-O)ₘ—(C₁-C₆-alkyl) wherein m is an integer from 1 to 10 (preferably from 1 to 5, more preferably from 2 to 3, even more preferably 2).

In another embodiment, R₃ is selected from —C₁-C₆-alkylene-cycloalkyl, —C₃-C₆-cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl. Still more preferably, R₃ is selected from —C₁-C₆-alkylene-(oxygen-containing saturated heterocyclyl) and oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

Accordingly, preferably R₃ is selected from —C₁-C₆-alkylene-tetrahydro-2-furanyl

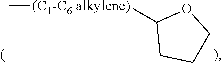

—C₁-C₆-alkylene-tetrahydro-2H-pyran-4-yl

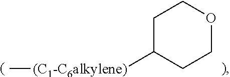

tetrahydrofuran-3-yl

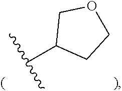 [SMILES: *C1CCOC1]

tetrahydro-2H-pyran-4-yl

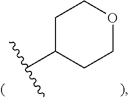 [SMILES: *C1CCOCC1]

oxepan-4-yl

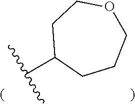 [SMILES: *C1CCCOCC1]

and 8-oxabicyclo[3.2.1]octan-3-yl

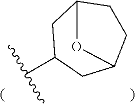 [SMILES: *C1CC2CCC(C1)O2]

wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl, said tetrahydrofuran-3-yl, said tetrahydro-2H-pyran-4-yl, said oxepan-4-yl, and said 8-oxabicyclo[3.2.1]octan-3-yl are each optionally substituted with one or more —C₁-C₆-alkyl, more preferably, R₃ is selected from —C₁-C₆-alkylene-tetrahydro-2-furanyl

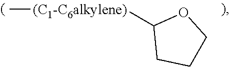

—C₁-C₆-alkylene-tetrahydro-2H-pyran-4-yl

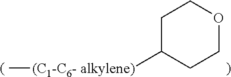

and tetrahydro-2H-pyran-4-yl

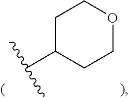 [SMILES: *C1CCOCC1]

wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl and said tetrahydro-2H-pyran-4-yl are each optionally substituted with one or more —C₁-C₆-alkyl.

Even more preferably, R₃ is selected from (tetrahydrofuran-2-yl)methyl

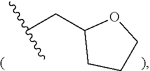 [SMILES: *CC1CCCO1]

tetrahydrofuran-3-yl

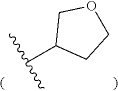 [SMILES: *C1CCOC1]

and tetrahydro-2H-pyran-4-yl

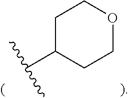 [SMILES: *C1CCOCC1]

Most preferably, R₃ is tetrahydro-2H-pyran-4-yl

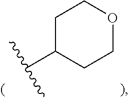 [SMILES: *C1CCOCC1]

In a further aspect, the present invention relates to a compound of formula (I)

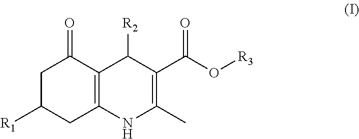

or a pharmaceutically acceptable salt, stereoisomer, enantiomer, or isomers and mixtures thereof,

wherein
R₁ is selected from phenyl and thienyl, wherein said phenyl is optionally substituted with —O—C₁-C₆-alkyl;
R₂ is 3-hydroxyphenyl;
R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Preferably R₁ is phenyl optionally substituted with methoxy. Most preferably R₁ is selected from 2-methoxyphenyl and phenyl.

It is to be understood that cycloalkyl is preferably a C₃-C₁₁-cycloalkyl, more preferably a C₃-C₆ cycloalkyl.

Preferably, R₃ is selected from —C₁-C₆-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

More preferably, R₃ is selected from —C₁-C₆-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Even more preferably, R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

Even more preferably, R₃ is selected from —C₁-C₆-alkyl, —(C₁-C₆-alkylene)-O—C₁-C₆-alkyl and —C₁-C₆-alkylene-(oxygen-containing saturated heterocyclyl) and oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

In one embodiment, R₃ is selected from —C₁-C₈-alkyl, —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). Preferably, R₃ is selected from —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). More preferably, R₃ is selected from —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2). Even more preferably, R₃ is selected from —(C₂-C₄-alkylene-O)ₘ—(C₁-C₆-alkyl) wherein m is an integer from 1 to 10 (preferably from 1 to 5, more preferably from 2 to 3, even more preferably 2).

In another embodiment, R₃ is selected from —C₁-C₆-alkylene-cycloalkyl, —C₃-C₆-cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl. Still more preferably, R₃ is selected from —C₁-C₆-alkylene-(oxygen-containing saturated heterocyclyl) and oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

Accordingly, preferably R₃ is selected from —C₁-C₆-alkylene-tetrahydro-2-furanyl

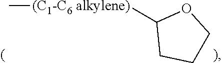

—C₁-C₆-alkylene-tetrahydro-2H-pyran-4-yl

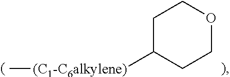

tetrahydrofuran-3-yl

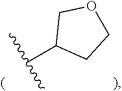 [SMILES: *C1CCOC1]

tetrahydro-2H-pyran-4-yl

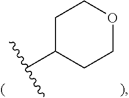 [SMILES: *C1CCOCC1]

oxepan-4-yl

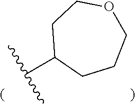 [SMILES: *C1CCCOCC1]

and 8-oxabicyclo[3.2.1]octan-3-yl

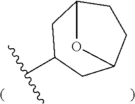 [SMILES: *C1CC2CCC(C1)O2]

wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl, said tetrahydrofuran-3-yl, said tetrahydro-2H-pyran-4-yl, said oxepan-4-yl, and said 8-oxabicyclo[3.2.1]octan-3-yl are each optionally substituted with one or more —C₁-C₆-alkyl, more preferably, R₃ is selected from —C₁-C₆-alkylene-tetrahydro-2-furanyl

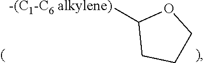

—C₁-C₆-alkylene-tetrahydro-2H-pyran-4-yl

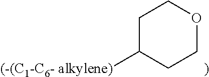

and tetrahydro-2H-pyran-4-yl

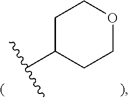 [SMILES: *C1CCOCC1]

wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl and said tetrahydro-2H-pyran-4-yl are each optionally substituted with one or more —C₁-C₆-alkyl.

Even more preferably, R₃ is selected from (tetrahydrofuran-2-yl)methyl

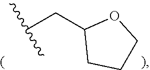 [SMILES: *CC1CCCO1]

tetrahydrofuran-3-yl

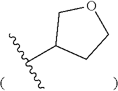 [SMILES: *C1CCOC1]

and tetrahydro-2H-pyran-4-yl

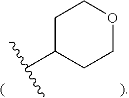 [SMILES: *C1CCOCC1]

Most preferably, R₃ is tetrahydro-2H-pyran-4-yl

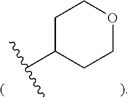 [SMILES: *C1CCOCC1]

Preferably, the compound of formula (I) is selected from
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl 4-(3-hydroxyphenyl)-2-methyl-5-oxo-7-phenyl-1,4,5,6,7,8-hexahydroquinoline-3-carboxylate (4);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20);
tert-butyl 4-(3-hydroxyphenyl)-7-(2-methoxyphenyl)-2-methyl-5-oxo-1,4,5,6,7,8-hexahydroquinoline-3-carboxylate (21);
methyl 7-(4-chlorophenyl)-4-(3-hydroxyphenyl)-2-methyl-5-oxo-1,4,5,6,7,8-hexahydroquinoline-3-carboxylate (22);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
2,2,6,6-tetramethyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (52);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53);
oxepan-4-yl 4-(3-hydroxyphenyl)-7-(2-methoxyphenyl)-2-methyl-5-oxo-1,4,5,6,7,8-hexahydroquinoline-3-carboxylate (54);
hexahydrofuro[2,3-b]furan-3-yl 4-(3-hydroxyphenyl)-7-(2-methoxyphenyl)-2-methyl-5-oxo-1,4,5,6,7,8-hexahydroquinoline-3-carboxylate (55);
cyclopentyl 4-(3-hydroxyphenyl)-7-(2-methoxyphenyl)-2-methyl-5-oxo-1,4,5,6,7,8-hexahydroquinoline-3-carboxylate (56);
cyclohexyl 4-(3-hydroxyphenyl)-7-(2-methoxyphenyl)-2-methyl-5-oxo-1,4,5,6,7,8-hexahydroquinoline-3-carboxylate (57);
ethyl 4-(3-hydroxyphenyl)-7-(2-methoxyphenyl)-2-methyl-5-oxo-1,4,5,6,7,8-hexahydroquinoline-3-carboxylate (58);
butyl 4-(3-hydroxyphenyl)-7-(2-methoxyphenyl)-2-methyl-5-oxo-1,4,5,6,7,8-hexahydroquinoline-3-carboxylate (59);
neopentyl 4-(3-hydroxyphenyl)-7-(2-methoxyphenyl)-2-methyl-5-oxo-1,4,5,6,7,8-hexahydroquinoline-3-carboxylate (60);
2-ethylbutyl 4-(3-hydroxyphenyl)-7-(2-methoxyphenyl)-2-methyl-5-oxo-1,4,5,6,7,8-hexahydroquinoline-3-carboxylate (61);
2,2-dimethylbutyl 4-(3-hydroxyphenyl)-7-(2-methoxyphenyl)-2-methyl-5-oxo-1,4,5,6,7,8-hexahydroquinoline-3-carboxylate (62);
4,4-dimethylpentyl 4-(3-hydroxyphenyl)-7-(2-methoxyphenyl)-2-methyl-5-oxo-1,4,5,6,7,8-hexahydroquinoline-3-carboxylate (63);
2-(2-ethoxyethoxy)ethyl 4-(3-hydroxyphenyl)-7-(2-methoxyphenyl)-2-methyl-5-oxo-1,4,5,6,7,8-hexahydroquinoline-3-carboxylate (64);
2-(2-(2-(hexyloxy)ethoxy)ethoxy)ethyl 4-(3-hydroxyphenyl)-7-(2-methoxyphenyl)-2-methyl-5-oxo-1,4,5,6,7,8-hexahydroquinoline-3-carboxylate (65);
tetrahydro-2H-pyran-4-yl 4-(4-fluoro-3-hydroxyphenyl)-7-(2-methoxyphenyl)-2-methyl-5-oxo-1,4,5,6,7,8-hexahydroquinoline-3-carboxylate (66) and
tetrahydro-2H-pyran-4-yl 4-(2,4-difluoro-3-hydroxyphenyl)-7-(2-methoxyphenyl)-2-methyl-5-oxo-1,4,5,6,7,8-hexahydroquinoline-3-carboxylate (67).

More preferably, the compound of formula (I) is selected from
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20); and
tert-butyl 4-(3-hydroxyphenyl)-7-(2-methoxyphenyl)-2-methyl-5-oxo-1,4,5,6,7,8-hexahydroquinoline-3-carboxylate (21).

Even more preferably, the compound of formula (I) is selected from
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19); and
tert-butyl 4-(3-hydroxyphenyl)-7-(2-methoxyphenyl)-2-methyl-5-oxo-1,4,5,6,7,8-hexahydroquinoline-3-carboxylate (21).

In one embodiment, the compound of formula (I) is selected from:
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13); and
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50).

Particularly preferred compound of formula (I) is selected from the compounds 12, 13 and 50.

In one embodiment, the compound of formula (I) is selected from
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53) and
oxepan-4-yl 4-(3-hydroxyphenyl)-7-(2-methoxyphenyl)-2-methyl-5-oxo-1,4,5,6,7,8-hexahydroquinoline-3-carboxylate (54).

It is to be understood herein, that preferably in the compound of formula (I) R₁ and R₂ substituents are present on the opposite faces of the ring system. Thus, preferably, the compound of formula (I) is a compound of formula:

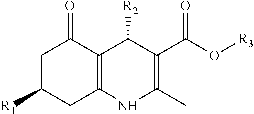

or a compound of formula:

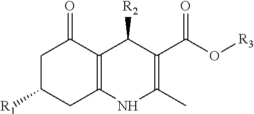

More preferably, the compound of formula (I) has an absolute configuration of its stereogenic centers as shown in the formula:

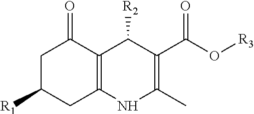

Thus, in a further embodiment, the compound of formula (I) is selected from:
tetrahydro-2-furanylmethyl (4S, 7R)-7-(2-methoxyphenyl)-2-methyl-5-oxo-4-(2-thienyl)-1,4,5,6,7,8-hexahydro-3-quinolinecarboxylate (1a-S, & 1b-R);
methyl (4S, 7R)-4-(4-fluorophenyl)-7-(2-methoxyphenyl)-2-methyl-5-oxo-1,4,5,6,7,8-hexahydro-3-quinolinecarboxylate (2a);
2-(ethylthio)ethyl (4S, 7R)-7-(2-methoxyphenyl)-2-methyl-5-oxo-4-(3-thienyl)-1,4,5,6,7,8-hexahydro-3-quinolinecarboxylate (3a);
Methyl (4S, 7R)-4-(3-hydroxyphenyl)-2-methyl-5-oxo-7-phenyl-1,4,5,6,7,8-hexahydroquinoline-3-carboxylate (4a);
methyl (4S, 7R)-4-(3-hydroxyphenyl)-2-methyl-5-oxo-7-(2-thienyl)-1,4,5,6,7,8-hexahydro-3-quinolinecarboxylate (5a);
tetrahydro-2-furanylmethyl (4S, 7R)-2-methyl-4-(3-nitrophenyl)-5-oxo-7-(2-thienyl)-1,4,5,6,7,8-hexahydro-3-quinolinecarboxylate (6a-S, & 6b-R);
tetrahydro 2-furanylmethyl (4S, 7R)-2-methyl-5-oxo-7-(2-thienyl)-4-(3-thienyl)-1,4,5,6,7,8-hexahydro-3-quinolinecarboxylate (7a-S, & 7b-R);
methyl (4S, 7R)-4-(3-hydroxyphenyl)-7-(4-methoxyphenyl)-2-methyl-5-oxo-1,4,5,6,7,8-hexahydro-3-quinolinecarboxylate (8a);
methyl (4S, 7R)-7-(2-methoxyphenyl)-2-methyl-5-oxo-4-(2-thienyl)-1,4,5,6,7,8-hexahydro-3-quinolinecarboxylate (9a);
tetrahydro-2-furanylmethyl (4S, 7R)-4-(2-fluorophenyl)-7-(2-methoxyphenyl)-2-methyl-5-oxo-1,4,5,6,7,8-hexahydro-3-quinolinecarboxylate (10a-S, & 10b-R);
methyl (4S, 7R)-4-(3-hydroxyphenyl)-7-(2-methoxyphenyl)-2-methyl-5-oxo-1,4,5,6,7,8-hexahydro-3-quinolinecarboxylate (11c);
tetrahydro-2-furanylmethyl (4S, 7R)-4-(3-hydroxyphenyl)-7-(2-methoxyphenyl)-2-methyl-5-oxo-1,4,5,6,7,8-hexahydro-3-quinolinecarboxylate (12e-S, & 12g-R);
tetrahydro-2H-pyran-4-yl (4S, 7R)-4-(3-hydroxyphenyl)-7-(2-methoxyphenyl)-2-methyl-5-oxo-1,4,5,6,7,8-hexahydro-3-quinolinecarboxylate (13a);
methyl (4S, 7R)-7-(2-methoxyphenyl)-2-methyl-5-oxo-4-phenyl-1,4,5,6,7,8-hexahydroquinoline-3-carboxylate (14a);
methyl (4S, 7R)-7-(2-methoxyphenyl)-4-(3-methoxyphenyl)-2-methyl-5-oxo-1,4,5,6,7,8-hexahydroquinoline-3-carboxylate (15a);
methyl (4S, 7R)-4-(2-hydroxyphenyl)-7-(2-methoxyphenyl)-2-methyl-5-oxo-1,4,5,6,7,8-hexahydroquinoline-3-carboxylate (16a);
4-methoxybutyl (4S, 7R)-4-(3-hydroxyphenyl)-7-(2-methoxyphenyl)-2-methyl-5-oxo-1,4,5,6,7,8-hexahydroquinoline-3-carboxylate (17a);
(tetrahydro-2H-pyran-4-yl)methyl (4S, 7R)-4-(3-hydroxyphenyl)-7-(2-methoxyphenyl)-2-methyl-5-oxo-1,4,5,6,7,8-hexahydroquinoline-3-carboxylate (18a);
(2,2,6,6-tetramethyltetrahydro-2H-pyran-4-yl)methyl (4S, 7R)-4-(3-hydroxyphenyl)-7-(2-methoxyphenyl)-2-methyl-5-oxo-1,4,5,6,7,8-hexahydroquinoline-3-carboxylate (19a);
oxetan-3-yl (4S, 7R)-4-(3-hydroxyphenyl)-7-(2-methoxyphenyl)-2-methyl-5-oxo-1,4,5,6,7,8-hexahydroquinoline-3-carboxylate (20a);
tert-butyl (4S, 7R)-4-(3-hydroxyphenyl)-7-(2-methoxyphenyl)-2-methyl-5-oxo-1,4,5,6,7,8-hexahydroquinoline-3-carboxylate (21a);
methyl (4S,7R)-7-(4-chlorophenyl)-4-(3-hydroxyphenyl)-2-methyl-5-oxo-1,4,5,6,7,8-hexahydroquinoline-3-carboxylate (22a);
tetrahydrofuran-3-yl (4S, 7R)-4-(3-hydroxyphenyl)-7-(2-methoxyphenyl)-2-methyl-5-oxo-1,4,5,6,7,8-hexahydroquinoline-3-carboxylate (50a-S, & 50b-R);
4-methyltetrahydro-2H-pyran-4-yl (4S, 7R)-4-(3-hydroxyphenyl)-7-(2-methoxyphenyl)-2-methyl-5-oxo-1,4,5,6,7,8-hexahydroquinoline-3-carboxylate (51a);
2,2,6,6-tetramethyltetrahydro-2H-pyran-4-yl (4S, 7R)-4-(3-hydroxyphenyl)-7-(2-methoxyphenyl)-2-methyl-5-oxo-1,4,5,6,7,8-hexahydroquinoline-3-carboxylate (52a);
8-oxabicyclo[3.2.1]octan-3-yl (4S, 7R)-4-(3-hydroxyphenyl)-7-(2-methoxyphenyl)-2-methyl-5-oxo-1,4,5,6,7,8-hexahydroquinoline-3-carboxylate (53a);
oxepan-4-yl (4S, 7R)-4-(3-hydroxyphenyl)-7-(2-methoxyphenyl)-2-methyl-5-oxo-1,4,5,6,7,8-hexahydroquinoline-3-carboxylate (54b-S, & 54c-R);
(3R,3aS,6aR)-hexahydrofuro[2,3-b]furan-3-yl (4S, 7R)-4-(3-hydroxyphenyl)-7-(2-methoxyphenyl)-2-methyl-5-oxo-1,4,5,6,7,8-hexahydroquinoline-3-carboxylate (55a);
cyclopentyl (4S, 7R)-4-(3-hydroxyphenyl)-7-(2-methoxyphenyl)-2-methyl-5-oxo-1,4,5,6,7,8-hexahydroquinoline-3-carboxylate (56a);
cyclohexyl (4S, 7R)-4-(3-hydroxyphenyl)-7-(2-methoxyphenyl)-2-methyl-5-oxo-1,4,5,6,7,8-hexahydroquinoline-3-carboxylate (57a);
ethyl (4S, 7R)-4-(3-hydroxyphenyl)-7-(2-methoxyphenyl)-2-methyl-5-oxo-1,4,5,6,7,8-hexahydroquinoline-3-carboxylate (58a);
butyl (4S, 7R)-4-(3-hydroxyphenyl)-7-(2-methoxyphenyl)-2-methyl-5-oxo-1,4,5,6,7,8-hexahydroquinoline-3-carboxylate (59a);
neopentyl (4S, 7R)-4-(3-hydroxyphenyl)-7-(2-methoxyphenyl)-2-methyl-5-oxo-1,4,5,6,7,8-hexahydroquinoline-3-carboxylate (60a);
2-ethylbutyl (4S, 7R)-4-(3-hydroxyphenyl)-7-(2-methoxyphenyl)-2-methyl-5-oxo-1,4,5,6,7,8-hexahydroquinoline-3-carboxylate (61a);
2,2-dimethylbutyl (4S, 7R)-4-(3-hydroxyphenyl)-7-(2-methoxyphenyl)-2-methyl-5-oxo-1,4,5,6,7,8-hexahydroquinoline-3-carboxylate (62a);
4,4-dimethylpentyl (4S, 7R)-4-(3-hydroxyphenyl)-7-(2-methoxyphenyl)-2-methyl-5-oxo-1,4,5,6,7,8-hexahydroquinoline-3-carboxylate (63a);
2-(2-ethoxyethoxy)ethyl (4S, 7R)-4-(3-hydroxyphenyl)-7-(2-methoxyphenyl)-2-methyl-5-oxo-1,4,5,6,7,8-hexahydroquinoline-3-carboxylate (64a);
2-(2-(2-(hexyloxy)ethoxy)ethoxy)ethyl (4S, 7R)-4-(3-hydroxyphenyl)-7-(2-methoxyphenyl)-2-methyl-5-oxo-1,4,5,6,7,8-hexahydroquinoline-3-carboxylate (65a);
tetrahydro-2H-pyran-4-yl (4S,7R)-4-(4-fluoro-3-hydroxyphenyl)-7-(2-methoxyphenyl)-2-methyl-5-oxo-1,4,5,6,7,8-hexahydroquinoline-3-carboxylate (66a); and
tetrahydro-2H-pyran-4-yl (4S,7R)-4-(2,4-difluoro-3-hydroxyphenyl)-7-(2-methoxyphenyl)-2-methyl-5-oxo-1,4,5,6,7,8-hexahydroquinoline-3-carboxylate (67a).

In one preferred embodiment compounds of formula (I) are
tetrahydro-2-furanylmethyl (4S, 7R)-4-(3-hydroxyphenyl)-7-(2-methoxyphenyl)-2-methyl-5-oxo-1,4,5,6,7,8-hexahydro-3-quinolinecarboxylate (12e-S, & 12g-R);
tetrahydro-2H-pyran-4-yl (4S, 7R)-4-(3-hydroxyphenyl)-7-(2-methoxyphenyl)-2-methyl-5-oxo-1,4,5,6,7,8-hexahydro-3-quinolinecarboxylate (13a);
(tetrahydro-2H-pyran-4-yl)methyl (4S, 7R)-4-(3-hydroxyphenyl)-7-(2-methoxyphenyl)-2-methyl-5-oxo-1,4,5,6,7,8-hexahydroquinoline-3-carboxylate (18a);
(2,2,6,6-tetramethyltetrahydro-2H-pyran-4-yl)methyl (4S, 7R)-4-(3-hydroxyphenyl)-7-(2-methoxyphenyl)-2-methyl-5-oxo-1,4,5,6,7,8-hexahydroquinoline-3-carboxylate (19a);
tetrahydrofuran-3-yl (4S, 7R)-4-(3-hydroxyphenyl)-7-(2-methoxyphenyl)-2-methyl-5-oxo-1,4,5,6,7,8-hexahydroquinoline-3-carboxylate (50a-S, & 50b-R);
4-methyltetrahydro-2H-pyran-4-yl (4S, 7R)-4-(3-hydroxyphenyl)-7-(2-methoxyphenyl)-2-methyl-5-oxo-1,4,5,6,7,8-hexahydroquinoline-3-carboxylate (51a);
8-oxabicyclo[3.2.1]octan-3-yl (4S, 7R)-4-(3-hydroxyphenyl)-7-(2-methoxyphenyl)-2-methyl-5-oxo-1,4,5,6,7,8-hexahydroquinoline-3-carboxylate (53a) and
oxepan-4-yl (4S, 7R)-4-(3-hydroxyphenyl)-7-(2-methoxyphenyl)-2-methyl-5-oxo-1,4,5,6,7,8-hexahydroquinoline-3-carboxylate (54b-S, & 54c-R).

In another preferred embodiment compounds of formula (I) are
methyl (4S, 7R)-4-(3-hydroxyphenyl)-7-(2-methoxyphenyl)-2-methyl-5-oxo-1,4,5,6,7,8-hexahydro-3-quinolinecarboxylate (11c);
methyl (4S, 7R)-7-(2-methoxyphenyl)-2-methyl-5-oxo-4-phenyl-1,4,5,6,7,8-hexahydroquinoline-3-carboxylate (14a);
4-methoxybutyl (4S, 7R)-4-(3-hydroxyphenyl)-7-(2-methoxyphenyl)-2-methyl-5-oxo-1,4,5,6,7,8-hexahydroquinoline-3-carboxylate (17a);
tert-butyl (4S, 7R)-4-(3-hydroxyphenyl)-7-(2-methoxyphenyl)-2-methyl-5-oxo-1,4,5,6,7,8-hexahydroquinoline-3-carboxylate (21a) and
2-(2-ethoxyethoxy)ethyl (4S, 7R)-4-(3-hydroxyphenyl)-7-(2-methoxyphenyl)-2-methyl-5-oxo-1,4,5,6,7,8-hexahydroquinoline-3-carboxylate (64a).

Very preferred compounds of formula (I) are
tetrahydro-2-furanylmethyl (4S, 7R)-4-(3-hydroxyphenyl)-7-(2-methoxyphenyl)-2-methyl-5-oxo-1,4,5,6,7,8-hexahydro-3-quinolinecarboxylate (12e-S, & 12g-R);
tetrahydro-2H-pyran-4-yl (4S, 7R)-4-(3-hydroxyphenyl)-7-(2-methoxyphenyl)-2-methyl-5-oxo-1,4,5,6,7,8-hexahydro-3-quinolinecarboxylate (13a); and
tetrahydrofuran-3-yl (4S, 7R)-4-(3-hydroxyphenyl)-7-(2-methoxyphenyl)-2-methyl-5-oxo-1,4,5,6,7,8-hexahydroquinoline-3-carboxylate (50a-S, & 50b-R).

In a first specific embodiment of the compound of formula (I), the compound has an absolute configuration of its stereogenic centers as shown in the formula:

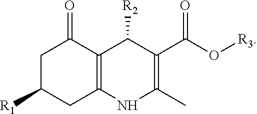

In this first specific embodiment, R₁, R₂ and R₃ are as defined for formula (I) including the preferred definitions of R₁, R₂ and R₃ given above.

In this first specific embodiment, R₁ is selected from phenyl and thienyl, wherein said phenyl is optionally substituted with one or more optional substituents independently selected from —OH, —NO₂, halogen (preferably chloro) and —O—C₁-C₆-alkyl (preferably methoxy), preferably —O—C₁-C₆-alkyl (preferably methoxy). In one embodiment, R₁ is thienyl. In another embodiment, R₁ is phenyl, wherein said phenyl is optionally substituted with one or more optional substituents independently selected from halogen (preferably chloro) and —O—C₁-C₆-alkyl (preferably methoxy), preferably —O—C₁-C₆-alkyl (preferably methoxy). Even more preferably, R₁ is phenyl wherein said phenyl is optionally substituted with one or more —O—C₁-C₆-alkyl (preferably methoxy). Even more preferably, R₁ is selected from 2-methoxyphenyl, and phenyl.

In this first specific embodiment, R₂ is selected from phenyl and thienyl, wherein said phenyl is optionally substituted with one or more optional substituents independently selected from —OH, —NO₂, -halogen and —O—C₁-C₆-alkyl (such as —O—CH₃). In one embodiment, R₂ is thienyl. In another embodiment, R₂ is phenyl, wherein said phenyl is optionally substituted with one or more optional substituents independently selected from —OH, —NO₂, -halogen and —O—C₁-C₆-alkyl (such as —O—CH₃). Preferably, R₂ is phenyl, wherein said phenyl is optionally substituted with one or more optional substituents independently selected from —OH, —NO₂, and —O—C₁-C₆-alkyl (such as —O—CH₃), even more preferably R₂ is phenyl, wherein said phenyl is optionally substituted with one or more optional substituents independently selected from —OH, and —O—C₁-C₆-alkyl (such as —O—CH₃). In a further preferred embodiment, R₂ is phenyl, wherein said phenyl is optionally substituted with one or more optional substituents independently selected from —OH. In one embodiment, R₂ is 3-hydroxyphenyl optionally substituted with one or more optional substituents independently selected from —OH, —NO₂, -halogen and —O—C₁-C₆-alkyl (such as —O—CH₃). In a further embodiment, R₂ is 3-hydroxyphenyl optionally substituted with one or more optional substituents independently selected from —OH, —NO₂, and -halogen. Even more preferably R₂ is 3-hydroxyphenyl.

In one embodiment, R₂ is selected from phenyl and thienyl, wherein said phenyl is optionally substituted with one or more optional substituents independently selected from —OH, —NO₂, and -halogen. Preferably, R₂ is phenyl, wherein said phenyl is optionally substituted with one or more optional substituents independently selected from —OH, —NO₂, and -halogen. More preferably R₂ is 3-hydroxyphenyl optionally substituted with one or more optional substituents independently selected from —OH, —NO₂, and -halogen. Even more preferably R₂ is 3-hydroxyphenyl.

In this first specific embodiment, R₃ is —C₁-C₆-alkylene-(oxygen-containing saturated heterocyclyl) and oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl. Suitable oxygen-containing saturated heterocyclyl moieties include tetrahydrofuran-3-yl, 8-oxabicyclo[3.2.1]octan-3-yl, oxepan-4-yl, hexahydrofuro[2,3-b]furan-3-yl (in particular (3R,3aS,6aR)-(hexahydrofuro[2,3-b]furan-3-yl), tetrahydro-2-furanyl, tetrahydro-2H-pyran-4-yl, and oxetan-3-yl,

Preferably, in this first specific embodiment, R₃ is selected from —C₁-C₆-alkylene-tetrahydro-2-furanyl, —C₁-C₆-alkylene-tetrahydro-2H-pyran-4-yl, tetrahydrofuran-3-yl tetrahydro-2H-pyran-4-yl, oxepan-4-yl and 8-oxabicyclo[3.2.1]octan-3-yl wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl, said tetrahydrofuran-3-yl, said tetrahydro-2H-pyran-4-yl, said oxepan-4-yl and said 8-oxabicyclo[3.2.1]octan-3-yl are each optionally substituted with one or more —C₁-C₆-alkyl.

Most preferably, in this first specific embodiment, R₃ is selected from (tetrahydrofuran-2-yl)methyl, tetrahydrofuran-3-yl and tetrahydro-2H-pyran-4-yl.

In a second specific embodiment of the compound of formula (I), the compound has an absolute configuration of its stereogenic centes as shown in the formula:

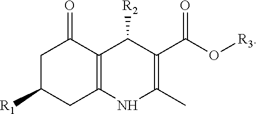

In this second specific embodiment, R₁ is selected from 2-methoxyphenyl, and phenyl. Preferably, R₁ is 2-methoxyphenyl.

In this second specific embodiment, R₂ is 3-hydroxyphenyl.

In this second specific embodiment, R₃ is selected from —C₁-C₈-alkyl, —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10 (preferably from 1 to 5, more preferably from 2 to 3, even more preferably 2).

Preferably, in this second specific embodiment, R₃ is selected from —C₁-C₆-alkyl, —(C₁-C₆-alkylene)-S—C₁-C₆-alkyl, and —(C₁-C₆-alkylene)-O—C₁-C₆-alkyl.

Even more preferably, in this second specific embodiment, R₃ is —C₁-C₆-alkyl.

Particularly suitable, in this second specific embodiment, —(C₂-C₄-alkylene-O)ₘ—(C₁-C₆-alkyl) include 2-(2-ethoxyethoxy)ethyl and 2-(2-(2-(hexyloxy)ethoxy)ethoxy)ethyl.

The compounds of the present invention are obtainable according to the following scheme of Hantzsch cyclization:

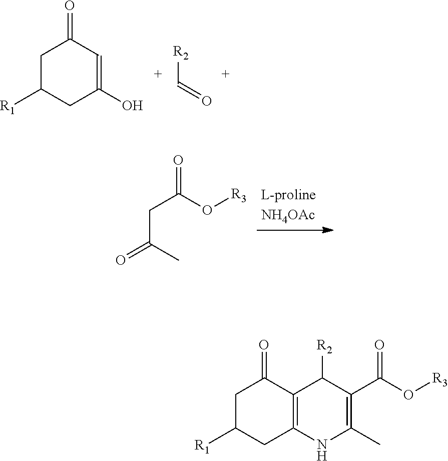

wherein R₁ to R₃ are defined in formula (I). Further details are given in Example 1.

Asymmetric synthesis of the compounds of the invention can be performed as in, or analogously to, the synthetic method described in Examples 2, 3 and 5, or as described hereinbelow.

Alternatively, enantiopure compounds can also be obtained by late-stage ester exchange via hydrolysis and esterification, the synthetic method described in Example 4, or as described hereinbelow. Accordingly, the present invention further relates to the method for preparing the compounds of formula (I), preferably in their enantiopure forms.

For example, the enantiopure compounds according to the present invention can be obtained either via A) diastereoselective synthesis followed by chromatographic separation or B) asymmetric synthesis as are depicted by the following reactions or C) late-stage ester exchange via hydrolysis and esterification (Scheme 1):

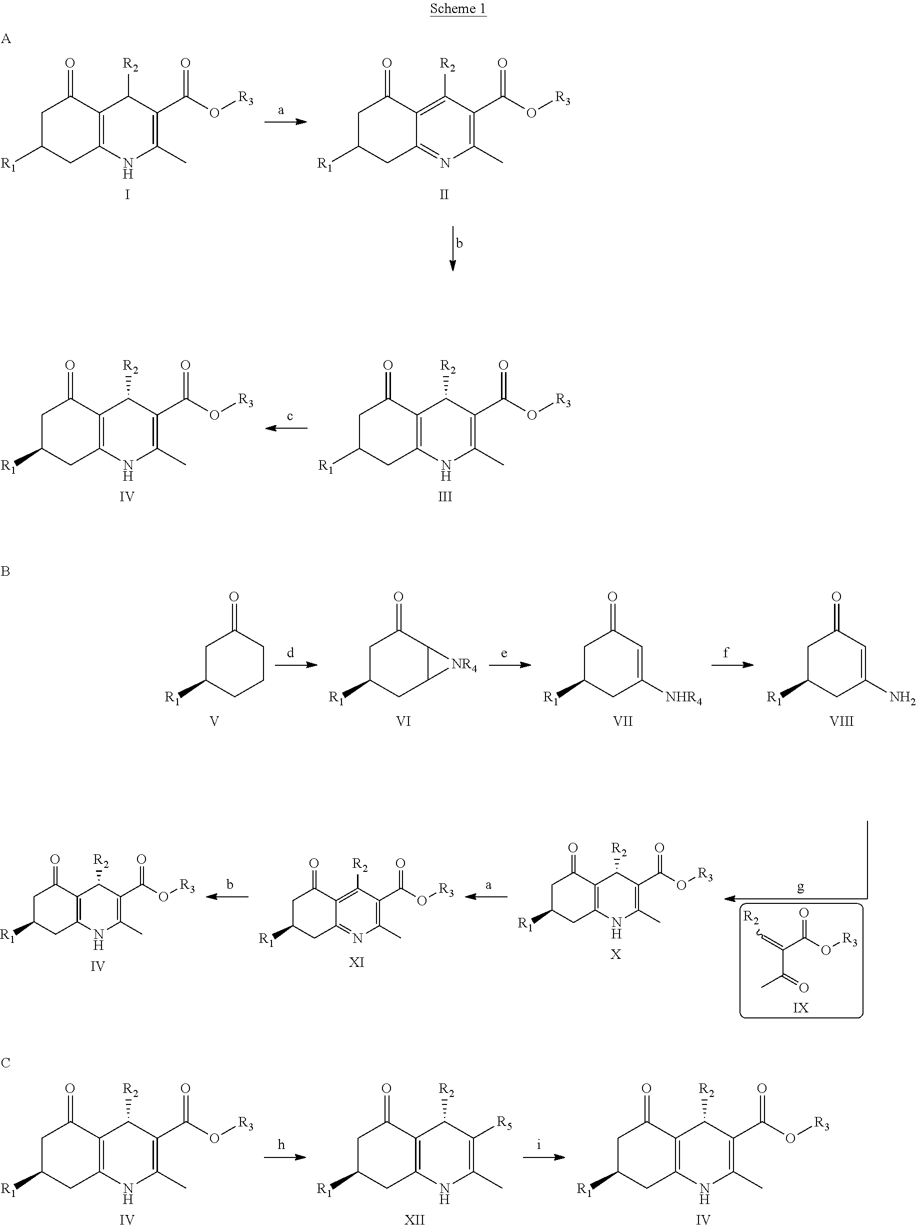

wherein R₁ to R₃ are defined in formula (I).
R₄ is selected from is selected from the group consisting of amine protecting groups, most preferably a tert-butyloxycarbonyl group. The skilled person is in position to select the correct group for use as R₄.
R₅ is an activated ester equivalent or a carboxylic acid, preferably a carboxylic acid. Said ester equivalents include, but are not limited to carboxylic acid chloride, carboxylic acid bromide and carboxylic acid anhydride. The skilled person is in a position to select the correct group for the use as R₅. Accordingly, R₅ may be selected from carboxylic acid, carboxylic acid chloride, carboxylic acid bromide and carboxylic acid anhydride, preferably R₅ is carboxylic acid.

The reaction shown can involve the following reaction steps:
Reactions:
a) Conversion of a 1,4-dihydropyridine motive to the corresponding pyridine motive by oxidation;
b) Asymmetric reduction of the pyridine motive via enantioselective partial transfer hydrogenation (chiral phosphoric acid, Hantzsch ester);
c) Separation of the diastereomers via preparative HPLC or flash chromatography;
d) Introduction of alpha, beta unsaturation via silyl enol ether formation followed by oxidation (base, R₃SiX, hypervalent iodine(V)) or selenoxide elimination (RSeX, oxidant) or direct dehydrogenation of cyclohohexanones (Pd^{II}, O₂);
   followed by aziridine formation via aziridination (N-protected-p-toluenesulfonamide, bisamine, base);
e) Aziridine opening via photochemical irradiation (hν);
f) Optional nitrogen deprotection, unless R₄ is hydrogen;
g) Condensation of intermediates VIII and IX;
h) Ester hydrolysis and optionally formation of active ester equivalent; and
i) Esterification.

Suitable reaction conditions for steps a, f-i are known to the skilled person.

Alternatively, steps h and i can be replaced by a direct transesterification step.

Accordingly, the present invention further relates to a method of preparing the compound of formula (I), as defined hereinabove, the method comprising the step (b) of asymmetric reduction of the pyridine motive via enantioselective partial transfer hydrogenation. Exemplary methods of the present invention are shown in Scheme 1 A and B. In one embodiment, the method further comprises the step (e) of aziridine opening via photochemical irradiation (hν). Preferably, the compound of formula (I) prepared according to the method of the invention is a compound of formula:

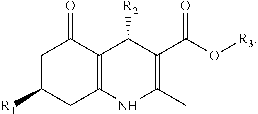

For the step (b) of asymmetric reduction of the pyridine motive via enantioselective partial transfer hydrogenation, conditions have been adapted from: Org. Lett. 2014, 16, 2982 and ACIE, 2020, 59, 23107.

Further details are given in Examples 2, 3 and 5.

The present invention further relates to intermediates in the process for preparing compound as shown in the formula:

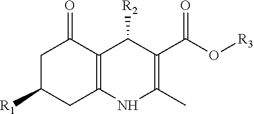

wherein R₁, R₂ and R₃ are as defined in formula (I), wherein the method comprises the step (b) of asymmetric reduction of the pyridine motive in compound IX:

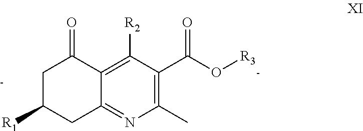

via enantioselective partial transfer hydrogenation.

Preferably, the intermediate is selected from the group consisting of compounds III, VI VII, X, XI and XII, preferably from the group consisting of compounds III, VI, X XI and XII:

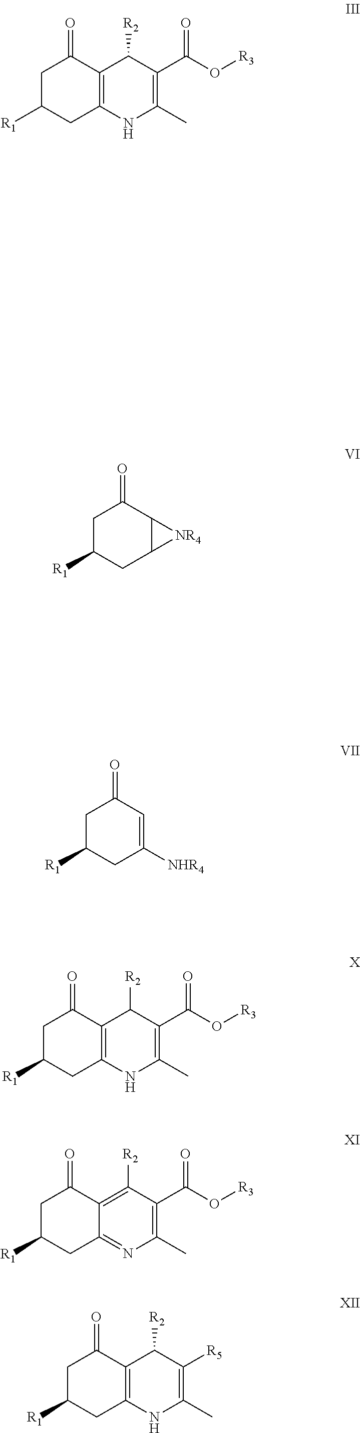

R₁, R₂ and R₃ are as defined in formula (I). R₄ and R₅ are defined as above.

Said compounds of Formula (I) are useful for inhibiting Coronin 1 expression.

Further, said compounds of the invention are useful for inhibiting Coronin 1 promoter activity (without being bound to the theory, preferably said inhibiting of Coronin 1 activity occurs through binding to the BRD3). Thus, said compounds of formula (I) are useful in treating, preventing and/or alleviating the symptoms of a disease associated with (or caused by) Coronin 1 expression. Further, said compounds of formula (I) are useful in treating, preventing and/or alleviating the symptoms of a disease associated with (or caused by) Coronin 1 promoter activity. The disease associated with (or caused by) Coronin 1 expression, and/or associated with (or caused by) Coronin 1 promoter activity can be selected from the group consisting of transplant rejection, autoimmune diseases, inflammatory diseases, infectious diseases, and lymphoproliferative disorders. Further, the compounds of formula (I) are useful as a medicament for inhibiting Coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders.

In a further aspect, the present invention relates to a compound of formula (I)

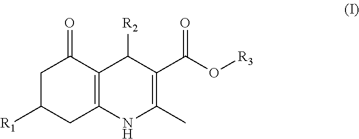

or a pharmaceutically acceptable salt, stereoisomer, diastereoisomer, enantiomer, polymorph, racemic mixture, solvate or isomers and mixtures thereof, as described hereinabove, for use as a medicament.

In a further aspect, the present invention relates to a compound of formula (I)

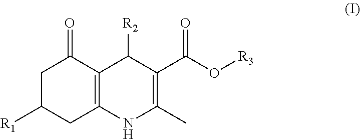

or a pharmaceutically acceptable salt, stereoisomer, diastereoisomer, enantiomer, polymorph, racemic mixture, solvate or isomers and mixtures thereof, as described hereinabove, for use as a medicament for inhibiting Coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders.

In a preferred embodiment, said compound of formula (I)

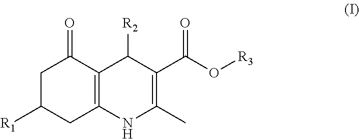

as defined herein, is used as a medicament for inhibiting Coronin 1 expression in the treatment and/or prevention of a disease or disorder selected from the group consisting of transplant rejection, autoimmune diseases, inflammatory diseases, infectious diseases, and lymphoproliferative disorders.

In even more preferred embodiment, the present invention provides a compound of formula (I) for use as a medicament for inhibiting coronin 1 expression in the induction of immunosuppression or the treatment and/or prevention of a disease or disorder selected from the group consisting of transplant rejection, autoimmune diseases, inflammatory diseases, infectious diseases, and lymphoproliferative disorders, wherein said compound is selected from
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl 4-(3-hydroxyphenyl)-2-methyl-5-oxo-7-phenyl-1,4,5,6,7,8-hexahydroquinoline-3-carboxylate (4);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20);
tert-butyl 4-(3-hydroxyphenyl)-7-(2-methoxyphenyl)-2-methyl-5-oxo-1,4,5,6,7,8-hexahydroquinoline-3-carboxylate (21);
methyl 7-(4-chlorophenyl)-4-(3-hydroxyphenyl)-2-methyl-5-oxo-1,4,5,6,7,8-hexahydroquinoline-3-carboxylate (22);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
2,2,6,6-tetramethyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (52);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53);
oxepan-4-yl 4-(3-hydroxyphenyl)-7-(2-methoxyphenyl)-2-methyl-5-oxo-1,4,5,6,7,8-hexahydroquinoline-3-carboxylate (54);
hexahydrofuro[2,3-b]furan-3-yl 4-(3-hydroxyphenyl)-7-(2-methoxyphenyl)-2-methyl-5-oxo-1,4,5,6,7,8-hexahydroquinoline-3-carboxylate (55);
cyclopentyl 4-(3-hydroxyphenyl)-7-(2-methoxyphenyl)-2-methyl-5-oxo-1,4,5,6,7,8-hexahydroquinoline-3-carboxylate (56);
cyclohexyl 4-(3-hydroxyphenyl)-7-(2-methoxyphenyl)-2-methyl-5-oxo-1,4,5,6,7,8-hexahydroquinoline-3-carboxylate (57);
ethyl 4-(3-hydroxyphenyl)-7-(2-methoxyphenyl)-2-methyl-5-oxo-1,4,5,6,7,8-hexahydroquinoline-3-carboxylate (58);
butyl 4-(3-hydroxyphenyl)-7-(2-methoxyphenyl)-2-methyl-5-oxo-1,4,5,6,7,8-hexahydroquinoline-3-carboxylate (59);
neopentyl 4-(3-hydroxyphenyl)-7-(2-methoxyphenyl)-2-methyl-5-oxo-1,4,5,6,7,8-hexahydroquinoline-3-carboxylate (60);
2-ethylbutyl 4-(3-hydroxyphenyl)-7-(2-methoxyphenyl)-2-methyl-5-oxo-1,4,5,6,7,8-hexahydroquinoline-3-carboxylate (61);
2,2-dimethylbutyl 4-(3-hydroxyphenyl)-7-(2-methoxyphenyl)-2-methyl-5-oxo-1,4,5,6,7,8-hexahydroquinoline-3-carboxylate (62);
4,4-dimethylpentyl 4-(3-hydroxyphenyl)-7-(2-methoxyphenyl)-2-methyl-5-oxo-1,4,5,6,7,8-hexahydroquinoline-3-carboxylate (63);
2-(2-ethoxyethoxy)ethyl 4-(3-hydroxyphenyl)-7-(2-methoxyphenyl)-2-methyl-5-oxo-1,4,5,6,7,8-hexahydroquinoline-3-carboxylate (64);
2-(2-(2-(hexyloxy)ethoxy)ethoxy)ethyl 4-(3-hydroxyphenyl)-7-(2-methoxyphenyl)-2-methyl-5-oxo-1,4,5,6,7,8-hexahydroquinoline-3-carboxylate (65);
tetrahydro-2H-pyran-4-yl 4-(4-fluoro-3-hydroxyphenyl)-7-(2-methoxyphenyl)-2-methyl-5-oxo-1,4,5,6,7,8-hexahydroquinoline-3-carboxylate (66); and
tetrahydro-2H-pyran-4-yl 4-(2,4-difluoro-3-hydroxyphenyl)-7-(2-methoxyphenyl)-2-methyl-5-oxo-1,4,5,6,7,8-hexahydroquinoline-3-carboxylate (67),

preferably selected from:
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20); and
tert-butyl 4-(3-hydroxyphenyl)-7-(2-methoxyphenyl)-2-methyl-5-oxo-1,4,5,6,7,8-hexahydroquinoline-3-carboxylate (21).

Thus, in said even more preferred embodiment, the present invention provides a compound of formula (I) for use as a medicament for inhibiting coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders, wherein said compound is selected from the compounds of any one of the formulae 1 to 22 or 50 to 65, and, wherein again further preferably said compound is selected from the compounds of formulae 11, 12, 13, 14, 17, 18, 19 and 21. Alternatively, said compound is selected from 12, 13, 18, 19, 50, 51, 53 and 54. Throughout the present invention, transplant rejection can be allograft rejection.

In a further preferred embodiment, the present invention relates to the use of a compound of the invention for the manufacture of a medicament for inhibiting coronin 1 expression in the induction of immunosuppression or in the treatment and/or prevention of a disease or disorder selected from the group consisting of transplant rejection, autoimmune diseases, inflammatory diseases, infectious diseases, and lymphoproliferative disorders.

In a further preferred embodiment, the present invention relates to a method for inhibiting coronin 1 expression in the induction of immunosuppression or in the treatment and/or prevention and/or alleviation of symptoms of a disease or disorder selected from the group consisting of transplant rejection, autoimmune diseases, inflammatory diseases, infectious diseases, and lymphoproliferative disorders comprising administering a compound of the invention to a subject in need thereof. In this method, a therapeutically effective amount of the compound of the invention is typically administered.

In a preferred embodiment, a compound of the present invention is used as a medicament for inhibiting coronin 1 expression. In another preferred embodiment, a compound of the present invention is used as a medicament for inhibiting coronin 1 promoter activity. In another preferred embodiment, a compound of the present invention is used as a medicament for depleting coronin 1 in a subject.

In another preferred embodiment, a compound of the invention is used as a medicament for inhibiting coronin 1 expression in the induction of immunosuppression or in the treatment and/or prevention of a disease or disorder selected from transplant rejection, autoimmune diseases, inflammatory diseases, infectious diseases, and lymphoproliferative disorders. In another preferred embodiment, a compound of the invention is used as a medicament for inhibiting coronin 1 expression in the induction of immunosuppression or in the treatment and/or prevention of a disease or disorder selected from the group consisting of transplant rejection, autoimmune diseases, and lymphoproliferative disorders. In another preferred embodiment, a compound of the invention is used as a medicament for inhibiting coronin 1 expression in the treatment and/or prevention of a disease or disorder selected from the group consisting of transplant rejection, autoimmune diseases, inflammatory diseases, infectious diseases, and lymphoproliferative disorders. In another preferred embodiment, a compound of the invention is used as a medicament for inhibiting coronin 1 expression in the treatment and/or prevention of a disease or disorder selected from the group consisting of transplant rejection, autoimmune diseases, and lymphoproliferative disorders. In another preferred embodiment, a compound of the invention is used as a medicament for inhibiting coronin 1 expression in the treatment and/or prevention of transplant rejection and/or lymphoproliferative disorders. In another preferred embodiment, a compound of the invention is used as a medicament for inhibiting coronin 1 expression in the treatment and/or prevention of inflammatory diseases and/or infectious diseases. In another preferred embodiment, a compound of the invention is used as a medicament for inhibiting coronin 1 expression in the induction of immunosuppression. In another preferred embodiment, a compound of the invention is used as a medicament for inhibiting coronin 1 expression in the treatment and/or prevention of transplant rejection. In another preferred embodiment, a compound of the invention is used as a medicament for inhibiting coronin 1 expression in the treatment and/or prevention of autoimmune diseases. In another preferred embodiment, a compound of the invention is used as a medicament for inhibiting coronin 1 expression in the treatment and/or prevention of lymphoproliferative disorders. In another preferred embodiment, a compound of the invention is used as a medicament for inhibiting coronin 1 expression in the treatment and/or prevention of inflammatory diseases. In another preferred embodiment, a compound of the invention is used as a medicament for inhibiting coronin 1 expression in the treatment and/or prevention of infectious diseases.

In another preferred embodiment, a compound of the invention is used as a medicament in the treatment and/or prevention of a disease or disorder selected from transplant rejection, autoimmune diseases, inflammatory diseases, infectious diseases, and lymphoproliferative disorders. In another preferred embodiment, a compound of the invention is used as a medicament in the treatment and/or prevention of a disease or disorder selected from the group consisting of transplant rejection, autoimmune diseases, and lymphoproliferative disorders. In another preferred embodiment, a compound of the invention is used as a medicament in the treatment and/or prevention of a disease or disorder selected from the group consisting of transplant rejection, autoimmune diseases, inflammatory diseases, infectious diseases, and lymphoproliferative disorders. In another preferred embodiment, a compound of the invention is used as a medicament in the treatment and/or prevention of a disease or disorder selected from the group consisting of transplant rejection, autoimmune diseases, and lymphoproliferative disorders. In another preferred embodiment, a compound of the invention is used as a medicament in the treatment and/or prevention of transplant rejection and/or lymphoproliferative disorders. In another preferred embodiment, a compound of the invention is used as a medicament in the treatment and/or prevention of inflammatory diseases and/or infectious diseases. In another preferred embodiment, a compound of the invention is used as a medicament in the induction of immunosuppression. In another preferred embodiment, a compound of the invention is used as a medicament in the treatment and/or prevention of transplant rejection. In another preferred embodiment, a compound of the invention is used as a medicament in the treatment and/or prevention of autoimmune diseases. In another preferred embodiment, a compound of the invention is used as a medicament in the treatment and/or prevention of lymphoproliferative disorders. In another preferred embodiment, a compound of the invention is used as a medicament in the treatment and/or prevention of inflammatory diseases. In another preferred embodiment, a compound of the invention is used as a medicament in the treatment and/or prevention of infectious diseases.

In preferred embodiment, a compound of the invention is used as a medicament in the induction of immunosuppression or in the treatment and/or prevention of a disease or disorder selected from the group consisting of transplant rejection, autoimmune diseases, inflammatory diseases, infectious diseases, and lymphoproliferative disorders, wherein said disease or disorder to be treated and/or prevented is caused or promoted by coronin 1 expression.

In preferred embodiment, a compound of the invention is used as a medicament in the induction of immunosuppression or in the treatment and/or prevention of a disease or disorder selected from the group consisting of transplant rejection, autoimmune diseases, inflammatory diseases, infectious diseases, and lymphoproliferative disorders, wherein said induction, treatment and/or prevention is based on coronin 1 depletion. In preferred embodiment, a compound of the invention is used as a medicament in the induction of immunosuppression or in the treatment and/or prevention of a disease or disorder selected from the group consisting of transplant rejection, autoimmune diseases, inflammatory diseases, infectious diseases, and lymphoproliferative disorders, wherein said induction, treatment and/or prevention is based on inhibition of coronin 1 expression.

Preferably, said autoimmune disease is selected from the group consisting of psoriasis, vitiligo, multiple sclerosis, systemic lupus erythematosus, Hashimoto's thyroiditis, rheumatoid arthritis, primary sclerosing cholangitis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis. More preferably, said, said autoimmune disease is selected from the group consisting of psoriasis, multiple sclerosis, systemic lupus erythematosus, Hashimoto's thyroiditis, rheumatoid arthritis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis.

Preferably, said transplant rejection is selected from the group consisting of acute or chronic rejection of cells, tissue, organ, allografts and xenografts, poor graft functional states, graft versus host disease. Preferably, said transplant rejection is selected from the group consisting of rejection of cardiac transplant, skin transplant, renal transplant, liver transplant, islet transplant, pancreas transplant, lung transplant, bowel transplant, corneal transplant, vascular transplant, adrenal transplant, hair transplant, bone transplant, cartilage transplant and ligamental transplant.

Preferably, said inflammatory disease is selected from the group consisting of inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, prurigo nodularis, hidradenitis suppurativa, eosinophilic esophagitis, fibrotic disorders, cardio vasculopathy, allergic disorders, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome, preferably consisting of inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, haemorrhage shock, traumatic shock, toxic shock, septic shock and adult respiratory distress syndrome. More preferably, said inflammatory disease is selected from the group consisting of inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, fibrotic disorders, allergic disorders, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome, preferably consisting of inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, haemorrhage shock, traumatic shock, toxic shock, septic shock and adult respiratory distress syndrome.

Preferably, said lymphoproliferative disorder is T cell lymphoma or T cell leukaemia.

Preferably, said infectious disease is selected from the group consisting of tuberculosis, preferably caused by mycobacteria, Salmonella sp. infections, Helicobacter sp. infections, retroviral infections, preferably HIV or HTLV, cytomegalo viral infection, candida infection, Staphylococcus infections, lympho-choriomeningitis viral infections and viral hepatitis.

Said Mycobacteria include and preferably are Mycobacterium tuberculosis, Mycobacterium leprae, Mycobacterium marinum, Mycobacterium bovis, Mycobacterium avium, Mycobacterium intracellulare, Mycobacterium kansasii, Mycobacterium malmoense, Mycobacterium simiae, Mycobacterium szulgai, Mycobacterium xenopi, Mycobacterium scrofulaceum, Mycobacterium abscessus, Mycobacterium chelonae, Mycobacterium haemophilum, and/or Mycobacterium ulcerans.

In a further embodiment, the present invention relates to a compound of formula (I) or a pharmaceutically acceptable salt, stereoisomer, diastereoisomer, enantiomer, polymorph, racemic mixture, solvate or isomers and mixtures thereof, as described hereinabove, for use in the treatment or prevention of a disease that can benefit from BRD3 inhibition, either directly or indirectly via reduced expression of Coronin 1.

The disease that can benefit from BRD3 inhibition (either directly or indirectly via reduced expression of Coronin 1) can preferably be understood as a disease amenable for therapeutic intervention through direct inhibition of BRD3 or via modulation of coronin 1 expression through BRD3 inhibition. Preferably, said disease is selected from transplant rejection, autoimmune diseases, inflammatory diseases, infectious diseases, and lymphoproliferative disorders. The recited diseases are as disclosed hereinabove. Furthermore, BRD3-driven diseases are preferably malignant diseases and their metastasis, such as, NMC, OCCC, colorectal carcinoma or rhabdomyosarcoma, preferably NMC, OCCC or rhabdomyosarcoma.

Accordingly, the present invention provides the compound of formula (I) for use in treatment or prevention of transplant rejection, autoimmune diseases, inflammatory diseases, infectious diseases, and lymphoproliferative disorders (as defined herein). The present invention further provides the compound of formula (I) for use in treatment or prevention of NMC, colorectal carcinoma, OCCC or rhabdomyosarcoma and their metastasis, preferably NMC, OCC or rhabdomyosarcoma.

As it is to be understood herein, BRD3 inhibition relates to blocking of the binding site of BRD3, thereby preventing BRD3 from binding its natural ligands (acetylated N-terminal tails of histones and other acetylated transcription factors like GATA_1, RelA, STAT3 etc). Accordingly, the compounds referred to as BRD3-inhibitors can also be referred to as BRD3-blockers, in particular BRD3-bromodomain blockers.

The present inventors have found that, surprisingly, the compounds of the present invention inhibit the expression of coronin 1, by targeting selectively the bromodomains of BRD3. The target BRD3 is known to drive the development of a highly invasive malignancy in 1/3 of the patients diagnosed to have NUT (Nuclear protein of Testis)-Midline Cancers (NMC) (Kervarrec, T., et al., Reply to: Expanding the Spectrum of Primary Cutaneous Carcinoma With BRD3-NUTM1 Fusion. Am J Surg Pathol, 2021. 45(11): p. 1584-1586). NMC is considered to be one of the most highly aggressive malignancies known with over 80% of the diagnosed patients dying within the first one year. Currently, there exists no specific cure except for surgical resection if detected early prior to significant metastasis or using a therapy with one of the non-selective BET inhibitors or side effect prone chemotherapy for NMC (Shapiro, G. I., et al., Br J Cancer, 2021. 124(4): p. 744-753). The non-selective BET inhibitors also block BRD4 and BRD2, which not only results in toxicity but also induces blanket immunosuppression due to inhibition of BRD4 and BRD2 dependent immune-inflammatory responses. Targeting of BRD3 has been reported to play a critical role in the control of a rare type of gynecological malignancy termed Ovarian Clear Cell Carcinoma (OCCC) (Shigeta, S., et al., Mol Cancer Ther, 2021. 20(4): p. 691-703). As with NMC, the mortality rate of patients with ovarian cancer is the highest among major gynecologic malignancies. The patients with OCCC are often treated with platinum-based chemotherapies causing numerous side effects and toxicity if not for the recently approved PARP inhibitors. Many of these patients become refractory to these therapies and hence there is a dire need for safe and targeted therapy against these cancers. Thus, the present invention relates to the compounds of the present invention for use in the treatment or prevention of a disease that can benefit from BRD3 inhibition either directly or indirectly via reduced expression of coronin 1 and coronin 1 activity. Preferably, the present invention relates to the compound of the present invention for use in the treatment of prevention of NMC or OCCC, preferably for use in the treatment or prevention of BRD3-driven NMC or BRD3-driven OCCC. Likewise, targeting BRD3 has been shown to play a critical role in eradication of cancer metastasis in a colorectal carcinoma model by being a critical phosphorylated substrate for TYRO3, thereby regulating anti-apoptosis gene expression and epithelial mesenchymal transition (Hsu, P L., et al., Sci Adv, 2023. 9, eade3422).

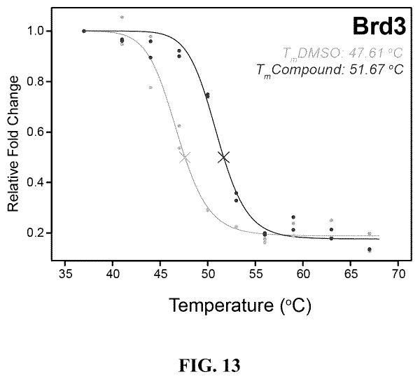

According to the present inventors, exemplary compounds of formula (I) are selective BRD3-bromodomain inhibitors (blockers). As preferably understood herein, a BRD3-selective bromodomain blocker is defined as a compound that significantly stabilizes BRD3 and does not significantly stabilize other bromodomain and extra-terminal (BET) family of bromodomain-containing proteins, as determined according to Thermal Proteome Profiling. Preferably, the BRD3-selective bromodomain blocker significantly stabilizes BRD3 and it does not significantly stabilize BRD2 and BRD4, as determined according to Thermal Proteome Profiling. Thermal Proteome Profiling is a technique well known to the skilled person. Suitable concentration range of compounds for Thermal Proteome Profiling are 1-6 μM. An exemplary result of a Thermal Proteome Profiling is shown in FIG. 13, and exemplary experimental details of applying such a method are disclosed in Example 7. Preferably, Thermal Proteome Profiling is to be performed at a concentration of the compound at 6 μM using RBL cells.

Preferably, the compound of formula (I), as defined herein, is a selective BRD3-bromodomain inhibitor (BRD3-bromodomain blocker), as defined herein. In other words, the compound of formula (I) preferably binds only to BRD3 and not to other bromodomain and extra-terminal (BET) family of bromodomain-containing proteins, as determined according to Thermal Proteome Profiling.

Further according to the present inventors, it is postulated that the medical applications of the compounds of the present invention which relate to the inhibition/blocking of BRD3 (i.e., the bromodomains of BRD3) are not limited to the compounds of formula (I), as provided herein, but can be practiced with any BRD3-selective bromodomain inhibitor (blocker). Accordingly, the present invention further provides a BRD3-selective bromodomain inhibitor for use in treatment or prevention of transplant rejection, autoimmune diseases, inflammatory diseases, infectious diseases, and lymphoproliferative disorders (as defined herein). The present invention further provides a BRD3-selective bromodomain inhibitor for use in treatment or prevention of NMC, colorectal carcinoma, OCCC or rhabdomyosarcoma, preferably NMC, OCCC or rhabdomyosarcoma. The exemplary and preferred a BRD3-selective bromodomain inhibitor is a compound of formula (I), as disclosed herein.

In a further aspect of the invention, said compounds of the present invention are used for inhibiting coronin 1 expression in vitro, preferably in a cell-based assay. Further, said compounds of the present invention are used for inhibiting coronin 1 promoter activity in vitro, preferably in a cell-based assay. Further, said compounds of the present invention are used for depleting coronin 1 in vitro, preferably in a cell-based assay. Said cells are preferably vertebrate cells, more preferably mammalian cells, again more preferably mammalian immune cells, again more preferably human, mouse or rat immune cells. Said cells are preferably selected from the group consisting of CD4, CD8 T cells, B cells, neutrophils, macrophages, dendritic cells, Langerhans cells, eosinophils, NK cells, follicular antigen presenting cells, monocytes, neuronal cells, glial cells, or basophil leukemia (RBL) cells, preferably of human, rat and mouse origin.

Further examples and embodiments are disclosed in the following numbered items.
1. A compound of formula (I)

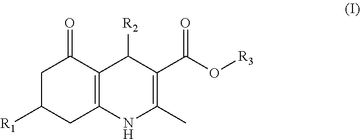

or a pharmaceutically acceptable salt, stereoisomer, diastereoisomer, enantiomer, or isomers and mixtures thereof,
   wherein
   R₁ is selected from phenyl and thienyl, wherein said phenyl is optionally substituted with one or more —O—C₁-C₆-alkyl;
   R₂ is selected from phenyl and thienyl, wherein said phenyl is optionally substituted with one or more optional substituents independently selected from —OH, —NO₂, and -halogen;
   R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.
2. The compound of item 1, wherein R₁ is phenyl optionally substituted with methoxy.
3. The compound of item 1 or 2, wherein R₁ is selected from 2-methoxyphenyl and phenyl.
4. The compound of any one of items 1 to 3, wherein R₂ is 3-hydroxyphenyl.
5. The compound of any one of items 1 to 4, wherein R₃ is —C₁-C₆-alkylene-(oxygen-containing saturated heterocyclyl) or oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.
6. The compound of any one of items 1 to 4, wherein R₃ is selected from —C₁-C₆-alkylene-tetrahydro-2-furanyl, C₁-C₆-alkylene-tetrahydro-2H-pyran-4-yl and tetrahydro-2H-pyran-4-yl, wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl and said tetrahydro-2H-pyran-4-yl are each optionally substituted with one or more —C₁-C₆-alkyl.
7. The compound of any one of items 1 to 6, wherein R₃ is tetrahydro-2H-pyran-4-yl.
8. The compound of item 1, selected from the group consisting of:
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20); and
tert-butyl 4-(3-hydroxyphenyl)-7-(2-methoxyphenyl)-2-methyl-5-oxo-1,4,5,6,7,8-hexahydroquinoline-3-carboxylate (21).
   9. The compound of item 1, selected from the group consisting of: methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12),
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19); and
tert-butyl 4-(3-hydroxyphenyl)-7-(2-methoxyphenyl)-2-methyl-5-oxo-1,4,5,6,7,8-hexahydroquinoline-3-carboxylate (21).
   10. The compound of any one of items 1 to 9, wherein the compound of formula (I) has an absolute configuration of its stereogenic centers as shown in the formula:

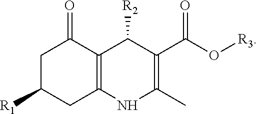

11. A pharmaceutical composition comprising a compound of any one of items 1 to 10 and a pharmaceutically acceptable carrier.
12. The compound of any one of items 1 to 10 or the pharmaceutical composition of item 11 for use as a medicament.
13. The compound of any one of items 1 to 10 or the pharmaceutical composition of item 11 for use in the induction of immunosuppression or in the treatment and/or prevention of a disease or disorder selected from the group consisting of transplant rejection, autoimmune diseases, inflammatory diseases, infectious diseases, and lymphoproliferative disorders.
14. The compound for use or the pharmaceutical composition for use of item 13,
   wherein said autoimmune diseases is selected from the group consisting of psoriasis, multiple sclerosis, systemic lupus erythematosus, Hashimoto's thyroiditis, rheumatoid arthritis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis;
   wherein said transplant rejection is selected from the group consisting of acute or chronic rejection of cells, tissue, organ, allografts and xenografts, poor graft functional states, graft versus host disease; rejection of cardiac transplant, skin transplant, renal transplant, liver transplant, islet transplant, pancreas transplant, lung transplant, bowel transplant, corneal transplant, vascular transplant, adrenal transplant, hair transplant, bone transplant, cartilage transplant and ligamental transplant;
   wherein said inflammatory disease is selected from the group consisting of inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, fibrotic disorders, allergic disorders, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome;
   wherein said lymphoproliferative disorder is T cell lymphoma or T cell leukaemia;
   wherein said infectious disease is selected from the group consisting of tuberculosis, preferably caused by mycobacteria, Salmonella sp. infections, Helicobacter sp. infections, retroviral infections, preferably HIV or HTLV, cytomegalo viral infection, candida infection, Staphylococcus infections, lympho-choriomeningitis viral infections and viral hepatitis.
15. The compound for use or the pharmaceutical composition for use of item 13 or 14, wherein the compound of formula (I) inhibits coronin 1 expression.
16. A vector comprising a coronin 1 (coro1a) promoter element, wherein in a vertebrate genome, said coronin 1 promoter element starts directly upstream from a transcription starting site (TSS) of a coronin 1 gene and spans a sequence stretch of at least about 700 bp in said genome.
17. The vector of item 16, further comprising a coronin 1 promoter reporter gene, wherein said coronin 1 promoter element is operably linked to said coronin 1 promoter reporter gene.
18. The vector of item 16 or 17, wherein said coronin 1 promoter element spans a sequence of at least about 700 bp to about 1500 bp in said genome, preferably said coronin 1 promoter element spans a sequence stretch of at least about 700 bp in said genome.
19. The vector of any one of items 16 to 18, wherein said coronin 1 promoter element has an identity of at least 40%, preferably of at least 50%, more preferably of at least 60%, again more preferably of at least 70%, again more preferably of at least 80%, again more preferably of at least 90%, again more preferably of at least 95%, again more preferably of at least 98%, again more preferably of at least 99% with a sequence of SEQ ID NO: 1-6.
20. A method for identifying compounds that modulate coronin 1 promoter activity comprising the steps of:
   a. providing a host cell comprising said vector of any one of the items 15 to 17, wherein said host cell is capable of expressing said promoter reporter genes of said vector;
   b. subjecting said host cells to a compound to be tested; and
   c. measuring expression of said coronin 1 promoter reporter gene in said host cell subjected to said compound to be tested.
21. A method of preparing the compound of formula (I) as defined in item 10, the method comprising the step (b) of asymmetric reduction of the pyridine motive via enantioselective partial transfer hydrogenation.
22. The method of item 21, further comprising the step (e) of aziridine opening via photochemical irradiation (hν).

Further examples and embodiments are disclosed in the following numbered clauses.
1. A compound of formula (I)

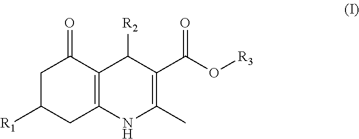

or a pharmaceutically acceptable salt, stereoisomer, diastereoisomer, enantiomer, or isomers and mixtures thereof, for use in the treatment or prevention of a disease that can benefit from BRD3 inhibition, either directly or indirectly via reduced expression of Coronin 1,
   wherein
   R₁ is selected from phenyl and thienyl, wherein said phenyl is optionally substituted with one or more —O—C₁-C₆-alkyl;
   R₂ is selected from phenyl and thienyl, wherein said phenyl is optionally substituted with one or more optional substituents independently selected from —OH, —NO₂, and -halogen;
   R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.
2. The compound for use of clause 1, wherein R₁ is phenyl optionally substituted with methoxy.
3. The compound for use of clause 1 or 2, wherein R₁ is selected from 2-methoxyphenyl and phenyl.
4. The compound for use of any one of clauses 1 to 3, wherein R₂ is 3-hydroxyphenyl.
5. The compound for use of any one of clauses 1 to 4, wherein R₃ is —C₁-C₆-alkylene-(oxygen-containing saturated heterocyclyl) or oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.
6. The compound for use of any one of clauses 1 to 4, wherein R₃ is selected from —C₁-C₆-alkylene-tetrahydro-2-furanyl, C₁-C₆-alkylene-tetrahydro-2H-pyran-4-yl and tetrahydro-2H-pyran-4-yl, wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl and said tetrahydro-2H-pyran-4-yl are each optionally substituted with one or more —C₁-C₆-alkyl.
7. The compound for use of any one of clauses 1 to 6, wherein R₃ is tetrahydro-2H-pyran-4-yl.
8. The compound for use of clause 1, wherein the compound is selected from the group consisting of:
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12),
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13),
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20); and
tert-butyl 4-(3-hydroxyphenyl)-7-(2-methoxyphenyl)-2-methyl-5-oxo-1,4,5,6,7,8-hexahydroquinoline-3-carboxylate (21).
   9. The compound of any one of clauses 1 to 8, wherein the compound of formula (I) has an absolute configuration of its stereogenic centers as shown in the formula:

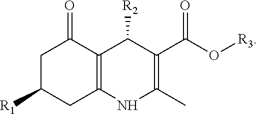

10. The compound for use of any one of clauses 1 to 9, wherein the disease that can benefit from BRD3 inhibition is amenable for therapeutic intervention through direct inhibition of BRD3 or via modulation of coronin 1 expression through BRD3 inhibition, preferably in transplant rejection, autoimmune diseases, inflammatory diseases, infectious diseases, and lymphoproliferative disorders.
11. The compound for use of any one of clauses 1 to 9, wherein the disease that can benefit from BRD3 inhibition is a BRD3-driven malignancy like NMC, OCCC or rhabdomyosarcoma.

### Compounds that Selectively Inhibit Coronin 1 Promoter Activity Identified by the Fluorescence-Based Screening Assay of the Invention

The compounds 1 to 12 and 22 depicted in Table 1 were purchased from ChemBridge Corporation; 11199 Sorrento Valley Rd, Suite 206; San Diego, CA 92121 (USA). The racemic syntheses of the compounds 11 to 21 are described in Example 1.

Diastereoselective synthesis is showcased for compound 13a and its isomers in Example 2.

Asymmetric synthesis is showcased for compound 11c in Example 3.

Syntheses of compounds 12e, 12g, 50a, 50b and 52a to 65a are described in Example 4.

Additional syntheses are showcased by the enantioselective synthesis of compound 51a and syntheses of compounds 66b and 67b in Example 5.

TABLE 1
Compounds 1-22 and 50-67

| | | | | diastereomeric ratio |
|---|---|---|---|---|
| | | | | (dr 4,7-anti:syn)* |
| | | | | determined by ¹H |
| | | | | NMR and |
| | | | | enantiomeric excess |
| | | | | (% ee) determined by |
| Cpd | Chemical | | | HPLC or stereo |
| No. | ID | Structure | Compound Name | comment |
| 1 | 6875817 | 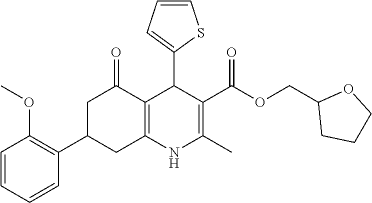 [SMILES: COc1ccccc1C1CC(=O)C2=C(C1)NC(C)=C(C(=O)OCC1CCCO1)C2c1cccs1] | tetrahydro-2- furanylmethyl-7-(2- methoxyphenyl)-2- methyl-5-oxo-4-(2- thienyl)-1,4,5,6,7,8- hexahydro-3- quinolinecarboxylate 1 | 1 dr not defined (no ee) |
| | | 1 | | |
| | | 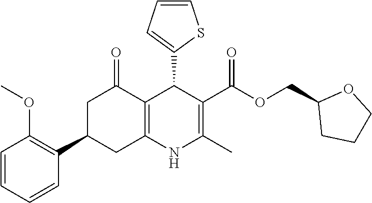 [SMILES: COc1ccccc1[C@H]1CC(=O)C2=C(C1)NC(C)=C(C(=O)OC[C@@H]1CCCO1)[C@H]2c1cccs1] | | |
| | | 1a | | |
| | | 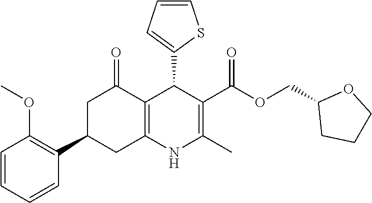 [SMILES: COc1ccccc1[C@H]1CC(=O)C2=C(C1)NC(C)=C(C(=O)OC[C@H]1CCCO1)[C@H]2c1cccs1] | | |
| | | 1b | | |
| 2 | 6875715 | 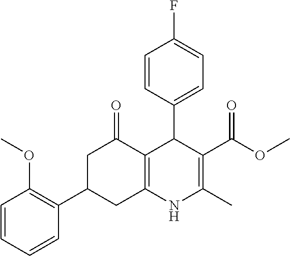 | methyl-4-(4- fluorophenyl)-7-(2- methoxyphenyl)-2- methyl-5-oxo- 1,4,5,6,7,8- hexahydro-3- quinolinecarboxylate | 2 dr not defined (no ee) |
| | | 2 | | |
| | | 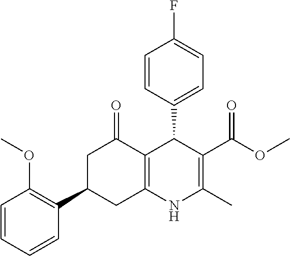 | | |
| | | 2a | | |
| 3 | 6875191 | 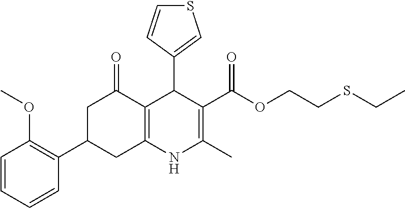 [SMILES: CCSCCOC(=O)C1=C(C)NC2=C(C(=O)CC(c3ccccc3OC)C2)C1c1ccsc1] | 2-(ethylthio)ethyl-7- (2-methoxyphenyl)- 2-methyl-5-oxo-4- (3-thienyl)- 1,4,5,6,7,8- hexahydro-3- quinolinecarboxylate | 3 dr not defined (no ee) |
| | | 3 | | |
| | | 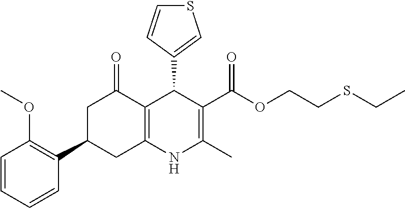 [SMILES: CCSCCOC(=O)C1=C(C)NC2=C(C(=O)C[C@H](c3ccccc3OC)C2)[C@@H]1c1ccsc1] | | |
| | | 3a | | |
| 4 | 5713172 | 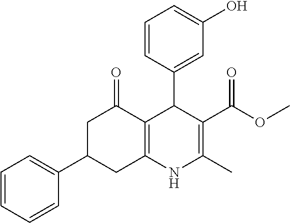 [SMILES: COC(=O)C1=C(C)NC2=C(C(=O)CC(c3ccccc3)C2)C1c1cccc(O)c1] | methyl 4-(3- hydroxyphenyl)-2- methyl-5-oxo-7- phenyl-1,4,5,6,7,8- hexahydroquinoline- 3-carboxylate | 4 dr not defined (no ee) |
| | | 4 | | |
| | | 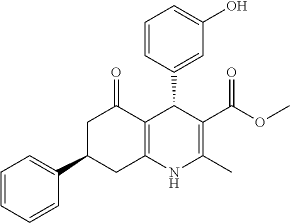 [SMILES: COC(=O)C1=C(C)NC2=C(C(=O)C[C@H](c3ccccc3)C2)[C@@H]1c1cccc(O)c1] | | |
| | | 4a | | |
| 5 | 6431706 | 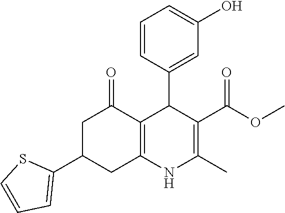 [SMILES: COC(=O)C1=C(C)NC2=C(C(=O)CC(c3cccs3)C2)C1c1cccc(O)c1] | methyl-4-(3- hydroxyphenyl)-2- methyl-5-oxo-7-(2- thienyl)-1,4,5,6,7,8- hexahydro-3- quinolinecarboxylate | 5 dr not defined (no ee) |
| | | 5 | | |
| | | 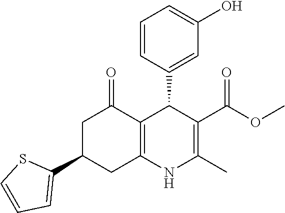 [SMILES: COC(=O)C1=C(C)NC2=C(C(=O)C[C@H](c3cccs3)C2)[C@@H]1c1cccc(O)c1] | | |
| | | 5a | | |
| 6 | 6631152 | 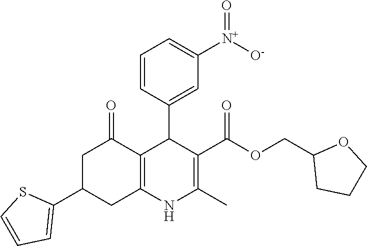 [SMILES: CC1=C(C(=O)OCC2CCCO2)C(c2cccc([N+](=O)[O-])c2)C2=C(CC(c3cccs3)CC2=O)N1] | tetrahydro-2- furanylmethyl-2- methyl-4-(3- nitrophenyl)-5-oxo- 7-(2-thienyl)- 1,4,5,6,7,8- hexahydro-3- quinolinecarboxylate | 6 dr not defined (no ee) |
| | | 6 | | |
| | | 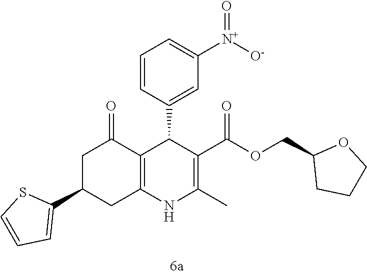 [SMILES: CC1=C(C(=O)OC[C@@H]2CCCO2)[C@@H](c2cccc([N+](=O)[O-])c2)C2=C(C[C@@H](c3cccs3)CC2=O)N1] | | |
| | | 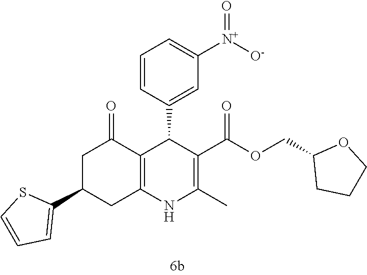 [SMILES: CC1=C(C(=O)OC[C@H]2CCCO2)[C@@H](c2cccc([N+](=O)[O-])c2)C2=C(C[C@@H](c3cccs3)CC2=O)N1] | | |
| 7 | 6625673 | 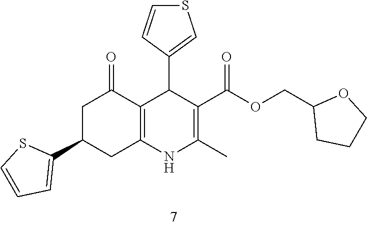 [SMILES: CC1=C(C(=O)OCC2CCCO2)C(c2ccsc2)C2=C(C[C@@H](c3cccs3)CC2=O)N1] | tetrahydro-2- furanylmethyl-2- methyl-5-oxo-7-(2- thienyl)-4-(3- thienyl)-1,4,5,6,7,8- hexahydro-3- quinolinecarboxylate | 7 dr not defined (no ee) |
| | | 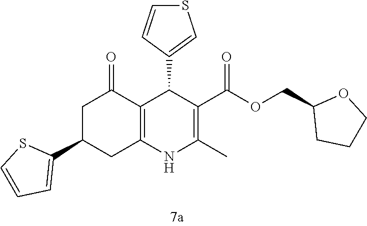 [SMILES: CC1=C(C(=O)OC[C@@H]2CCCO2)[C@@H](c2ccsc2)C2=C(C[C@@H](c3cccs3)CC2=O)N1] | | |
| | | 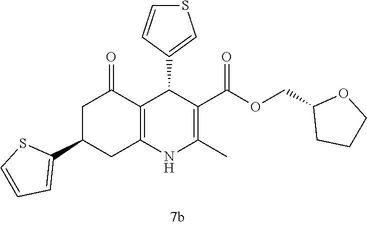 [SMILES: CC1=C(C(=O)OC[C@H]2CCCO2)[C@@H](c2ccsc2)C2=C(C[C@@H](c3cccs3)CC2=O)N1] | | |
| 8 | 5707813 | 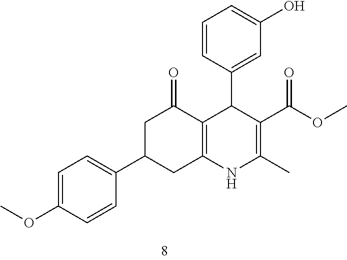 | methyl-4-(3- hydroxyphenyl)-7- (4-methoxyphenyl)- 2-methyl-5-oxo- 1,4,5,6,7,8- hexahydro-3- quinolinecarboxylate | 8 dr not defined (no ee) |
| | | 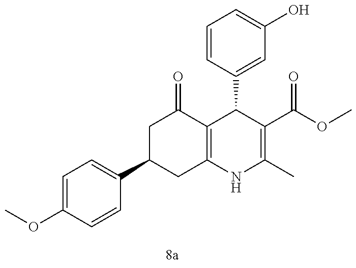 | | |
| 9 | 6874651 | 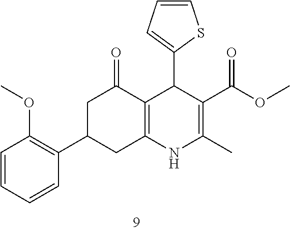 [SMILES: COC(=O)C1=C(C)NC2=C(C(=O)CC(c3ccccc3OC)C2)C1c1cccs1] | methyl-7-(2- methoxyphenyl)-2- methyl-5-oxo-4-(2- thienyl)-1,4,5,6,7,8- hexahydro-3- quinolinecarboxylate | 9 dr not defined (no ee) |
| | | 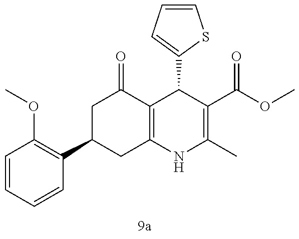 [SMILES: COC(=O)C1=C(C)NC2=C(C(=O)C[C@H](c3ccccc3OC)C2)[C@@H]1c1cccs1] | | |
| 10 | 6943117 | 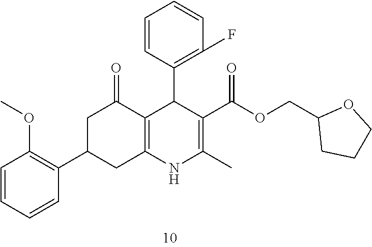 [SMILES: COc1ccccc1C1CC(=O)C2=C(C1)NC(C)=C(C(=O)OCC1CCCO1)C2c1ccccc1F] | tetrahydro-2- furanylmethyl-4-(2- fluorophenyl)-7-(2- methoxyphenyl)-2- methyl-5-oxo- 1,4,5,6,7,8- hexahydro-3- quinolinecarboxylate | 10 dr not defined (no ee) |
| | | 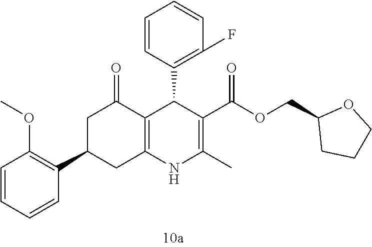 [SMILES: COc1ccccc1[C@H]1CC(=O)C2=C(C1)NC(C)=C(C(=O)OC[C@@H]1CCCO1)[C@H]2c1ccccc1F] | | |
| | | 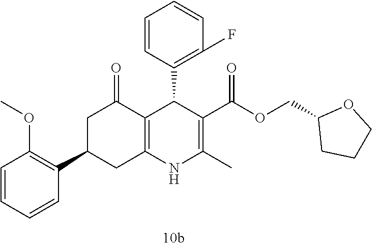 [SMILES: COc1ccccc1[C@H]1CC(=O)C2=C(C1)NC(C)=C(C(=O)OC[C@H]1CCCO1)[C@H]2c1ccccc1F] | | |
| 11 | 6945999 | 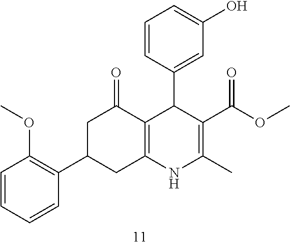 [SMILES: COC(=O)C1=C(C)NC2=C(C(=O)CC(c3ccccc3OC)C2)C1c1cccc(O)c1] | methyl-4-(3- hydroxyphenyl)-7- (2-methoxyphenyl)- 2-methyl-5-oxo- 1,4,5,6,7,8- hexahydro-3- quinolinecarboxylate | 11 dr 1.0:1.3 (no ee) 11a 4,7-anti dr 1.0:0.0 (no ee) 11b 4,7-syn dr 0.0:1.0 (no ee) 11c (4S,7R) ddr 1.0:0.0 (≥96% ee) 11d (4R,7s) dr 1.0:0.0 (≥96% ee) 11e (7R) dr 1.0:1.0 (≥96% ee) |
| | | 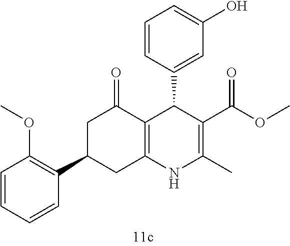 [SMILES: COC(=O)C1=C(C)NC2=C(C(=O)C[C@H](c3ccccc3OC)C2)[C@@H]1c1cccc(O)c1] | | |
| 12 | 29007 | 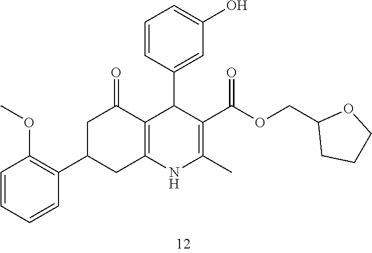 [SMILES: COc1ccccc1C1CC(=O)C2=C(C1)NC(C)=C(C(=O)OCC1CCCO1)C2c1cccc(O)c1] | tetrahydro-2- furanylmethyl-4-(3- hydroxyphenyl)-7- (2-methoxyphenyl)- 2-methyl-5-oxo- 1,4,5,6,7,8- hexahydro-3- quinolinecarboxylate | 12 not defined (no ee) 12a 4,7-anti dr 1.0:1.0:0.0:0.0 (no ee) 12b 4,7-syn dr 0.0:0.0:1.0:1.0 (no ee) 12c (S) dr 1.0:0.0:1.0:0.0 (≥96% ee) 12d (R) dr 0.0:1.0:0.0:1.0 (≥96% ee) 12e (S,4S,7R) dr 1.0:0.0:0.0:0.0 (≥96% ee) 12f (S,4R,7S) dr 0.0:0.0:1.0:0.0 (≥96% |
| | | 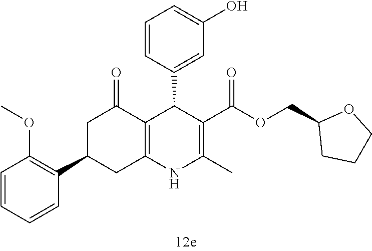 [SMILES: COc1ccccc1[C@H]1CC(=O)C2=C(C1)NC(C)=C(C(=O)OC[C@@H]1CCCO1)[C@H]2c1cccc(O)c1] | | ee) 12g (R,4S,7R) single isomer |
| | | 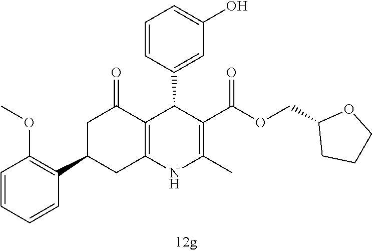 [SMILES: COc1ccccc1[C@H]1CC(=O)C2=C(C1)NC(C)=C(C(=O)OC[C@H]1CCCO1)[C@H]2c1cccc(O)c1] | | |
| 13 | — | 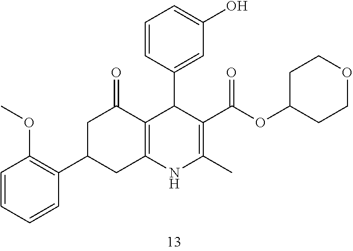 [SMILES: COc1ccccc1C1CC(=O)C2=C(C1)NC(C)=C(C(=O)OC1CCOCC1)C2c1cccc(O)c1] | tetrahydro-2H- pyran-4-yl-4-(3- hydroxyphenyl)-7- (2-methoxyphenyl)- 2-methyl-5-oxo- 1,4,5,6,7,8- hexahydro-3- quinolinecarboxylate | 13 dr 5.0:1.0 (no ee) 13a (4S,7R) dr 1.0:0.0 (≥98% ee) 13b dr 1.0:1.0 (no ee) 13c (4S) dr 1.0:1.0 (≥96% ee) 13d (4R,7S) dr 1.0:0.0 (≥96% ee) 13e (4S,7S) dr 0.0:1.0 (≥96% ee) 13f (4R,7R) dr 0.0:1.0 (≥96% ee) |
| | | 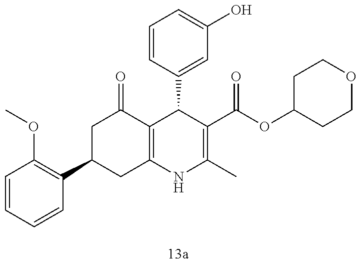 [SMILES: COc1ccccc1[C@H]1CC(=O)C2=C(C1)NC(C)=C(C(=O)OC1CCOCC1)[C@H]2c1cccc(O)c1] | | |
| 14 | — | 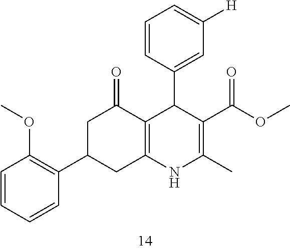 | methyl 7-(2- methoxyphenyl)-2- methyl-5-oxo-4- phenyl-1,4,5,6,7,8- hexahydroquinoline- 3-carboxylate | 14 dr > 20:1 |
| | | 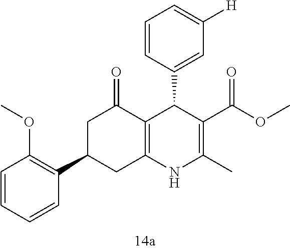 | | |
| 15 | — | 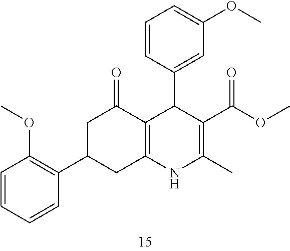 | methyl 7-(2- methoxyphenyl)-4- (3-methoxyphenyl)- 2-methyl-5-oxo- 1,4,5,6,7,8- hexahydroquinoline- 3-carboxylate | 15 dr 4.4:1.0 |
| | | 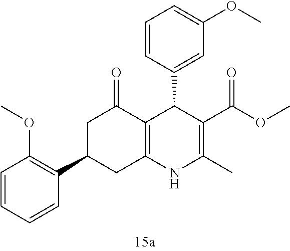 | | |
| 16 | — | 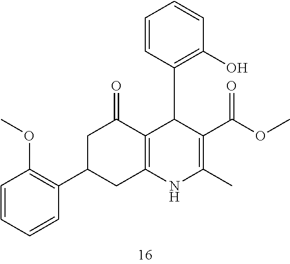 | methyl 4-(2- hydroxyphenyl)-7- (2-methoxyphenyl)- 2-methyl-5-oxo- 1,4,5,6,7,8- hexahydroquinoline- 3-carboxylate | 16 dr 6.7:1.0 |
| | | 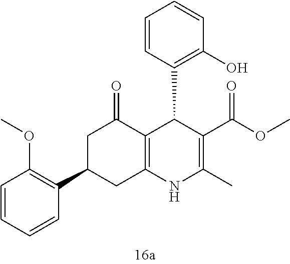 | | |
| 17 | — | 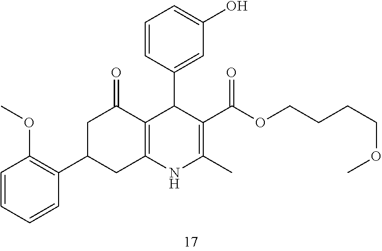 [SMILES: COCCCCOC(=O)C1=C(C)NC2=C(C(=O)CC(c3ccccc3OC)C2)C1c1cccc(O)c1] | 4-methoxybutyl 4- (3-hydroxyphenyl)- 7-(2- methoxyphenyl)-2- methyl-5-oxo- 1,4,5,6,7,8- hexahydroquinoline- 3-carboxylate | 17 dr 5.0:1.0 |
| | | 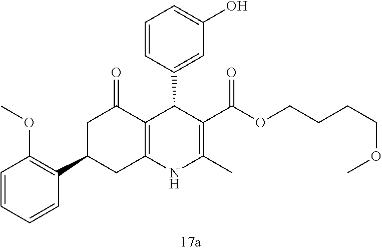 [SMILES: COCCCCOC(=O)C1=C(C)NC2=C(C(=O)C[C@H](c3ccccc3OC)C2)[C@@H]1c1cccc(O)c1] | | |
| 18 | — | 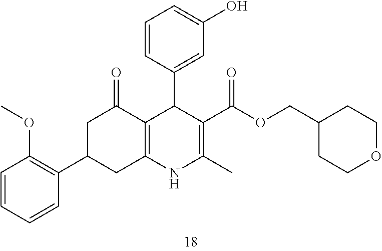 [SMILES: COc1ccccc1C1CC(=O)C2=C(C1)NC(C)=C(C(=O)OCC1CCOCC1)C2c1cccc(O)c1] | (tetrahydro-2H- pyran-4-yl)methyl 4- (3-hydroxyphenyl)- 7-(2- methoxyphenyl)-2- methyl-5-oxo- 1,4,5,6,7,8- hexahydroquinoline- 3-carboxylate | 18 dr 5.3:1.0 |
| | | 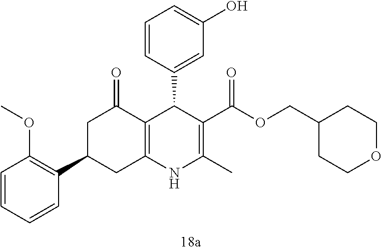 [SMILES: COc1ccccc1[C@H]1CC(=O)C2=C(C1)NC(C)=C(C(=O)OCC1CCOCC1)[C@H]2c1cccc(O)c1] | | |
| 19 | — | 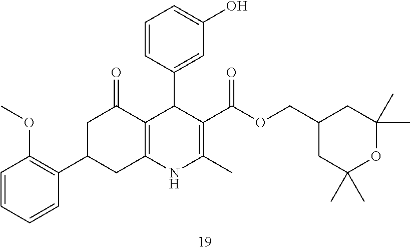 | (2,2,6,6- tetramethyltetrahydro- 2H-pyran-4- yl)methyl 4-(3- hydroxyphenyl)-7- (2-methoxyphenyl)- 2-methyl-5-oxo- 1,4,5,6,7,8- hexahydroquinoline- 3-carboxylate | 19 dr 7.7:1.0 |
| | | 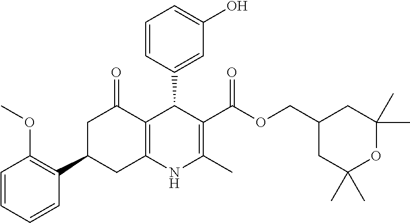 [SMILES: COc1ccccc1[C@H]1CC(=O)C2=C(C1)NC(C)=C(C(=O)OCC1CC(C)(C)OC(C)(C)C1)[C@H]2c1cccc(O)c1] | | |
| | | 19a | | |
| 20 | — | 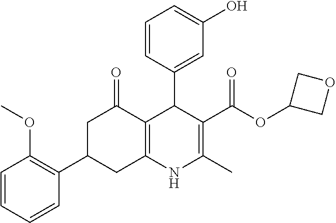 [SMILES: COc1ccccc1C1CC(=O)C2=C(C1)NC(C)=C(C(=O)OC1COC1)C2c1cccc(O)c1] | oxetan-3-yl 4-(3- hydroxyphenyl)-7- (2-methoxyphenyl)- 2-methyl-5-oxo- 1,4,5,6,7,8- hexahydroquinoline- 3-carboxylate | 20 dr 3.8:1.0 |
| | | 20 | | |
| | | 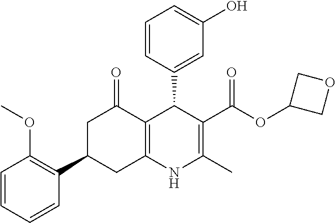 [SMILES: COc1ccccc1[C@H]1CC(=O)C2=C(C1)NC(C)=C(C(=O)OC1COC1)[C@H]2c1cccc(O)c1] | | |
| | | 20a | | |
| 21 | — | 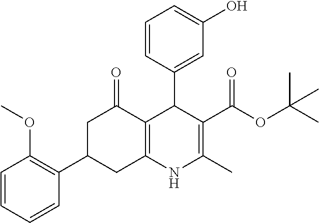 [SMILES: COc1ccccc1C1CC(=O)C2=C(C1)NC(C)=C(C(=O)OC(C)(C)C)C2c1cccc(O)c1] | tert-butyl 4-(3- hydroxyphenyl)-7- (2-methoxyphenyl)- 2-methyl-5-oxo- 1,4,5,6,7,8- hexahydroquinoline- 3-carboxylate | 21 dr 5.9:1.0 |
| | | 21 | | |
| | | 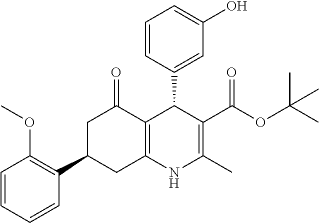 [SMILES: COc1ccccc1[C@H]1CC(=O)C2=C(C1)NC(C)=C(C(=O)OC(C)(C)C)[C@H]2c1cccc(O)c1] | | |
| | | 21a | | |
| 22 | 5722562 | 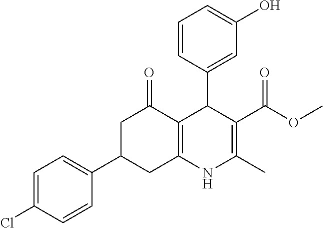 [SMILES: COC(=O)C1=C(C)NC2=C(C(=O)CC(c3ccc(Cl)cc3)C2)C1c1cccc(O)c1] | methyl 7-(4- chlorophenyl)-4-(3- hydroxyphenyl)-2- methyl-5-oxo- 1,4,5,6,7,8- hexahydroquinoline- 3-carboxylate | 22 dr not defined (no ee) |
| | | 22 | | |
| | | 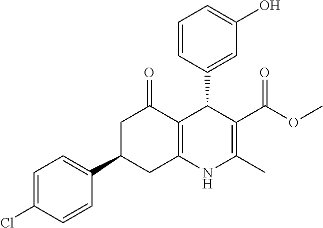 [SMILES: COC(=O)C1=C(C)NC2=C(C(=O)C[C@H](c3ccc(Cl)cc3)C2)[C@@H]1c1cccc(O)c1] | | |
| | | 22a | | |
| 50 | NXI- 000225 NXI- 000224 | 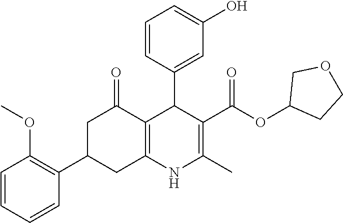 [SMILES: COc1ccccc1C1CC(=O)C2=C(C1)NC(C)=C(C(=O)OC1CCOC1)C2c1cccc(O)c1] | tetrahydrofuran-3-yl 4-(3- hydroxyphenyl)-7- (2-methoxyphenyl)- 2-methyl-5-oxo- 1,4,5,6,7,8- hexahydroquinoline- 3-carboxylate | 50a (S,4S,7R) single isomer 50b (R,4S,7R) single isomer |
| | | 50 | | |
| | | 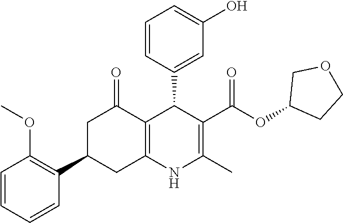 [SMILES: COc1ccccc1[C@H]1CC(=O)C2=C(C1)NC(C)=C(C(=O)O[C@H]1CCOC1)[C@H]2c1cccc(O)c1] | | |
| | | 50a | | |
| | | 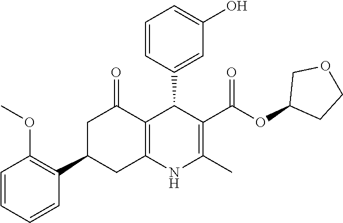 [SMILES: COc1ccccc1[C@H]1CC(=O)C2=C(C1)NC(C)=C(C(=O)O[C@@H]1CCOC1)[C@H]2c1cccc(O)c1] | | |
| | | 50b | | |
| 51 | NXI- 000280 | 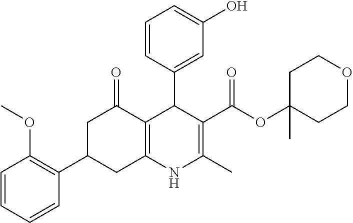 [SMILES: COc1ccccc1C1CC(=O)C2=C(C1)NC(C)=C(C(=O)OC1(C)CCOCC1)C2c1cccc(O)c1] | 4-methyltetrahydro- 2H-pyran-4-yl 4-(3- hydroxyphenyl)-7- (2-methoxyphenyl)- 2-methyl-5-oxo- 1,4,5,6,7,8- hexahydroquinoline- 3-carboxylate | 51a (4S,7R) single isomer 51b (7R) dr 1.0:1.0 |
| | | 51 | | |
| | | 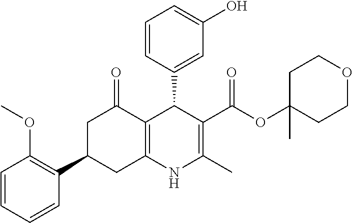 [SMILES: COc1ccccc1[C@H]1CC(=O)C2=C(C1)NC(C)=C(C(=O)OC1(C)CCOCC1)[C@H]2c1cccc(O)c1] | | |
| | | 51a | | |
| 52 | NXI- 000202 | 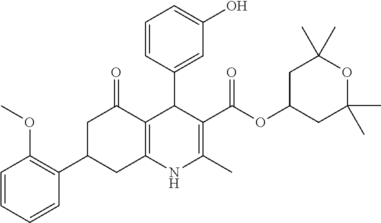 [SMILES: COc1ccccc1C1CC(=O)C2=C(C1)NC(C)=C(C(=O)OC1CC(C)(C)OC(C)(C)C1)C2c1cccc(O)c1] | 2,2,6,6- tetramethyltetrahydro- 2H-pyran-4-yl 4- (3-hydroxyphenyl)- 7-(2- methoxyphenyl)-2- methyl-5-oxo- 1,4,5,6,7,8- hexahydroquinoline- 3-carboxylate | 52a (4S,7R) single isomer |
| | | 52 | | |
| | | 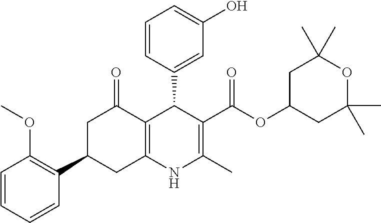 [SMILES: COc1ccccc1[C@H]1CC(=O)C2=C(C1)NC(C)=C(C(=O)OC1CC(C)(C)OC(C)(C)C1)[C@H]2c1cccc(O)c1] | | |
| | | 52a | | |
| 53 | NXI- 000201 | 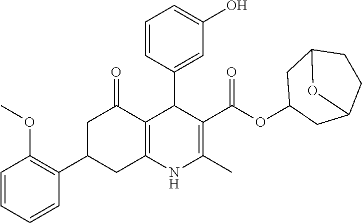 [SMILES: COc1ccccc1C1CC(=O)C2=C(C1)NC(C)=C(C(=O)OC1CC3CCC(C1)O3)C2c1cccc(O)c1] | 8-oxa- bicyclo[3.2.1]octan- 3-yl 4-(3- hydroxyphenyl)-7- (2-methoxyphenyl)- 2-methyl-5-oxo- 1,4,5,6,7,8- hexahydroquinoline- 3-carboxylate | 53a (4S,7R) single isomer |
| | | 53 | | |
| | | 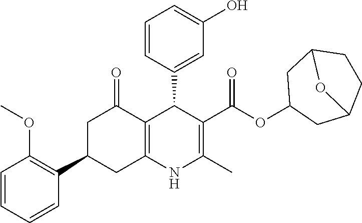 [SMILES: COc1ccccc1[C@H]1CC(=O)C2=C(C1)NC(C)=C(C(=O)OC1CC3CCC(C1)O3)[C@H]2c1cccc(O)c1] | | |
| | | 53a | | |
| 54 | NXI- 000294 | 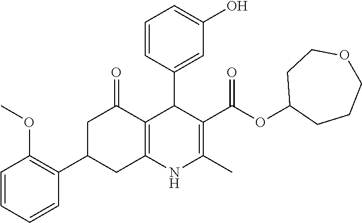 [SMILES: COc1ccccc1C1CC(=O)C2=C(C1)NC(C)=C(C(=O)OC1CCCOCC1)C2c1cccc(O)c1] | oxepan-4-yl 4-(3- hydroxyphenyl)-7- (2-methoxyphenyl)- 2-methyl-5-oxo- 1,4,5,6,7,8- hexahydroquinoline- 3-carboxylate | 54a (4S,7R) mixture of isomers |
| | | 54 | | |
| | | 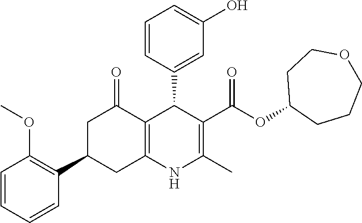 [SMILES: COc1ccccc1[C@H]1CC(=O)C2=C(C1)NC(C)=C(C(=O)O[C@H]1CCCOCC1)[C@H]2c1cccc(O)c1] | | |
| | | 54b | | |
| | | 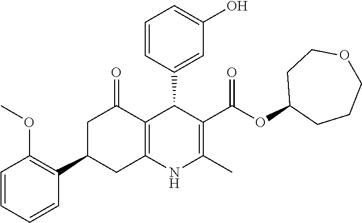 [SMILES: COc1ccccc1[C@H]1CC(=O)C2=C(C1)NC(C)=C(C(=O)O[C@@H]1CCCOCC1)[C@H]2c1cccc(O)c1] | | |
| | | 54c | | |
| 55 | NXI- 000248 | 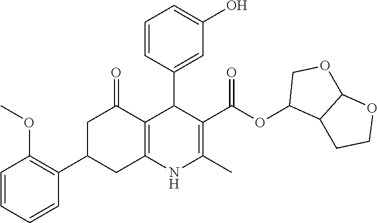 [SMILES: COc1ccccc1C1CC(=O)C2=C(C1)NC(C)=C(C(=O)OC1COC3OCCC13)C2c1cccc(O)c1] | hexahydrofuro[2,3- b]furan-3-yl (4-(3- hydroxyphenyl)-7- (2-methoxyphenyl)- 2-methyl-5-oxo- 1,4,5,6,7,8- hexahydroquinoline- 3-carboxylate | 55a (3R,3aS,6aR)- (4S,7R) single isomer |
| | | 55 | | |
| | | 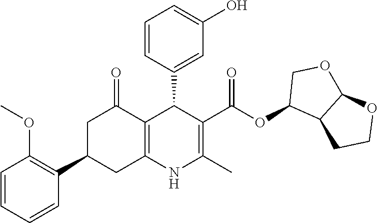 [SMILES: COc1ccccc1[C@H]1CC(=O)C2=C(C1)NC(C)=C(C(=O)O[C@H]1CO[C@H]3OCC[C@H]31)[C@H]2c1cccc(O)c1] | | |
| | | 55a | | |
| 56 | NXI- 000281 | 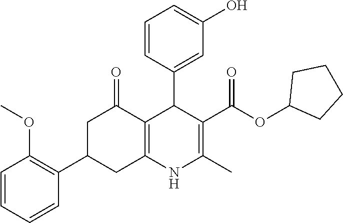 [SMILES: COc1ccccc1C1CC(=O)C2=C(C1)NC(C)=C(C(=O)OC1CCCC1)C2c1cccc(O)c1] | cyclopentyl 4-(3- hydroxyphenyl)-7- (2-methoxyphenyl)- 2-methyl-5-oxo- 1,4,5,6,7,8- hexahydroquinoline- 3-carboxylate | 56a (4S,7R) single isomer |
| | | 56 | | |
| | | 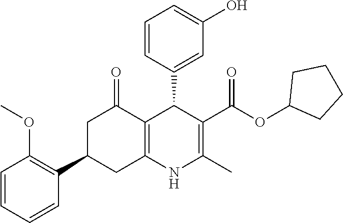 [SMILES: COc1ccccc1[C@H]1CC(=O)C2=C(C1)NC(C)=C(C(=O)OC1CCCC1)[C@H]2c1cccc(O)c1] | | |
| | | 56a | | |
| 57 | NXI- 000269 | 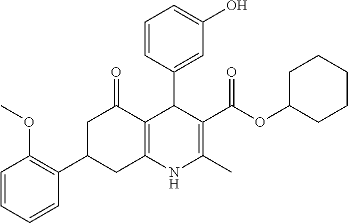 [SMILES: COc1ccccc1C1CC(=O)C2=C(C1)NC(C)=C(C(=O)OC1CCCCC1)C2c1cccc(O)c1] | cyclohexyl 4-(3- hydroxyphenyl)-7- (2-methoxyphenyl)- 2-methyl-5-oxo- 1,4,5,6,7,8- hexahydroquinoline- 3-carboxylate | 57a (4S,7R) single isomer |
| | | 57 | | |
| | | 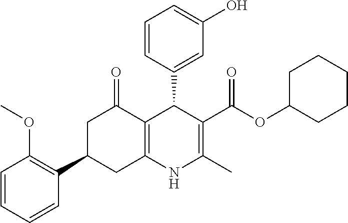 [SMILES: COc1ccccc1[C@H]1CC(=O)C2=C(C1)NC(C)=C(C(=O)OC1CCCCC1)[C@H]2c1cccc(O)c1] | | |
| | | 57a | | |
| 58 | NXI- 000282 | 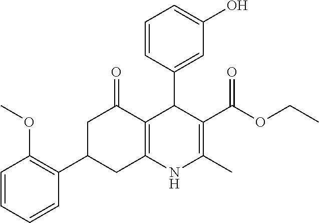 [SMILES: CCOC(=O)C1=C(C)NC2=C(C(=O)CC(c3ccccc3OC)C2)C1c1cccc(O)c1] | ethyl 4-(3- hydroxyphenyl)-7- (2-methoxyphenyl)- 2-methyl-5-oxo- 1,4,5,6,7,8- hexahydroquinoline- 3-carboxylate | 58a (4S,7R) single isomer |
| | | 58 | | |
| | | 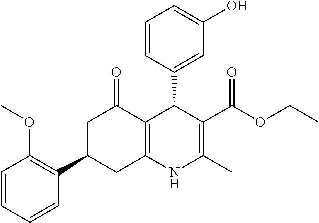 [SMILES: CCOC(=O)C1=C(C)NC2=C(C(=O)C[C@H](c3ccccc3OC)C2)[C@@H]1c1cccc(O)c1] | | |
| | | 58a | | |
| 59 | NXI- 000283 | 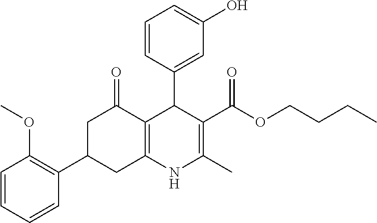 [SMILES: CCCCOC(=O)C1=C(C)NC2=C(C(=O)CC(c3ccccc3OC)C2)C1c1cccc(O)c1] | butyl 4-(3- hydroxyphenyl)-7- (2-methoxyphenyl)- 2-methyl-5-oxo- 1,4,5,6,7,8- hexahydroquinoline- 3-carboxylate | 59a (4S,7R) single isomer |
| | | 59 | | |
| | | 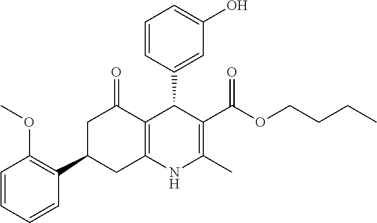 [SMILES: CCCCOC(=O)C1=C(C)NC2=C(C(=O)C[C@H](c3ccccc3OC)C2)[C@@H]1c1cccc(O)c1] | | |
| | | 59a | | |
| 60 | NXI- 000289 | 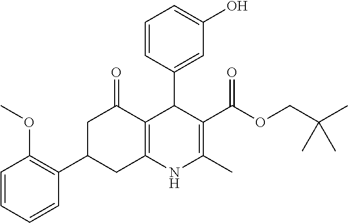 [SMILES: COc1ccccc1C1CC(=O)C2=C(C1)NC(C)=C(C(=O)OCC(C)(C)C)C2c1cccc(O)c1] | neopentyl 4-(3- hydroxyphenyl)-7- (2-methoxyphenyl)- 2-methyl-5-oxo- 1,4,5,6,7,8- hexahydroquinoline- 3-carboxylate | 60a (4S,7R) single isomer |
| | | 60 | | |
| | | 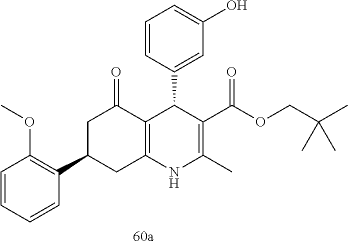 [SMILES: COc1ccccc1[C@H]1CC(=O)C2=C(C1)NC(C)=C(C(=O)OCC(C)(C)C)[C@H]2c1cccc(O)c1] | | |
| 61 | NXI- 000290 | 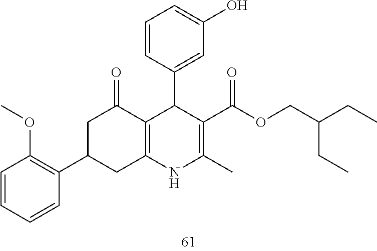 [SMILES: CCC(CC)COC(=O)C1=C(C)NC2=C(C(=O)CC(c3ccccc3OC)C2)C1c1cccc(O)c1] | 2-ethylbutyl 4-(3- hydroxyphenyl)-7- (2-methoxyphenyl)- 2-methyl-5-oxo- 1,4,5,6,7,8- hexahydroquinoline- 3-carboxylate | 61a (4S,7R) single isomer |
| | | 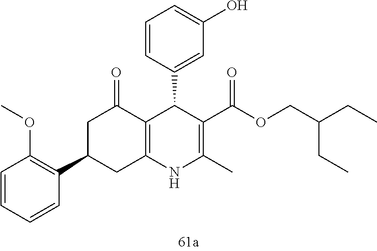 [SMILES: CCC(CC)COC(=O)C1=C(C)NC2=C(C(=O)C[C@H](c3ccccc3OC)C2)[C@@H]1c1cccc(O)c1] | | |
| 62 | NXI- 000293 | 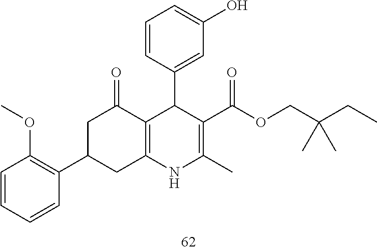 [SMILES: CCC(C)(C)COC(=O)C1=C(C)NC2=C(C(=O)CC(c3ccccc3OC)C2)C1c1cccc(O)c1] | 2,2-dimethylbutyl 4- (3-hydroxyphenyl)- 7-(2- methoxyphenyl)-2- methyl-5-oxo- 1,4,5,6,7,8- hexahydroquinoline- 3-carboxylate | 62a (4S,7R) single isomer |
| | | 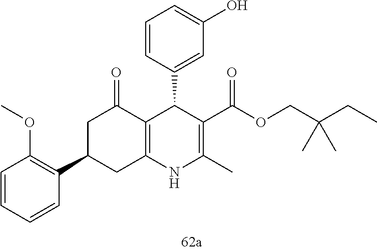 [SMILES: CCC(C)(C)COC(=O)C1=C(C)NC2=C(C(=O)C[C@H](c3ccccc3OC)C2)[C@@H]1c1cccc(O)c1] | | |
| 63 | NXI- 000295 | 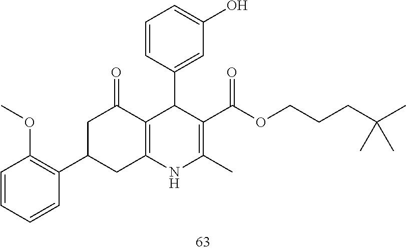 [SMILES: COc1ccccc1C1CC(=O)C2=C(C1)NC(C)=C(C(=O)OCCCC(C)(C)C)C2c1cccc(O)c1] | 4,4-dimethylpentyl 4-(3- hydroxyphenyl)-7- (2-methoxyphenyl)- 2-methyl-5-oxo- 1,4,5,6,7,8- hexahydroquinoline- 3-carboxylate | 63a (4S,7R) single isomer |
| | | 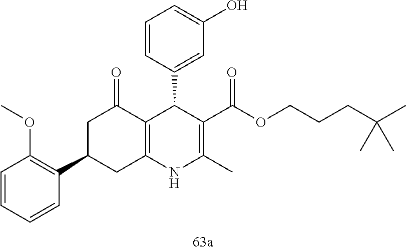 [SMILES: COc1ccccc1[C@H]1CC(=O)C2=C(C1)NC(C)=C(C(=O)OCCCC(C)(C)C)[C@H]2c1cccc(O)c1] | | |
| 64 | NXI- 000297 | 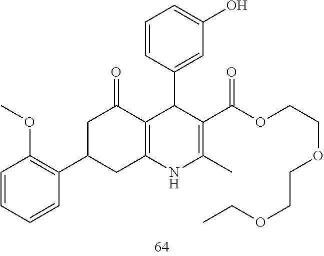 [SMILES: CCOCCOCCOC(=O)C1=C(C)NC2=C(C(=O)CC(c3ccccc3OC)C2)C1c1cccc(O)c1] | 2-(2- ethoxyethoxy)ethyl 4-(3- hydroxyphenyl)-7- (2-methoxyphenyl)- 2-methyl-5-oxo- 1,4,5,6,7,8- hexahydroquinoline- 3-carboxylate | 64a (4S,7R) single isomer |
| | | 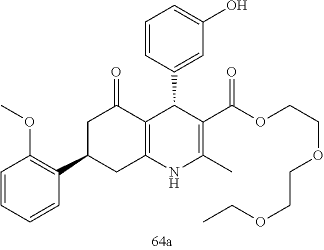 [SMILES: CCOCCOCCOC(=O)C1=C(C)NC2=C(C(=O)C[C@H](c3ccccc3OC)C2)[C@@H]1c1cccc(O)c1] | | |
| 65 | NXI- 000296 | 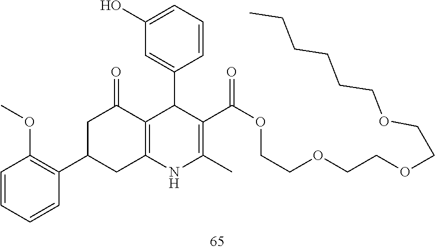 [SMILES: CCCCCCOCCOCCOCCOC(=O)C1=C(C)NC2=C(C(=O)CC(c3ccccc3OC)C2)C1c1cccc(O)c1] | 2-(2-(2- (hexyloxy)ethoxy)eth- oxy)ethyl 4-(3- hydroxyphenyl)-7- (2-methoxyphenyl)- 2-methyl-5-oxo- 1,4,5,6,7,8- hexahydroquinoline- 3-carboxylate | 65a (4S,7R) single isomer |
| | | 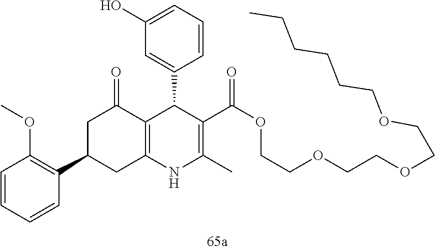 [SMILES: CCCCCCOCCOCCOCCOC(=O)C1=C(C)NC2=C(C(=O)C[C@H](c3ccccc3OC)C2)[C@@H]1c1cccc(O)c1] | | |
| 66 | NXI- 000298 | 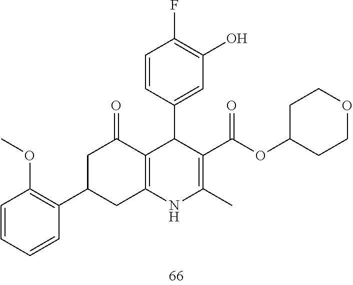 [SMILES: COc1ccccc1C1CC(=O)C2=C(C1)NC(C)=C(C(=O)OC1CCOCC1)C2c1ccc(F)c(O)c1] | tetrahydro-2H- pyran-4-yl 4-(4- fluoro-3- hydroxyphenyl)-7- (2-methoxyphenyl)- 2-methyl-5-oxo- 1,4,5,6,7,8- hexahydroquinoline- 3-carboxylate | 66b (7R) dr 1.0:1.0 |
| | | 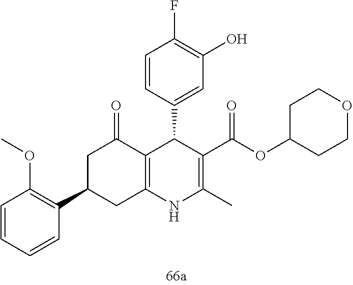 [SMILES: COc1ccccc1[C@H]1CC(=O)C2=C(C1)NC(C)=C(C(=O)OC1CCOCC1)[C@H]2c1ccc(F)c(O)c1] | | |
| 67 | NXI- 000303 | 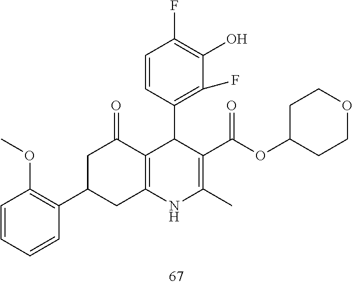 [SMILES: COc1ccccc1C1CC(=O)C2=C(C1)NC(C)=C(C(=O)OC1CCOCC1)C2c1ccc(F)c(O)c1F] | tetrahydro-2H- pyran-4-yl 4-(2,4- difluoro-3- hydroxyphenyl)-7- (2-methoxyphenyl)- 2-methyl-5-oxo- 1,4,5,6,7,8- hexahydroquinoline- 3-carboxylate | 67b (7R) dr 1.0:1.0 |
| | | 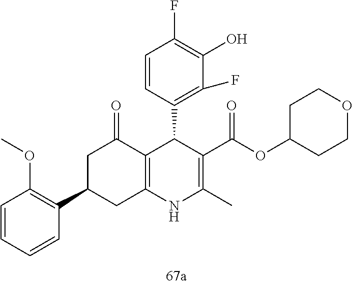 [SMILES: COc1ccccc1[C@H]1CC(=O)C2=C(C1)NC(C)=C(C(=O)OC1CCOCC1)[C@H]2c1ccc(F)c(O)c1F] | | |

*For [(R/S)-tetrahydrofuran-2-yl)methyl containing compounds dr are defined as (D1)-4,7-anti:(D2)-4,7-anti:(D1)-4,7-syn:(D2)-4,7-syn

### Example Intermediates for the Synthesis of Compounds that Selectively Inhibit Coronin 1 Promoter Activity

TABLE 2
Intermediates 27, 29, 31-38 and 70-74

| Cpd | | |
|---|---|---|
| No. | Structure | Compound Name |
| 27 | 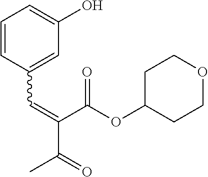 [SMILES: CC(=O)C(=Cc1cccc(O)c1)C(=O)OC1CCOCC1] | tetrahydro-2H-pyran-4-yl 2-(3- hydroxybenzylidene)-3-oxobutanoate |
| 29 | 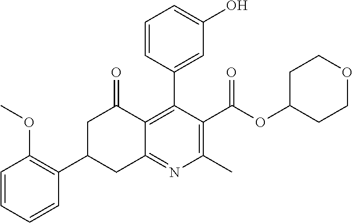 [SMILES: COc1ccccc1C1CC(=O)c2c(nc(C)c(C(=O)OC3CCOCC3)c2-c2cccc(O)c2)C1] | tetrahydro-2H-pyran-4-yl 4-(3- hydroxyphenyl)-7-(2-methoxyphenyl)- 2-methyl-5-oxo-5,6,7,8- tetrahydroquinoline-3-carboxylate |
| 31 | 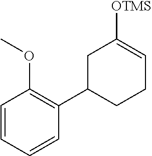 | ((2′-methoxy-1,2,5,6-tetrahydro-[1,1′- biphenyl]-3-yl)oxy)trimethylsilane |
| 32 | 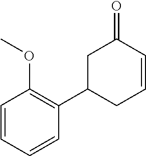 | 2′-methoxy-1,6-dihydro-[1,1′-biphenyl]- 3(2H)-one |
| 33 | 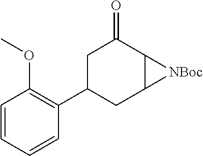 [SMILES: COc1ccccc1C1CC(=O)C2C(C1)N2C(=O)OC(C)(C)C] | tert-butyl 4-(2-methoxyphenyl)-2-oxo- 7-azabicyclo[4.1.0]heptane-7- carboxylate |
| 34 | 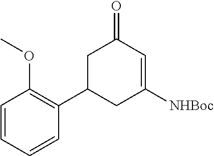 [SMILES: COc1ccccc1C1CC(=O)C=C(NC(=O)OC(C)(C)C)C1] | tert-butyl (2′-methoxy-5-oxo-1,2,5,6- tetrahydro-[1,1′-biphenyl]-3- yl)carbamate |
| 35 | 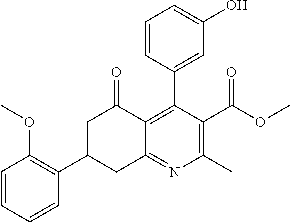 [SMILES: COC(=O)c1c(C)nc2c(c1-c1cccc(O)c1)C(=O)CC(c1ccccc1OC)C2] | methyl 4-(3-hydroxyphenyl)-7-(2- methoxyphenyl)-2-methyl-5-oxo- 5,6,7,8-tetrahydroquinoline-3- carboxylate |
| 36 | 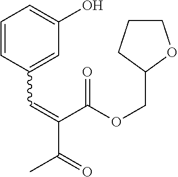 [SMILES: CC(=O)C(=Cc1cccc(O)c1)C(=O)OCC1CCCO1] | (tetrahydrofuran-2-yl)methyl 2-(3- hydroxybenzylidene)-3-oxobutanoate |
| 37 | 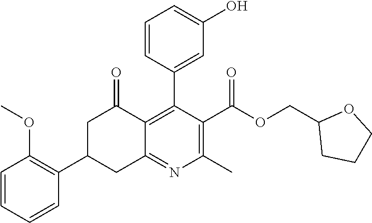 [SMILES: COc1ccccc1C1CC(=O)c2c(nc(C)c(C(=O)OCC3CCCO3)c2-c2cccc(O)c2)C1] | (tetrahydrofuran-2-yl)methyl 4-(3- hydroxyphenyl)-7-(2-methoxyphenyl)- 2-methyl-5-oxo-5,6,7,8- tetrahydroquinoline-3-carboxylate |
| 70 | 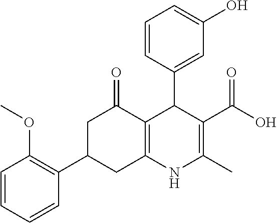 [SMILES: COc1ccccc1C1CC(=O)C2=C(C1)NC(C)=C(C(=O)O)C2c1cccc(O)c1] | 4-(3-hydroxyphenyl)-7-(2- methoxyphenyl)-2-methyl-5-oxo- 1,4,5,6,7,8-hexahydroquinoline-3- carboxylic acid |
| 71 | 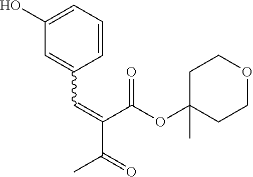 [SMILES: CC(=O)C(=Cc1cccc(O)c1)C(=O)OC1(C)CCOCC1] | 4-methyltetrahydro-2H-pyran-4-yl 2-(3- hydroxybenzylidene)-3-oxobutanoate |
| 72 | 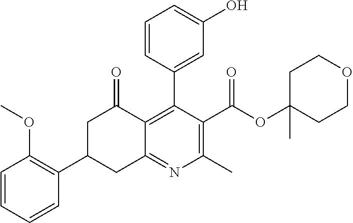 [SMILES: COc1ccccc1C1CC(=O)c2c(nc(C)c(C(=O)OC3(C)CCOCC3)c2-c2cccc(O)c2)C1] | 4-methyltetrahydro-2H-pyran-4-yl 4-(3- hydroxyphenyl)-7-(2-methoxyphenyl)- 2-methyl-5-oxo-5,6,7,8- tetrahydroquinoline-3-carboxylate |
| 73 | 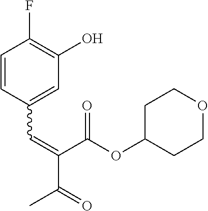 [SMILES: CC(=O)C(=Cc1ccc(F)c(O)c1)C(=O)OC1CCOCC1] | tetrahydro-2H-pyran-4-yl 2-(4-fluoro-3- hydroxybenzylidene)-3-oxobutanoate |
| 74 | 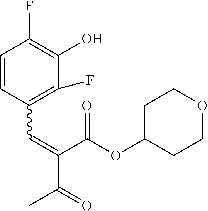 [SMILES: CC(=O)C(=Cc1ccc(F)c(O)c1F)C(=O)OC1CCOCC1] | tetrahydro-2H-pyran-4-yl 2-(2,4- difluoro-3-hydroxybenzylidene)-3- oxobutanoate |

TABLE 2a
Intermediates 28, 30 and 39.

| Cpd | | |
|---|---|---|
| No. | Structure | Compound Name |
| 28 | 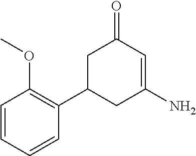 [SMILES: COc1ccccc1C1CC(=O)C=C(N)C1] | 5-amino-2′-methoxy- 1,6-dihydro-[1,1′- biphenyl]-3(2H)-one |
| 30 | 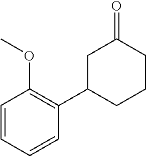 | 3-(2- methoxyphenyl)cyclo- hexan-1-one |
| 39 | 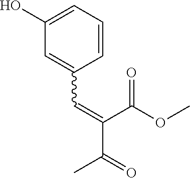 [SMILES: COC(=O)C(=Cc1cccc(O)c1)C(C)=O] | methy l2-(3- hydroxybenzylidene)-3- oxobutanoate |

### General Synthesis Methods for Examples 1-3

Solvents and reagents: Chemicals were purchased from ABCR, Alfa Aesar, ACROS, Sigma Aldrich, TCI, Strem, Combi-Blocks, or Fluorochem, and were used without further purification unless otherwise stated. Anhydrous solvents were purchased over molecular sieves or obtained using an LC Technology Solutions SP-1 solvent purification system. Deuterated solvents were purchased from Armar Chemicals or Cambridge Isotope Laboratories. Chromatographic purification was performed as flash column chromatography with 0.3-0.5 bar pressure using Sigma-Aldrich or SILICYCLE SiliaFlash® Silica Gel P60. The yields given refer to chromatographically purified and spectroscopically pure compounds, unless stated otherwise. NMR spectroscopy: Nuclear Magnetic Resonance spectra were recorded on BRUKER ASCEND, BRUKER AVIII, BRUKER DRX or BRUKER NEO (400 MHz/500 MHz/600 MHz for ¹H NMR) spectrometers. Measurements were carried out at room temperature or with cryoprobes. Chemical shifts (δ) are reported in ppm with the residual solvent signal as internal. The data is reported as (s=singlet, d=doublet, t=triplet, m=multiplet or unresolved, coupling constant(s), integration). For mixtures of diastereoisomers the spectroscopic signals of the major species were reported unless otherwise noted. Mass spectrometry: Mass spectrometric analyses were performed as high resolution ESI and EI measurements by the mass spectrometry service of the Laboratorium für Organische Chemie at ETH Zurich by L. Bertschi, M. Meier and D. Wirz under supervision of Dr. B. Gerrtis.

### Example 1

### Synthesis of Racemic Scaffolds for Evaluation

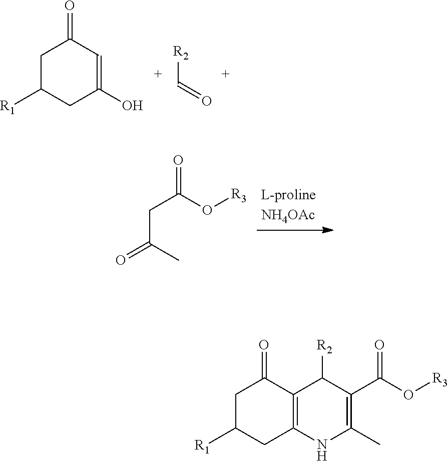

### General Procedure A:

Aldehyde (1.00 equiv), diketone (1.00 equiv), acetoacetate (1.20 equiv), and ammonium acetate (3.00 equiv) were loaded into a round-bottom flask coupled with a magnetic stirring bar. The heterogeneous mixture was heated to 175° C.; along that time it turned into a homogeneous, red molten solution and evaporation of volatile byproducts was noted. Once the evaporation reduced significantly the solution was left to cool to room temperature. The crude residue was dissolved in ethyl acetate and water and was transferred to a separatory funnel. The aqueous layer was extracted with ethyl acetate and the organic extracts were dried over MgSO₄, filtered and concentrated under reduced pressure to yield a crude red/orange oily residue. The crude material was purified via silica gel column chromatography (DCM:EtOAc=1:1) to yield the 1,4-DHP product as a yellow solid.

### General Procedure B:

Diketone (1.00 equiv), aldehyde (1.00 equiv), acetoacetate (1.00 equiv), ammonium acetate (1.5 equiv), and L-proline (0.100 equiv) were loaded into a round-bottom flask coupled with a magnetic stirring bar. Ethanol (1 M) was added and a red, homogeneous solution was observed, which was left to stir at room temperature until complete conversion was observed by TLC. The reaction mixture was poured into brine and extracted with ethyl acetate. The organic layer was dried over anhydrous magnesium sulfate and evaporated to dryness. A crude solid was obtained. The product was purified via silica gel column chromatography (EtOAc:DCM=1:4) to yield a yellow solid.

### Prepared Scaffolds

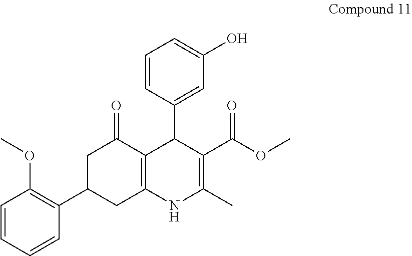

Synthesized according to General Procedure B from 5-(2-methoxyphenyl)cyclohexane-1,3-dione (1.1 g, 5.0 mmol, 1.00 equiv), 3-hydroxybenzaldehyde (611 mg, 5.00 mmol, 1.00 equiv), methyl 3-oxobutanoate (964 mg, 5.00 mmol, 1.00 equiv), ammonium acetate (385 mg, 5.00 mmol, 1.00 equiv), and L-Proline (58 mg, 0.5 mmol, 0.10 equiv) in 5 mL of ethanol. The reaction was left to stir overnight and the crude material was purified to obtain a yellow solid (1.4 g, 3.3 mmol, 67% yield, 5.1:1.0 d.r.). The compound 11 can be separated using preparative HPLC employing a nonchiral stationary phase resulting in 11a and 11b or a chiral stationary phase resulting in 11c and 11d.

¹H NMR (400 MHz, CDCl₃) δ: 7.24-7.17 (m, 1H), 7.07 (dd, J=7.7, 1.7 Hz, 1H), 7.02 (t, J=7.8 Hz, 1H), 6.98-6.94 (m, 1H), 6.92-6.80 (m, 4H), 6.65-6.53 (m, 1H), 5.14 (s, 1H), 3.72 (s, 3H), 3.59 (s, 3H), 3.58-3.48 (m, 1H), 2.79-2.43 (m, 4H), 2.32 (s, 3H).

HRMS (ESI): calculated for C₂₅H₂₆NO₅ [M+H]⁺ 420.1805. found 420.1807.

Nonchiral HPLC: Reprosil Gold 120 C18, H₂O:ACN+0.10% FA=75:25 to 65:35 over 10 min continuing for 3 min at 65:35, 26.5 mL/min, 125 mm×20 mm, 5 μm, t_{R} (D1; minor)=11.7 min; t_{R} (D2; major)=12.7 min.

Chiral HPLC: Dr. Maisch ReproSil Chiral NR, H₂O:ACN+0.1% FA=62:48, 1 mL/min, 250 mm×4.6 mm, 5 m, t_{R} (D1E1)=7.4 min; t_{R} (D1E2)=7.8 min, e.r.=>99:1 (>98% ee), t_{R} (D2E1)=8.6 min; t_{R} (D2E2)=10.1 min, e.r.=>99:1 (>98% ee).

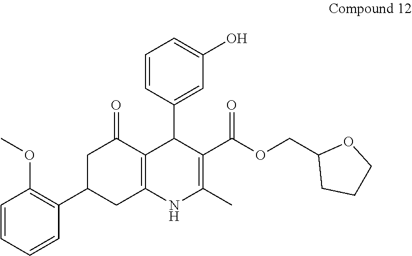 [SMILES: COc1ccccc1C1CC(=O)C2=C(C1)NC(C)=C(C(=O)OCC1CCCO1)C2c1cccc(O)c1]

Synthesized according to General Procedure A from 5-(2-methoxyphenyl)cyclohexane-1,3-dione (3.96 g, 16.5 mmol, 1.00 equiv), 3-hydroxybenzaldehyde (2.01 g, 16.5 mmol, 1.00 equiv), (tetrahydrofuran-2-yl)methyl 3-oxobutanoate (3.7 g, 20 mmol, 1.2 equiv), and ammonium acetate (3.8 g, 49 mmol, 3 equiv). The reaction was left to stir for 30 min and the crude material was purified to obtain a yellow solid (4.6 g, 9.4 mmol, 57% yield, 2:2:1:1 d.r.). The compound 12 can be separated using preparative HPLC employing a nonchiral stationary phase resulting in 12a and 12b. Using enantiopure (S) or (R)-(tetrahydrofuran-2-yl)methyl 3-oxobutanoate results in 12c and 12d respectively after separation by preparative HPLC. Using enantiopure (S)-(tetrahydrofuran-2-yl)methyl 3-oxobutanoate followed by preparative HPLC employing a chiral stationary phase results in 12e and 12f.

¹H-NMR 1H NMR (400 MHz, Methanol-d₄) δ 7.26-7.17 (m, 4H), 7.08-6.99 (m, 2H), 6.99-6.87 (m, 4H), 6.86-6.74 (m, 4H), 6.60-6.52 (m, 2H), 5.06 (s, 1H), 5.05 (s, 1H), 4.12-3.96 (m, 6H), 3.86-3.78 (m, 7H), 3.76-3.69 (m, 3H), 3.54 (tq, J=11.9, 4.0 Hz, 2H), 2.85-2.75 (m, 2H), 2.73-2.61 (m, 4H), 2.49-2.41 (m, 2H), 2.39 (s, 3H), 2.38 (s, 3H), 1.96-1.77 (m, 6H), 1.62-1.50 (m, 2H).

Representative analytical data employing enantiopure (S)-(tetrahydrofuran-2-yl)methyl 3-oxobutanoate derived compound 12c:

¹H-NMR (400 MHz, Methanol-d₄) δ: 7.28-7.14 (m, 2H), 7.03 (t, J=7.9 Hz, 1H), 6.99-6.88 (m, 2H), 6.84-6.73 (m, 2H), 6.60-6.53 (m, 1H), 5.05 (s, 1H), 4.12-3.94 (m, 3H), 3.81 (s, 3H), 3.70 (t, J=6.7, 2H) 3.60-3.47 (m, 1H), 2.91-2.59 (m, 3H), 2.52-2.40 (m, 1H), 2.39 (s, 3H), 1.95-1.76 (m, 3H), 1.64-1.47 (m, 1H).

HRMS (ESI): calculated for C₂₉H₃₁NNaO₆ [M+Na]⁺ 512.2044. found 512.2037.

Nonchiral HPLC: Reprosil Gold 120 C18, H₂O:ACN+0.10% FA=75:25 to 65:35 over 22 min continuing for 5 min at 65:35, 26.5 mL/min, 125 mm×20 mm, 5 μm, t_{R} (D1; minor)=18.9 min; t_{R} (D2; major)=20.9 min.

Chiral HPLC: Dr. Maisch ReproSil Chiral NR, H₂O:ACN+0.1% FA=56:44 to 40:60 over 10 min continuing for 3 min, 1 mL/min, 250 mm×4.6 mm, 5 μm, t_{R} (D1.1)=10.1 min; t_{R} (D1.2)=10.6 min, t_{R} (D2.1)=11.2 min; t_{R} (D2.2)=12.4 min.

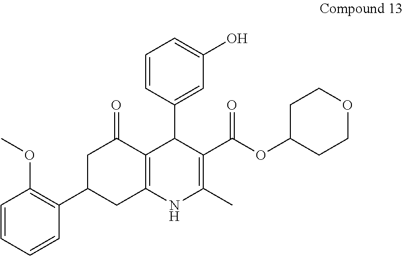 [SMILES: COc1ccccc1C1CC(=O)C2=C(C1)NC(C)=C(C(=O)OC1CCOCC1)C2c1cccc(O)c1]

Synthesized according to General Procedure B from 5-(2-methoxyphenyl)cyclohexane-1,3-dione (109 mg, 0.50 mmol, 1.00 equiv), 3-hydroxybenzaldehyde (61.1 mg, 0.50 mmol, 1.00 equiv), oxan-4-yl 3-oxobutanoate (94.1 mg, 0.50 mmol, 1.00 equiv), ammonium acetate (57.8 mg, 0.75 mmol, 1.50 equiv), and L-Proline (11 mg, 0.1 mmol, 0.2 equiv) in 0.5 mL of ethanol. The reaction was left to stir overnight, whereafter the product precipitated and was collected via vacuum filtration as a colorless solid (120 mg, 0.24 mmol, 49% yield, 5:1 d.r.).

¹H NMR (400 MHz, DMSO-d₆) δ: 9.20 (s, 1H), 9.16 (s, 1H), 7.35-7.13 (m, 2H), 7.11-6.87 (m, 3H), 6.76-6.55 (m, 2H), 6.49 (d, J=8.1 Hz, 1H), 4.89 (s, 1H), 4.86-4.75 (m, 1H), 3.87-3.66 (m, 4H), 3.63-3.50 (m, 1H), 3.49-3.39 (m, 2H), 3.37-3.29 (m, 1H), 2.86-2.65 (m, 1H), 2.66-2.52 (m, 2H), 2.43-2.22 (m, 4H), 1.88-1.75 (m, 1H), 1.72-1.61 (m, 1H), 1.60-1.46 (m, 1H), 1.44-1.30 (m, 1H).

HR-MS (ESI): calculated for C₂₉H₃₂NO₆ [M+H]⁺ 490.2224. found 490.2223.

Chiral HPLC: Dr. Maisch ReproSil Chiral NR, H₂O:ACN+0.1% FA=62:38, 1 mL/min, 250 mm×4.6 mm, 5 μm, t_{R} (D1E1, minor)=7.4 min; t_{R} (D1E2, minor)=7.8 min, t_{R} (D2E1, major)=8.6 min; t_{R} (D2E2, major)=10.1 min.

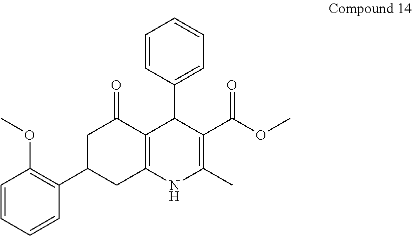

Synthesized according to General Procedure B from 5-(2-methoxyphenyl)cyclohexane-1,3-dione (218 mg, 1.00 mmol, 1.00 equiv), benzaldehyde (0.1 mL, 1.0 mmol, 1.0 equiv), methyl 3-oxobutanoate (116 mg, 1.00 mmol, 1.00 equiv), ammonium acetate (116 mg, 1.50 mmol, 1.50 equiv), and L-Proline (11 mg, 0.1 mmol, 0.1 equiv) in 1 mL of ethanol. The reaction was left to stir overnight, whereafter the product precipitated and was collected via vacuum filtration as a yellow solid (170 mg, 0.42 mmol, 42% yield, >20:1 d.r.).

¹H NMR (400 MHz, CDCl₃) δ: 7.40-7.30 (m, 2H), 7.27-7.18 (m, 3H), 7.17-7.08 (m, 2H), 6.97-6.82 (m, 2H), 6.22 (s, 1H), 5.17 (s, 1H), 3.77 (s, 3H), 3.69-3.52 (m, 4H), 2.83-2.70 (m, 1H), 2.68-2.49 (m, 3H), 2.40 (s, 3H).

HR-MS (ESI): calculated for C₂₅H₂₆NO₄ [M+H]⁺ 404.1856. found 404.1855.

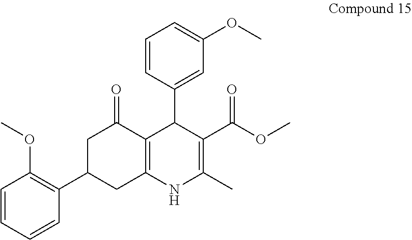

Synthesized according to General Procedure B from 5-(2-methoxyphenyl)cyclohexane-1,3-dione (227 mg, 1.00 mmol, 1.00 equiv), 3-metoxybenzaldehyde (136 mg, 1.00 mmol, 1.00 equiv), methyl 3-oxobutanoate (116 mg, 1.00 mmol, 1.00 equiv), ammonium acetate (116 mg, 1.50 mmol, 1.50 equiv), and L-Proline (11 mg, 0.1 mmol, 0.1 equiv) in 1 mL of ethanol. The reaction was left to stir overnight and the crude material was purified to obtain a yellow solid (260 mg, 0.6 mmol, 60% yield, 4.4:1 d.r.).

¹H NMR (400 MHz, CDCl₃) δ: 7.25-7.20 (m, 1H), 7.17-7.05 (m, 2H), 7.01-6.80 (m, 4H), 6.72-6.64 (m, 1H), 6.43 (s, 1H), 5.15 (s, 1H), 3.76 (d, J=3.3 Hz, 6H), 3.66-3.54 (m, 4H), 2.81-2.49 (m, 4H), 2.37 (s, 3H).

HR-MS (ESI): calculated for C₂₆H₂₈NO₅ [M+H]⁺ 434.1962. found 434.1960.

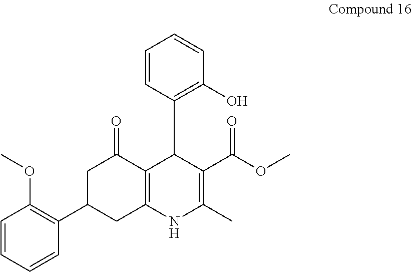 [SMILES: COC(=O)C1=C(C)NC2=C(C(=O)CC(c3ccccc3OC)C2)C1c1ccccc1O]

Synthesized according to General Procedure B from 5-(2-methoxyphenyl)cyclohexane-1,3-dione (227 mg, 1.00 mmol, 1.00 equiv), 2-hydroxybenzaldehyde (122 mg, 1.00 mmol, 1.00 equiv), methyl 3-oxobutanoate (116 mg, 1.00 mmol, 1.00 equiv), ammonium acetate (116 mg, 1.50 mmol, 1.50 equiv), and L-Proline (11 mg, 0.1 mmol, 0.1 equiv) in 1 mL of ethanol. The reaction was left to stir overnight and the crude material was purified to obtain a yellow solid (130 mg, 0.3 mmol, 31% yield, 6.7:1 d.r.).

¹H NMR (400 MHz, CDCl₃) δ: 9.49 (s, 1H), 7.25-7.17 (m, 1H), 7.15-7.03 (m, 2H), 6.99 (dd, J=7.7, 1.7 Hz, 1H), 6.96-6.90 (m, 2H), 6.90-6.79 (m, 2H), 5.19 (s, 1H), 3.79 (s, 3H), 3.74-3.56 (m, 1H), 3.54 (s, 3H), 2.87-2.53 (m, 4H), 2.51 (s, 3H).

HR-MS (ESI): calculated for C₂₅H₂₅NNaO₅ [M+Na]⁺ 442.1625. found 442.1622.

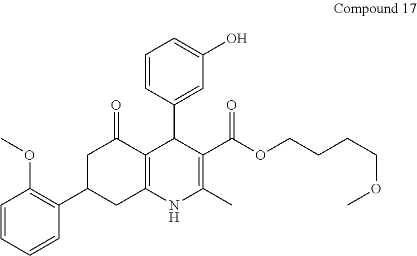 [SMILES: COCCCCOC(=O)C1=C(C)NC2=C(C(=O)CC(c3ccccc3OC)C2)C1c1cccc(O)c1]

Synthesized according to General Procedure B from 5-(2-methoxyphenyl)cyclohexane-1,3-dione (218 mg, 1.00 mmol, 1.00 equiv), 3-hydroxybenzaldehyde (122 mg, 1.00 mmol, 1.00 equiv), 4-methoxybutyl 3-oxobutanoate (245 mg, 1.00 mmol, 1.00 equiv), ammonium acetate (116 mg, 1.50 mmol, 1.50 equiv), and L-Proline (11 mg, 0.1 mmol, 0.1 equiv) in 1 mL of ethanol. The reaction was left to stir overnight and the crude material was purified to obtain a yellow solid (460 mg, 0.72 mmol, 72% yield, 5:1 d.r.).

¹H NMR (400 MHz, Methanol-d₄) δ: 7.26-7.20 (m, 2H), 7.08-6.89 (m, 3H), 6.85-6.77 (m, 2H), 6.62-6.55 (m, 1H), 5.04 (s, 1H), 4.10-3.97 (m, 2H), 3.81 (s, 3H), 3.60-3.47 (m, 1H), 3.38-3.32 (m, 2H), 3.30 (s, 3H), 2.86-2.56 (m, 3H), 2.51-2.41 (m, 1H), 2.40 (s, 3H), 1.73-1.57 (m, 2H), 1.54-1.43 (m, 2H).

HR-MS (ESI): calculated for C₂₉H₃₄NO₆ [M+H]⁺ 492.2381. found 492.2376.

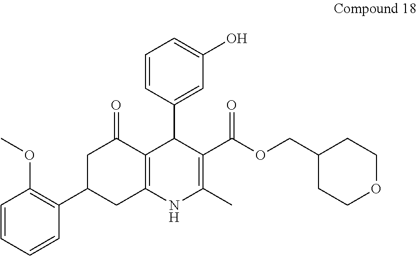 [SMILES: COc1ccccc1C1CC(=O)C2=C(C1)NC(C)=C(C(=O)OCC1CCOCC1)C2c1cccc(O)c1]

Synthesized according to General Procedure B from 5-(2-methoxyphenyl)cyclohexane-1,3-dione (125 mg, 0.57 mmol, 1.00 equiv), 3-hydroxybenzaldehyde (69.9 mg, 0.57 mmol, 1.00 equiv), oxan-4-ylmethyl 3-oxobutanoate (115 mg, 0.57 mmol, 1.00 equiv), ammonium acetate (66.2 mg, 0.86 mmol, 1.50 equiv), and L-Proline (13 mg, 0.1 mmol, 0.2 equiv) in 0.75 mL of ethanol. The reaction was left to stir overnight and the crude material was purified to obtain a yellow solid (113 mg, 0.22 mmol, 39% yield, 5.3:1 d.r.).

¹H NMR (400 MHz, DMSO-d₆) δ: 7.23 (t, J=7.6 Hz, 2H), 7.12-7.01 (m, 1H), 6.99-6.88 (m, 2H), 6.81 (dd, J=7.1, 1.5 Hz, 2H), 6.64-6.54 (m, 1H), 5.04 (s, 1H), 3.99 (dd, J=10.8, 6.9 Hz, 1H), 3.93-3.82 (m, 2H), 3.87-3.75 (m, 4H), 3.59-3.46 (m, 1H), 3.42-3.27 (m, 2H), 2.88-2.55 (m, 3H), 2.51-2.41 (m, 1H), 2.41 (s, 3H), 1.90-1.73 (m, 1H), 1.53-1.44 (m, 1H), 1.40-1.30 (m, 1H), 1.24-1.08 (m, 2H).

HR-MS (ESI): calculated for C₃₀H₃₄NO₆ [M+H]⁺ 504.2381. found 504.2378.

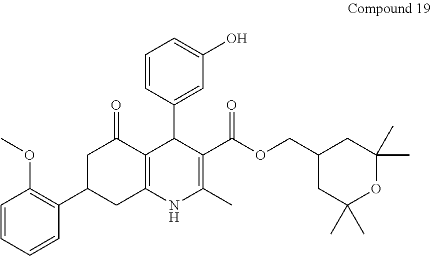

Synthesized according to General Procedure B from 5-(2-methoxyphenyl)cyclohexane-1,3-dione (109 mg, 0.50 mmol, 1.00 equiv), 3-hydroxybenzaldehyde (61.1 mg, 0.50 mmol, 1.00 equiv), (2,2,6,6-tetramethyltetrahydro-2H-pyran-4-yl)methyl 3-oxobutanoate (128 mg, 0.50 mmol, 1.00 equiv), ammonium acetate (57.8 mg, 0.75 mmol, 1.50 equiv), and L-Proline (11 mg, 0.1 mmol, 0.2 equiv) in 0.5 mL of ethanol. The reaction was left to stir overnight, whereafter the product precipitated and was collected via vacuum filtration as a yellow solid (172 mg, 0.31 mmol, 61% yield, 7.7:1 d.r.).

¹H NMR (400 MHz, DMSO-d₆) δ: 9.20 (s, 1H), 9.12 (s, 1H), 7.31-7.19 (m, 2H), 7.04-6.90 (m, 3H), 6.70-6.63 (m, 2H), 6.54-6.47 (m, 1H), 4.93 (s, 1H), 3.93 (dd, J=10.8, 6.4 Hz, 1H), 3.76 (s, 3H), 3.65 (dd, J=10.7, 6.3 Hz, 1H), 3.40 (td, J=13.1, 12.5, 4.0 Hz, 1H), 2.73 (dd, J=17.2, 11.8 Hz, 1H), 2.62-2.51 (m, 2H), 2.36 (s, 3H), 2.32-2.22 (m, 1H), 2.10-1.97 (m, 1H), 1.43-1.26 (m, 2H), 1.12 (s, 3H), 1.10 (s, 3H), 1.03 (d, J=2.9 Hz, 6H), 0.87-0.70 (m, 2H).

HR-MS (ESI): calculated for C₃₄H₄₁NNaO₆ [M+Na]⁺ 582.2826. found 582.2822.

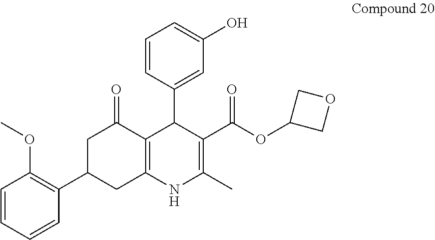 [SMILES: COc1ccccc1C1CC(=O)C2=C(C1)NC(C)=C(C(=O)OC1COC1)C2c1cccc(O)c1]

Synthesized according to General Procedure B from 5-(2-methoxyphenyl)cyclohexane-1,3-dione (35.9 mg, 0.16 mmol, 1.00 equiv), 3-hydroxybenzaldehyde (20.1 mg, 0.16 mmol, 1.00 equiv), oxetan-3-yl 3-oxobutanoate (26 mg, 0.16 mmol, 1.00 equiv), ammonium acetate (19.0 mg, 0.25 mmol, 1.50 equiv), and L-Proline (3.78 mg, 0.03 mmol, 0.20 equiv) in 0.3 mL of ethanol. The reaction was left to stir overnight and the crude material was purified to obtain a yellow solid (46 mg, 0.1 mmol, 61% yield, 3.8:1 d.r.).

¹H NMR (400 MHz, DMSO-d₆) δ: 9.30 (s, 1H), 9.19 (s, 1H), 7.35-7.20 (m, 2H), 7.09-6.88 (m, 3H), 6.72-6.64 (m, 2H), 6.54-6.47 (m, 1H), 5.36-5.25 (m, 1H), 4.90 (s, 1H), 4.79-4.67 (m, 2H), 4.54-4.42 (m, 1H), 4.31 (ddd, J=7.3, 5.2, 0.9 Hz, 1H), 3.78 (s, 3H), 3.53-3.40 (m, 1H), 2.86-2.69 (m, 1H), 2.67-2.51 (m, 2H), 2.35-2.25 (m, 4H).

HR-MS (ESI): calculated for C₂₇H₂₈NO₆ [M+H]⁺ 462.1911. found 462.1906.

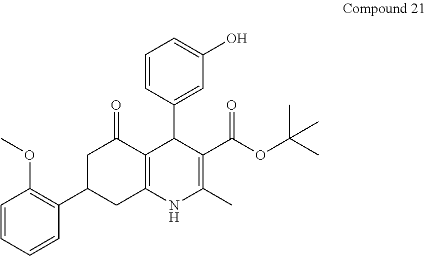 [SMILES: COc1ccccc1C1CC(=O)C2=C(C1)NC(C)=C(C(=O)OC(C)(C)C)C2c1cccc(O)c1]

Synthesized according to General Procedure B from 5-(2-methoxyphenyl)cyclohexane-1,3-dione (455 mg, 2.00 mmol, 1.00 equiv), 3-hydroxybenzaldehyde (244 mg, 2.00 mmol, 1.00 equiv), tert-butyl 3-oxobutanoate (316 mg, 2.00 mmol, 1.00 equiv), ammonium acetate (231 mg, 3.00 mmol, 1.50 equiv), and L-Proline (23 mg, 0.2 mmol, 0.1 equiv) in 2 mL of ethanol. The reaction was left to stir overnight and the crude material was purified to obtain a yellow solid (503 mg, 1.1 mmol, 54% yield, 5.9:1 d.r.).

¹H NMR (400 MHz, Methanol-d₄) δ: 7.25-7.18 (m, 2H), 7.03 (t, J=7.8 Hz, 1H), 6.98-6.66 (m, 4H), 6.60-6.52 (m, 1H), 4.95 (s, 1H), 3.80 (s, 3H), 3.60-3.47 (m, 1H), 2.86-2.39 (m, 4H), 2.32 (s, 3H), 1.37 (s, 9H).

HR-MS (ESI): calculated for C₂₈H₃₁NNaO₅ [M+Na]⁺ 484.2094. found 484.2093.

### Reference Compound 70

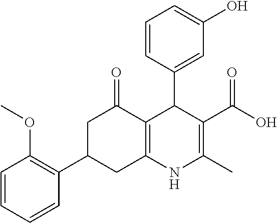 [SMILES: COc1ccccc1C1CC(=O)C2=C(C1)NC(C)=C(C(=O)O)C2c1cccc(O)c1]

To a solution of methyl 4-(3-hydroxyphenyl)-7-(2-methoxyphenyl)-2-methyl-5-oxo-4,6,7,8-tetrahydro-1H-quinoline-3-carboxylate (419 mg, 1.00 mmol, 1.00 equiv) in THF (4 mL, 0.25 M) at room temperature was added LiOH (2 M aqueous solution) (5.0 ml, 10 mmol). Then the reaction was heated to reflux and stirred overnight. The mixture was diluted with ethyl acetate and extracted with water. The aqueous layer was acidified to pH 1 with concentrated HCl. The aqueous layer was then extracted with ethyl acetate. The combined organic layers were dried (sodium sulfate), filtered and concentrated to give the product as a yellow solid (270 mg, 0.66 mmol, 67% yield, 7:1 d.r.).

¹H NMR and LC-MS data vide infra.

### Example 2

### Diastereoselective Synthesis of Compound 13a and its Isomers

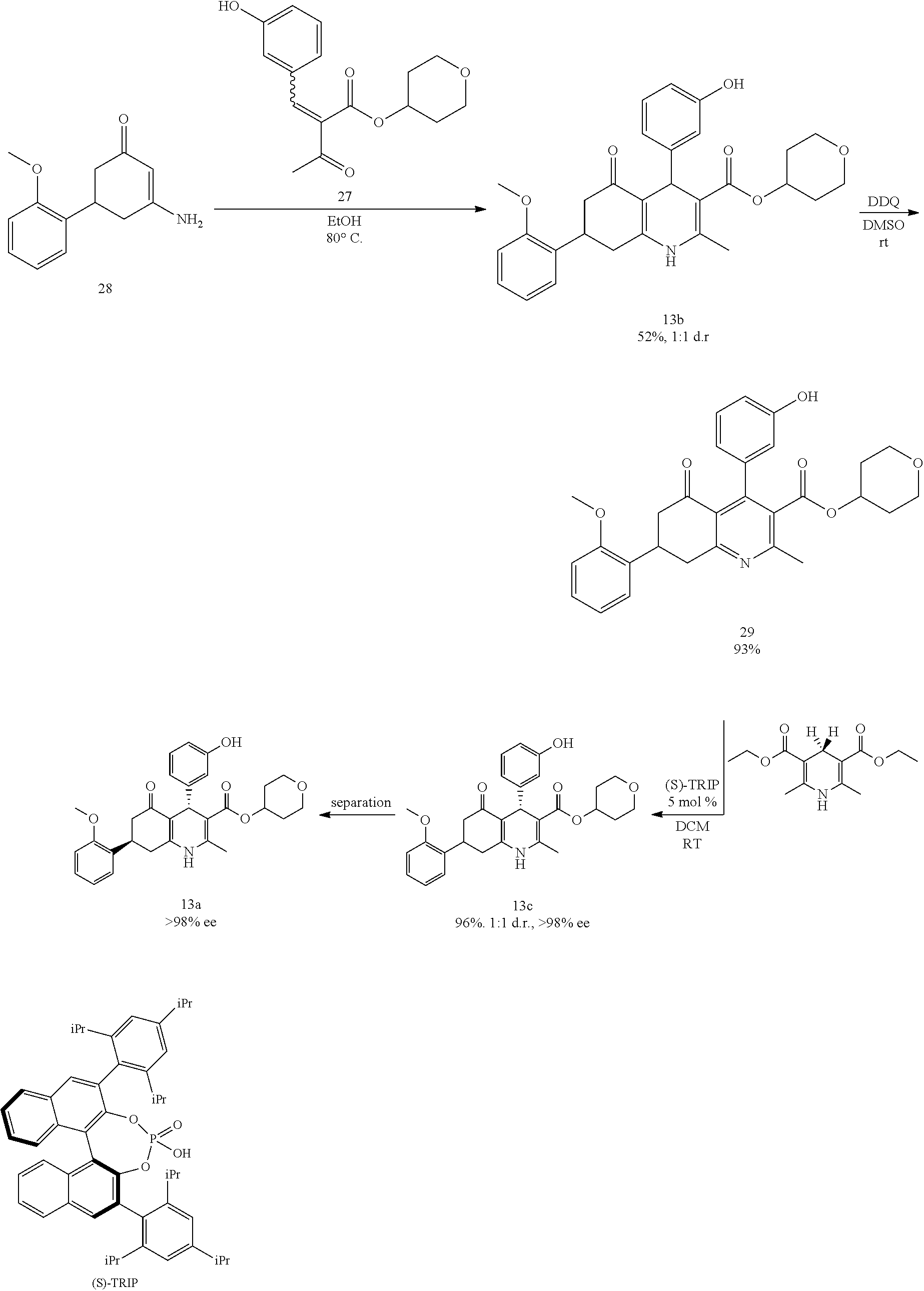

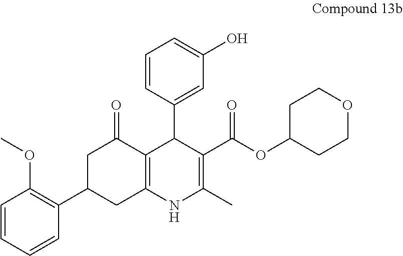 [SMILES: COc1ccccc1C1CC(=O)C2=C(C1)NC(C)=C(C(=O)OC1CCOCC1)C2c1cccc(O)c1]

Racemic enaminone 28 (82.0 mg, 282 μmol, 1.00 equiv) was dissolved in EtOH (0.25 mL, 1M) and transferred into an vial coupled with a magnetic stirring bar. Condensation of aldehyde and β-keto esters conducted according to reported procedures e.g: Chemical and Pharmaceutical Bulletin, 1986, 34, 1589. Tetrahydro-2H-pyran-4-yl 2-(3-hydroxybenzylidene)-3-oxobutanoate (27) (61.4 mg, 282 μmol, 1.00 equiv) was added and the mixture was heated to 80° C. and stirred for 36 h. The solvent was removed under reduced pressure and the residue was purified via flash silica gel column chromatography (DCM:MeOH=20:1), to yield the product as a yellow solid (72.0 mg, 147 μmol, 52%, 1:1 d.r.).

### Alternative:

Synthesized according to General Procedure B from 5-(2-methoxyphenyl)cyclohexane-1,3-dione (4.04 g, 18.5 mmol, 1.00 equiv), 3-hydroxybenzaldehyde (2.26 g, 18.5 mmol, 1.00 equiv), oxan-4-yl 3-oxobutanoate (3.44 g, 18.5 mmol, 1.00 equiv), ammonium acetate (2.14 g, 27.7 mmol, 1.50 equiv), and L-Proline (426 mg, 0.37 mmol, 0.2 equiv) in of ethanol (18.5 mL, 1 M). The reaction was left to stir overnight, whereafter the product partially precipitated as an off-white solid (5.26 g, 10.7 mmol, 71% yield, 3:1 d.r.). The filtrate was concentrated under reduced pressure and the residue was purified via flash silica gel column chromatography (DCM:MeOH=20:1) to yield the product as an off-white solid (1.20 g, 2.57 mmol, 14%, 4:1 d.r). Combined a yield of 85% was observed.

¹H NMR (400 MHz, CD30D) δ: 7.27-7.18 (m, 2H), 7.17-7.11 (m, 1H), 7.04 (t, J=7.8 Hz, 1H), 7.00-6.87 (m, 5H), 6.84-6.77 (m, 2H), 6.76-6.67 (m, 3H), 6.60-6.48 (m, 2H), 5.04 (s, 1H), 5.00 (s, 1H), 4.92-4.84 (m, 3H), 3.94-3.69 (m, 10H), 3.66-3.46 (m, 5H), 3.46-3.38 (m, 2H), 2.86-2.50 (m, 8H), 2.48-2.30 (m, 6H), 1.98-1.80 (m, 2H), 1.78-1.57 (m, 4H), 1.52-1.41 (m, 2H).

HR-MS (ESI): vide supra

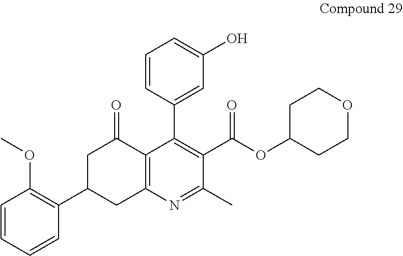 [SMILES: COc1ccccc1C1CC(=O)c2c(nc(C)c(C(=O)OC3CCOCC3)c2-c2cccc(O)c2)C1]

A suspension of the Hantzsch 1,4-DHP 13b (1.00 g, 2.04 mmol, 1.00 equiv) and 2,3-dichloro-5,6-dicyano-1,4-benzoquinone (0.52 g, 2.25 mmol, 1.10 equiv) in DMSO (10.2 mL, 0.2 M) was stirred at rt for 15 min. The crude product was purified by flash silica gel column chromatography (DCM:MeOH=10:1) to yield the product as an off-white solid (0.93 g, 1.90 mmol, 93%).

¹H NMR (400 MHz, CD30D) δ: 7.31-7.13 (m, 3H), 7.06-6.89 (m, 2H), 6.78 (ddd, J=8.2, 2.5, 1.0 Hz, 1H), 6.70-6.53 (m, 2H), 4.95-4.90 (m, 1H), 3.92-3.82 (m, 4H), 3.73-3.58 (m, 2H), 3.54-3.38 (m, 4H), 3.06-2.90 (m, 1H), 2.84-2.71 (m, 1H), 2.57 (s, 3H), 1.78-1.52 (m, 2H), 1.42-1.22 (m, 2H).

HR-MS (ESI): calculated for C₂₉H₃₀NO₆ [M+H]⁺ 488.2068. found 488.2064.

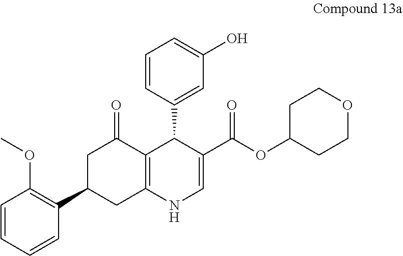 [SMILES: COc1ccccc1[C@H]1CC(=O)C2=C(C1)NC=C(C(=O)OC1CCOCC1)[C@H]2c1cccc(O)c1]

Pyridine 29 (37.6 mg, 0.08 mmol, 1.00 equiv) was loaded into a vial coupled with a magnetic stirring bar and dissolved in dry DCM (0.8 mL, 0.1 M). Diethyl 2,6-dimethyl-1,4-dihydropyridine-3,5-dicarboxylate (44.2 mg, 0.16 mmol, 2.00 equiv) and (S)-TRIP (2.9 mg, 0.004 mmol, 0.05 equiv) were added and the mixture was stirred for 42 h at rt. The solvent was removed under reduced pressure and the residue was purified via flash silica gel column chromatography (DCM:EtOAc=2:3) to yield the product as an off-white solid (37 mg, 0.07 mmol, 96%, 1:1 d.r., >98% ee). The two diastereoisomers of compound 13c (13a and 13e) can be separated using a Buchi Pure Chromatography Systems with silica gel column (hexanes:EtOAc=9:1 to 1:9) resulting in pure 13a and 13e.

### Compound 13a

¹H NMR (500 MHz, CD3OD) δ: 7.24-7.19 (m, 2H), 7.04 (t, J=7.8 Hz, 1H), 7.00-6.89 (m, 2H), 6.84-6.77 (m, 2H), 6.58-6.55 (m, 1H), 5.04 (s, 1H), 4.92-4.86 (m, 1H), 3.88-3.79 (m, 4H), 3.63-3.49 (m, 3H), 3.46-3.39 (m, 1H), 2.79 (dd, J=17.6, 11.9 Hz, 1H), 2.72-2.63 (m, 3H), 2.48-2.35 (m, 4H), 1.95-1.85 (m, 1H), 1.78-1.60 (m, 2H), 1.50-1.41 (m, 1H).

HR-MS (ESI): vide supra

Chiral HPLC: Dr. Maisch ReproSil Chiral NR, H₂O:ACN+0.1% FA=62:38, 1 mL/min, 250 mm×4.6 mm, 5 μm, t_{R} (D1E1, minor)=7.4 min; t_{R} (D1E2, minor)=7.8 min, t_{R} (D2E1, major)=8.6 min; t_{R} (D2E2, major)=10.1 min.

### Compound 13e

¹H NMR (400 MHz, MeOD) δ 7.21-7.13 (m, 1H), 7.00 (dd, J=36.5, 8.0 Hz, 1H), 6.96-6.92 (m, 2H), 6.77-6.69 (m, 3H), 6.55 (ddd, J=8.1, 2.5, 1.0 Hz, 1H), 5.02 (s, 1H), 4.91 (dd, J=7.4, 3.6 Hz, 1H), 3.91-3.82 (m, 4H), 3.82-3.76 (m, 1H), 3.67-3.54 (m, 2H), 3.45 (ddd, J=11.5, 7.6, 3.6 Hz, 1H), 2.82-2.74 (m, 2H), 2.64-2.56 (m, 2H), 2.40 (s, 3H), 1.99-1.86 (m, 1H), 1.79-1.63 (m, 3H), 1.56-1.42 (m, 1H).

The other diastereoisomers of compound 13 (13d, 13f) can be obtained accordingly using (R)-TRIP.

### Example 3

### Enantioselective Synthesis of Compound 11c

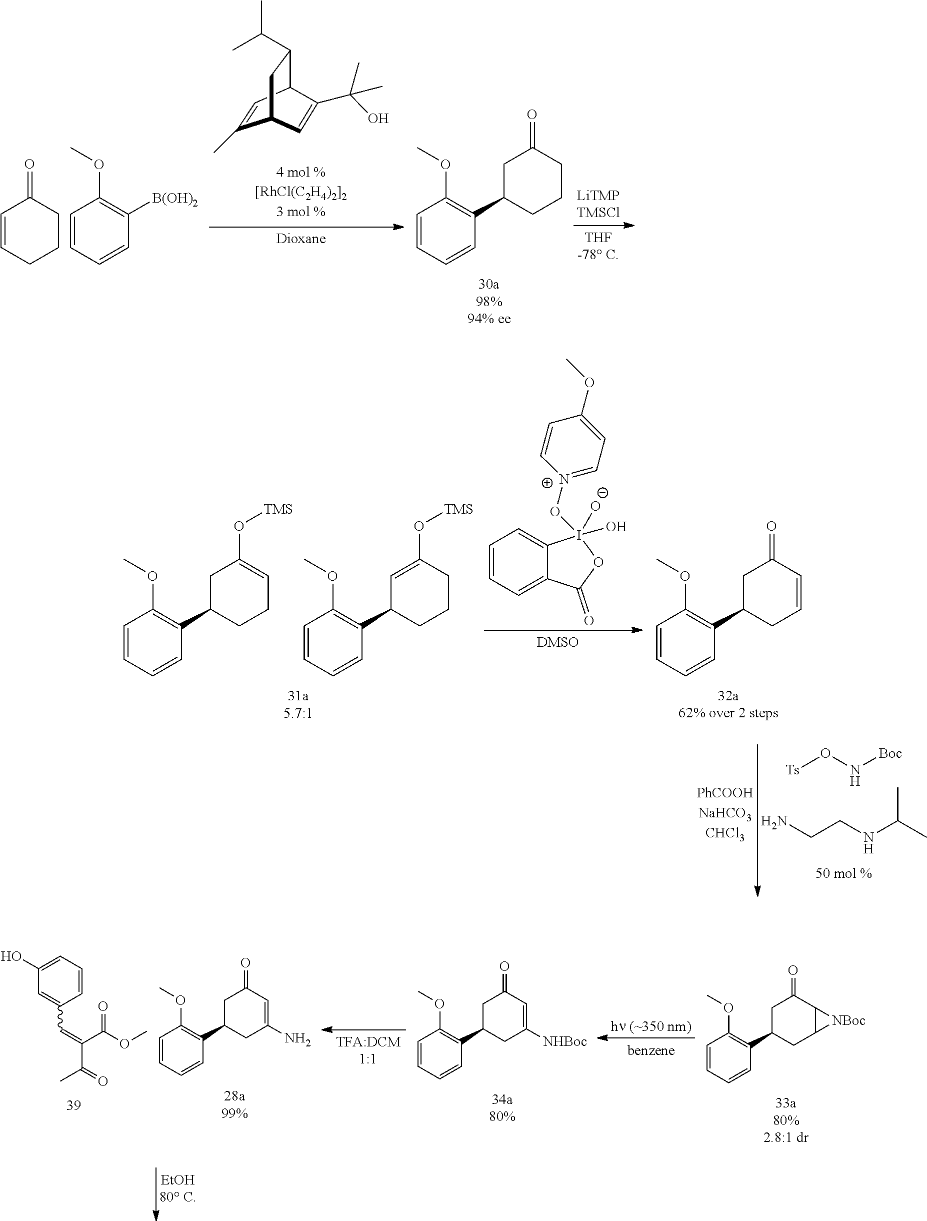

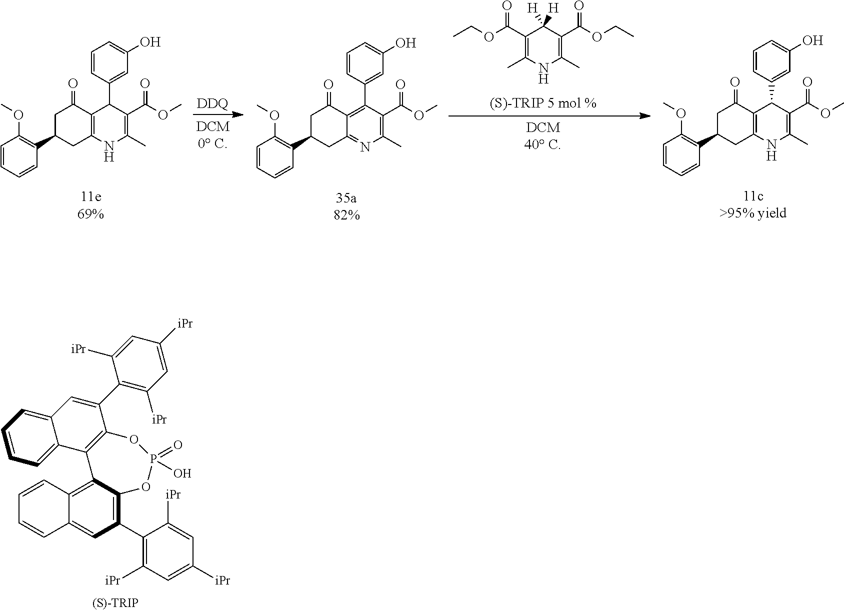

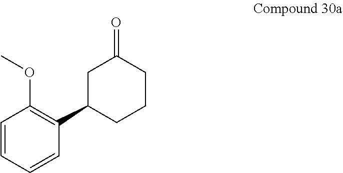

A solution of [RhCl(C₂H₄)₂]₂ (152 mg, 0.39 mmol, 0.03 equiv, prepared from RhCl3·xH2O according to: https://doi.org/10.1002/047084289X.rn01715) and 2-((1R,4R,7R)-7-isopropyl-5-methylbicyclo[2.2.2]octa-2,5-dien-2-yl)propan-2-ol (115 mg, 0.52 mmol, 0.04 equiv) in 1,4-dioxane (22 mL) was stirred for 5 min at room temperature. The synthesis of the ligand and this exact transformation has be described in “Org. Lett. 2008, 10, 19, 4387-4389”. 1.5 M aq. KOH (4.3 mL, 6.5 mmol, 0.5 equiv) was added, and the resulting solution was stirred at room temperature for an additional 5 min. (2-methoxyphenyl)boronic acid (2.96 g, 19.5 mmol, 1.50 equiv) and cyclohexenone (1.26 mL, 13.0 mmol, 1.00 equiv) were added to it with additional 1,4-dioxane (21 mL, 0.3 M overall), and the resulting mixture was stirred at room temperature overnight (15 h). The reaction mixture was directly passed through a pad of silica gel with Et₂O, and the solvent was removed under vacuum. The residue was purified via flash silica gel column chromatography (hexane:EtOAc=4:1) to yield the product as an orange oil (2.60 g, 12.7 mmol, 98%).

¹H NMR and HR-MS (ESI) data is in accordance with literature spectra.

Chiral HPLC: Daicel Chiralpak OD-H, hexane:ⁱPrOH=97:3, 1 mL/min, λ=254, t_{R} (major)=11.2 min; t_{R} (minor)=9.7 min, e.r.=97:3 (94% ee).

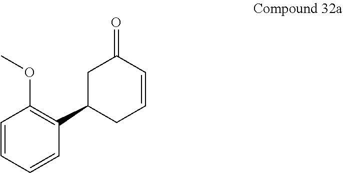

To a solution of 2,2,6,6-tetramethylpiperidine (2.28 mL, 13.4 mmol, 1.07 equiv) in THF (88 mL) was added a 1.43 M hexane solution of n-BuLi (11 mL, 16.8 mmol, 1.34 equiv) at 4° C. (ice-water bath). After stirring for 1 h at the same temperature, the reaction mixture was cooled to −78° C. TMSCl (2.22 mL, 17.5 mmol, 1.4 equiv) was then added, followed by addition of a solution of 30a (2.55 g, 12.5 mmol, 1.00 equiv) in THF (88 mL, overall 0.07 M). After stirring for 2 h at the same temperature, the reaction mixture was quenched by addition of saturated NaHCO₃ aqueous solution, extracted with Et₂O, dried over Na₂SO₄, and concentrated to afford crude enol silyl ether 31a, which was used for the next step without further purification.

The silyl-enol ether was dissolved in a minimal amount of DMSO and the IBX·MPO complex (52.8 mL, 0.4 M in DMSO, 21.1 mmol, 2.00 equiv) was added at room temperature and the solution was left to stir until completion was observed via TLC. Upon completion, the reaction mixture was diluted with aqueous NaHCO₃ (5%) and extracted with diethyl ether (3×60 mL). The combined organic phases were washed with saturated aqueous NaHCO₃, water, and brine. After drying (MgSO₄), the solvent was removed in vacuo to yield the crude product, which was purified via flash column chromatography (hexane:EtOAc=9:1) to yield the product as a pale-yellow oil (1.57 g, 7.78 mmol, 62%).

¹H NMR (400 MHz, CDCl₃) δ: 7.24 (ddd, J=8.1, 7.4, 1.8 Hz, 1H), 7.18 (dd, J=7.6, 1.7 Hz, 1H), 7.10-7.04 (m, 1H), 6.95 (td, J=7.5, 1.2 Hz, 1H), 6.89 (dd, J=8.2, 1.1 Hz, 1H), 6.16-6.06 (m, 1H), 3.83 (s, 3H), 3.78-3.66 (m, 1H), 2.82-2.54 (m, 4H).

HR-MS (ESI): calculated for C₁₃H₁₄NaO₂ [M+Na]⁺ 225.0886. found 225.0888.

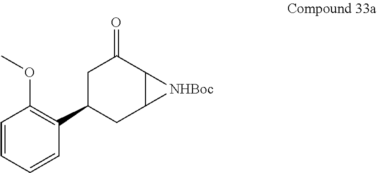

To a stirred solution of benzoic acid (246 mg, 2.01 mmol, 1.00 equiv), sodium bicarbonate (846 mg, 10.1 mmol, 5.00 equiv), tert-butyl (tosyloxy)carbamate (579 mg, 2.01 mmo, 1.00 equivl) in CHCl₃ (15 mL), N1,N1-dimethylethane-1,2-diamine (0.13 mL, 1.01 mmol, 0.5 equiv) was added at room temperature, under nitrogen atmosphere. Enone 32a (489 g, 2.42 mmol, 1.20 equiv) in CHCl₃ (5 mL) was added in one portion and the resulting mixture was stirred at room temperature overnight (15 h). Water was added (30 mL) and the aqueous solution was extracted with CHCl₃ (3×20 mL) and the combined organic layers were washed once with saturated brine, dried over NaSO₄ and concentrated in vacuo. The resulting oily residue was purified by column chromatography (hexane:EtOAc=4:1) to yield the product as a off-white solid (654 mg, 1.61 mmol, 73%, 2.8:1 d.r.).

¹H NMR (400 MHz, CDCl₃) δ: 7.24-7.16 (m, 1H), 7.15-7.05 (m, 1H), 6.97-6.81 (m, 2H), 3.81 (s, 3H), 3.77-3.51 (m, 1H), 3.24-3.09 (m, 1H), 3.05-2.62 (m, 2H), 2.48-2.05 (m, 3H), 1.48 (s, 9H).

HR-MS (ESI): calculated for C₁₈H₂₃NNaO₄ [M+Na]⁺ 340.1519. found 340.1515.

Chiral HPLC: Daicel Chiralpak OD-R, H₂O:ACN+0.1% FA=1:1, 1 mL/min, λ=254, t_{R} (D1, major)=32.3 min; t_{R} (D1, minor)=28.5 min; t_{R} (D2, major)=19.1 min; t_{R} (D2, minor)=23.7 min, e.r.=97:3 (94% ee).

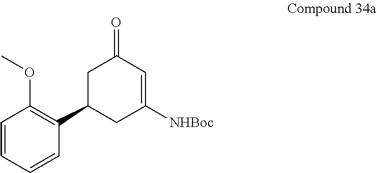 [SMILES: COc1ccccc1[C@H]1CC(=O)C=C(NC(=O)OC(C)(C)C)C1]

The BOC-protected aziridine 33a (78% pure, enone impurity, 132 mg, 0.32 mmol, 1.00 equiv) was split into two 20 mL heat-dried schlenk tubes and degassed benzene (19 mL, 0.008M) was added to both. The vessels were transferred to a UV reactor and were irradiated for 5 h at ˜350 nm. The solutions were combined and concentrated under reduced pressure. The crude residue was purified via column chromatography (hexane:EtOAc=3:2) to yield the product as a colorless solid (84 mg, 0.26 mmol, 82%).

¹H NMR (400 MHz, CDCl₃) δ: 7.26-7.20 (m, 1H), 7.15 (dd, J=7.6, 1.7 Hz, 1H), 6.93 (td, J=7.5, 1.1 Hz, 1H), 6.88 (dd, J=8.2, 1.1 Hz, 1H), 6.55 (br s, 1H), 6.44 (d, J=1.6 Hz, 1H), 3.81 (s, 3H), 3.78-3.65 (m, 1H), 2.84-2.51 (m, 5H), 1.48 (s, 9H).

HR-MS (ESI): calculated for C₁₈H₂₃NNaO₄ [M+Na]⁺ 340.1519. found 340.1518.

Chiral HPLC: Daicel Chiralpak OD-R, H₂O:ACN+0.1% FA=1:1, 1 mL/min, λ=254, t_{R} (major)=15.5 min; t_{R} (minor)=18.1 min, e.r.=98:2 (96% ee).

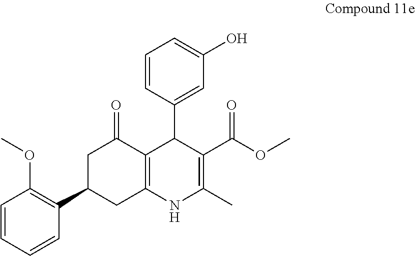

The BOC-protected enaminone 34a (81.0 mg, 0.25 mmol, 1.00 equiv) was loaded into a 10 mL flask coupled with a magnetic stirring bar and was dissolved in a 1:1 mixture of DCM:TFA (2.2 mL, 0.2 M). The solution was stirred at room temperature for 1H and the solvent was removed under reduced pressure. The residue was redissolved in ethyl acetate and washed with sat. aq. sodium bicarbonate and brine, dried over magnesium sulfate and concentrated under reduced pressure to yield the crude, deprotected enaminone 28a which was employed in the next step without further purification.

Analytical Data LC-MS/1H NMR of 28a

rt=0.84 min, 218.1 [M+H]+ (76% UV Abs).

¹H NMR (400 MHz, DMSO-d₆) δ 7.27-7.17 (m, 2H), 7.04-6.41 (m, 4H), 4.98 (d, J=0.9 Hz, 1H), 3.79 (s, 3H), 3.50 (tt, J=11.4, 4.3 Hz, 1H), 2.59-2.51 (m, 1H), 2.45-2.29 (m, 2H), 2.14 (ddd, J=16.0, 4.4, 1.4 Hz, 1H).

The crude deprotected enaminone was dissolved in EtOH (0.25 mL, 1M) and transferred into an HPLC vial coupled with a magnetic stirring bar. Methyl (E/Z)2-(3-hydroxybenzylidene)-3-oxobutanoate (73.2 mg, 0.33 mmol, 1.33 equiv) was added and the mixture was heated to 80° C. and stirred for 20 h. The solvent was removed under reduced pressure and the residue was purified via flash silica gel column chromatography (hexane:EtOAc=1:1), to yield the product as a yellow solid (72.3 mg, 0.17 mmol, 69%, 1:1 d.r.). Methyl (E/Z)2-(3-hydroxybenzylidene)-3-oxobutanoate has previously prepared, see: Chemical and Pharmaceutical Bulletin, 1986, 34, 1589.

¹H NMR and HR-MS (ESI) data vide supra.

Chiral HPLC: Daicel Chiralpak OD-R, H₂O:ACN+0.1% FA=58:42, 1 mL/min, λ=362, t_{R} (D1, major)=14.8 min; t_{R} (D1, minor)=17.3 min; t_{R} (D2, major)=16.4 min; t_{R} (D2, minor)=24.2 min, e.r.=98:2 (96% ee).

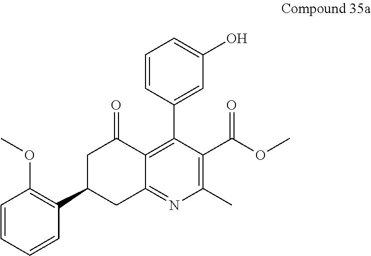

A suspension of the Hantzsch 1,4-DHP 11e (32.0 mg, 0.08 mmol, 1.00 equiv) and 2,3-dichloro-5,6-dicyano-1,4benzoquinone (17.3 mg, 0.08 mmol, 1.00 equiv) in dichloromethane (0.8 mL, 1M) was stirred at 0° C. for 30 min. The precipitate was filtered, washed with dichloromethane (2×5 mL) and the filtrate was concentrated under reduced pressure. The residue was purified by flash silica gel column chromatography (hexane:EtOAc=3:2) to yield the product as a off-white solid (26.0 mg, 0.06 mmol, 82%).

¹H NMR (400 MHz, CDCl₃) δ: 7.26-7.16 (m, 3H), 6.95 (td, J=7.5, 1.1 Hz, 1H), 6.89 (dd, J=8.3, 1.1 Hz, 1H), 6.82-6.73 (m, 1H), 6.66 (dd, J=14.0, 7.5 Hz, 1H), 6.57 (d, J=17.4 Hz, 1H), 3.93-3.83 (m, 1H), 3.82 (s, 3H), 3.56-3.36 (m, 5H), 2.92-2.81 (m, 2H), 2.60 (s, 3H).

HR-MS (ESI): calculated for C₂₅H₂₄NO₅ [M+H]⁺ 418.1649. found 418.1646.

Chiral HPLC: Daicel Chiralpak OD-R, H₂O:ACN+0.1% FA=58:42, 1 mL/min, λ=254, t_{R} (major)=26.1 min; t_{R} (minor)=24.0 min, e.r.=98:2 (96% ee).

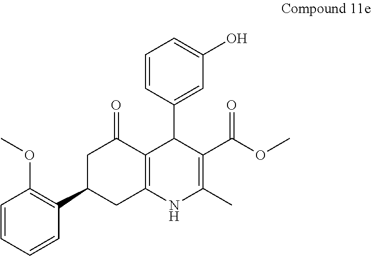

Pyridine 35a (22.0 mg, 0.05 mmol, 1.00 equiv) was loaded into an HPLC vial coupled with a magnetic stirring bar and dissolved in dry DCM (0.5 mL, 0.1 M). Diethyl 2,6-dimethyl-1,4-dihydropyridine-3,5-dicarboxylate (26.7 mg, 0.10 mmol, 2.00 equiv) and (S)-TRIP (1.984 mg, 0.003 mmol, 0.05 equiv) were added and the mixture was stirred for 16 h at 40° C. The solvent was removed under reduced pressure and the residue was purified via flash silica gel column chromatography (hexane:EtOAc=1:1) to yield the product as an off-white solid (21 mg, 0.05 mmol, 95%, >20:1 d.r.)

¹H NMR and HR-MS (ESI) data vide supra.

Chiral HPLC: Daicel Chiralpak OD-R, H₂O:ACN+0.1% FA=58:42, 1 mL/min, λ=362, t_{R} (major)=16.3 min; t_{R} (minor)=24.2 min, e.r.=>99:1 (>99% ee).

### Example 4

### Synthesis of Compounds 12e, 12g, 50a, 50b, 52a-65a

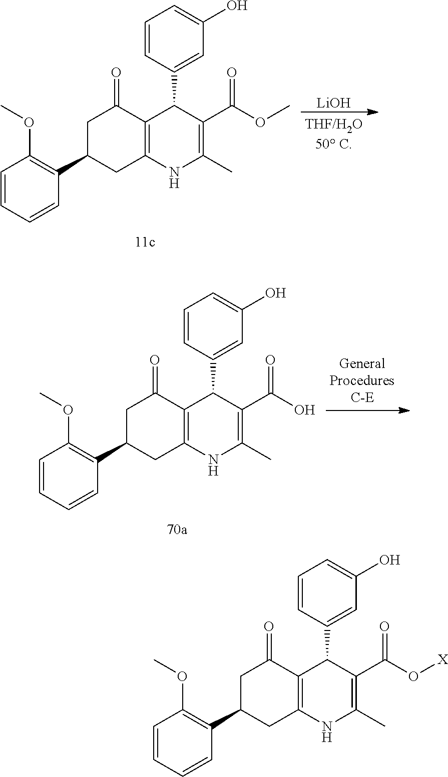

### General Procedure C:

To a stirred solution of acid 70a (0.07-0.19 mmol, 1.00 equiv) in acetonitrile (0.1 M) at RT were added alcohol (4.00 equiv), pyridine (4.00 equiv) and propanephosphonic acid anhydride (4.00 equiv, 50% w/w in DMF). The reaction mixture was heated to 40° C. and stirred for 3 h—overnight. The mixture was cooled down to RT, a saturated solution of NH₄Cl was added and the mixture was extracted with ethyl acetate. The combined organic layer was washed with water and solvent was removed under pressure to afford the crude product. The crude product was purified via silica gel column chromatography (acetone:toluene) resulting in product in 27-56% yield.

### General Procedure D:

To a stirred solution of acid 70a (0.12-0.20 mmol, 1.00 equiv) and alcohol (3.00 equiv) in a mixture of DCM/NMP (3:1, 0.08 M) N,N′-diisopropylmethanediimine (3.00 equiv) was added dropwise at RT. The reaction mixture was stirred at 40° C. for 1 h-overnight. Ethyl acetate was added to the reaction mixture and the organic layer was washed with water, dried using a phase separator and concentrated under vacuum. The crude product was purified via silica gel column chromatography (DCM/MeOH) and by reverse-phase flash chromatography (Water/ACN) optionally followed extraction (DCM/Water) resulting product in 14-28% yield.

### General Procedure E:

To a stirred solution of acid 70a (0.07 mmol, 1.00 equiv) in alcohol (0.25 M) at RT was added N,N′-diisopropylmethanediimine (3.00 equiv). The reaction mixture was stirred at RT overnight. The mixture was concentrated under vacuum. The crude product was purified by reverse-phase flash chromatography (Water/ACN) resulting in product in 52-60% yield.

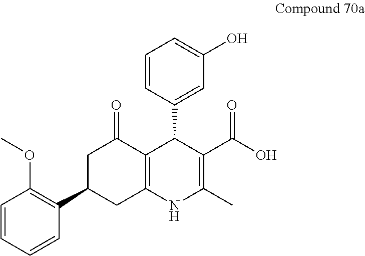 [SMILES: COc1ccccc1[C@H]1CC(=O)C2=C(C1)NC(C)=C(C(=O)O)[C@H]2c1cccc(O)c1]

To a stirred solution of ester 11c (2.17 g, 5.17 mmol, 1.00 equiv) in mixture of Water/THF (1:1, 0.14 M) was added LiOH·H₂O (2.00 g, 46.5 mmol, 9.00 equiv) at rt. Then the reaction was heated at 50° C. and stirred at this temperature overnight. The reaction mixture was diluted with ethyl acetate and the two phases were separated. The aqueous phase was washed with ethyl acetate. The aqueous layer was acidified to pH 1 with concentrated HCl. The aqueous layer was extracted with ethyl acetate. The combined organic layers were dried using a phase separator and concentrated under vacuum to afford the expected compound as a beige powder (1.4 g, 63%).

Analytical Data LC-MS/1H NMR

rt=1.25 min, 406.0 [M+H]+ (91% UV Abs).

1H NMR (400 MHz, DMSO-d6) δ 11.72 (s, 1H), 9.12 (s, 1H), 9.06 (s, 1H), 7.29 (dd, J=7.6, 1.7 Hz, 1H), 7.26-7.19 (m, 1H), 6.99 (ddd, J=8.0, 4.5, 3.3 Hz, 2H), 6.94 (td, J=7.4, 1.1 Hz, 1H), 6.67-6.60 (m, 2H), 6.49 (ddd, J=8.0, 2.4, 1.1 Hz, 1H), 4.88 (s, 1H), 3.78 (s, 3H), 3.44 (t, J=12.7 Hz, 1H), 2.75 (dd, J=17.1, 11.8 Hz, 1H), 2.64-2.52 (m, 2H), 2.34-2.24 (m, 4H).

TABLE 3
Synthesis and analytical data for compounds 12e, 12g, 50a, 50b, 52a-65a

| | | | General | |
|---|---|---|---|---|
| | Chemi- | | Procedure | |
| Cpd | cal | | and | |
| No. | ID | Structure | Modifications | Analytical Data LC-MS/1H NMR |
| 12e | NXI- 000241 | 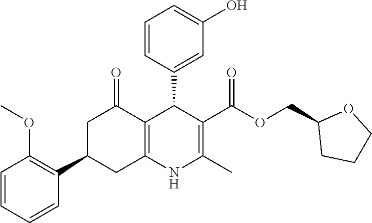 [SMILES: COc1ccccc1[C@H]1CC(=O)C2=C(C1)NC(C)=C(C(=O)OC[C@@H]1CCCO1)[C@H]2c1cccc(O)c1] | C | rt = 2.25 min, 490.0 [M + H]+, 100% UV 220 nm 1H NMR (600 MHz, DMSO-d6) δ 9.19 (s, 1H), 9.12 (s, 1H), 7.28 (dd, J = 7.6, 1.7 Hz, 1H), 7.25-7.21 (m, 1H), 7.00- 6.91 (m, 3H), 6.65-6.61 (m, 2H), 6.51- 6.46 (m, 1H), 4.89 (s, 1H), 4.00-3.93 (m, 2H), 3.92-3.87 (m, 1H), 3.77 (s, 3H), 3.66-3.59 (m, 2H), 3.48-3.41 (m, 1H), 2.79-2.72 (m, 1H), 2.62- 2.52 (m, 2H), 2.31-2.26 (m, 4H), 1.87- 1.71 (m, 3H), 1.49-1.42 (m, 1H). |
| 12g | NXI- 000240 | 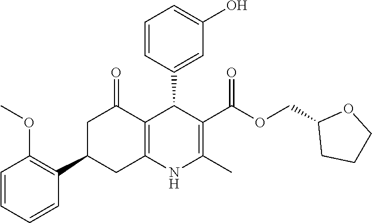 [SMILES: COc1ccccc1[C@H]1CC(=O)C2=C(C1)NC(C)=C(C(=O)OC[C@H]1CCCO1)[C@H]2c1cccc(O)c1] | C | rt = 1.61 min, 490.2 [M + H]+, 97% UV 220 nm 1H NMR (600 MHz, DMSO-d6) δ 9.19 (s, 1H), 9.13 (s, 1H), 7.28 (dd, J = 7.6, 1.7 Hz, 1H), 7.26-7.20 (m, 1H), 7.00- 6.97 (m, 2H), 6.95-6.92 (m, 1H), 6.66- 6.61 (m, 2H), 6.51-6.46 (m, 1H), 4.91 (s, 1H), 3.97-3.91 (m, 3H), 3.77 (s, 3H), 3.73-3.69 (m, 1H), 3.64-3.60 (m, 1H), 3.47-3.41 (m, 1H), 2.78- 2.72 (m, 1H), 2.62-2.52 (m, 2H), 2.32- 2.27 (m, 4H), 1.83-1.70 (m, 3H), 1.47-1.41 (m, 1H). |
| 50a | NXI- 000225 | 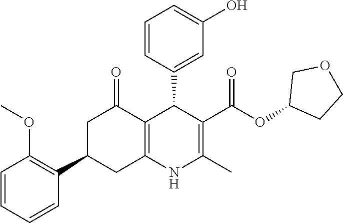 [SMILES: COc1ccccc1[C@H]1CC(=O)C2=C(C1)NC(C)=C(C(=O)O[C@H]1CCOC1)[C@H]2c1cccc(O)c1] | C | rt = 1.91 min, 476.2 [M + H]+, 96.2% UV 220 nm 1H NMR (600 MHz, DMSO-d6) δ 9.18 (s, 1H), 9.13 (s, 1H), 7.28 (dd, J = 7.6, 1.6 Hz, 1H), 7.23 (ddd, J = 8.2, 7.4, 1.7 Hz, 1H), 7.00-6.97 (m, 2H), 6.93 (t, J = 7.3 Hz, 1H), 6.64-6.62 (m, 2H), 6.49 (ddd, J = 8.0, 2.4, 1.2 Hz, 1H), 5.17- 5.13 (m, 1H), 4.83 (s, 1H), 3.77-3.75 (m, 4H), 3.70-3.67 (m, 2H), 3.63- 3.61 (m, 1H), 3.44 (tt, J = 12.4, 4.1 Hz, 1H), 2.78-2.70 (m, 1H), 2.62-2.51 (m, 2H), 2.30 (s, 3H), 2.29-2.26 (m, 1H), 2.11-1.97 (m, 1H), 1.67-1.62 |
| | | | | (m, 1H) |
| 50b | NXI- 000224 | 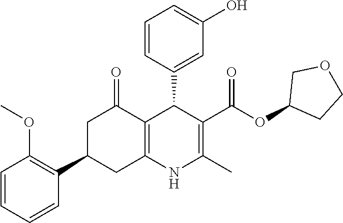 [SMILES: COc1ccccc1[C@H]1CC(=O)C2=C(C1)NC(C)=C(C(=O)O[C@@H]1CCOC1)[C@H]2c1cccc(O)c1] | C | rt = 1.91 min, 476.1 [M + H]+, 96.3% UV 220 nm 1H NMR (600 MHz, DMSO-d6) δ 9.18 (s, 1H), 9.12 (s, 1H), 7.28 (dd, J = 7.6, 1.6 Hz, 1H), 7.23 (ddd, J = 8.2, 7.4, 1.7 Hz, 1H), 7.02-6.96 (m, 2H), 6.93 (td, J = 7.5, 1.0 Hz, 1H), 6.66-6.60 (m, 2H), 6.52-6.47 (m, 1H), 5.14 (ddt, J = 6.5, 4.7, 1.9 Hz, 1H), 4.83 (s, 1H), 3.77 (s, 3H), 3.76-3.67 (m, 3H), 3.50-3.39 (m, 2H), 2.76 (dd, J = 17.1, 11.8 Hz, 1H), 2.60 (ddd, J = 17.1, 4.4, 1.5 Hz, 1H), 2.57-2.51 (m, 1H), 2.29 (s, 4H), 2.08 (dtd, J = 13.5, 8.4, 6.5 Hz, 1H), 1.89- |
| | | | | 1.78 (m, 1H) |
| 52a | NXI- 000202 | 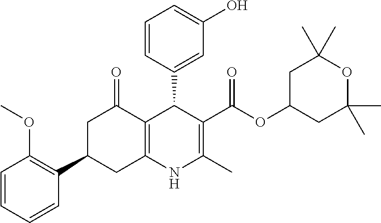 [SMILES: COc1ccccc1[C@H]1CC(=O)C2=C(C1)NC(C)=C(C(=O)OC1CC(C)(C)OC(C)(C)C1)[C@H]2c1cccc(O)c1] | D | rt = 2.46 min, 546.3 [M + H]+, 94.8% UV 220 nm 1H NMR (600 MHz, DMSO-d6) δ ppm 9.15 (s, 1 H) 9.11 (s, 1 H) 7.28 (dd, J = 7.63, 1.61 Hz, 1 H) 7.21-7.25 (m, 1 H) 6.97-7.01 (m, 2 H) 6.93 (td, J = 7.52, 1.10 Hz, 1 H) 6.60-6.65 (m, 2H) 6.49 (ddd, J = 8.00, 2.20, 1.25 Hz, 1 H) 5.07 (tt, J = 11.28, 4.27 Hz, 1 H) 4.84 (s, 1 H) 3.77 (s, 3 H) 3.40-3.49 (m, 1 H) 2.76 (dd, J = 17.02, 11.88 Hz, 1 H) 2.57-2.63 (m, 1 H) 2.53 (dd, J = 16.14, 13.20 Hz, 1 H) 2.29 (s, 3 H) 2.29 (br s, 1 H) 1.67- 1.91 (m, 2 H) 1.29 (t, J = 11.74 Hz, 1 H) |
| | | | | 1.19 (d, J = 15.70 Hz, 6 H) 1.09-1.17 (m, |
| | | | | 7 H) |
| 53a | NXI- 000201 | 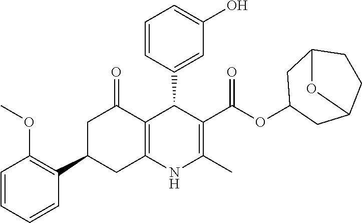 [SMILES: COc1ccccc1[C@H]1CC(=O)C2=C(C1)NC(C)=C(C(=O)OC1CC3CCC(C1)O3)[C@H]2c1cccc(O)c1] | D | rt = 7.09 min, 516.2 [M + H]+, 96.2% UV 220 nm 1H NMR (600 MHz, DMSO-d6) δ 9.04- 9.25 (m, 2 H) 7.17-7.43 (m, 2 H) 6.84- 7.06 (m, 3 H) 6.57-6.69 (m, 2 H) 6.48 (ddd, J = 8.0, 2.4, 1.0 Hz, 1 H) 4.88-5.00 (m, 1 H) 4.80 (s, 1 H) 4.28-4.42 (m, 2 H) 3.70-3.91 (m, 3 H) 3.35-3.51 (m, 1 H) 2.52-2.85 (m, 3 H) 2.15-2.32 (m, 4 H) 1.36-1.94 (m, 8 H) ppm |
| 54a | NXI- 000294 | 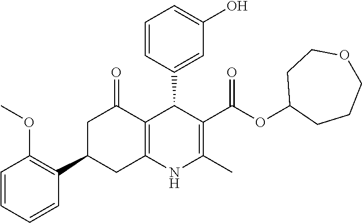 [SMILES: COc1ccccc1[C@H]1CC(=O)C2=C(C1)NC(C)=C(C(=O)OC1CCCOCC1)[C@H]2c1cccc(O)c1] | C | 2 Diastereoisomers at 50/50 rt = 2.36 and 2.37 min, 504.1 [M + H]+, 97.9% UV 220 nm 1H NMR (500 MHz, DMSO-d6) δ ppm 1.36-1.95 (m, 6 H) 2.24-2.38 (m, 4 H) 2.52-2.81 (m, 3 H) 3.38-3.65 (m, 5 H) 3.78 (s, 3 H) 4.89 (d, J = 5.38 Hz, 2 H) 6.46-6.52 (m, 1 H) 6.62-6.69 (m, 2 H) 6.91-6.97 (m, 1 H) 6.97-7.04 (m, 2 H) 7.21-7.26 (m, 1 H) 7.28 (dd, J = 7.63, 1.50 Hz, 1 H) 9.12 (s, 1 H) 9.15 (s, 1 H) |
| 55a | NXI- 000248 | 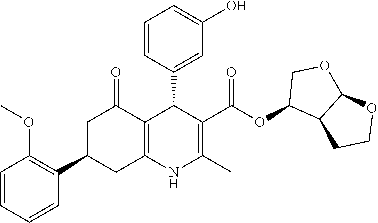 [SMILES: COc1ccccc1[C@H]1CC(=O)C2=C(C1)NC(C)=C(C(=O)O[C@H]1CO[C@H]3OCC[C@H]31)[C@H]2c1cccc(O)c1] | C | rt = 2.13 min, 518.0 [M + H]+, 96.2% UV 220 nm 1H NMR (DMSO) δ: 9.26 (s, 1H), 9.15 (s, 1H), 7.29-7.22 (m, 2H), 7.01- 6.97 (m, 2H), 6.95-6.92 (m, 1H), 6.64- 6.60 (m, 2H), 6.52-6.47 (m, 1H), 5.57 (d, J = 5.1 Hz, 1H), 5.09-5.02 (m, 1H), 4.88 (s, 1H), 3.87-3.83 (m, 1H), 3.80- 3.76 (m, 4H), 3.60-3.54 (m, 1H), 3.46- 3.39 (m, 1H), 3.39-3.35 (m, 1H), 3.00- 2.93 (m, 1H), 2.79-2.73 (m, 1H), 2.62-2.52 (m, 2H), 2.34-2.24 (m, 4H), 1.81-1.69 (m, 2H) |
| 56a | NXI- 000281 | 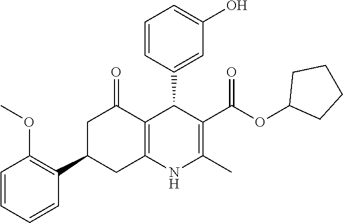 [SMILES: COc1ccccc1[C@H]1CC(=O)C2=C(C1)NC(C)=C(C(=O)OC1CCCC1)[C@H]2c1cccc(O)c1] | C | rt = 2.34 min, 474.4 [M + H]+, 98.4% UV 220 nm 1H NMR (DMSO-d6, 500 MHz) δ 9.11 (s, 2H), 7.28 (dd, 1H, J = 1.4, 7.7 Hz), 7.2-7.3 (m, 1H), 7.0-7.1 (m, 2H), 6.94 (dt, 1H, J = 0.9, 7.5 Hz), 6.6-6.7 (m, 2H), 6.49 (ddd, 1H, J = 1.1, 2.4, 8.1 Hz), 5.02 (tt, 1H, J = 2.7, 5.6 Hz), 4.83 (s, 1H), 3.78 (s, 3H), 3.4-3.5 (m, 1H), 2.75 (dd, 1H, J = 11.8, 16.9 Hz), 2.5-2.6 (m, 2H), 2.2- 2.3 (m, 4H), 1.3-1.9 (m, 8H) |
| 57a | NXI- 000269 | 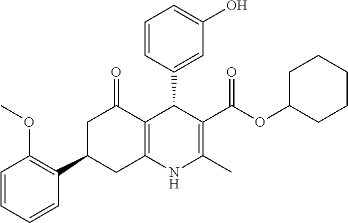 [SMILES: COc1ccccc1[C@H]1CC(=O)C2=C(C1)NC(C)=C(C(=O)OC1CCCCC1)[C@H]2c1cccc(O)c1] | C | rt = 2.48 min, 488.4 [M + H]+, 99.4% UV 220 nm 1H NMR (DMSO-d6, 600 MHz) δ 9.12 (s, 1H), 9.10 (s, 1H), 7.28 (dd, 1H, J = 1.5, 7.6 Hz), 7.23 (dt, 1H, J = 1.6, 7.8 Hz), 6.9-7.0 (m, 3H), 6.6-6.7 (m, 2H), 6.48 (ddd, 1H, J = 1.0, 2.4, 8.0 Hz), 4.88 (s, 1H), 4.6-4.7 (m, 1H), 3.77 (s, 3H), 3.4- 3.5 (m, 1H), 2.75 (dd, 1H, J = 11.8, 17.1 Hz), 2.5-2.6 (m, 2H), 2.2-2.3 (m, 4H), 1.2-1.8 (m, 10H) |
| 58a | NXI- 000282 | 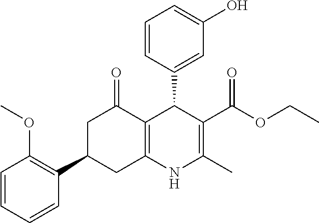 [SMILES: CCOC(=O)C1=C(C)NC2=C(C(=O)C[C@H](c3ccccc3OC)C2)[C@@H]1c1cccc(O)c1] | E | rt = 2.05 min, 434.3 [M + H]+, 97.2% UV 220 nm 1H NMR (DMSO-d6, 500 MHz) δ 9.15 (s, 1H), 9.13 (s, 1H), 7.29 (dd, 1H, J = 1.5, 7.6 Hz), 7.23 (dd, 1H, J = 1.1, 8.0 Hz), 7.0-7.0 (m, 2H), 6.94 (t, 1H, J = 7.5 Hz), 6.6-6.7 (m, 2H), 6.4-6.5 (m, 1H), 4.89 (s, 1H), 4.00 (q, 2H, J = 7.0 Hz), 3.78 (s, 3H), 3.4-3.5 (m, 1H), 2.77 (dd, 1H, J = 11.8, 17.0 Hz), 2.5-2.7 (m, 2H), 2.30 (s, 4H), 1.14 (t, 3H, J = 7.1 Hz) |
| 59a | NXI- 000283 | 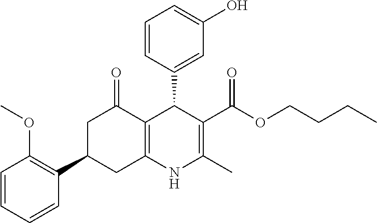 [SMILES: CCCCOC(=O)C1=C(C)NC2=C(C(=O)C[C@H](c3ccccc3OC)C2)[C@@H]1c1cccc(O)c1] | E | rt = 2.35 min, 462.4 [M + H]+, 98.6% UV 220 nm 1H NMR (DMSO-d6, 500 MHz) δ 9.16 (s, 1H), 9.12 (s, 1H), 7.29 (dd, 1H, J = 1.6, 7.6 Hz), 7.2-7.3 (m, 1H), 7.0-7.0 (m, 2H), 6.94 (dt, 1H, J = 0.9, 7.5 Hz), 6.6-6.7 (m, 2H), 6.4-6.5 (m, 1H), 4.89 (s, 1H), 3.9-4.0 (m, 2H), 3.78 (s, 3H), 3.4- 3.5 (m, 1H), 2.76 (dd, 1H, J = 11.6, 17.0 Hz), 2.5-2.7 (m, 2H), 2.2-2.3 (m, 4H), 1.4-1.5 (m, 2H), 1.2-1.3 (m, 2H), 0.84 (t, 3H, J = 7.4 Hz) |
| 60a | NXI- 000289 | 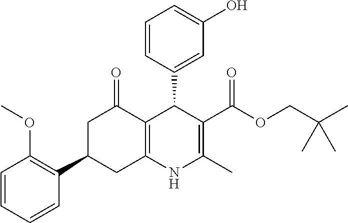 [SMILES: COc1ccccc1[C@H]1CC(=O)C2=C(C1)NC(C)=C(C(=O)OCC(C)(C)C)[C@H]2c1cccc(O)c1] | C | rt = 2.74 min, 476.3 [M + H]+, 97.6% UV 220 nm 1H NMR (600 MHz, DMSO-d6) δ 9.18 (s, 1H), 9.10 (s, 1H), 7.27 (dd, J = 7.6, 1.6 Hz, 1H), 7.22 (t, J = 7.6 Hz, 1H), 6.99-6.96 (m, 2H), 6.93 (t, J = 7.3 Hz, 1H), 6.66-6.64 (m, 2H), 6.48 (ddd, J = 8.0, 2.3, 1.2 Hz, 1H), 4.95 (s, 1H), 3.76 (s, 3H), 3.68 (d, J = 10.6 Hz, 1H), 3.60 (d, J = 10.6 Hz, 1H), 3.40 (tt, J = 12.6, 4.0 Hz, 1H), 2.73 (dd, J = 17.2, 11.9 Hz, 1H), 2.60-2.52 (m, 2H), 2.35 (s, 3H), 2.30-2.24 (m, 1H), 0.80 (s, 9H) |
| 61a | NXI- 000290 | 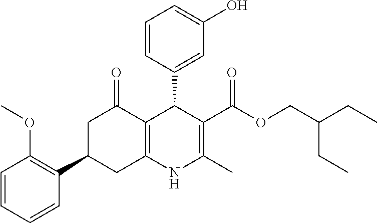 [SMILES: CCC(CC)COC(=O)C1=C(C)NC2=C(C(=O)C[C@H](c3ccccc3OC)C2)[C@@H]1c1cccc(O)c1] | C | rt = 2.9 min, 490.3 [M + H]+, 97.5% UV 220 nm 1H NMR (DMSO-d6, 600 MHz) δ 9.16 (s, 1H), 9.09 (s, 1H), 7.27 (dd, 1H, J = 1.3, 7.6 Hz), 7.2-7.2 (m, 1H), 6.9-7.0 (m, 3H), 6.6-6.7 (m, 2H), 6.48 (td, 1H, J = 1.2, 8.0 Hz), 4.90 (s, 1H), 3.8-3.9 (m, 2H), 3.77 (s, 3H), 3.4-3.5 (m, 1H), 2.74 (dd, 1H, J = 11.9, 17.0 Hz), 2.5-2.6 (m, 2H), 2.32 (s, 3H), 2.2-2.3 (m, 1H), 1.3- 1.4 (m, 1H), 1.20 (dd, 4H, J = 7.0, 14.1 Hz), 0.77 (td, 6H, J = 7.5, 11.6 Hz) |
| 62a | NXI- 000293 | 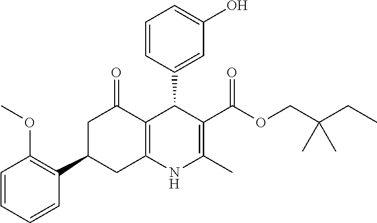 [SMILES: CCC(C)(C)COC(=O)C1=C(C)NC2=C(C(=O)C[C@H](c3ccccc3OC)C2)[C@@H]1c1cccc(O)c1] | C | rt = 2.87 min, 490.1 [M + H]+, 97.1% UV 220 nm 1H NMR (500 MHz, DMSO-d6) δ ppm 0.71 (t, J = 7.50 Hz, 3 H) 0.75 (d, J = 4.75 Hz, 6 H) 1.15 (q, J = 7.59 Hz, 2 H) 2.25- 2.32 (m, 1 H) 2.36 (s, 3 H) 2.53-2.79 (m, 3 H) 3.36-3.46 (m, 1 H) 3.60-3.73 (m, 2 H) 3.77 (s, 3 H) 4.95 (s, 1 H) 6.49 (ddd, J = 7.94, 2.31, 0.88 Hz, 1 H) 6.66 (dd, J = 4.25, 1.88 Hz, 2 H) 6.94 (t, J = 7.44 Hz, 1 H) 6.96-7.04 (m, 2 H) 7.20- 7.26 (m, 1 H) 7.28 (dd, J = 7.63, 1.63 Hz, 1 H) 9.10 (s, 1 H) 9.19 (s, 1 H) |
| 63a | NXI- 000295 | 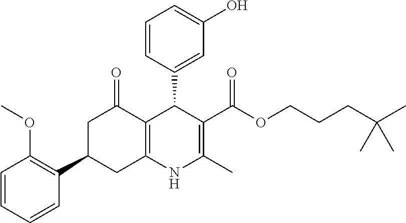 [SMILES: COc1ccccc1[C@H]1CC(=O)C2=C(C1)NC(C)=C(C(=O)OCCCC(C)(C)C)[C@H]2c1cccc(O)c1] | C | rt = 3.03 min, 504.1 [M + H]+, 97.3% UV 220 nm 1H NMR (500 MHz, DMSO-d6) δ ppm 0.80-0.84 (m, 9 H) 1.03-1.09 (m, 2 H) 1.42-1.49 (m, 2 H) 2.27-2.31 (m, 1 H) 2.33 (s, 3 H) 2.52-2.62 (m, 2 H) 2.75 (dd, J = 17.13, 11.88 Hz, 1 H) 3.40- 3.47 (m, 1 H) 3.78 (s, 3 H) 3.87 (dt, J = 10.76, 6.32 Hz, 1 H) 4.00 (dt, J = 10.76, 6.44 Hz, 1 H) 4.91 (s, 1 H) 6.46-6.53 (m, 1 H) 6.63-6.65 (m, 2 H) 6.92-7.01 (m, 3 H) 7.24 (t, J = 7.61 Hz, 1 H) 7.29 (dd, J = 7.63, 1.75 Hz, 1 H) 9.11 (s, 1 H) 9.16 (s, 1 H) |
| 64a | NXI- 000297 | 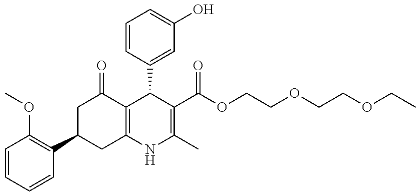 [SMILES: CCOCCOCCOC(=O)C1=C(C)NC2=C(C(=O)C[C@H](c3ccccc3OC)C2)[C@@H]1c1cccc(O)c1] | C | rt = 2.37 min, 522.1 [M + H]+, 99.2% UV 220 nm 1H NMR (DMSO-d6, 600 MHz): δ (ppm) 9.17 (s, 1H), 9.11 (s, 1H), 7.28 (dd, J = 7.6, 1.6 Hz, 1H), 7.20-7.25 (m, 1H), 6.91-7.00 (m, 3H), 6.63-6.66 (m, 1H), 6.60-6.63 (m, 1H), 6.49 (ddd, J = 8.0, 2.5, 1.0 Hz, 1H), 4.89 (s, 1H), 4.83- 4.86 (m, 1H), 4.01-4.12 (m, 2H), 3.78 (s, 3H), 3.52-3.62 (m, 2H), 3.36-3.51 (m, 7H), 2.76 (dd, J = 17.1, 11.8 Hz, 1H), 2.51-2.65 (m, 2H), 2.24-2.32 (m, 4H), 1.09 (t, J = 7.0 Hz, 3H) |
| 65a | NXI- 000296 | 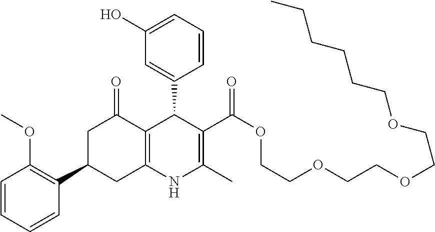 [SMILES: CCCCCCOCCOCCOCCOC(=O)C1=C(C)NC2=C(C(=O)C[C@H](c3ccccc3OC)C2)[C@@H]1c1cccc(O)c1] | C | rt = 2.73 min, 622.4 [M + H]+, 99.3% UV 220 nm 1H NMR (DMSO-d6, 600 MHz): δ (ppm) 9.17 (s, 1H), 9.10 (br s, 1H), 7.28 (dd, J = 7.6, 1.6 Hz, 1H), 7.23 (ddd, J = 8.1, 7.4, 1.8 Hz, 1H), 6.95-7.01 (m, 2H), 6.93 (td, J = 7.5, 1.0 Hz, 1H), 6.64 (dd, J = 7.6, 1.2 Hz, 1H), 6.59-6.63 (m, 1H), 6.48 (ddd, J = 8.1, 2.5, 1.0 Hz, 1H), 4.89 (s, 1H), 4.02-4.11 (m, 2H), 3.77 (s, 3H), 3.56 (td, J = 4.8, 2.4 Hz, 2H), 3.452-3.52 (m, 9H), 3.35 (t, J = 6.7 Hz, 2H), 2.76 (dd, J = 17.0, 11.9 Hz, 1H), 2.61 (ddd, J = 17.1, 4.3, 1.3 Hz, 1H), 2.54 (dd, J = 16.2, 13.3 Hz, 1H), 2.23-2.35 (m, 4H), |
| | | | | 1.41-1.52 (m, 2H), 1.19-1.32 (m, 6H), |
| | | | | 0.82-0.88 (m, 3H) |

### Example 5

Enantioselective Syntheses of Compound 51a and Syntheses of Compounds 66b and 67b

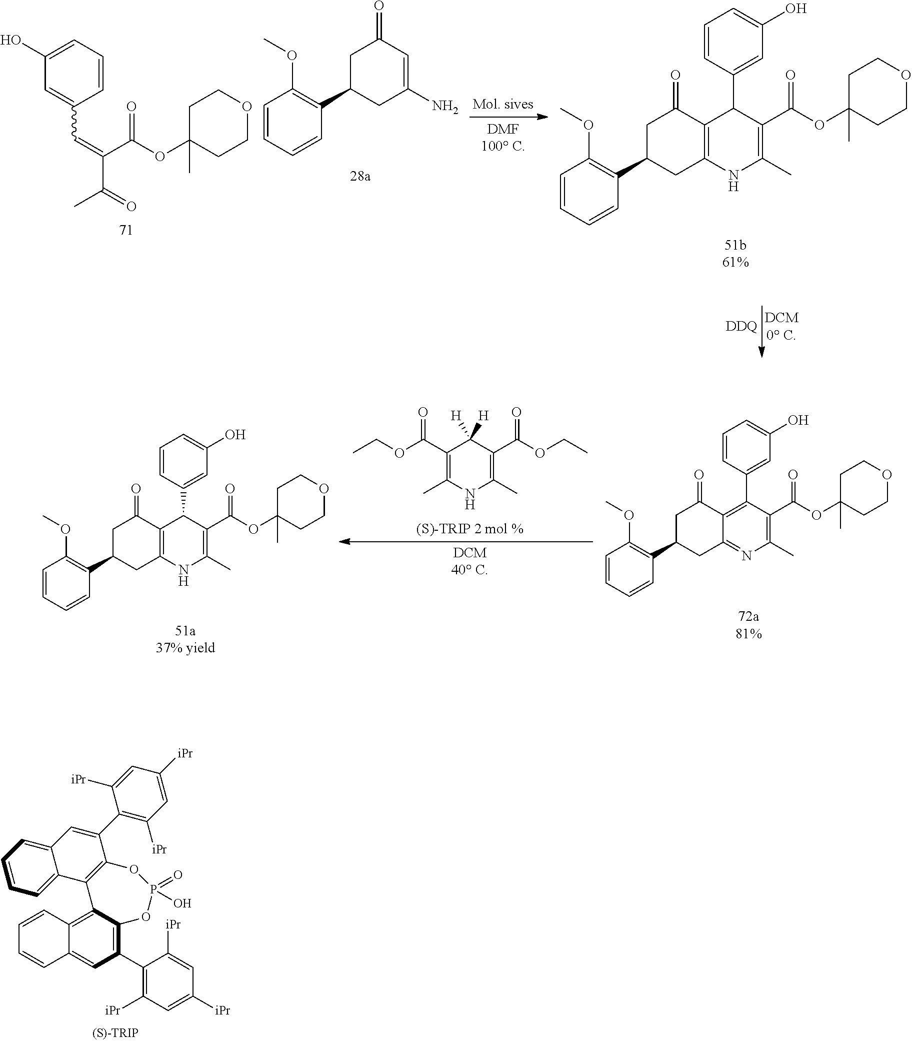

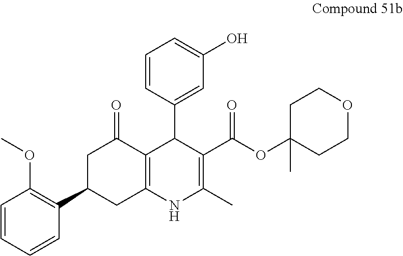 [SMILES: COc1ccccc1[C@H]1CC(=O)C2=C(C1)NC(C)=C(C(=O)OC1(C)CCOCC1)C2c1cccc(O)c1]

A 1 ml vial was successively charged with 28a (55 mg, 0.25 mmol, 1.00 equiv). Condensation of aldehyde and β-keto esters conducted according to reported procedures e.g: Chemical and Pharmaceutical Bulletin, 1986, 34, 1589. (4-methyltetrahydropyran-4-yl) 2-[(3-hydroxyphenyl)methylene]-3-oxo-butanoate (71) (101 mg, 0.33 mmol, 1.30 equiv) in DMF (0.28 mL, 0.92 M) and Molecular sieves were added. The reaction mixture was stirred at 100° C. overnight. The reaction mixture was concentrated in vacuo. The crude product was purified via silica gel column chromatography (acetone:toluene) resulting in the expected compound (86 mg, 61%, 1:1 d.r).

Analytical Data LC-MS/1H NMR

2 Diastereoisomers at 50/50

rt=0.85 and 0.86 min, 504.2 and 504.2 [M+H]+, combined 90% Uv Abs

1H NMR (DMSO) δ: 9.12 (d, J=6.5 Hz, 1H), 9.02 (d, J=12.8 Hz, 1H), 7.34-7.10 (m, 1H), 7.05-6.42 (m, 7H), 4.88 (d, J=18.5 Hz, 1H), 3.78 (d, J=7.5 Hz, 3H), 3.70-3.46 (m, 3H), 3.29-3.23 (m, 2H), 2.68 (d, J=6.9 Hz, 1H), 2.61-2.52 (m, 1H), 2.49-2.38 (m, 1H), 2.31 (d, J=6.2 Hz, 4H), 1.97 (d, J=13.2 Hz, 2H), 1.56 (d, J=10.8 Hz, 2H), 1.34 (d, J=1.3 Hz, 3H)

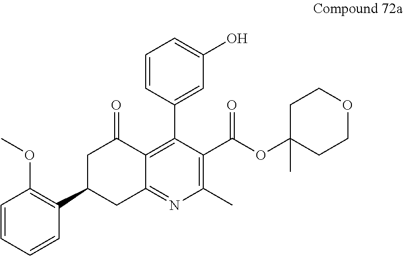 [SMILES: COc1ccccc1[C@H]1CC(=O)c2c(nc(C)c(C(=O)OC3(C)CCOCC3)c2-c2cccc(O)c2)C1]

A suspension of the Hantzsch 1,4-DHP 51b (86 mg, 90%, 0.15 mmol, 1.00 equiv) and 2,3-dichloro-5,6-dicyano-1,4-benzoquinone (35 mg, 0.15 mmol, 1.00 equiv) in DCM (1.5 mL, 0.10 M) was stirred at 0° C. for 1 h. The reaction mixture was filtered, the solid was washed with DCM and the filtrate was concentrated under reduced pressure to obtain a brown residue. The crude product was purified via silica gel column chromatography (acetone:toluene) resulting in the expected compound (67 mg, 81%).

Analytical Data LC-MS/1H NMR

rt=0.94 min, 502.1 [M+H]+ (89% UV Abs) (impurity from LCMS at rt=1.06 min, 6% so real purity=93%)

1H NMR (DMSO) δ: 9.42 (s, 1H), 7.30-7.22 (m, 2H), 7.21-7.11 (m, 1H), 7.06-7.00 (m, 1H), 6.95 (td, J=7.5, 1.1 Hz, 1H), 6.74 (dd, J=8.3, 2.4 Hz, 1H), 6.57-6.46 (m, 2H), 3.81 (s, 4H), 3.53-3.34 (m, 3H), 3.26 (d, J=16.6 Hz, 3H), 3.04-2.86 (m, 1H), 2.71-2.57 (m, 1H), 2.53 (s, 3H), 1.78 (s, 2H), 1.56 (s, 2H), 1.19 (s, 3H)

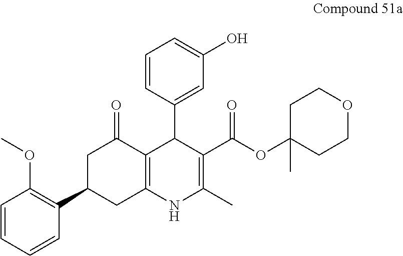 [SMILES: COc1ccccc1[C@H]1CC(=O)C2=C(C1)NC(C)=C(C(=O)OC1(C)CCOCC1)C2c1cccc(O)c1]

In a vial, pyridine 72a (67 mg, 90%, 0.120 mmol, 1.00 equiv) was dissolved in DCM (0.60 mL, 0.20 M). Then, diethyl 2,6-dimethyl-1,4-dihydropyridine-3,5-dicarboxylate (64 mg, 95%, 0.24 mmol, 2.00 equiv) and S-TRIP (1.9 mg, 97%, 2.40 μmol, 0.02 equiv) were added. The reaction mixture was stirred overnight at 40° C. The solvent was removed under reduced pressure and the residue was purified via silica gel column chromatography (acetone:toluene) resulting in the expected compound (23 mg, 37%).

Analytical Data LC-MS/1H NMR

rt=2.38 min, 504.3 [M+H]+, 98% UV 220 nm

1H NMR (DMSO-d6, 500 MHz) δ 9.12 (d, 2H, J=7.5 Hz), 7.28 (dd, 1H, J=1.5, 7.6 Hz), 7.23 (dt, 1H, J=1.6, 7.8 Hz), 7.0-7.0 (m, 2H), 6.94 (t, 1H, J=7.4 Hz), 6.6-6.7 (m, 2H), 6.5-6.5 (m, 1H), 4.91 (s, 1H), 3.77 (s, 3H), 3.5-3.6 (m, 2H), 3.4-3.5 (m, 1H), 3.2-3.3 (m, 2H), 2.73 (dd, 1H, J=11.7, 17.1 Hz), 2.5-2.6 (m, 2H), 2.2-2.4 (m, 4H), 1.98 (br d, 2H, J=14.1 Hz), 1.5-1.7 (m, 2H), 1.35 (s, 3H)

Chiral SFC: Pirkle Whelk-01 (R,R), CO₂:MeOH+0.5% IPAm=6:3, 2.4 mL/min, 104 bar, λ=254, t_{R} (minor)=3.9 min; t_{R} (major)=4.7 min, d.r.=98:2.

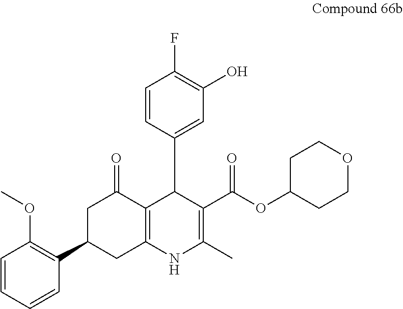 [SMILES: COc1ccccc1[C@H]1CC(=O)C2=C(C1)NC(C)=C(C(=O)OC1CCOCC1)C2c1ccc(F)c(O)c1]

A 1 ml vial was successively charged with 28a (35 mg, 0.16 mmol, 1.00 equiv). Condensation of aldehyde and β-keto esters conducted according to reported procedures e.g: Chemical and Pharmaceutical Bulletin, 1986, 34, 1589. tetrahydro-2H-pyran-4-yl 2-(4-fluoro-3-hydroxybenzylidene)-3-oxobutanoate (73) (74 mg, 0.21 mmol, 1.30 equiv, 87%) in DMF (0.18 mL, 0.92 M) and Molecular sieves were added. The reaction mixture was stirred at 100° C. overnight. The reaction mixture was concentrated in vacuo. The crude product was purified by reverse phase column chromatography (water: acetonitrile) resulting in the expected compound (47 mg, 57%, 1:1 d.r).

Analytical Data LC-MS/1H NMR

2 Diastereoisomers at 50/50

rt=2.04 and 2.08 min, 508.4 [M+H]+, 48+50.8% UV 220 nm

1H NMR (DMSO-d6, 600 MHz): δ (ppm) 9.40-9.62 (m, 1H), 9.00-9.31 (m, 1H), 7.21-7.28 (m, 1H), 7.12-7.19 (m, 1H), 6.92-7.00 (m, 2H), 6.83-6.89 (m, 1H), 6.70-6.82 (m, 1H), 6.50-6.63 (m, 1H), 4.86 (s, 1H), 4.77-4.83 (m, 1H), 3.76-3.80 (m, 3H), 3.69-3.76 (m, 1H), 3.34-3.68 (m, 4H), 2.65-2.78 (m, 1H), 2.51-2.61 (m, 1H), 2.38-2.49 (m, 1H), 2.27-2.33 (m, 4H), 1.77-1.83 (m, 1H), 1.62-1.69 (m, 1H), 1.49-1.56 (m, 1H), 1.31-1.38 (m, 1H)

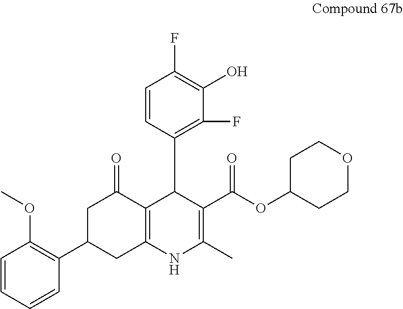 [SMILES: COc1ccccc1C1CC(=O)C2=C(C1)NC(C)=C(C(=O)OC1CCOCC1)C2c1ccc(F)c(O)c1F]

A 1 ml vial was successively charged with 28a (40 mg, 0.18 mmol, 1.00 equiv). Condensation of aldehyde and β-keto esters conducted according to reported procedures e.g: Chemical and Pharmaceutical Bulletin, 1986, 34, 1589. tetrahydro-2H-pyran-4-yl 2-(2,4-difluoro-3-hydroxybenzylidene)-3-oxobutanoate (74) (79 mg, 0.24 mmol, 1.30 equiv) in DMF (0.20 mL, 0.92 M) and Molecular sieves were added. The reaction mixture was stirred at 100° C. overnight. The reaction mixture was concentrated in vacuo. The crude product was purified by reverse phase column chromatography (water: acetonitrile) resulting in the expected compound (33 mg, 34%, 1:1 d.r).

Analytical Data LC-MS/1H NMR

2 Diastereoisomers at 50/50

rt=2.06 and 2.1 min, 526.3 and 526.4 [M+H]+, 45.5 and 54.5% UV 220 nm

1H NMR (DMSO-d6, 500 MHz): δ (ppm) 9.69-9.82 (m, 1H), 9.11-9.25 (m, 1H), 6.84-7.34 (m, 4H), 6.75-6.82 (m, 1H), 6.50-6.70 (m, 1H), 5.04 (d, J=18.0 Hz, 1H), 4.75 (td, J=8.9, 4.4 Hz, 1H), 3.76-3.82 (m, 4H), 3.62-3.73 (m, 2H), 3.33-3.47 (m, 3H), 2.67-2.81 (m, 1H), 2.52-2.65 (m, 1H), 2.39-2.49 (m, 1H), 2.23-2.31 (m, 3H), 1.77-1.82 (m, 1H), 1.52-1.63 (m, 2H), 1.33 (dt, J=12.8, 3.3 Hz, 1H)

### Example 6

### Screening Assay for Identifying Inhibitors of Coronin 1 Promoter Activity and Biological Characterization

Towards identification of compounds with coronin 1 promoter inhibitory activity, initially the coronin 1 promoter was characterization by cloning in to promoterless plasmids driving luciferase or fluorescence constructs as read-out for promoter activity. Subsequently, we screening various compounds from libraries to identify the coronin 1 promoter inhibitory compounds. This was subsequently validated by means of qPCR, western blotting and taken for SAR based optimization, safety assessment and immunosuppressive activity assessment in an autoimmune-inflammatory model of psoriasis. These procedures detailed below;

### Vector Design, Transfection and Compound Identification Via Fluorescence Read-Out

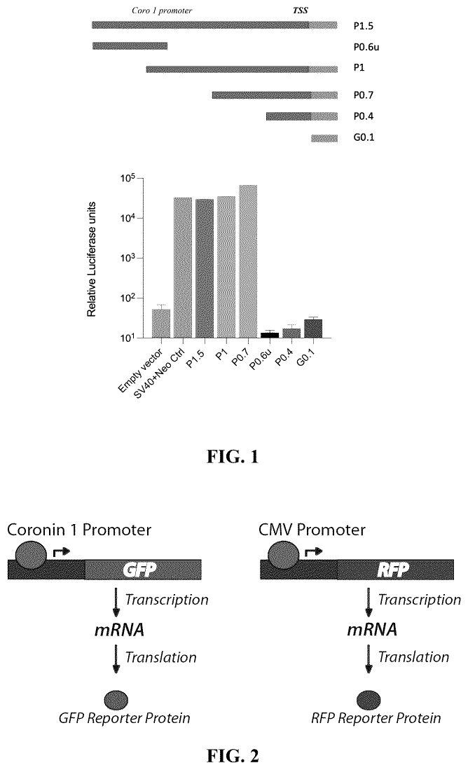

The inventors designed a fluorescence-based screening assay to identify compounds that selectively inhibit coronin 1 promoter activity. The vector used for the assay comprises a promoter sequence which consists of a 3 kb or 1.53 kb (P1.5; SEQ ID NO: 1-3) or 1 kb or 737 bp (P.7; SEQ ID NO: 4-6) sequence located upstream from the TSS of coronin 1 gene in a plasmid lacking a promoter (promoter-less vector) but containing as a read-out luciferase expressing gene. J774 macrophages or RBL were transfected with the plasmid and assessed for luciferase activity (FIG. 1). Instead of macrophages or RBL cells any other non-immune or preferably immune cell type can be used. The 1530 base pair fragment of SEQ ID NO: 1-3 located 5′ (upstream) to the coronin 1 transcription start site gave the best coronin 1 promoter activity in a luciferase activity assay. Further, the fragments P0.7 also showed coronin 1 promoter activity in the luciferase activity assay (FIG. 1). However, the 1530 bp fragment was further characterized and used for developing the screening assay taking into consideration the presence of additional regulatory elements.

The murine coronin 1 promoter sequences of SEQ ID NO: 1-6 were each cloned into a plasmid lacking a promoter (promoterless) but containing a destabilized Green Fluorescence Protein (GFP) cassette. Rat Basophil Leukemia (RBL) cells were transfected with the plasmids, stable GFP expressing cells were enriched, incubated with various compounds and assessed for coronin 1 promoter inhibition by assessing green fluorescence values (FIG. 2).

As an unrelated promoter, the early cytomegaloviral promoter element was cloned into a promoterless Red Fluorescence Protein (RFP) expression plasmid. Rat Basophil Leukemia (RBL) cells were transfected with the plasmid, stable RFP expressing cells enriched, incubated with various compounds and were assessed for red fluorescence values. Analyzing the inhibition of an unrelated promoter (in this case, early cytomegalovirus promoter) that drove Red Fluorescence Protein (RFP) expression assessed non-specificity of the compound under analysis.

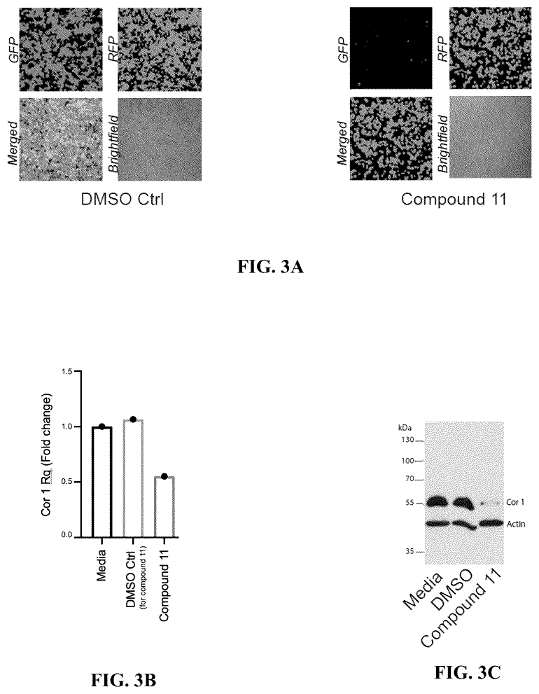

The plasmids were independently transfected into immune cells (Rat basophil Leukemia, RBL cells) to generate green and red fluorescent RBL cells that were subsequently enriched by means of flow cytometry. These cells were then mixed in equal ratio (1:1) and incubated in a 96 well plate at 100,000 cells/well in a volume of 200 μl of phenol red free RPMI supplemented with 8% fetal calf serum, L-glutamate and antibiotics (penicillin, streptomycin) and various compounds (3 and 5 μg/ml concentrations) from available chemical libraries. At defined time points (8 h, 24 h and 48 h), the GFP and RFP fluorescence was measured using a 96 well microplate reader (Synergy, BioTek Instruments). The top hit compounds identified from such an initial screen of 12,000 compounds that selectively reduced GFP fluorescence are listed in Table 5. Selected compounds show different levels of modulation of coronin 1 promoter driven GFP and CMV promoter driven RFP fluorescence. GFP and RFP fluorescence were analyzed with a microplate reader upon incubation with the top coronin 1 promoter inhibiting compounds. Shown are percentage of GFP reduction (left column) and percentage of RFP reduction (right column) for the compounds at 5 μg/ml concentration at 48 h. Cycloheximide (CHX) serves as positive control whereas media and DMSO serve as negative controls. FIG. 3A-C show the top hit of the initial screen, compound 11, to selectively reduce GFP values. Cells were imaged using a fluorescence microscope to validate fluorescence inhibition (FIG. 3A) and as well assess morphology and viability (in addition to GFP down regulation confirmation). In addition, quantitative Polymerase Chain Reaction (qPCR) for coronin 1 mRNA transcripts reveal reduction with compound 11 (FIG. 3B). The primers used for the qPCR on RBL cells are listed below as follows;

Corla forward primer:
(SEQ ID NO.: 7)
5′ GTG ACA GCT CTA TCC GGT ATT T 3′
Corla reverse primer:
(SEQ ID NO.: 8)
5′ ACG TTG AGA CTC CTT GGA AC 3′
GAPDH forward primer:
(SEQ ID NO.: 9)
5′ GGG AAA CCC ATC ACC ATC TT 3′
GAPDH reverse primer:
(SEQ ID NO.: 10)
5′ CCA GTA GAC TCC ACG ACA TAC T 3′

GAPDH was used as the house-keeping gene for normalization purpose. SYBR green based qPCR assessment was performed.

### Western Blotting

RBL Cells that had been incubated with the compound (10 μg/ml) for 96 h were lysed in Triton-X 100 buffer with 0.2% SDS containing protease and phosphatase inhibitors (Roche) at 4° C., followed by protein determination (BCA, Pierce) and SDS-PAGE with equal protein amounts transferred onto nitrocellulose and probed using antibodies against the indicated proteins (actin and coronin 1) followed by HRP-labeled secondary antibodies and developed using an enhanced chemi-luminescence imager (Fuji) (FIG. 3C).

Splenocytes from mice that had been administered with either compound 11 (150 mg/kg body weight, B.D, S.C route) or vehicle control (DMSO) for 5 days were lysed in Triton-X 100 buffer with 0.2% SDS containing protease and phosphatase inhibitors (Roche) at 4° C., followed by protein determination (BCA, Pierce) and SDS-PAGE, transferred onto nitrocellulose and probed using antibodies against the indicated proteins (actin and coronin 1) followed by infrared dye tagged secondary antibodies and imaged using a Licor system (FIG. 7, 11).

Human PBMCs that had been incubated with the indicated compounds (20 μg/ml concentrations) for 96 h were lysed in Triton-X 100 buffer with 0.2% SDS containing protease and phosphatase inhibitors (Roche) at 4° C., followed by protein determination (BCA, Pierce) and SDS-PAGE, transferred onto nitrocellulose and probed using antibodies against the indicated proteins (actin and coronin 1) followed by HRP-labeled secondary antibodies and developed using an enhanced chemi-luminescence imager (Fuji) (FIG. 8A).

Coronin 1 Promoter Driven GFP Fluorescence Analysis of RBL Cells Incubated with Calcium Channel Blockers:

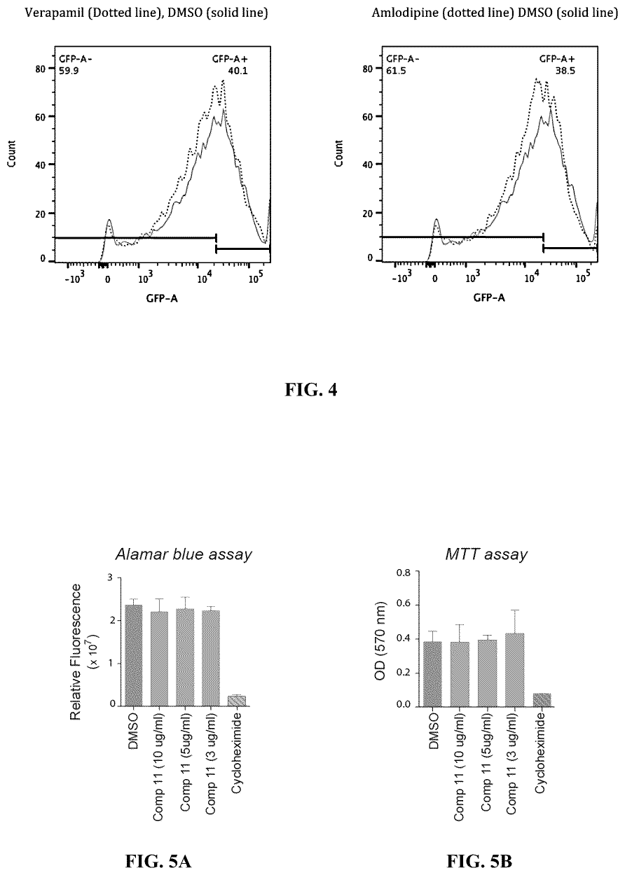

RBL cells that had been incubated with the indicated calcium channel blockers (amlodipine (3.125 μM) and verapamil (8 μM) concentrations for 48 h were analyzed by flow cytometry for any alteration in coronin 1 promoter driven GFP fluorescence. The results reveal no effect of calcium channel blockers on coronin 1 promoter activity (FIG. 4).

### EC₅₀ Analysis for Coronin 1 Promoter Driven GFP Inhibition (Specific Activity), CMV Promoter Driven RFP Inhibition (Non-Specific Activity) and Cell Viability Using Live-Dead Staining for Cytotoxicity:

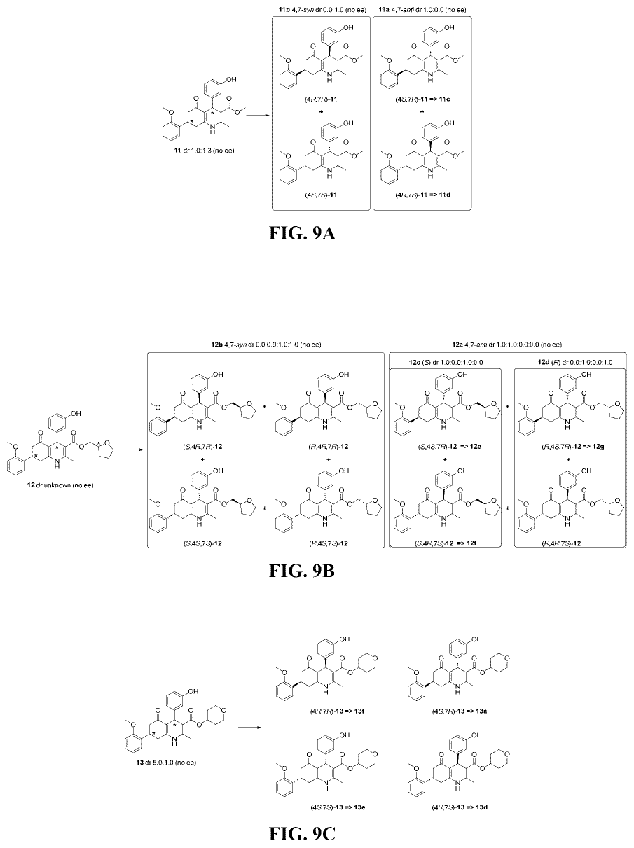

RBL cells expressing GFP under coronin 1 promoter and optionally with RBL cells expressing RFP under CMV promoter were mixed in equal numbers and incubated with the compounds at indicated concentrations for 48-65 h and analyzed by flow cytometry for alteration in coronin 1 promoter (GFP fluorescence), CMV promoter (RFP fluorescence) values. In addition, the cells were labelled with live-dead marker for assessing cell viability status by flow cytometry. Cycloheximide (CHX) serves as positive control and DMSO serves as negative controls. Effective Concentration (EC₅₀) values for coronin 1 promoter inhibition via GFP fluorescence analysis, non-specific promoter inhibition via CMV promoter driven-RFP fluorescence analysis and cell viability via live/dead staining as a measure for toxicity in flow cytometry for compounds (10-22 and 50a-65a) are shown in Table 6 and Table 7. Based on the EC₅₀ analysis of the inhibition of coronin 1 promoter driven GFP fluorescence the (4S,7R)-configurated eutomers were identified (Structures FIG. 9A-C). This is exemplified for compounds containing two stereocenters by eutomers 11c and 13a. For compounds with more than two stereocenters the most active core configuration is as well (4S,7R) shown for compound 12e. Subsequent EC₅₀ evaluations (Table 7) were performed with compound 13a as standard for relative potency normalized to a value of 1.00 (relative potency calculated as [EC₅₀ GFP inhibition 13a]/[EC₅₀ GFP inhibition compound of interest]).

In Vitro Toxicity Assessment with Alamar Blue Assay:

In vitro cytotoxicity tests were performed using Alamar blue (Invitrogen, USA) following manufacturers protocol. In brief, RBL cells pretreated with coronin 1 expression inhibitor or vehicle controls were reseeded and cultured in a 96-well plate at a concentration of 1×10⁴ cells/ml of culture media. Alamar blue was added to the wells and further incubated at 37° C. for 4 h. The absorbance was measured at 600 nm using a microplate reader (Synergy, BioTek Instruments). DMSO and cycloheximide served as the vehicle and positive control respectively (FIG. 5A).

In Vitro Toxicity Assessment with MTT Assay:

MTT assay was performed with 3-(4,5-dimethylthiazol-2-yl)-2,5-diphenyltetra-zolium bromide (MTT, Cayman chemicals) using the manufacturers protocol. In brief, RBL cells pretreated with coronin 1 expression inhibitor or vehicle controls were reseeded and cultured in a 96-well plate at a concentration of 1×10′ cells/ml of culture media, 10 μL of MTT was added and incubated for 4 h at 37° C. At the end, culture supernatant was removed and the formazan crystals were dissolved with 100 μl of crystal dissolving solution and the absorbance measured at 570 nm using a plate reader (Synergy) (FIG. 5B).

### In Vivo Administration of Coronin 1 Promoter Inhibitor for Modulation of Body Weight as a Measure of Toxicity and ADVIA Analysis:

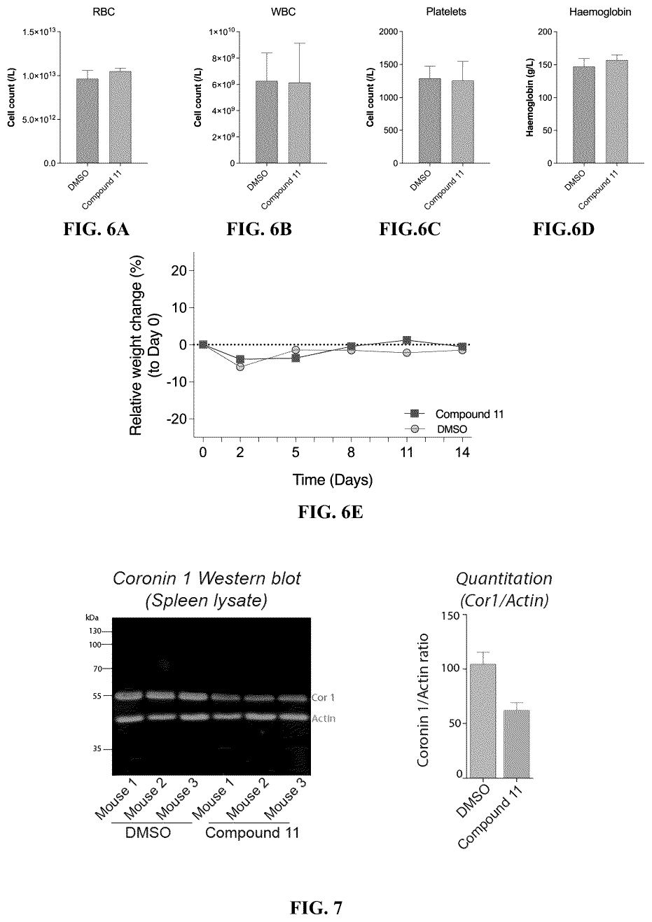

Coronin 1 expression inhibitor (compound 11) was initially dissolved in DMSO at 100 mg/ml concentration. In parallel, a 50:50 solution of miglyol 812 and kolliphor EL (M+K) was prepared. Coronin 1 expression inhibitor: M+K was mixed at a ratio of 30:70 and vortexed for 5 minutes to make a homogenous suspension such that a final concentration of 30 mg/ml was obtained. 100 μl/mouse was subsequently administered either intraperitoneally or subcutaneously using an insulin syringe. The mice used for the study were separated into age and sex matched cohorts, weighed prior to start of the study and regularly monitored for body weight, behavioural changes, fur changes, faecal consistency and urine colour. The application of compounds was continued for 14 days at the end of which the animals were sacrificed and the blood parameters assessed using the ADVIA platform (FIG. 6A-E).

### In Vivo Administration of Coronin 1 Promoter Inhibitor as a Measure for In Vivo Coronin 1 Reduction:

Coronin 1 expression inhibitor was initially dissolved in DMSO at 100 mg/ml concentration. In parallel, a 50:50 solution of miglyol 812 and kolliphor EL (M+K) was prepared. Coronin 1 expression inhibitor: M+K was mixed at a ratio of 30:70 and vortexed for 5 minutes to make a homogenous suspension such that a final concentration of 30 mg/ml was obtained. 100 l/mouse was subsequently administered either intraperitoneally or subcutaneously using an insulin syringe. The mice used for the study were separated into age- and sex-matched cohorts, weighed prior to start of the study and regularly monitored for body weight, behavioural changes, fur changes, faecal consistency and urine colour. For coronin 1 down regulation studies, the administration was continued for 5 days and subsequently the spleens harvested and analysed by western blotting (FIG. 7).

### Human Peripheral Blood Mononuclear Cell Culture for Coronin 1 Western Blotting and Proinflammatory Cytokine Production:

Heparinized or EDTA treated blood or buffy coat from healthy volunteers were enriched for monocytes using the standard Histopaque 1.077 gradient centrifugation methodology. Post enrichment, the monocyte fraction was washed extensively with plain RPMI and subsequently counted and seeded in 96 well plate at a density of 0.2 million cells/well in a volume of 200 μl RPMI-1640 (Sigma) containing 1× penicillin and streptomycin (Gibco), 2 mM L-glutamine (Gibco), 10% heat inactivated FCS (PAA) and heparin 50 units/ml along with the vehicle (DMSO) or coronin 1 inhibitor at indicated concentrations. Cells were incubated in a 37° C. humidified incubator with a 5% CO₂ for five days with the expression inhibitor and vehicle replenished in between at 48 hours post seeding by replacing 50% of the media. Subsequently, the cells were assessed for coronin 1 down regulation by Western blotting (FIG. 8A) and in parallel stimulated with anti-CD3 (2 μg/ml) and anti-CD28 (10 μg/ml) antibodies overnight and treated with brefeldin A for a duration of 4 h and subjected to surface staining (for CD3, CD4, CD8 and CD19) along with live-dead marker and fixed with 4% formaldehyde, permeabilized with 0.1% saponin and subjected to intracellular staining for interleukin-2 (FIG. 8B,C).

### Human Peripheral Blood Mononuclear Cell Culture for Mixed Lymphocyte Reaction:

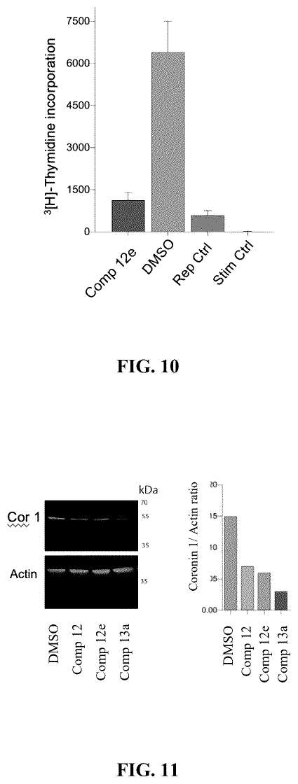

Mixed Lymphocyte Reaction (MLR) is an in vitro model for assessment of alloantigen-driven immune response, hence considered as an in vitro model for transplant rejection responses (or alloimmune responses). For assessment of the suppression of alloresponses by coronin 1 modulators using the MLR methodology, heparinized or EDTA treated blood or buffy coat from two healthy volunteers were enriched for monocytes using the standard Histopaque 1.077 gradient centrifugation methodology as detailed above. Post enrichment, one of the donors PBMCs was considered as “responders” and the other as “stimulators”. The “stimulators” were treated with mitomycin to prevent proliferation while the “responders” PBMCs were left untreated. The stimulator and responder PBMCs were washed extensively with plain RPMI and subsequently counted and seeded in 96 well plate at a density of 0.1 million cells/well each (responders plus stimulators=0.2 million cells/well) in a volume of 200 μl RPMI-1640 (Sigma) containing 1× penicillin and streptomycin (Gibco), 2 mM L-glutamine (Gibco) and 10% heat inactivated FCS (PAA) along with the vehicle (DMSO) or coronin 1 expression inhibitor at indicated concentrations. Cells were incubated in a 37° C. humidified incubator with a 5% CO₂ for five days with the expression inhibitor and at the end of which 0.5 μCi of tritiated thymidine was added to all wells and further incubated for 20 h followed by harvesting on a GF/C filter and measuring the DNA-incorporated counts using a Packard instrument as a measure of immune responses (FIG. 10).

### Assessment of Therapeutic Activity of the Compounds in Imiquimod-Induced Psoriasis:

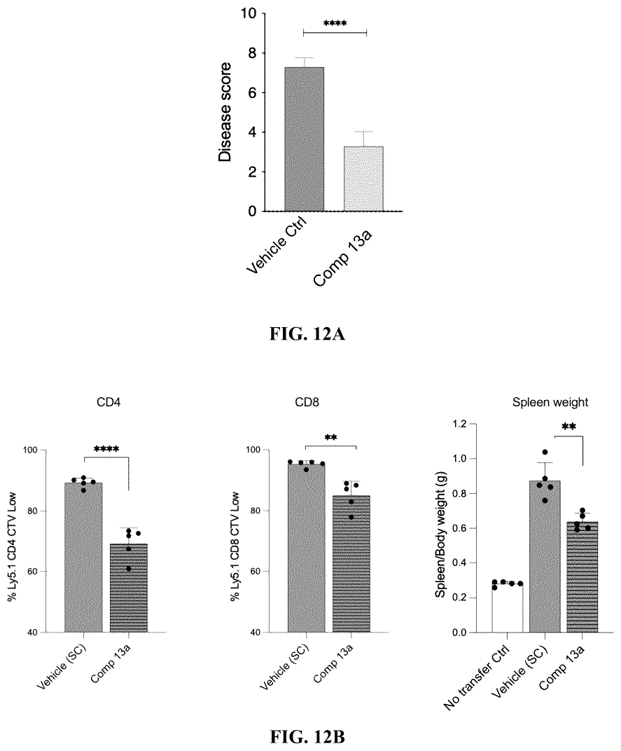

Wild type mice (Balb/c strain, 8-12 weeks), were age and sex matched. The dorsal skin was shaved with a trimmer on an area of ˜2 cm² and subjected to topical application of 5% imiquimod cream (Aldara) starting from day 1 and continued for up to 7 days in the morning. For compound administration, mice were applied topically with the compound, twice a day starting 48 hours before the first Imiquimod application and all along of the experiment up to day 7. In the morning, compound was applied 2 h prior to the imiquimod application (FIG. 12A). Mice were scored every day for the inflammatory lesion and general health status as follows:

| Grade | Erythema | Scales | Skin thickening |
|---|---|---|---|
| 0 | No | No | Normal skin |
| 1 | Mild | Thin flakes | Mild |
| 2 | Moderate | Moderate flakes | Moderate |
| 3 | Severe (<1 cm2) | Thick flakes (<1 cm2) | Rough and hard |
| 4 | Severe (>1 cm2) | Thick flakes (>1 cm2) | Un-foldable |

### Assessment of Therapeutic Activity of the Compounds in K5.Stat3-Induced Psoriasis:

Wild type K5.Stat3 mice (FVB strain) were anesthetized and subjected to tape stripping on an area ˜2 cm², 30 strokes with transparent scotch tape, on the dorsum. For compound administration, compound 11 (75 mg/kg body weight per administration) in a vehicle preparation with PPG400 and Kolliphor EL (3:1 ratio) was topically applied two days prior to the tape stripping procedure, and subsequently followed it up with twice a day administration until the end of the study. The lesions were scored for Erythema, scales and thickening with a graded score between 0-4 as follows.

| Grade | Erythema | Scales | Skin thickening |
|---|---|---|---|
| 0 | No | No | Normal skin |
| 1 | Mild | Thin flakes | Mild |
| 2 | Moderate | Moderate flakes | Moderate |
| 3 | Severe (<1 cm²) | Thick flakes (<1 cm²) | Rough and hard |
| 4 | Severe (>1 cm²) | Thick flakes (>1 cm²) | Un-foldable |

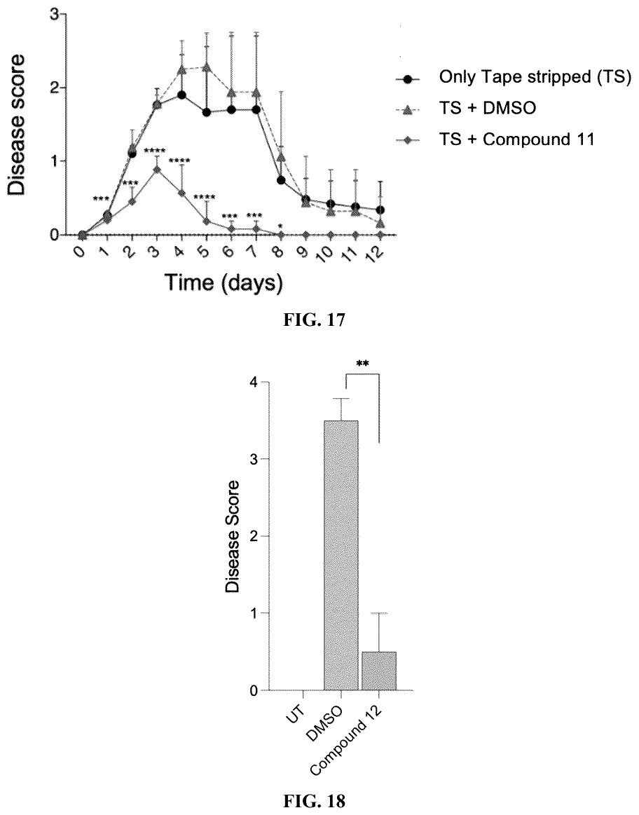

The time score of disease score is shown in FIG. 17.

### Assessment of Therapeutic Activity of the Compounds in DSS Colitis Model:

Wild type, mice were fed with either 2.5 or 3% DSS (MP Biomedicals, 36,000-50,000 M.wt.) #02160110 Colitis Grade) in drinking water ad libitum for a duration of 5 to 7 days as indicated and the kinetics of colitis disease progression monitored. The DSS solution was changed every third day with fresh solution. Administration of compounds was initiated two days prior to the provision of DSS in water (compound 12, 100 mg/kg body weight per administration). It was subcutaneously administered twice every day throughout the course of the study (BID; bis in die). Mice were regularly assessed for fur signs, lethargy, anal/perianal inflammation, perianal staining, stool consistency and weight loss. The scoring was based on the following three major criteria: stool consistency, blood in stools and weight loss. Stools was scored as follows; normal stools (score 0), semisolid stools (score 1), mushy without shape (Score 2), Loose stools (score 3) and watery stools (score 4). Stool blood was scored as follows; No blood (score 0), occult (score 1), orange red discoloration (Score 2), red stains (score 3) and bloody (score 4). Weight loss was scored as follows; 0% loss (score 0), 8-10% (score 1), 10-15% (Score 2), 15-19% (score 3) and 20% and above (score 4). The disease scores were plotted wherein the scores from all the three criteria were added and shown. The study was terminated when mice reached 20% weight loss. The colons were dissected out from the ileocecal junction until anus and length measured on a nonabsorbent surface with a ruler. Care was taken not to stretch the intestine.

The results are shown in FIG. 18.

### Assessment of Therapeutic Activity of the Compounds in Foreign Body Infection Model:

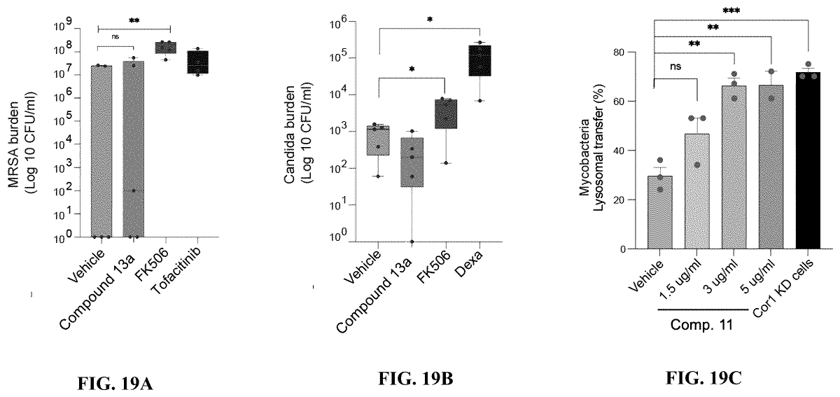

To investigate whether compound 13a can maintain anti-pathogen responses while inducing suppression of autoimmune responses, we used the foreign-body infection model which was established with the approval of the Kantonale Veterinaeramt Basel-Stadt, Switzerland (permit no. 1710). Experiments were conducted according to the regulations of Swiss veterinary law and performed in the animal house of the Department of Biomedicine, University Hospital Basel, Switzerland. Healthy wild-type female C57BL/6 mice at 13 weeks of age (Janvier Labs, France) kept under specific pathogen-free conditions (biosafety level 2) were anesthetized followed by the subcutaneous implantation of sterile cylindrical Teflon tissue cages (32×10 mm; volume: 1.9 mL) with 130 regularly spaced holes (Angst+Pfister AG, Zurich, Switzerland). Upon complete wound healing, cages were tested for sterility. Prophylaxis was started 12 h prior to infection with either the vehicle control or the compound 13a (50 MPK, BID). The injection of either methicillin resistant Staphylococcus aureus (MRSA) ATCC43300 (526 CFU/cage), or Candida albicans ATCC5341 (2000 CFU/cage) was given directly into the lumen of each cage. Treatment started directly after the infection for 8 days twice a day. On days 1, 3, and 8 post-infection, tissue cage fluid was collected to assess planktonic bacterial load by plating. On day 8, mice were sacrificed and the tissue cage of each mouse was explanted under aseptic conditions. The explanted tissue cages were washed twice with phosphate-buffered saline followed by 30 s vortexing, sonication for 3 min at 130 W and another 30 s vortexing to release adherent bacteria from the biofilm. Quantification of adherent bacteria was performed by plating appropriated dilutions. The presence of re-growth of the adherent bacteria was investigated by further incubation of the sonicated cage in the appropriate growth medium for the pathogens for 48 hours at 37° C. Any visualization of a positive culture was defined as treatment failure. The results of this study are summarized in FIG. 19A-B.

### Assessment of Therapeutic Activity of the Compound in Mycobacterial Infection Model:

Murine macrophages (J774, from ATCC) was cultured for 4 days with compound 11 (3 and 5 mg/ml) in culture media (10% heat inactivated FCS containing DMEM), with compound refreshment every 48 h, following which the cells were seeded on a 10 well slide, infected with M. bovis BCG expressing GFP at 0.02 OD for 1 h at 37° C., 5% CO₂. Subsequently, the cells were washed thrice to remove free bacteria followed by a chase for 3 h in complete media at 37° C., 5% CO₂. The cells were washed with PBS and fixed in cold methanol (−20° C.) for 4 minutes following which they were blocked (5% FCS containing PBS) and stained with primary antibodies for coronin 1 (rabbit serum 1002), LAMP-1 (Rat, 1D4B) followed by Alexa-fluor goat anti-rabbit 633 and Alexa-fluor goat anti-rat 568 labeled secondary antibodies and mounted using antifade (Biorad). Slides were imaged using the confocal laser scanning microscope LSM510 Meta (Zeiss) and processed with the corresponding software. The results are shown in FIG. 19C.

### Assessment of Therapeutic Activity of the Compounds in Graft Versus Host Disease (GvHD);

Assessment of GvHD responses were performed as follows. In brief, total T cells were isolated from WT C57BL/6 Ly5.1 (I-A^{b}) mice by means of negative selection using Stem cell technologies kit (#19851). The isolated cells were labelled with cell trace violet (Thermo Fisher) and transferred intravenously (˜14×10⁶ cells) by means of tail vein injection into recipient BDF1 Ly5.2 (I-A^{bd}) mice (day 0). Mice were subsequently divided into groups receiving vehicle, or compound 13a (50 MPK, BD, SC) for 7 days continuously from day 1 to day 7. CTV dilution-based proliferation of transferred T cells was analyzed on day 7 by harvesting the spleen. Single cells were prepared and stained for the CD3, CD4, CD8, Ly5.1, and a viability marker and subjected to flow cytometry analysis. Percentages of Ly5.1+ CTV low cells (the divided cells) was extracted using FlowJo software (FIG. 12B).

TABLE 4
Sequences

| SEQ | |
|---|---|
| ID NO: | Nucleic acid sequence |
| 1 | Human P1.5 |
| | AGGTTCAGAGAGATTAGCCCTCTTGGCCAAGATCGCACAGCCAGTCAGTGGCAGAGGTGGGACTTG |
| | AACCCGTCCCTTGCTGTTCCTTGAACTTCTCCACACGACAGGGCTGTCCCTCAGCCTACAGGTCAC |
| | CCCTCTAGCCTTGCCTGTTGGCTCTTCCTAATCCTTTCAGGCCCAGCCCAGATACTTTTTAAACAA |
| | GAATGATGAGAGCTAACTTTTTTTAAAATGAGACGGTCTTGCTCTTTCACCCAGAGAAGACTGGGT |
| | GATAACAGTGGCTCAATCTTGGCTCACTGCAGCCTCTGCCTCCAGGGTTCAAGCTATTCTCATGCC |
| | TTAGCCTCCTCAGTAGCTGGGATTACAGGGGTGCATCACTACACCACGCTAATTTTTGTATTTTTA |
| | GTAGAGATGCGGTTTTACCATGTTGGCCAGGCTGGTCTCGAACTCCTGACCTCAGGTGATCCGCCA |
| | GCCTTGGCCTCCCAAAGTGCTGGGATTACAGGCGTGAGTCACTACGCATGGCTGAGAGCTAACGTT |
| | TATTAAGCACCCGCCACTTTCTAGATGAGGTCCTTACTGACTTCCTTGGGTTATTTTAGGTAATCA |
| | TCCCCATACCTCTTACGTGGCAGGTATGACTGTTACAGAGGGAGAAACCGAGGCTGAGAAAGGCTA |
| | AGTAGCGGCCACACCTCACTCAGCAGGTGACAGGCTGCGGTCGCACTCATGACATAGTCTGACTCT |
| | AGAGCAGGCCTTTCCTGCCCGCTGTTGCTGCCTCCAGAAGCCTCTCTGAATCCAAGGGTGGGAACT |
| | ATTCCTCCTTGCCCTCGAGCCTTCGCCAAAGCCAGGGCTTGGAGCGGCCGTGGGCTCCTCCTCCTT |
| | CCCCCAGGCTGGGCTCTTCAGGGGCGGAAACCTGCCTGGCTTGCCTGCCTTGCTCGACCCTGTAGC |
| | CCCTGGCACGAAGTAGGTGCCCGTGATGCCGGATTATTTCAGCAGGAGGGCAGGGGTGTTCCCGAT |
| | GAAGCCAGGAGATCCAGGGAGGAGAAGCGACTCCCGGGGCCACCCATGCTTAGATCTCAGAGCCAG |
| | GCAAGAGCCAGGTCCCGGGGGTCCAGGAGCGCGCTCGGGGCTCCTCCTCGCCCGCGCCCCCCGCGC |
| | CCCTCGCGCCCCCCGCGCCCCCCGCGCCCCCTGCGCTCGCGTCTGCGGGGCCGGGAGGAAGGGGCG |
| | GCGCAGCCGGGCCGGGGCGGGCACCGAGGCCTGGCGGGCGGGCCTGGGGCGCTTTCGTGCCTGCCA |
| | GGGAAAGGCTGGCGAGGAGGCTGGGGGCGGACGCAGGGAGGAAAGAAGCAATGGGGTGCTCTTGCG |
| | TTTTGACATTTTAAAAAGAAGTTTAGATTCGCTTCCCGGAGCCGGGATGGCAGCCTGCGCTATGGT |
| | TGGGGACCAACGTGGTGCACGGGCAGGGGCCGGGGAGAGAGGCGGCCGCAGCGGGAGCCCGGGAGC |
| | GCAGGGCGGGCCTGGAAGAGCTCCGCCCCAAGGGGCGCGGCCACCCCGGAGGCGGGCGCACGG |
| 2 | Murine P1.5: |
| | AAATAAATCCTGGTTCTCTGCAAGAACAGTGAGCGCTCTTCACCACTGAGCTGTCTCTCTAGCCCG |
| | GCACCTAGCTTATGTGATGCTGGAGGCCATGCCTTTATGCATGCTATGAGGTGTTCTGCCAGCTGA |
| | ACTCCATTCTCAGCCCCATTACTGTTATCACTTTAGCTGGAGACTCTAGAGTCGCTGGCTACAGTC |
| | ACAGAGCCTCAGGCACTATCCAACCCTGTACTGAGTAACTGCAGAACACTTCAGACTACACCTTAC |
| | GCACCGCACCGTTAGCTGTGAGGTGGGCAGGACTTCTTGTTTCTCTCCTACAGAAACTGAGGTTCA |
| | GGGAGGTGGGGCCTCTTGGCCAAGATCACACGGCTAATCAGTACAACTGGAGCCCCTTCTGTTCTT |
| | TCGAACTACTTCTACCTTAAAGAAGAGTTTCTTCTGACTGGGTCTTGCTATGTAGCCCAGGCTGGC |
| | CTCCTGCCTCAGCTTCCTGACTACAGATGACTGTCAGCACACCTGTGTCCTTGAGCTTTGCTGACA |
| | CTGCGCCTCAGCCCCCTACCCTTCTCCAGAGCCACTTGTGGTGTTTCCTCTTCCTCCCAGGATAGC |
| | CCAAAGCTTTGGTAAATGGCAATAATGAAAAGAACCAACATCTTTTGGGTTTTTGAAACAGGGTCT |
| | CACTATGTAGCCTTGGTCTGGAGCTTACATGTGGACCAGGCTGGCCTTGGTTTCACTGACCCTTCT |
| | GCCTCTGCCTCCGTAGTAATGGATTTACAGGTGTGGGCCACCATATCTGACTGTAAGACCCCAACA |
| | CTAATTAATACCTGCCACTCTGTAGATGCAAATCCAGCTATCCTTGGAGTGACCATCTCCATACGA |
| | AGGCAGGTAAGACTGTTATAGACAGGAAACTGGGGCTGAGATTAAATTAACCTGCCCAAGGTCACT |
| | CAAATGAGTGACAGCCAGGAGTCACAACAATGGTTCCAGCAAAGGCCTTTCTTCCTCTGCCCTTGC |
| | TACCGTGACAACCCACTCCCCTCAAGGGACAGGAATGGACCACAGCCCAGCTGTGTGTGATATCAG |
| | ACTTCTGTGTGTACTCAGTGGCTTTTAAACTGTGTCAGACGGACTCCCGACTCCCTCTTCTCATGG |
| | CCCGAGAGCTCTCTAGAGGGGGAGCACTACTCATTTGCCTCCATAGCCCCAGCACAGAACAGGTGC |
| | TCCATAATGCCCAGTTATTTTACTTCTTCCGGGCCTCAATGCCACGAAGCAGAGGACAAGGGTGTC |
| | CCCGCTGAAGCCCTAAATCCCTAGAAAGGAGAAATGACCCCCCCAAGGCCTCCCAGAAATCCATTT |
| | GGGGTAACGACAGGAGAGGTCCCTCCTGTAGCAGAGAAGGCTCTCAAGCTGGTTGAAGCCGTTCTA |
| | AAGCTTTCTCCGGTGCCAGGGAAAGGACCTCGAGGAGGCAGCGGGCGGCTGGGGGAGGGATGAAGG |
| | AACGGGATATTTTTGCGTTCTGACATTAAAAGAAGAGTTGATTCGCGTCAGCATCTGTTCGGGGGT |
| | TGGAGCTGACAGCATGGGGAGCGGTCCCAGTCTTGCAGCCCTGGAGCGAAGGGCCGGGCCTCCATG |
| | AGCTCCGCCCAAAGGGGCGCTACCACCCGGAGGAGGCCGGCGCACGGCCGCTGCTCATTCATTGTC |
| | TTGACAGAGCA |
| 3 | Rat P1.5: |
| | CTTCCTCAGGCTGTGCACGAATTCTGCCAGCTGAGCTGCATTCTCAGCCCAGTACTGTTAGCACTT |
| | TAGCTGCAGACTGCCGAGTCCCTGGCCACGATCACAGAGCCTCAGGCACAATCCAACCCTGTTGAG |
| | TAACTATAGAACACTGCAGGCTACAGAACAGTCAAATGCTTCATTTGAGCCCAGACAGCTGTGAGG |
| | TGGGCAGGGCGCCTTGTTCCTCTCCTACAGAAACTGAGGCTTGGAGAGGAGGGGCCTCTTGGTCAA |
| | GATCACACAGCTAATCGGTACGATTGGAGTCAGGCCCCTTTTGTTCCTTTGGACTACTTCTACCTG |
| | GAAGAACGGTTTCCTCTGACAGGGTCTTGCTGTGTAGCCCAGGCTGTGAGGGCTCCTGCCTCAGTT |
| | TCCTGAGCACAGATGACTATCAGCACACCTGTGTCTTTGAGCTTTGCTGTCACCGAGCCTCAGCTC |
| | CTGCCCTTCTCCAAAGCCACATCTGCTATTTCCCTCTTCTTCCCAGGATAGCCCAAAGCTTTGGTA |
| | AATGACAATTATGAAAGGGACCAACATCTTTTAGGTTTTTGAAACAGGGTCTCACTATGTAGCCAG |
| | GCCTGGAACTTTTATGTGGACCAGGCTGGCCTCGGCTTCACTGTGATCCTTAGGCCTCTGCCTCCC |
| | CAGTAATGGATTCATAGGTGTGGGCCACCACATTGGCTGTAAGATCCAACATTAGTTATCGCCTGA |
| | CGCTCTGTAGATGTGCATCCAGCTATTTTTGGAGTGAACGTCTCCACATTGAGGCAGGTAAGACTG |
| | TTAGAGAGGGGGAGTTAGGGCTGAGATTAAATTAACCTGTCCAAGTCATCTGAGTGACCACCAGGA |
| | GTCACAAGGTTCCAGCAAAGGCCTTTCTTGCCCTGACAACCCACCACCCCCTAAGGGACAGGAATG |
| | GTTAAGAGCACTTCTGCGTGTGGTATCAGACTTCTGCGTGTACTCAGTGGCCTTTAAACTGTGGCA |
| | GACATACTCCCCACTCCACTTGCCACAGGCCTGAGGGCTCTCTAGTGGCGGGAGCACGACTCACTT |
| | CCCTCCTCAGCCCCAGCACAAAACAGGTGCTCCATAATGCCCAGTTATTCTACTTCTTCCGGGCCT |
| | CAACCCCGTGAAGCAGAGGTCAACGGCATCCCCGCTGAAGCCCTGAACCCCTAGAAAGGAGAAATG |
| | AACCCCCCCTAAGGCATCCCAGAAATCCATCTGGGTCATCGACAAGAGAGATCCTTCCTGTCGCAG |
| | AGAAGGCTCTCAAGCTGGTTGTAGCCCTTCTGGCGCTTTCTCGGGTGCCAGGGAAAGGACCTCGAG |
| | GAGGCTGCGGGCGGCTGGGGGAGGGATGAAGGAACGGATATTTTTTGCGTTTTGACATTAAAAGAA |
| | GAGTTGATTCGCGTCAGCATCTGTTCGGGGGTTGGAGCTGACAGCATGGGAGCGGGTCCCAGCTTT |
| | GCCGCCCCAGAGCGAAGGGCCGGGCCTCCATGAGCTCCGCCCAAAGGGGCGCTGCCACCCCAGAGG |
| | AGGCCGGCGCACGGCCGCTGCTCATTCATTGTCTTGACAGAGCA |
| 4 | Human P0.7 |
| | TTCCTCCTTGCCCTCGAGCCTTCGCCAAAGCCAGGGCTTGGAGCGGCCGTGGGCTCCTCCTCCTTC |
| | CCCCAGGCTGGGCTCTTCAGGGGCGGAAACCTGCCTGGCTTGCCTGCCTTGCTCGACCCTGTAGCC |
| | CCTGGCACGAAGTAGGTGCCCGTGATGCCGGATTATTTCAGCAGGAGGGCAGGGGTGTTCCCGATG |
| | AAGCCAGGAGATCCAGGGAGGAGAAGCGACTCCCGGGGCCACCCATGCTTAGATCTCAGAGCCAGG |
| | CAAGAGCCAGGTCCCGGGGGTCCAGGAGCGCGCTCGGGGCTCCTCCTCGCCCGCGCCCCCCGCGCC |
| | CCTCGCGCCCCCCGCGCCCCCCGCGCCCCCTGCGCTCGCGTCTGCGGGGCCGGGAGGAAGGGGCGG |
| | CGCAGCCGGGCCGGGGGGGGCACCGAGGCCTGGCGGGCGGGCCTGGGGCGCTTTCGTGCCTGCCAG |
| | GGAAAGGCTGGCGAGGAGGCTGGGGGCGGACGCAGGGAGGAAAGAAGCAATGGGGTGCTCTTGCGT |
| | TTTGACATTTTAAAAAGAAGTTTAGATTCGCTTCCCGGAGCCGGGATGGCAGCCTGCGCTATGGTT |
| | GGGGACCAACGTGGTGCACGGGCAGGGGCCGGGGAGAGAGGCGGCCGCAGCGGGAGCCCGGGAGCG |
| | CAGGGCGGGCCTGGAAGAGCTCCGCCCCAAGGGGCGCGGCCACCCCGGAGGCGGGCGCACGG |
| 5 | Murine P0.7: |
| | CAAATGAGTGACAGCCAGGAGTCACAACAATGGTTCCAGCAAAGGCCTTTCTTCCTCTGCCCTTGC |
| | TACCGTGACAACCCACTCCCCTCAAGGGACAGGAATGGACCACAGCCCAGCTGTGTGTGATATCAG |
| | ACTTCTGTGTGTACTCAGTGGCTTTTAAACTGTGTCAGACGGACTCCCGACTCCCTCTTCTCATGG |
| | CCCGAGAGCTCTCTAGAGGGGGAGCACTACTCATTTGCCTCCATAGCCCCAGCACAGAACAGGTGC |
| | TCCATAATGCCCAGTTATTTTACTTCTTCCGGGCCTCAATGCCACGAAGCAGAGGACAAGGGTGTC |
| | CCCGCTGAAGCCCTAAATCCCTAGAAAGGAGAAATGACCCCCCCAAGGCCTCCCAGAAATCCATTT |
| | GGGGTAACGACAGGAGAGGTCCCTCCTGTAGCAGAGAAGGCTCTCAAGCTGGTTGAAGCCGTTCTA |
| | AAGCTTTCTCCGGTGCCAGGGAAAGGACCTCGAGGAGGCAGCGGGCGGCTGGGGGAGGGATGAAGG |
| | AACGGGATATTTTTGCGTTCTGACATTAAAAGAAGAGTTGATTCGCGTCAGCATCTGTTCGGGGGT |
| | TGGAGCTGACAGCATGGGGAGCGGTCCCAGTCTTGCAGCCCTGGAGCGAAGGGCCGGGCCTCCATG |
| | AGCTCCGCCCAAAGGGGCGCTACCACCCGGAGGAGGCCGGCGCACGGCCGCTGCTCATTCATTGTC |
| | TTGACAGAGCA |
| 6 | Rat P0.7: |
| | TCTGAGTGACCACCAGGAGTCACAAGGTTCCAGCAAAGGCCTTTCTTGCCCTGACAACCCACCACC |
| | CCCTAAGGGACAGGAATGGTTAAGAGCACTTCTGCGTGTGGTATCAGACTTCTGCGTGTACTCAGT |
| | GGCCTTTAAACTGTGGCAGACATACTCCCCACTCCACTTGCCACAGGCCTGAGGGCTCTCTAGTGG |
| | CGGGAGCACGACTCACTTCCCTCCTCAGCCCCAGCACAAAACAGGTGCTCCATAATGCCCAGTTAT |
| | TCTACTTCTTCCGGGCCTCAACCCCGTGAAGCAGAGGTCAACGGCATCCCCGCTGAAGCCCTGAAC |
| | CCCTAGAAAGGAGAAATGAACCCCCCCTAAGGCATCCCAGAAATCCATCTGGGTCATCGACAAGAG |
| | AGATCCTTCCTGTCGCAGAGAAGGCTCTCAAGCTGGTTGTAGCCCTTCTGGCGCTTTCTCGGGTGC |
| | CAGGGAAAGGACCTCGAGGAGGCTGCGGGCGGCTGGGGGAGGGATGAAGGAACGGATATTTTTTGC |
| | GTTTTGACATTAAAAGAAGAGTTGATTCGCGTCAGCATCTGTTCGGGGGTTGGAGCTGACAGCATG |
| | GGAGCGGGTCCCAGCTTTGCCGCCCCAGAGCGAAGGGCCGGGCCTCCATGAGCTCCGCCCAAAGGG |
| | GCGCTGCCACCCCAGAGGAGGCCGGCGCACGGCCGCTGCTCATTCATTGTCTTGACAGAGCA |
| SEQ ID | |
| NO.: | Sequence: |
| 1 | Human P1.5 promoter fragment from the 5′ upstream sequence of the coronin 1a |
| | transcription start site (TSS). TSS is shown in bold. The first nucleotide residue of |
| | the promoter upstream of the TSS is bold and underlined. |
| 2 | Mouse P1.5 promoter fragment from the 5′ upstream sequence of the coronin 1a |
| | transcription start site (TSS). TSS is shown in bold. The first nucleotide residue of |
| | the promoter upstream of the TSS is bold and underlined. |
| 3 | Rat P1.5 promoter fragment from the 5′ upstream sequence of the coronin 1a |
| | transcription start site (TSS). TSS is shown in bold. The first nucleotide residue of |
| | the promoter upstream of the TSS is bold and underlined. |
| 4 | Human PO.7 promoter fragment from the 5′ upstream sequence of the coronin 1a |
| | transcription start site (TSS). TSS is shown in bold. The first nucleotide residue of |
| | the promoter upstream of the TSS is bold and underlined. |
| 5 | Mouse PO.7 promoter fragment from the 5′ upstream sequence of the coronin 1a |
| | transcription start site (TSS). TSS is shown in bold. The first nucleotide residue of |
| | the promoter upstream of the TSS is bold and underlined. |
| 6 | Rat PO.7 promoter fragment from the 5′ upstream sequence of the coronin 1a |
| | transcription start site (TSS). TSS is shown in bold. The first nucleotide residue of |
| | the promoter upstream of the TSS is bold and underlined. |

TABLE 5
Percent changes in GFP/RFP fluorescence upon compound treatment.
Particularly desirable is selective reduction of GFP values,
with minimal alterations of RFP values.

| | GFP | RFP |
|---|---|---|
| | values (%) | values (%) |
| Media | 118 | 115 |
| DMSO | 100 | 100 |
| CHX | 21 | 40 |
| Comp. 1 | 35 | 66 |
| Comp. 2 | 52 | 81 |
| Comp. 3 | 63 | 80 |
| Comp. 4 | 60 | 85 |
| Comp. 5 | 74 | 108 |
| Comp. 6 | 68 | 75 |
| Comp. 7 | 77 | 95 |
| Comp. 8 | 87 | 97 |
| Comp. 9 | 58 | 97 |
| Comp. 10 | 48 | 110 |
| Comp. 11 | 35 | 111 |
| Comp. 12 | 46 | 153 |
| Comp. 22 | 55 | 70 |

TABLE 6
Effective Concentration (EC₅₀) values for coronin 1 promoter inhibition

| | | EC₅₀ | | | | | |
|---|---|---|---|---|---|---|---|
| Compound | | GFP | | RFP | | | |
| number | Structure | inhibition | | alteration | | Toxicity | |
| 10 | 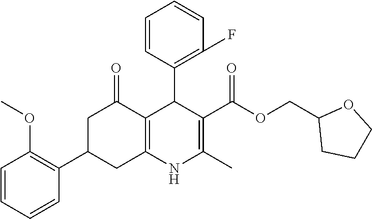 [SMILES: COc1ccccc1C1CC(=O)C2=C(C1)NC(C)=C(C(=O)OCC1CCCO1)C2c1ccccc1F] | 0.8 | μM | 1 | μM | >7.5 | μM |
| 11 | 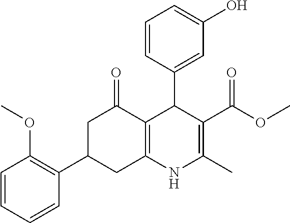 | 1.2 | μM | >9 | μM | >9 | μM |
| 11a | 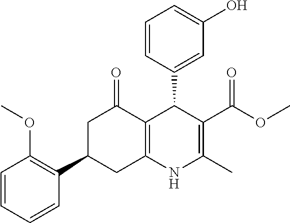 | 0.5 | μM | n.d. | | >9 | μM |
| | and | | | | | | |
| | 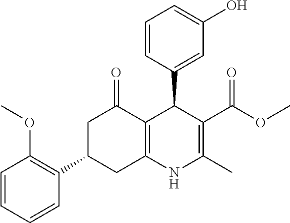 | | | | | | |
| 11b | 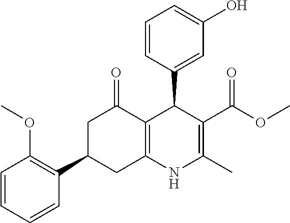 | >36 | μM | n.d. | | >36 | μM |
| | and | | | | | | |
| | 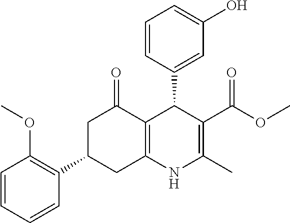 | | | | | | |
| 11c | 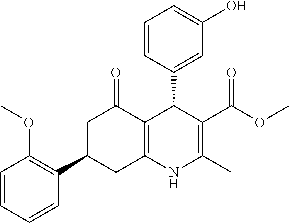 | 0.175 | μM | n.d. | | >9 | μM |
| 11d | 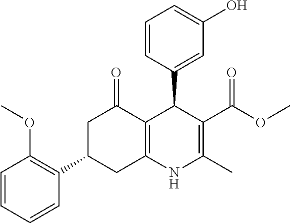 | 2 | μM | n.d. | | >18 | μM |
| 12 | 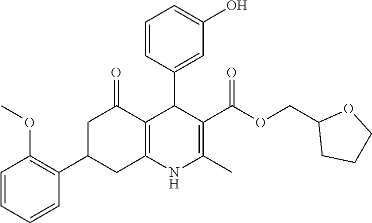 [SMILES: COc1ccccc1C1CC(=O)C2=C(C1)NC(C)=C(C(=O)OCC1CCCO1)C2c1cccc(O)c1] | 0.34 | μM | >1 | μM | >3 | μM |
| 12a | 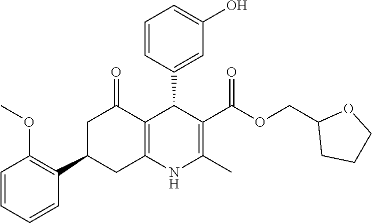 [SMILES: COc1ccccc1[C@H]1CC(=O)C2=C(C1)NC(C)=C(C(=O)OCC1CCCO1)[C@H]2c1cccc(O)c1] | 0.2 | μM | n.d. | | >30 | μM |
| | and | | | | | | |
| | 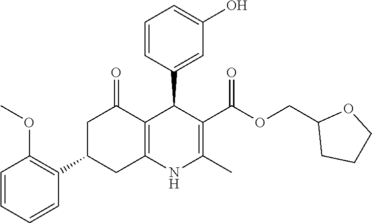 [SMILES: COc1ccccc1[C@@H]1CC(=O)C2=C(C1)NC(C)=C(C(=O)OCC1CCCO1)[C@@H]2c1cccc(O)c1] | | | | | | |
| 12b | 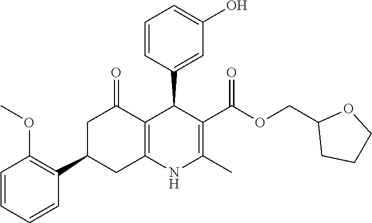 [SMILES: COc1ccccc1[C@H]1CC(=O)C2=C(C1)NC(C)=C(C(=O)OCC1CCCO1)[C@@H]2c1cccc(O)c1] | >30 | μM | n.d. | | >30 | μM |
| | and | | | | | | |
| | 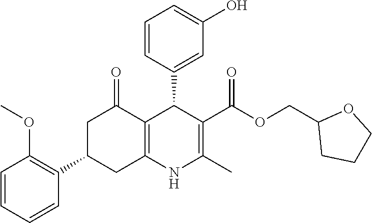 [SMILES: COc1ccccc1[C@@H]1CC(=O)C2=C(C1)NC(C)=C(C(=O)OCC1CCCO1)[C@H]2c1cccc(O)c1] | | | | | | |
| 12c | 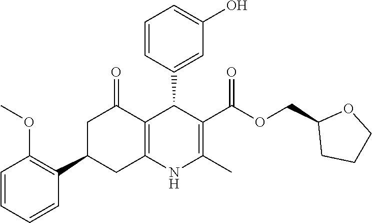 [SMILES: COc1ccccc1[C@H]1CC(=O)C2=C(C1)NC(C)=C(C(=O)OC[C@@H]1CCCO1)[C@H]2c1cccc(O)c1] | 0.2 | μM | n.d. | | >4 | μM |
| | and | | | | | | |
| | 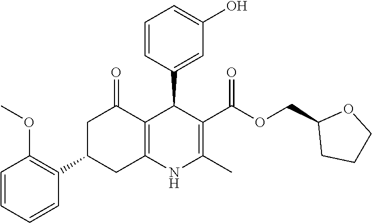 [SMILES: COc1ccccc1[C@@H]1CC(=O)C2=C(C1)NC(C)=C(C(=O)OC[C@@H]1CCCO1)[C@@H]2c1cccc(O)c1] | | | | | | |
| 12e | 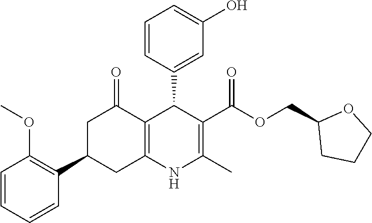 [SMILES: COc1ccccc1[C@H]1CC(=O)C2=C(C1)NC(C)=C(C(=O)OC[C@@H]1CCCO1)[C@H]2c1cccc(O)c1] | 0.07 | μM | n.d. | | >20 | μM |
| 12f | 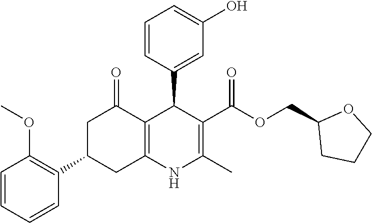 [SMILES: COc1ccccc1[C@@H]1CC(=O)C2=C(C1)NC(C)=C(C(=O)OC[C@@H]1CCCO1)[C@@H]2c1cccc(O)c1] | 2.5 | μM | n.d. | | >2 | μM |
| 13 | 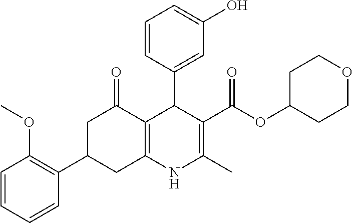 [SMILES: COc1ccccc1C1CC(=O)C2=C(C1)NC(C)=C(C(=O)OC1CCOCC1)C2c1cccc(O)c1] | 0.12 | μM | >2.5 | μM | >2.5 | μM |
| 13a | 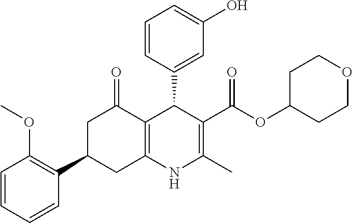 [SMILES: COc1ccccc1[C@H]1CC(=O)C2=C(C1)NC(C)=C(C(=O)OC1CCOCC1)[C@H]2c1cccc(O)c1] | 0.065 | μM | >2.5 | μM | >2.5 | μM |
| 14 | 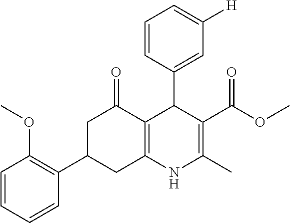 | 0.25 | μM | n.d. | | >6 | μM |
| 15 | 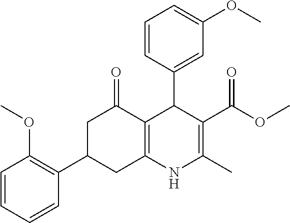 | 1.2 | μM | n.d. | | >15 | μM |
| 16 | 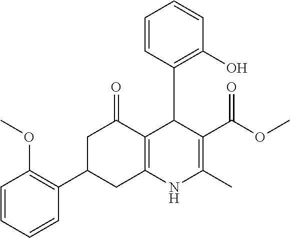 | 0.9 | μM | n.d. | | >7.5 | μM |
| 17 | 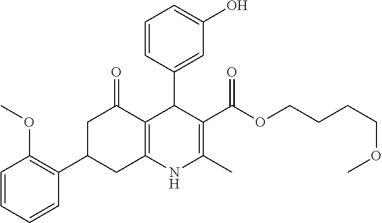 [SMILES: COCCCCOC(=O)C1=C(C)NC2=C(C(=O)CC(c3ccccc3OC)C2)C1c1cccc(O)c1] | 0.1 | μM | n.d. | | >5 | μM |
| 18 | 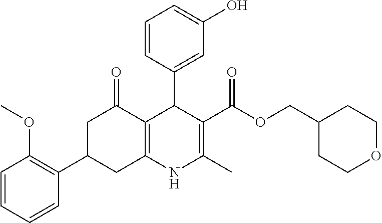 [SMILES: COc1ccccc1C1CC(=O)C2=C(C1)NC(C)=C(C(=O)OCC1CCOCC1)C2c1cccc(O)c1] | 0.2 | μM | >2.5 | μM | >2.5 | μM |

| | | | | | | |
|---|---|---|---|---|---|---|
| 19 | 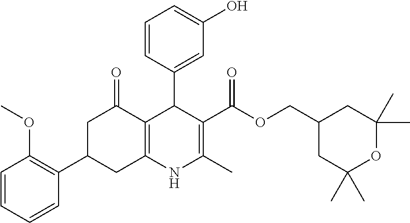 [SMILES: COc1ccccc1C1CC(=O)C2=C(C1)NC(C)=C(C(=O)OCC1CC(C)(C)OC(C)(C)C1)C2c1cccc(O)c1] | 0.16 | μM | n.d. | >2.5 | μM |
| 20 | 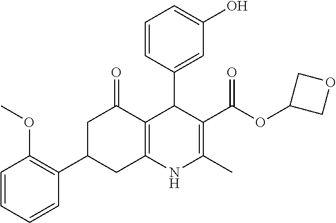 [SMILES: COc1ccccc1C1CC(=O)C2=C(C1)NC(C)=C(C(=O)OC1COC1)C2c1cccc(O)c1] | 0.5 | μM | n.d. | >2.5 | μM |
| 21 | 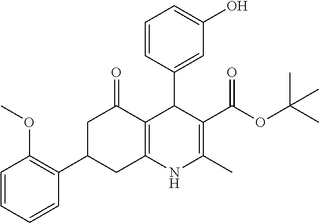 [SMILES: COc1ccccc1C1CC(=O)C2=C(C1)NC(C)=C(C(=O)OC(C)(C)C)C2c1cccc(O)c1] | 0.28 | μM | n.d. | >2.5 | μM |

n.d. not determined

TABLE 7
Relative potency values for coronin 1 promoter inhibition

| | | rel. potency* |
|---|---|---|
| Compound number | Structure | GFP inhibition |
| 12g | 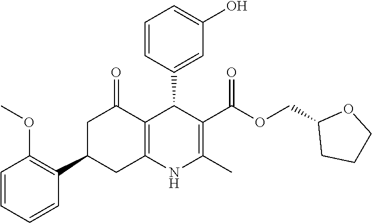 [SMILES: COc1ccccc1[C@H]1CC(=O)C2=C(C1)NC(C)=C(C(=O)OC[C@H]1CCCO1)[C@H]2c1cccc(O)c1] | 1.00 |
| 13a | 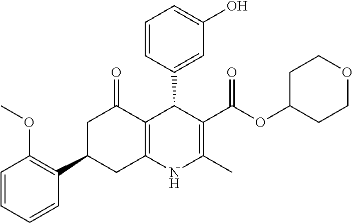 [SMILES: COc1ccccc1[C@H]1CC(=O)C2=C(C1)NC(C)=C(C(=O)OC1CCOCC1)[C@H]2c1cccc(O)c1] | 1.00 |
| 13d | 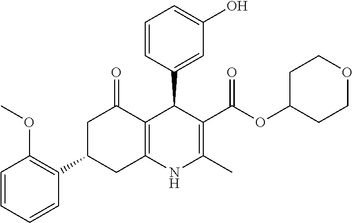 [SMILES: COc1ccccc1[C@@H]1CC(=O)C2=C(C1)NC(C)=C(C(=O)OC1CCOCC1)[C@@H]2c1cccc(O)c1] | 0.09 |
| 13e | 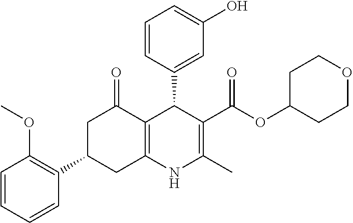 [SMILES: COc1ccccc1[C@@H]1CC(=O)C2=C(C1)NC(C)=C(C(=O)OC1CCOCC1)[C@H]2c1cccc(O)c1] | <0.01 |
| 13f | 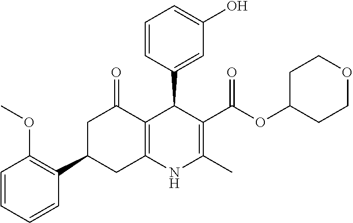 [SMILES: COc1ccccc1[C@H]1CC(=O)C2=C(C1)NC(C)=C(C(=O)OC1CCOCC1)[C@@H]2c1cccc(O)c1] | <0.01 |
| 50a | 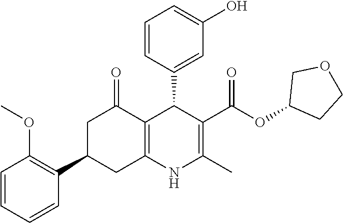 [SMILES: COc1ccccc1[C@H]1CC(=O)C2=C(C1)NC(C)=C(C(=O)O[C@H]1CCOC1)[C@H]2c1cccc(O)c1] | 0.73 |
| 50b | 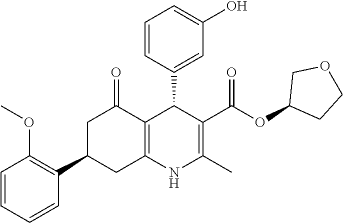 [SMILES: COc1ccccc1[C@H]1CC(=O)C2=C(C1)NC(C)=C(C(=O)O[C@@H]1CCOC1)[C@H]2c1cccc(O)c1] | 1.23 |
| 51a | 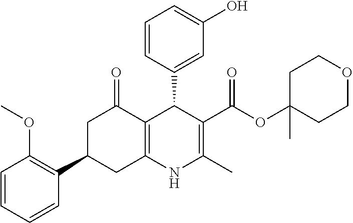 [SMILES: COc1ccccc1[C@H]1CC(=O)C2=C(C1)NC(C)=C(C(=O)OC1(C)CCOCC1)[C@H]2c1cccc(O)c1] | 1.13 |
| 52a | 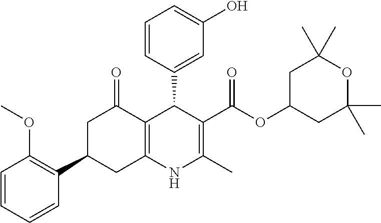 [SMILES: COc1ccccc1[C@H]1CC(=O)C2=C(C1)NC(C)=C(C(=O)OC1CC(C)(C)OC(C)(C)C1)[C@H]2c1cccc(O)c1] | 0.62 |
| 53a | 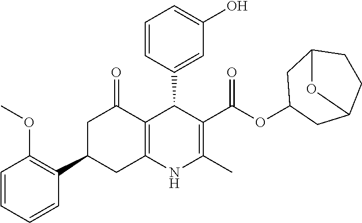 [SMILES: COc1ccccc1[C@H]1CC(=O)C2=C(C1)NC(C)=C(C(=O)OC1CC3CCC(C1)O3)[C@H]2c1cccc(O)c1] | 1.42 |
| 54a | 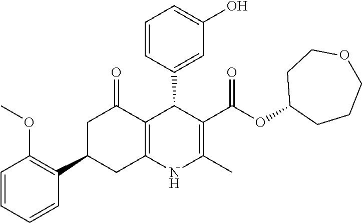 [SMILES: COc1ccccc1[C@H]1CC(=O)C2=C(C1)NC(C)=C(C(=O)O[C@H]1CCCOCC1)[C@H]2c1cccc(O)c1] | 0.88 |
| | and | |
| | 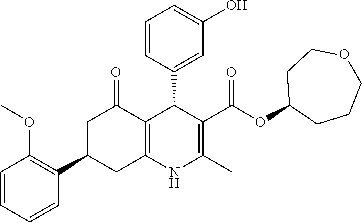 [SMILES: COc1ccccc1[C@H]1CC(=O)C2=C(C1)NC(C)=C(C(=O)O[C@@H]1CCCOCC1)[C@H]2c1cccc(O)c1] | |
| 55a | 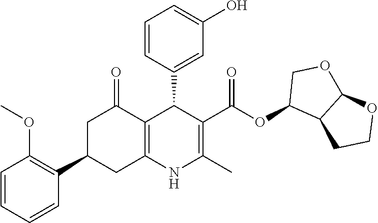 [SMILES: COc1ccccc1[C@H]1CC(=O)C2=C(C1)NC(C)=C(C(=O)O[C@H]1CO[C@H]3OCC[C@H]31)[C@H]2c1cccc(O)c1] | 0.61 |
| 56a | 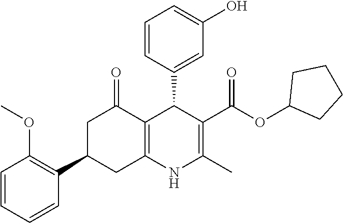 [SMILES: COc1ccccc1[C@H]1CC(=O)C2=C(C1)NC(C)=C(C(=O)OC1CCCC1)[C@H]2c1cccc(O)c1] | 0.21 |
| 57a | 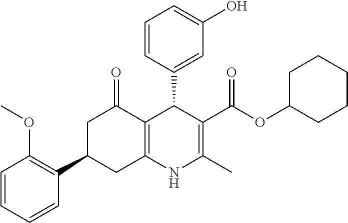 [SMILES: COc1ccccc1[C@H]1CC(=O)C2=C(C1)NC(C)=C(C(=O)OC1CCCCC1)[C@H]2c1cccc(O)c1] | 0.24 |
| 58a | 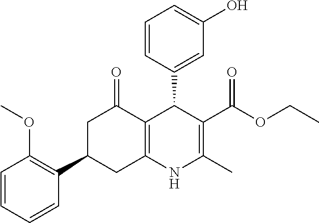 [SMILES: CCOC(=O)C1=C(C)NC2=C(C(=O)C[C@H](c3ccccc3OC)C2)[C@@H]1c1cccc(O)c1] | 0.36 |
| 59a | 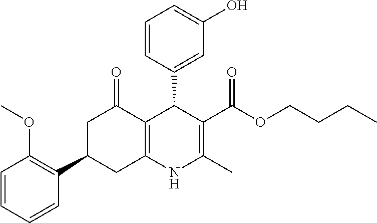 [SMILES: CCCCOC(=O)C1=C(C)NC2=C(C(=O)C[C@H](c3ccccc3OC)C2)[C@@H]1c1cccc(O)c1] | 0.18 |
| 60a | 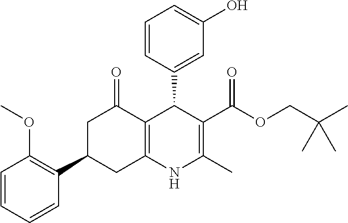 [SMILES: COc1ccccc1[C@H]1CC(=O)C2=C(C1)NC(C)=C(C(=O)OCC(C)(C)C)[C@H]2c1cccc(O)c1] | 0.20 |
| 61a | 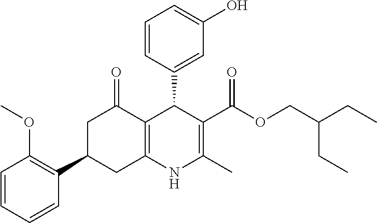 [SMILES: CCC(CC)COC(=O)C1=C(C)NC2=C(C(=O)C[C@H](c3ccccc3OC)C2)[C@@H]1c1cccc(O)c1] | 0.10 |
| 62a | 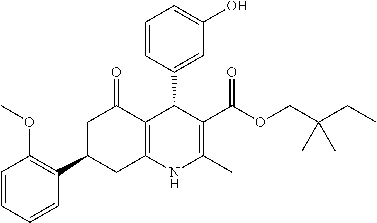 [SMILES: CCC(C)(C)COC(=O)C1=C(C)NC2=C(C(=O)C[C@H](c3ccccc3OC)C2)[C@@H]1c1cccc(O)c1] | 0.10 |
| 63a | 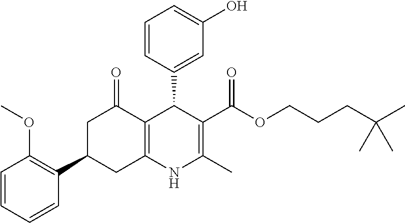 [SMILES: COc1ccccc1[C@H]1CC(=O)C2=C(C1)NC(C)=C(C(=O)OCCCC(C)(C)C)[C@H]2c1cccc(O)c1] | 0.07 |
| 64a | 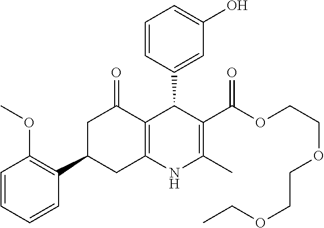 [SMILES: CCOCCOCCOC(=O)C1=C(C)NC2=C(C(=O)C[C@H](c3ccccc3OC)C2)[C@@H]1c1cccc(O)c1] | 0.62 |
| 65a | 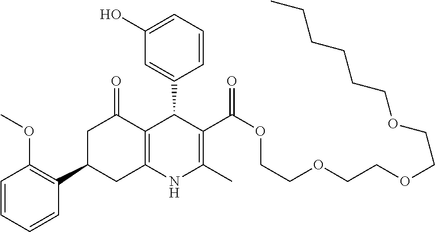 [SMILES: CCCCCCOCCOCCOCCOC(=O)C1=C(C)NC2=C(C(=O)C[C@H](c3ccccc3OC)C2)[C@@H]1c1cccc(O)c1] | 0.12 |
| 66b | 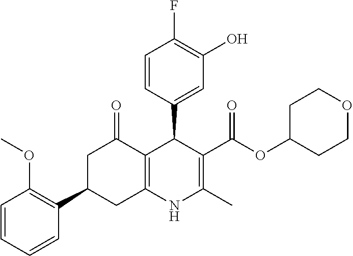 [SMILES: COc1ccccc1[C@H]1CC(=O)C2=C(C1)NC(C)=C(C(=O)OC1CCOCC1)[C@@H]2c1ccc(F)c(O)c1] | 0.25 |
| | and | |
| | 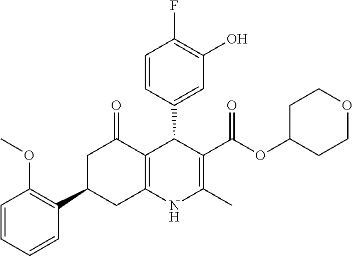 [SMILES: COc1ccccc1[C@H]1CC(=O)C2=C(C1)NC(C)=C(C(=O)OC1CCOCC1)[C@H]2c1ccc(F)c(O)c1] | |
| 67b | 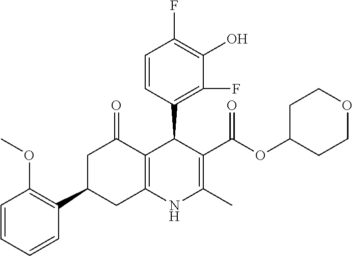 [SMILES: COc1ccccc1[C@H]1CC(=O)C2=C(C1)NC(C)=C(C(=O)OC1CCOCC1)[C@@H]2c1ccc(F)c(O)c1F] | 0.25 |
| | and | |
| | 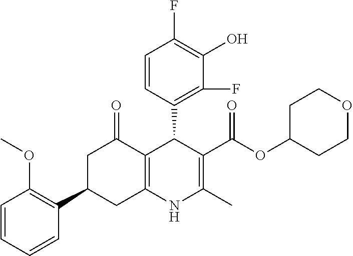 [SMILES: COc1ccccc1[C@H]1CC(=O)C2=C(C1)NC(C)=C(C(=O)OC1CCOCC1)[C@H]2c1ccc(F)c(O)c1F] | |

*relative potency calculated as [EC₅₀ GFP inhibition 13a]/[EC₅₀ GFP inhibition compound of interest]

The characterization of the screening assay identified compounds together with the validation studies done in in vitro and in vivo biological assays reveals that the screening assay identified compounds are immunosuppressive in nature and are capable of inhibiting coronin 1 promoter activity.

### Example 7

To identify the target of the coronin 1 modulatory compounds, Thermal Proteome Profiling (TPP), a methodology based on the property of proteins to alter their thermal stability upon interactions with small molecules (Savitski, M. M., et al., Science, 2014. 346(6205): p. 1255784), was employed. To that end, Rat Basophil Leukemia (RBL) cells were incubated with the compound 12e followed by gradient thermal denaturation (37/41/44/47/50/53/56/59/63/67 degree C.) and mass spectrometric assessment of proteins that get either stabilized or destabilized upon interacting with the compound performed. This procedure led to the identification of Bromodomain-containing 3 (BRD3) protein as the top hit to be stabilized by the compound (FIG. 13). Interestingly, this was the only member to get stabilized amongst the bromodomain and extra-terminal (BET) family of proteins. Currently there exists no compound that selectively targets BRD3, nor is an effect of selective BRD3 inhibition or BRD3 depletion through gene knock-out in vivo in animal models known.

The BET family comprises consists of four members (BDR2, BRD3, BRD4 and BRDT) that are characterized by the presence of two bromodomains, namely Bromodomain 1 (BD1) and Bromodomain (BD2), that recognize acetylated N-terminal tails of histones thereby acting as readers of lysine acetylation state of chromatin. Moreover, by interacting with components of the transcriptional machinery and chromatin remodeling enzymes (Taniguchi, Y., Int J Mol Sci, 2016. 17(11)), they regulate diverse transcriptional processes involving cell cycle, organogenesis, oncogenic and inflammatory pathways. The bromodomains of the four BET family members are highly conserved, about −110 amino acids in length that fold in to bromodomain modules comprising of a left-handed bundle of four alpha helices (αZ, αA, αB, αC), linked by loop regions (ZA and BC loop) that contribute to substrate specificity (Fujisawa, T. and P. Filippakopoulos, Nat Rev Mol Cell Biol, 2017. 18(4): p. 246-262). While BRD2 and BRD4 are involved in cell cycle regulation, learning and memory and inflammation (Korb, E., et al., Nat Neurosci, 2015. 18(10): p. 1464-73, Belkina, A. C., et. al, J Immunol, 2013. 190(7): p. 3670-8, LeRoy, et. al, Mol Cell, 2008. 30(1): p. 51-60), a function for BRD3 is less clear and has been suggested to be redundant with BRD2 (Stonestrom, A. J., et al., Blood, 2015. 125(18): p. 2825-34).

“BET inhibitors” are compounds that interact with these bromodomains BD1 and BD2 to inhibit their function. Compounds that interact with these bromodomains non-selectively by binding to both BD1 and BD2 have been identified with the small molecule JQ1 being the predecessor (Filippakopoulos, P., et al., Nature, 2010. 468(7327): p. 1067-73). However, such compounds have toxicities due to non-selectivity and inhibition of all the BET family members (Shorstova, T., et. Al. Br J Cancer, 2021. 124(9): p. 1478-1490, Qi, J. and Y. Shi, Cancer Cell, 2020. 37(6): p. 764-766). To minimize these issues, compounds that selectively bind with either BD1 of all the BET family or BD2 of the BET family have been recently developed and characterized (Gilan, O., et al., Science, 2020. 368(6489): p. 387-394, Faivre, E. J., et al., Nature, 2020. 578(7794): p. 306-310). However, no compound that selectively targets BRD3 is reported in the literature to the best of our knowledge. Interestingly, with our compound 12e, BRD3 was the only member to get stabilized in a statistically significant manner with a q value of 0.0007 amongst the BET family of proteins. The TPP profiles for BRD2 and BRD4 revealed insignificant changes. This observation, strikingly contrasts with the reported TPP data for other BET inhibitory compounds (JQ1, IBET-BD1, IBET-BD2, RVX-208, IBET-151) that trigger thermal stabilization of BRD2, BRD3 and BRD4 proteins by binding to their bromodomains BD1 and/or BD2. Currently, there exists no compound that selectively targets BRD3, nor is an effect of selective BRD3 inhibition or BRD3 depletion through gene knock-out in vivo in animal models known.

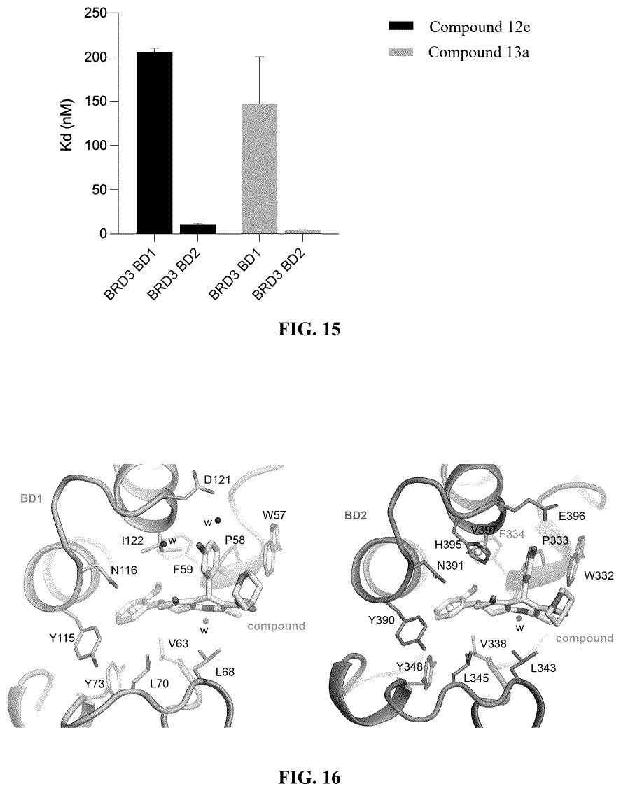

Following the initial data from TPP, to further confirm the interaction of compound with BRD3, an in-vitro assay system named bromoscan (Eurofins) was employed to analyse the affinity of the (4S,7R)-configurated compound 12e against the two bromodomains of BRD3. This revealed compound 12e to preferentially bind to BD2 (Kd˜10 nM), with far weaker binding to BD1 (Kd˜200 nM), see FIG. 15. In this assay, the compound 13a (Short Oxanyl D2E1, also named as SOD2E1) had a lower Kd value of 4 nM for BD2 and 150 nM for BD1 showing a higher potency together with a -40-fold selectivity towards BD2 (FIG. 15).

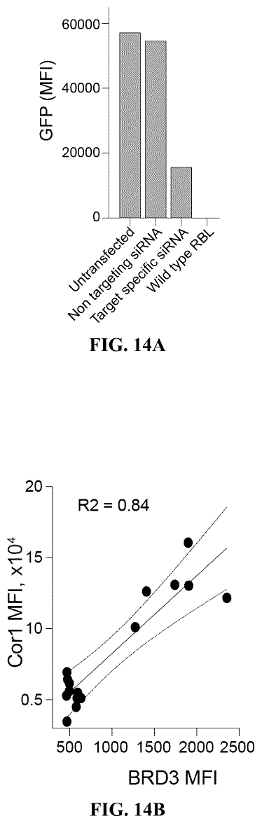

To assess the role of BRD3 in regulating coronin 1 expression, RBL cells expressing GFP under the control of the coronin 1 promoter were treated with siRNA targeting brd3 that resulted in significant downregulation of coronin 1 promoter driven GFP (FIG. 14A). Together these data suggest that the compounds bind to BRD3 thereby repressing coronin 1 transcription.

To understand the mode of interaction of the compound with the bromodomains of BRD3, we co-crystalized compound 13a with its bromodomains BD1 and BD2 and obtained crystal diffraction data. The electron density map of BRD3 BD1 (FIG. 16, left panel) and BRD3 BD2 (FIG. 16, right panel) bound with compound 13a was resolved at a resolution of 1.4 A and 2 A respectively. The amino acids in the hydrophobic pocket of ZA loop that interacts with compound 13a was identified, thereby confirming the molecular mechanism of binding of the compound with the two bromodomains of BRD3.

These data confirm the molecular target of the compound to be BRD3, through which the expression of coronin 1 and immune responses are regulated in immune cells. As we have demonstrated the immunosuppressive potential of coronin 1 promoter inhibitors in the context of autoimmune-inflammatory diseases and alloimmune responses, the information provided here reveals the possibility of targeting BRD3 to control of unwanted immune responses in the context of auto- and allo-antigen mediated disorders in addition to BRD3-driven diseases.

### Materials and Methods

### Thermal Proteome Profiling:

Thermal proteome profiling was performed as described (Savitski, M. M., et al., Science, 2014. 346(6205): p. 1255784). In brief, RBL cells (50 millions/condition) were incubated with the vehicle DMSO or the compound 12e at 6, 3 or 0 μM concentration for a period of 1 h at the end of which, the cells were washed in ice cold PBS, counted and subjected to thermal denaturation (37/41/44/47/50/53/56/59/63/67° C.) for 3 minutes, lysed by freeze thawing in liquid nitrogen and supernatant containing the soluble proteins separated by centrifugation at 100,000 g for 30 minutes. Equal volumes of the supernatant were taken, subjected to tryptic digestion and labelling with 10 plex-TMT (Thermo Fisher) and the labelled peptides analyzed and quantitated using mass spectrometry (FIG. 13).

### BRD3 Bromoscan Analysis:

T7 phage strains displaying bromodomains were grown in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 2 μM non-biotinylated affinity ligand) and incubated at room temperature with shaking for 30 minutes. The bromodomain concentration in the eluates was measured by qPCR. The bromoscan analysis was done at Eurofins (FIG. 15).

BRD3 siRNA Analysis:

Accel siRNAs against brd3 (target specific siRNA) and a control siRNA (non-targeting siRNA) were purchased from Dharmacon Horizon Discovery and 100 mM stocks prepared in the supplied siRNA buffer. In parallel, 5000 cells (WT RBL or GFP RBL cells) were seeded per well with 200 mL of Accell media in a 48 well plate. siRNA was added from the prepared stock into appropriately labelled wells at a final concentration of 1 μM. The plate was incubated at 37° C. for 72h with 5% CO₂ and at the end, washed with FACS buffer (PBS with 2% Fetal calf serum and 10 mM EDTA) and further incubated for 20 minutes on ice with Live-dead marker (Thermofisher) in FACS buffer. The cells were washed once again in FACS buffer and acquired using a flow cytometer (BD Fortessa) and the GFP fluorescence analysed using the program FlowJo (TreeStar) and the suppression of coronin 1 promoter activity assessed as a measure of GFP reduction (FIG. 14A).

### BRD3 Gene Knock Our RBL Cells Using CRISPR/Cas9 Approach:

The plasmid containing two gRNA targeting rat Brd3 gene for generating brd3 knockouts in RBL cells was generated at VectorBuilder (Neu-Isenburg, Germany) using as gRNA #1 (TGGGATGCCAAGCCTTCCCG) (SEQ ID NO.: 11) and gRNA #6771 (AGGGCTTCGCTGCCGATATC) (SEQ ID NO.: 12) followed by a protospacer adjacent motif (PAM) from Streptococcus pyogenes targeting exon 2 and exon 7, respectively. RBL cells (2.5×10⁵) were transfected with 0.5 μg of plasmid by electroporation at 1200V, 20 ms and 2 pulses in 10 μL Neon™ Transfection System pipette using the Neon™ Transfection System (Invitrogen) according to the manufacturer guidelines.

Transfected puromycin resistant cells were selected and single cells clones expanded in 96-well tissue culture plate and screened for BRD3 and coronin 1 expression by flow cytometric analysis of the median fluorescence intensity (MFI) from intracellularly-stained cells (FIG. 14B).

### Protein Expression

The His6-BRD3BD1 and His6-BRD3BD2 plasmids were a gift from Nicola Burgess-Brown (Addgene plasmids #38940 and #38941). Plasmids were extracted from the delivered transformed Machi cells following the ZR Plasmid Miniprep kit protocol (Zymo Research) and used to transform chemically competent Rosetta2(DE3) cells (Novagen). Rosetta2(DE3) colonies were grown at 37° C. on agar plates prepared with lysogeny broth (LB; 10 g tryptone, 5 g yeast extract, 10 g NaCl) supplemented with 50 μg/ml Kanamycin and 30 μg/ml Chloramphenicol (LB-Kan-Cm). For protein expression, adequate amounts of LB-Kan-Cm media were inoculated with 1% pre-culture of transformed cells and incubated at 37° C. Grown cultures were induced with 0.25 mM isopropyl 1-thio-o-D-galactopyranoside (IPTG) at an OD600 of 0.6-0.7. The incubation temperature was reduced to 28° C. for overnight expression. Cells were harvested by centrifugation at 8,000 RCF for 10 minutes at 4° C.

### Protein Purification

Purification was entirely done at 4° C. Cell pellets were homogenized in lysis buffer containing immobilized metal affinity chromatography (IMAC) loading buffer (50 mM HEPES, pH 7.5, 500 mM NaCl, 10 mM Imidazole, 5% Glycerol, 0.5 mM TCEP) complemented with 1 mM PMSF, 100 μg/ml Lysozyme, 0.1% Triton X-100 and DNase. Mechanical lysis was performed with a microfluidizer device set at 10,000 psi. The lysate was centrifuged at 14,000 RCF for 1 h, to remove cell debris and suspended particles. The clear supernatant was applied on a 5 mL Ni-NTA column (Cytiva) pre-equilibrated with IMAC loading buffer. Bound proteins were eluted with a linear gradient of IMAC elution buffer (50 mM HEPES, pH 7.5, 500 mM NaCl, 500 mM Imidazole, 5% Glycerol, 0.5 mM TCEP) using an AKTA Pure system (Cytiva). Fractions containing the desired protein were pooled and mixed with 40 μg/ml TEV protease. The mix was dialyzed overnight using SnakeSkin dialysis tubing with a 3,500 Da molecular weight cut-off (Thermo Scientific) in dialysis buffer (50 mM HEPES, pH 7.5, 250 mM NaCl, 5% Glycerol, 0.5 mM TCEP). The mix was loaded on a gravity flow column packed with 2.5 mL Ni Sepharose resin (Cytiva) pre-equilibrated with IMAC loading buffer. Cleaved protein was collected from the flow-through and concentrated to a volume of 3 mL or less using Amicon Ultra-15 centrifugal filters with a 3,000 Da molecular weight cut-off. The concentrated sample was loaded on a HiLoad 16/600 Superdex 200 μg gel filtration column (Cytiva) pre-equilibrated with SEC buffer (10 mM HEPES, pH 7.5, 200 mM NaCl, 5% Glycerol). Fractions containing the desired protein were pooled and stored at −80° C.

### Protein Crystallization

BRD3BD1 and BRD3BD2 solubilized in SEC buffer (see above) were co-crystallized with SOD2E1 using the sitting-drop vapor diffusion method. Sets of 3-drop MRC plates were prepared with a Gryphon robot (Art Robbins Instruments). BRD3BD1 was crystallized at 20° C. at an initial concentration of 11 mg/ml in presence of compound 13a dissolved in PEG 400 (1:1.5 molar ratio) in mother liquor consisting of 0.1 μM TRIS, pH 8.5 and 8% w/v PEG 8,000 (Crystal Screen HT C₁₂, Hampton Research). BRD3BD2 was crystallized at 20° C. at an initial concentration of 17 mg/ml in presence of compound 13a dissolved in PEG 400 (1:1.5 molar ratio) in a mother liquor consisting of 0.1 μM MES/imidazole, pH 6.5, 0.02 μM of each amino acid, 10% w/v PEG 20,000 and 20% v/v PEG MME 550 (Morpheus HT-96 H1, Molecular Dimensions). Crystals were fished after 9.5 days of growth and flash-frozen in LN₂.

### X-Ray Data Collection and Structure Determination

X-ray diffraction data were collected at the Swiss Light Source (SLS; Paul Scherrer Institut, Villigen, Switzerland). Data were indexed, integrated, scaled and merged using XDS and the CCP4i2 suite. The crystal structures of BRD3_{BD1} and BRD3_{BD2} were solved by molecular replacement with Phaser, using pre-existing structures (PDB codes 3S91 and 3S92) as first search models. For both crystal structures, phases and models were further improved by multiple cycles of refinement using REFMAC5 and by manual modeling using Coot. The model and the restraints dictionary for compound 13a were built with eLBOW. Figures were prepared with PyMOL version 2.4.2 (Schrödinger, LLC) (FIG. 16).

1. A compound of formula (I) 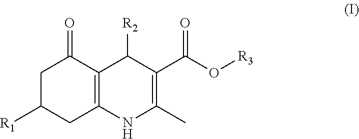
or a pharmaceutically acceptable salt, stereoisomer, diastereoisomer, enantiomer, polymorph, racemic mixture, solvate or isomers and mixtures thereof,
wherein
R₁ is selected from phenyl and thienyl, wherein said phenyl is optionally substituted with one or more optional substituents independently selected from —OH, —NO₂, halogen and —O—C₁-C₆-alkyl;
R₂ is selected from phenyl and thienyl, wherein said phenyl is optionally substituted with one or more optional substituents independently selected from —OH, —NO₂, -halogen and —O—C₁-C₆-alkyl;
R₃ is selected from —C₁-C₈-alkyl, —(C₁-C₆-alkylene)-S—C₁-C₆-alkyl, —(C₁-C₆-alkylene)-O—C₁-C₆-alkyl, —(C₂-C₄-alkylene-O)ₘ—(C₁-C₆-alkyl) wherein m is an integer from 1 to 10 (preferably from 1 to 5, more preferably from 2 to 3, even more preferably 2), —C₁-C₆-alkylene-cycloalkyl, cycloalkyl, —C₁-C₆-alkylene-(oxygen-containing saturated heterocyclyl) and oxygen-containing saturated heterocyclyl, wherein said cycloalkyl, the cycloalkyl in the —C₁-C₆-alkylene-cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

2. The compound of claim 1, wherein
R₁ is selected from phenyl and thienyl, wherein said phenyl is optionally substituted with one or more —O—C₁-C₆-alkyl;
R₂ is selected from phenyl and thienyl, wherein said phenyl is optionally substituted with one or more optional substituents independently selected from —OH, —NO₂, and -halogen; and
R₃ is selected from —C₁-C₆-alkyl, —(C₁-C₆-alkylene)-S—C₁-C₆-alkyl, —(C₁-C₆-alkylene)-O—C₁-C₆-alkyl, —C₃-C₆-cycloalkyl, —C₁-C₆-alkylene-(oxygen-containing saturated heterocyclyl) and oxygen-containing saturated heterocyclyl, wherein said cycloalkyl, the oxygen-containing saturated heterocyclyl moiety in said —C₁-C₆-alkylene-(oxygen-containing saturated heterocyclyl) and said oxygen-containing saturated heterocyclyl are each optionally substituted with one or more C₁-C₆-alkyl.

3. The compound of claim 1 or 2, wherein R₁ is phenyl optionally substituted with methoxy.

4. The compound of any one of claims 1 to 3, wherein R₁ is selected from 2-methoxyphenyl and phenyl.

5. The compound of any one of claims 1 to 4, wherein R₂ is phenyl optionally substituted with one or more optional substituents independently selected from —OH, —NO₂, and -halogen.

6. The compound of any one of claims 1 to 5, wherein R₂ is 3-hydroxyphenyl.

7. The compound of any one of claims 1 to 6, wherein R₃ is selected from —C₁-C₆-alkylene-(oxygen-containing saturated heterocyclyl) or oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

8. The compound of any one of claims 1 to 7, wherein R₃ is selected from —C₁-C₆-alkylene-tetrahydro-2-furanyl, —C₁-C₆-alkylene-tetrahydro-2H-pyran-4-yl, tetrahydrofuran-3-yl tetrahydro-2H-pyran-4-yl, oxepan-4-yl and 8-oxabicyclo[3.2.1]octan-3-yl wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl, the tetrahydrofuran-3-yl, tetrahydro-2H-pyran-4-yl, oxepan-4-yl and 8-oxabicyclo[3.2.1]octan-3-yl are each optionally substituted with one or more —C₁-C₆-alkyl, preferably selected from —C₁-C₆-alkylene-tetrahydro-2-furanyl, C₁-C₆-alkylene-tetrahydro-2H-pyran-4-yl and tetrahydro-2H-pyran-4-yl, wherein the tetrahydro-2-furanyl moiety in said —C₁-C₆-alkylene-tetrahydro-2-furanyl, the tetrahydro-2H-pyran-4-yl moiety in said —C₁-C₆-alkylene-tetrahydro-2H-pyran-4-yl and said tetrahydro-2H-pyran-4-yl are each optionally substituted with one or more —C₁-C₆-alkyl.

9. The compound of any one of claims 1 to 7, wherein R₃ is selected from (tetrahydrofuran-2-yl)methyl, tetrahydrofuran-3-yl and tetrahydro-2H-pyran-4-yl, preferably wherein R₃ is tetrahydro-2H-pyran-4-yl.

10. The compound of claim 1, selected from the group consisting of:
tetrahydro-2-furanylmethyl-7-(2-methoxyphenyl)-2-methyl-5-oxo-4-(2-thienyl)-1,4,5,6,7,8-hexahydro-3-quinolinecarboxylate (1);
methyl-4-(4-fluorophenyl)-7-(2-methoxyphenyl)-2-methyl-5-oxo-1,4,5,6,7,8-hexahydro-3-quinolinecarboxylate (2);
2-(ethylthio)ethyl-7-(2-methoxyphenyl)-2-methyl-5-oxo-4-(3-thienyl)-1,4,5,6,7,8-hexahydro-3-quinolinecarboxylate (3);
methyl 4-(3-hydroxyphenyl)-2-methyl-5-oxo-7-phenyl-1,4,5,6,7,8-hexahydroquinoline-3-carboxylate (4);
methyl-4-(3-hydroxyphenyl)-2-methyl-5-oxo-7-(2-thienyl)-1,4,5,6,7,8-hexahydro-3-quinolinecarboxylate (5);
tetrahydro-2-furanylmethyl-2-methyl-4-(3-nitrophenyl)-5-oxo-7-(2-thienyl)-1,4,5,6,7,8-hexahydro-3-quinolinecarboxylate (6);
tetrahydro-2-furanylmethyl-2-methyl-5-oxo-7-(2-thienyl)-4-(3-thienyl)-1,4,5,6,7,8-hexahydro-3-quinolinecarboxylate (7);
methyl-4-(3-hydroxyphenyl)-7-(4-methoxyphenyl)-2-methyl-5-oxo-1,4,5,6,7,8-hexahydro-3-quinolinecarboxylate (8);
methyl-7-(2-methoxyphenyl)-2-methyl-5-oxo-4-(2-thienyl)-1,4,5,6,7,8-hexahydro-3-quinolinecarboxylate (9);
tetrahydro-2-furanylmethyl-4-(2-fluorophenyl)-7-(2-methoxyphenyl)-2-methyl-5-oxo-1,4,5,6,7,8-hexahydro-3-quinolinecarboxylate (10);
methyl-4-(3-hydroxyphenyl)-7-(2-methoxyphenyl)-2-methyl-5-oxo-1,4,5,6,7,8-hexahydro-3-quinolinecarboxylate (11);
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
methyl 7-(2-methoxyphenyl)-2-methyl-5-oxo-4-phenyl-1,4,5,6,7,8-hexahydroquinoline-3-carboxylate (14);
methyl 7-(2-methoxyphenyl)-4-(3-methoxyphenyl)-2-methyl-5-oxo-1,4,5,6,7,8-hexahydroquinoline-3-carboxylate (15);
methyl 4-(2-hydroxyphenyl)-7-(2-methoxyphenyl)-2-methyl-5-oxo-1,4,5,6,7,8-hexahydroquinoline-3-carboxylate (16);
4-methoxybutyl 4-(3-hydroxyphenyl)-7-(2-methoxyphenyl)-2-methyl-5-oxo-1,4,5,6,7,8-hexahydroquinoline-3-carboxylate (17);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
oxetan-3-yl 4-(3-hydroxyphenyl)-7-(2-methoxyphenyl)-2-methyl-5-oxo-1,4,5,6,7,8-hexahydroquinoline-3-carboxylate (20);
tert-butyl 4-(3-hydroxyphenyl)-7-(2-methoxyphenyl)-2-methyl-5-oxo-1,4,5,6,7,8-hexahydroquinoline-3-carboxylate (21);
methyl 7-(4-chlorophenyl)-4-(3-hydroxyphenyl)-2-methyl-5-oxo-1,4,5,6,7,8-hexahydroquinoline-3-carboxylate (22);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
2,2,6,6-tetramethyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (52);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53);
oxepan-4-yl 4-(3-hydroxyphenyl)-7-(2-methoxyphenyl)-2-methyl-5-oxo-1,4,5,6,7,8-hexahydroquinoline-3-carboxylate (54);
hexahydrofuro[2,3-b]furan-3-yl 4-(3-hydroxyphenyl)-7-(2-methoxyphenyl)-2-methyl-5-oxo-1,4,5,6,7,8-hexahydroquinoline-3-carboxylate (55);
cyclopentyl 4-(3-hydroxyphenyl)-7-(2-methoxyphenyl)-2-methyl-5-oxo-1,4,5,6,7,8-hexahydroquinoline-3-carboxylate (56);
cyclohexyl 4-(3-hydroxyphenyl)-7-(2-methoxyphenyl)-2-methyl-5-oxo-1,4,5,6,7,8-hexahydroquinoline-3-carboxylate (57);
ethyl 4-(3-hydroxyphenyl)-7-(2-methoxyphenyl)-2-methyl-5-oxo-1,4,5,6,7,8-hexahydroquinoline-3-carboxylate (58);
butyl 4-(3-hydroxyphenyl)-7-(2-methoxyphenyl)-2-methyl-5-oxo-1,4,5,6,7,8-hexahydroquinoline-3-carboxylate (59);
neopentyl 4-(3-hydroxyphenyl)-7-(2-methoxyphenyl)-2-methyl-5-oxo-1,4,5,6,7,8-hexahydroquinoline-3-carboxylate (60);
2-ethylbutyl 4-(3-hydroxyphenyl)-7-(2-methoxyphenyl)-2-methyl-5-oxo-1,4,5,6,7,8-hexahydroquinoline-3-carboxylate (61);
2,2-dimethylbutyl 4-(3-hydroxyphenyl)-7-(2-methoxyphenyl)-2-methyl-5-oxo-1,4,5,6,7,8-hexahydroquinoline-3-carboxylate (62);
4,4-dimethylpentyl 4-(3-hydroxyphenyl)-7-(2-methoxyphenyl)-2-methyl-5-oxo-1,4,5,6,7,8-hexahydroquinoline-3-carboxylate (63);
2-(2-ethoxyethoxy)ethyl 4-(3-hydroxyphenyl)-7-(2-methoxyphenyl)-2-methyl-5-oxo-1,4,5,6,7,8-hexahydroquinoline-3-carboxylate (64);
2-(2-(2-(hexyloxy)ethoxy)ethoxy)ethyl 4-(3-hydroxyphenyl)-7-(2-methoxyphenyl)-2-methyl-5-oxo-1,4,5,6,7,8-hexahydroquinoline-3-carboxylate (65);
tetrahydro-2H-pyran-4-yl 4-(4-fluoro-3-hydroxyphenyl)-7-(2-methoxyphenyl)-2-methyl-5-oxo-1,4,5,6,7,8-hexahydroquinoline-3-carboxylate (66); and
tetrahydro-2H-pyran-4-yl 4-(2,4-difluoro-3-hydroxyphenyl)-7-(2-methoxyphenyl)-2-methyl-5-oxo-1,4,5,6,7,8-hexahydroquinoline-3-carboxylate (67).

11. The compound of claim 1, selected from the group consisting of:
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13);
(tetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (18);
(2,2,6,6-tetramethyltetrahydro-2H-pyran-4-yl)methyl 4-(3-hydroxyphenyl)-7-(2-methoxyphenyl)-2-methyl-5-oxo-1,4,5,6,7,8-hexahydroquinoline-3-carboxylate (19);
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50);
4-methyltetrahydro-2H-pyran-4-yl 4-(3-hydroxyphenyl)-7-(2-methoxyphenyl)-2-methyl-5-oxo-1,4,5,6,7,8-hexahydroquinoline-3-carboxylate (51);
8-oxabicyclo[3.2.1]octan-3-yl 4-(3-hydroxyphenyl)-7-(2-methoxyphenyl)-2-methyl-5-oxo-1,4,5,6,7,8-hexahydroquinoline-3-carboxylate (53) and
oxepan-4-yl 4-(3-hydroxyphenyl)-7-(2-methoxyphenyl)-2-methyl-5-oxo-1,4,5,6,7,8-hexahydroquinoline-3-carboxylate (54).

12. The compound of claim 1, selected from the group consisting of:
tetrahydro-2-furanylmethyl-4-(3-hydroxyphenyl)-7-(2-methoxyphenyl)-2-methyl-5-oxo-1,4,5,6,7,8-hexahydro-3-quinolinecarboxylate (12);
tetrahydro-2H-pyran-4-yl-4-(3-hydroxyphenyl)-7-(2-methoxyphenyl)-2-methyl-5-oxo-1,4,5,6,7,8-hexahydro-3-quinolinecarboxylate (13); and
tetrahydrofuran-3-yl 4-(3-hydroxyphenyl)-7-(2-methoxyphenyl)-2-methyl-5-oxo-1,4,5,6,7,8-hexahydroquinoline-3-carboxylate (50).

13. The compound of any one of claims 1 to 12, wherein the compound of formula (I) has an absolute configuration of its stereogenic centers as shown in the formula: 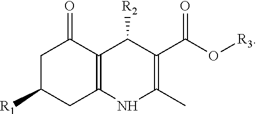
wherein R₁, R₂ and R₃ are as defined in any one of claims 1 to 12.

14. The compound of claim 1, wherein:
the compound has an absolute configuration of its stereogenic centers as shown in the formula: 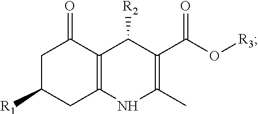
R₁ is selected from phenyl and thienyl, wherein said phenyl is optionally substituted with one or more optional substituents independently selected from —OH, —NO₂, -halogen and —O—C₁-C₆-alkyl;
R₂ is selected from phenyl and thienyl, wherein said phenyl is optionally substituted with one or more optional substituents independently selected from —OH, —NO₂, -halogen and —O—C₁-C₆-alkyl;
and
R₃ is —C₁-C₆-alkylene-(oxygen-containing saturated heterocyclyl) or oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

15. The compound of claim 1, wherein:
the compound has an absolute configuration of its stereogenic centers as shown in the formula: 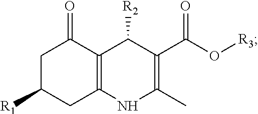
R₁ is selected from phenyl and thienyl, wherein said phenyl is optionally substituted with one or more —O—C₁-C₆-alkyl;
R₂ is selected from phenyl and thienyl, wherein said phenyl is optionally substituted with one or more optional substituents independently selected from —OH, —NO₂, and -halogen; and
R₃ is —C₁-C₆-alkylene-(oxygen-containing saturated heterocyclyl) or oxygen-containing saturated heterocyclyl, wherein the oxygen-containing saturated heterocyclyl moiety in said —C₁-C₆-alkylene-(oxygen-containing saturated heterocyclyl) and said oxygen-containing saturated heterocyclyl are each optionally substituted with one or more —C₁-C₆-alkyl.

16. The compound of claim 1, wherein:
the compound has an absolute configuration of its stereogenic centers as shown in the formula: 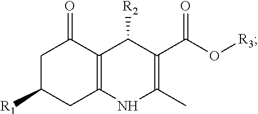
R₁ is selected from 2-methoxyphenyl, and phenyl;
R₂ is 3-hydroxyphenyl; and
R₃ is selected from —C₁-C₈-alkyl, —(C₁-C₆-alkylene)-S—C₁-C₆-alkyl, —(C₁-C₆-alkylene)-O—C₁-C₆-alkyl, and —(C₂-C₄-alkylene-O)ₘ—(C₁-C₆-alkyl) wherein m is an integer from 1 to 10, preferably from 1 to 5, more preferably from 2 to 3, even more preferably 2.

17. The compound of claim 16, wherein the compound is selected from:
methyl (4S, 7R)-4-(3-hydroxyphenyl)-7-(2-methoxyphenyl)-2-methyl-5-oxo-1,4,5,6,7,8-hexahydro-3-quinolinecarboxylate (tic);
4-methoxybutyl (4S, 7R)-4-(3-hydroxyphenyl)-7-(2-methoxyphenyl)-2-methyl-5-oxo-1,4,5,6,7,8-hexahydroquinoline-3-carboxylate (17a);
tert-butyl (4S, 7R)-4-(3-hydroxyphenyl)-7-(2-methoxyphenyl)-2-methyl-5-oxo-1,4,5,6,7,8-hexahydroquinoline-3-carboxylate (21a); and
2-(2-ethoxyethoxy)ethyl (4S, 7R)-4-(3-hydroxyphenyl)-7-(2-methoxyphenyl)-2-methyl-5-oxo-1,4,5,6,7,8-hexahydroquinoline-3-carboxylate (64a).

18. A pharmaceutical composition comprising a compound of any one of claims 1 to 17 or a pharmaceutically acceptable salt, stereoisomer, diastereoisomer, enantiomer, polymorph, racemic mixture, solvate or isomers and mixtures thereof and a pharmaceutically acceptable carrier.

19. The compound of any one of claims 1 to 17 or a pharmaceutically acceptable salt, stereoisomer, diastereoisomer, enantiomer, polymorph, racemic mixture, solvate or isomers and mixtures thereof, or the pharmaceutical composition of claim 18 for use as a medicament.

20. The compound of any one of claims 1 to 17 or a pharmaceutically acceptable salt, stereoisomer, diastereoisomer, enantiomer, polymorph, racemic mixture, solvate or isomers and mixtures thereof, or the pharmaceutical composition of claim 18 for use in the induction of immunosuppression or in the treatment and/or prevention of a disease or disorder selected from the group consisting of transplant rejection, autoimmune diseases, inflammatory diseases, infectious diseases, and lymphoproliferative disorders.

21. The compound for use or the pharmaceutical composition for use of claim 20,
wherein said autoimmune diseases is selected from the group consisting of psoriasis, vitiligo, primary sclerosing cholangitis, multiple sclerosis, systemic lupus erythematosus, Hashimoto's thyroiditis, rheumatoid arthritis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis;
wherein said transplant rejection is selected from the group consisting of acute or chronic rejection of cells, tissue, organ, allografts and xenografts, poor graft functional states, graft versus host disease; rejection of cardiac transplant, skin transplant, renal transplant, liver transplant, islet transplant, pancreas transplant, lung transplant, bowel transplant, corneal transplant, vascular transplant, adrenal transplant, hair transplant, bone transplant, cartilage transplant and ligamental transplant;
wherein said inflammatory disease is selected from the group consisting of inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, prurigo nodularis, eosinophilic esophagitis, hidradenitis suppurative, fibrotic disorders, cardio vasculopathy, allergic disorders, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome;
wherein said lymphoproliferative disorder is T cell lymphoma or T cell leukaemia;
wherein said infectious disease is selected from the group consisting of tuberculosis, preferably caused by mycobacteria, Salmonella sp. infections, Helicobacter sp. infections, retroviral infections, preferably HIV or HTLV, cytomegalo viral infection, candida infection, Staphylococcus infections, lympho-choriomeningitis viral infections and viral hepatitis.

22. The compound for use or the pharmaceutical composition for use of claim 20 or 21, wherein a disease or disorder is selected from the group consisting of infectious diseases, and lymphoproliferative disorders,
preferably wherein said lymphoproliferative disorder is T cell lymphoma or T cell leukaemia; and
preferably wherein said infectious disease is selected from the group consisting of tuberculosis, preferably caused by mycobacteria, Salmonella sp. infections, Helicobacter sp. infections, retroviral infections, preferably HIV or HTLV, cytomegalo viral infection, candida infection, Staphylococcus infections, lympho-choriomeningitis viral infections and viral hepatitis.

23. The compound for use or the pharmaceutical composition for use of any one of claims 20 to 22, wherein the compound of formula (I) inhibits coronin 1 expression.

24. A BRD3-selective bromodomain inhibitor for use in the treatment or prevention of a disease that can benefit from BRD3 inhibition, either directly or indirectly via modulation of coronin 1 promotor activity or via reduced expression of Coronin 1.

25. The BRD3-selective bromodomain inhibitor for use of claim 24, wherein the disease that can benefit from BRD3 inhibition is amenable for therapeutic intervention through direct inhibition of BRD3 or via modulation of coronin 1 promotor activity or via modulation of coronin 1 expression through BRD3 inhibition,

26. The BRD3-selective bromodomain inhibitor for use of claim 24 or 25, wherein the disease that can benefit from BRD3 inhibition is selected from transplant rejection, autoimmune diseases, inflammatory diseases, infectious diseases, and lymphoproliferative disorders.

27. The BRD3-selective bromodomain inhibitor for use of claim 26,
wherein said transplant rejection is selected from the group consisting of acute or chronic rejection of cells, tissue, organ, allografts and xenografts, poor graft functional states, graft versus host disease; rejection of cardiac transplant, skin transplant, renal transplant, liver transplant, islet transplant, pancreas transplant, lung transplant, bowel transplant, corneal transplant, vascular transplant, adrenal transplant, hair transplant, bone transplant, cartilage transplant and ligamental transplant;
wherein said autoimmune disease is selected from the group consisting of psoriasis, vitiligo, primary sclerosing cholangitis, multiple sclerosis, systemic lupus erythematosus, Hashimoto's thyroiditis, rheumatoid arthritis, myasthenia gravis, diabetes type I or II, disorders secondary to diabetes type I or II, vasculitis, pernicious anaemia, Sjogren syndrome, uveitis, Graves' ophthalmopathy, alopecia areata, allergic asthma, atopic dermatitis, allergic rhinitis, allergic conjunctivitis, myocarditis, hepatitis, and allergic contact dermatitis;
wherein said inflammatory disease is selected from the group consisting of inflammatory bowel disease, Crohn's disease, ulcerative colitis, intrinsic asthma, inflammatory lung injury, inflammatory liver injury, inflammatory glomerular injury, atherosclerosis, osteoarthritis, myositis, polymyositis, prurigo nodularis, eosinophilic esophagitis, hidradenitis suppurative, fibrotic disorders, cardio vasculopathy, allergic disorders, irritant contact dermatitis, eczematous dermatitis, seborrhoeic dermatitis, cutaneous manifestations of immunologically-mediated disorders, inflammatory eye diseases, keratoconjunctivitis, myocardial infarction, stroke, gut ischemia, renal failure, hemorrhage shock, traumatic shock, toxic shock, septic shock and adult respiratory distress syndrome;
wherein said infectious disease is selected from the group consisting of tuberculosis, preferably caused by mycobacteria, Salmonella sp. infections, Helicobacter sp. infections, retroviral infections, preferably HIV or HTLV, cytomegalo viral infection, candida infection, Staphylococcus infections, lympho-choriomeningitis viral infections and viral hepatitis;
wherein said lymphoproliferative disorder is T cell lymphoma or T cell leukaemia.

28. The BRD3-selective bromodomain inhibitor for use of claim 24 or 25, wherein the disease that can benefit from BRD3 inhibition is a BRD3-driven malignancy like NMC, OCCC, colorectal carcinoma, or rhabdomyosarcoma, or their metastasis.

29. The BRD3-selective bromodomain inhibitor for use of any one of claims 24 to 28, wherein the BRD3-selective bromodomain inhibitor is the compound of any one of claims 1 to 16 or a pharmaceutically acceptable salt, stereoisomer, diastereoisomer, enantiomer, polymorph, racemic mixture, solvate or isomers and mixtures thereof.

30. A vector comprising a coronin 1 (coro1a) promoter element, wherein in a vertebrate genome, said coronin 1 promoter element starts directly upstream from a transcription starting site (TSS) of a coronin 1 gene and spans a sequence stretch of at least about 700 bp in said genome.

31. The vector of claim 30, further comprising a coronin 1 promoter reporter gene, wherein said coronin 1 promoter element is operably linked to said coronin 1 promoter reporter gene.

32. The vector of claim 30 or 31, wherein said coronin 1 promoter element spans a sequence of at least about 700 bp to about 1500 bp in said genome, preferably said coronin 1 promoter element spans a sequence stretch of at least about 700 bp in said genome.

33. The vector of any one of claims 30 to 32, wherein said coronin 1 promoter element has an identity of at least 40%, preferably of at least 50%, more preferably of at least 60%, again more preferably of at least 70%, again more preferably of at least 80%, again more preferably of at least 90%, again more preferably of at least 95%, again more preferably of at least 98%, again more preferably of at least 99% with a sequence of SEQ ID NO: 1-6.

34. A cell comprising the vector of any one of claims 30 to 33.

35. A method for identifying compounds that modulate coronin 1 promoter activity comprising the steps of:
a. providing a host cell comprising said vector of any one of the claims 30 to 33, wherein said host cell is capable of expressing said promoter reporter genes of said vector;
b. subjecting said host cells to a compound to be tested; and
c. measuring expression of said coronin 1 promoter reporter gene in said host cell subjected to said compound to be tested.

36. A method of preparing the compound of formula (I) as defined in claim 13, the method comprising the step (b) of asymmetric reduction of the pyridine motive via enantioselective partial transfer hydrogenation.

37. The method of claim 35, comprising the step (b) of asymmetric reduction of the pyridine motive in compound XI: 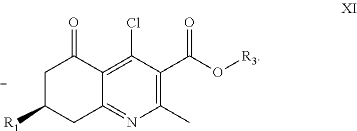
via enantioselective partial transfer hydrogenation, wherein R₁, R₂ and R₃ in compound XI are as defined in claim 1 for compound of formula (I).
